(19) United States
(12) Patent Application Publication
(10) Pub. No.: US 2024/0285701 A1
(43) Pub. Date: Aug. 29, 2024

(54) COMPOSITIONS AND METHODS FOR TREATING SKIN CONDITIONS

(71) Applicant: THE TRUSTEES OF COLUMBIA UNIVERSITY IN THE CITY OF NEW YORK, New York, NY (US)

(72) Inventors: Virginia Cornish, New York, NY (US); Amirhossein JAFARIYAN, New York, NY (US)

(73) Assignee: THE TRUSTEES OF COLUMBIA UNIVERSITY IN THE CITY OF NEW YORK, New York, NY (US)

(21) Appl. No.: 18/635,627

(22) Filed: Apr. 15, 2024

Related U.S. Application Data
(63) Continuation of application No. PCT/US2022/046921, filed on Oct. 17, 2022.
(60) Provisional application No. 63/320,522, filed on Mar. 16, 2022.
(60) Provisional application No. 63/256,484, filed on Oct. 15, 2021.

(51) Int. Cl. A61K 36/064; A61K 9/00; A61K 9/06; A61K 38/18; A61K 38/19; A61K 38/20; A61K 38/39; A61K 38/55; A61K 45/06; A61K 47/36; A61P 17/00; A61P 17/02; A61P 17/06

(52) U.S. Cl. CPC A61K 36/064; A61K 9/0014; A61K 9/06; A61K 38/1808; A61K 38/1841; A61K 38/1866; A61K 38/195; A61K 38/2026; A61K 38/39; A61K 38/55; A61K 45/06; A61K 47/36; A61P 17/00; A61P 17/02; A61P 17/06

(57) ABSTRACT

The disclosed subject matter provides for genetically modified cells, e.g., fungal cells, that autonomously generates and/or secretes one or more skin therapeutics, and methods of use thereof. In certain embodiments, the present disclosure provides genetically-engineered fungal cells that generate and secrete growth factors, protease inhibitors, cytokines and/or chemokines and methods of use thereof, e.g., for treating skin conditions.

### CROSS-REFERENCE TO RELATED APPLICATIONS

This application is a continuation of International Patent Application No. PCT/US2022/046921, filed Oct. 17, 2022, which claims priority to U.S. Provisional Application No. 63/256,484, filed Oct. 15, 2021, and U.S. Provisional Application No. 63/320,522, filed Mar. 16, 2022, the contents of each of which are incorporated herein by reference herein in their entirety.

### SEQUENCE LISTING

The instant application contains a Sequence Listing which has been submitted electronically in XML format and is hereby incorporated by reference in its entirety. Said XML copy, created on Apr. 15, 2024, is named 070050_6839.xml and is 69,502 bytes in size.

### TECHNICAL FIELD

The present disclosure relates to genetically-engineered fungal cells expressing a skin therapeutic for the treatment of skin conditions.

### BACKGROUND

The healing of topical wounds is a multi-step process involving migration, differentiation and proliferation of different cell types. These processes are controlled via an orchestrated action of various signaling molecules and wound-healing agents. The healing process of chronic wounds can be more challenging depending on the size and depth of the wound and the wound's microenvironment. In diabetic patients, lack of or lower levels of active wound healing agents such as growth factors and cytokines and a higher level of proteases in the wound site impedes the normal wound healing process and leads to an unhealed chronic and acute ulcer. It has been shown that topical application of wound healing agents can help reconstruct a healthy microenvironment and enhance wound healing.

Growth factors are among the most important agents in the wound healing process as they play regulatory and functional roles during the inflammation, tissue formation and tissue remodeling stages. The proteolytic microenvironment of the chronic and acute wounds, such as diabetic foot ulcers, results in the degradation of many important growth factors such as the epidermal growth factor (EGF), platelet-derived growth factor (PDGF), transforming growth factor-beta (TGF-β), and vascular endothelial growth factor (VEGF). This degradation can lead to decreased levels of these agents in the wound bed. Topical application of growth factors can supplement the decreased levels of these growth factors and improve the healing process.

Like growth factors, cytokines and chemokines also play important roles in the wound healing process due to their signaling functions. The stromal cell-derived factor 1 protein (CXCL12) is a key signaling molecule in cell recruitment and cell differentiation in the wound healing process. Moreover, it has been shown that in situ expression and delivery of CXCL12 can accelerate and enhance the healing of topical ulcers. Leptin (LEP), the hormone critical in regulating energy homeostasis, plays additional regulatory roles as a cytokine in the wound healing process, mainly in the inflammatory stage. Like CXCL12, topical application of LEP proved advantageous in accelerating the wound healing process.

However, daily topical application of purified therapeutic agents such as growth factors can lead to inconsistent or insufficient wound healing or unwanted side effects like melanoma. Therefore, there is a need in the art for more effective methods for administering skin therapeutics for treating a skin condition including a wound or a cancer.

### SUMMARY

The disclosed subject matter provides for genetically-engineered cells, e.g., genetically-engineered fungal cells, that autonomously generates and/or secretes one or more skin therapeutics. The present disclosure further provides pharmaceutical compositions including the disclosed genetically-engineered cells and methods of administering the disclosed genetically-engineered cells for treating a subject in need thereof.

In one aspect, the present disclosure provides pharmaceutical compositions of the disclosed genetically-engineered fungal cells. In certain embodiments, the pharmaceutical composition includes (i) a live fungal cell genetically engineered to express and secrete a skin therapeutic and (ii) a pharmaceutically acceptable carrier. In certain embodiments, the skin therapeutic is secreted from the fungal cell by a secretory pathway of the fungal cell.

In certain embodiments, the skin therapeutic is a protein or a functional fragment thereof. In certain embodiments, the skin therapeutic is selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof and a combination thereof.

In certain embodiments, the skin therapeutic is a growth factor or a derivative thereof or an inhibitor of a growth factor or a derivative thereof. In certain embodiments, the growth factor is selected from the group consisting of epidermal growth factor (EGF), platelet-derived growth factor (PDGF), fibroblast growth factor (FGF), vascular endothelial growth factor (VEGF), transforming growth factor-beta (TGF-β) and a combination thereof. In certain embodiments, the growth factor is EGF. In certain embodiments, the growth factor is PDGF. In certain embodiments, the growth factor is TGF-β. In certain embodiments, the growth factor is VEGF.

In certain embodiments, the skin therapeutic is a chemokine or a derivative thereof. In certain embodiments, the chemokine is CXCL12.

In certain embodiments, the skin therapeutic is a cytokine or a derivative thereof. In certain embodiments, the cytokine is selected from the group consisting of Leptin, IL-4 and a combination thereof.

In certain embodiments, the skin therapeutic is a protease inhibitor or a derivative thereof. In certain embodiments, the protease inhibitor is selected from the group consisting of TIMP1, TIMP2 and a combination thereof.

In certain embodiments, the skin therapeutic is an extracellular matrix protein or a derivative thereof. In certain embodiments, the skin therapeutic is selected from the group consisting of Type VII Collagen, elastin and a combination thereof.

In certain embodiments, the skin therapeutic is an antimicrobial and/or an anti-inflammatory peptide. In certain embodiments, the antimicrobial and/or anti-inflammatory peptide is selected from the group consisting of cathelicidin antimicrobial peptide (LL-37) or analogs thereof, RcA1b-PepI, RcA1b-PepII, RcA1b-PepII, lucifensin, lucifensin II, lucilin, pexiganan acetate (MSI-78), D2A21/D4E1, granulysin, a synthetic granulysin-derived peptide and a combination thereof.

In certain embodiments, the fungal cell is a species from a genus selected from the group consisting of Cladosporium, Aureobasidium, Aspergillus, Saccharomyces, Malassezia, Epicoccum, Candida, Penicillium, Wallemia, Pichia, Phoma, Cryptococcus, Fusarium, Clavispora, Cyberlindnera, Kluyveromyces and a combination thereof. In certain embodiments, the fungal cell is Saccharomyces cerevisiae or Pichia pastoris.

In certain embodiments, a pharmaceutical composition of the present disclosure further includes a second live fungal cell genetically engineered to express and secrete a second skin therapeutic. In certain embodiments, a pharmaceutical composition of the present disclosure further includes a third live fungal cell genetically engineered to express and secrete a third skin therapeutic. In certain embodiments, a pharmaceutical composition of the present disclosure further includes a fourth live fungal cell genetically engineered to express and secrete a fourth skin therapeutic. In certain embodiments, a pharmaceutical composition of the present disclosure further includes a fifth live fungal cell genetically engineered to express and secrete a fifth skin therapeutic. In certain embodiments, a pharmaceutical composition of the present disclosure further includes a sixth live fungal cell genetically engineered to express and secrete a sixth skin therapeutic. In certain embodiments, the pharmaceutical can further include a seventh, eighth, ninth and/or tenth live fungal cell that is genetically engineered to express and secrete a skin therapeutic, where each fungal cell expresses and secretes a different skin therapeutic.

In certain embodiments, the pharmaceutical composition is formulated for rectal administration, vaginal administration or topical administration. In certain embodiments, the pharmaceutical composition is formulated for topical administration.

In certain embodiments, the pharmaceutically acceptable carrier comprises a hydrogel. In certain embodiments, the hydrogel comprises from about 0.1% w/v to about 5.0% w/v of a polysaccharide. In certain embodiments, the hydrogel comprises from about 0.1% w/v to about 1.0% w/v of a polysaccharide. In certain embodiments, the polysaccharide is agarose.

In certain embodiments, the pharmaceutical composition includes a therapeutically effect amount of the live genetically-engineered fungal cell. In certain embodiments, the therapeutically effective amount is from about 1×10³ cells/ml to about 1×10¹⁰ cells/ml of the live genetically-engineered fungal cells. In certain embodiments, the therapeutically effective amount is from about 1×10⁴ cells/ml to about 1×10¹⁰ cells/ml of the live genetically-engineered fungal cells. In certain embodiments, the therapeutically effective amount is from about 1×10³ cells/ml to about 1×10⁹ cells/ml of the live genetically-engineered fungal cells. In certain embodiments, the therapeutically effective amount is from about 1×10⁴ cells/ml to about 1×10⁸ cells/ml of the live genetically-engineered fungal cells. In certain embodiments, the therapeutically effective amount is from about 1×10⁶ cells/ml to about 2×10⁷ cells/ml of the live genetically-engineered fungal cells. In certain embodiments, the therapeutically effective amount of live genetically-engineered fungal cells includes an amount of live genetically-engineered fungal cells that express and secrete from about 1 pg/ml and about 200,000 pg/ml of the skin therapeutic in about 24 hours or less. In certain embodiments, the therapeutically effective amount of live genetically-engineered fungal cells includes an amount of live genetically-engineered fungal cells that express and secrete from about 100 pg/ml and about 25,000 pg/ml of the skin therapeutic in about 24 hours or less. In certain embodiments, the therapeutically effective amount of live genetically-engineered fungal cells includes an amount of live genetically-engineered fungal cells that express and secrete from about 1 pg/ml and about 2,500 pg/ml of the skin therapeutic in about 24 hours or less. In certain embodiments, the therapeutically effective amount of live genetically-engineered fungal cells includes an amount of live genetically-engineered fungal cells that express and secrete from about 100 pg/ml to about 2,500 pg/ml of the skin therapeutic in about 24 hours or less.

In certain embodiments, the live genetically-engineered fungal cell secretes and expresses the skin therapeutic for about 24 hours to about 2 weeks, e.g., from about 120 hours, after administration. In certain embodiments, the live genetically-engineered fungal cell secretes and expresses the skin therapeutic for at least about 48 hours after administration. In certain embodiments, the live genetically-engineered fungal cell secretes and expresses the skin therapeutic for at least about 72 hours after administration. In certain embodiments, the live genetically-engineered fungal cell secretes and expresses the skin therapeutic for at least about 96 hours after administration. In certain embodiments, the live genetically-engineered fungal cell secretes and expresses the skin therapeutic for at least about 108 hours, at least about 120 hours, at least about 132 hours, at least about 144 hours, at least about 156 hours, at least about 165 hours, at least about 8 days, at least about 9 days, at least about 10 days, at least about 11 days, at least about 12 days, at least about 13 days or at least about 14 days after administration.

In certain embodiments, the live genetically-engineered fungal cell continuously secretes and expresses the skin therapeutic.

In certain embodiments, the pharmaceutical composition further includes one or more nutrients for the one or more genetically engineered fungal cells.

The present disclosure further provides a topical pharmaceutical composition. In certain embodiments, the topical pharmaceutical composition includes a hydrogel comprising a live fungal cell genetically engineered to express and secrete a skin therapeutic. In certain embodiments, the hydrogel comprises from about 0.1% w/v to about 10.0% w/v of a polysaccharide. In certain embodiments, the hydrogel comprises from about 0.1% w/v to about 5.0% w/v of a polysaccharide. In certain embodiments, the hydrogel comprises from about 0.1% w/v to about 1.0% w/v of a polysaccharide. In certain embodiments, the polysaccharide is agarose.

In certain embodiments, the topical pharmaceutical composition includes a bottom layer facing the skin and a top layer facing the air, wherein the hydrogel comprising the fungal cell genetically engineered to express and secrete the skin therapeutic is disposed between the bottom layer and the top layer. In certain embodiments, the genetically-engineered fungal cell cannot pass through the bottom layer, and wherein the skin therapeutic can pass through the bottom layer. In certain embodiments, the bottom layer has a pore size of about 2 μm, e.g., about 2 μm or less.

In certain embodiments, the topical pharmaceutical composition comprises a therapeutically effective amount of the live genetically-engineered fungal cell. In certain embodiments, the therapeutically effective amount is from about 1×10³ cells/ml to about 1×10¹⁰ cells/ml of the live genetically-engineered fungal cells. In certain embodiments, the therapeutically effective amount is from about 1×10⁴ cells/ml to about 1×10¹⁰ cells/ml of the live genetically-engineered fungal cells. In certain embodiments, the therapeutically effective amount is from about 1×10³ cells/ml to about 1×10⁹ cells/ml of the live genetically-engineered fungal cells. In certain embodiments, the therapeutically effective amount of live genetically-engineered fungal cells comprises from about 1×10⁴ cells/ml to about 1×10⁸ cells/ml of the live genetically-engineered fungal cells. In certain embodiments, the therapeutically effective amount of live genetically-engineered fungal cells includes an amount of live genetically-engineered fungal cells that express and secrete from about 1 pg/ml and about 200,000 pg/ml of the skin therapeutic in about 24 hours or less. In certain embodiments, the therapeutically effective amount of live genetically-engineered fungal cells includes an amount of live genetically-engineered fungal cells that express and secrete from about 100 pg/ml and about 25,000 pg/ml of the skin therapeutic in about 24 hours or less. In certain embodiments, the therapeutically effective amount of live genetically-engineered fungal cells comprises an amount of live genetically-engineered fungal cells that express and secrete from about 1 pg/ml and about 2,500 pg/ml of the skin therapeutic in about 24 hours or less.

In certain embodiments, the genetically-engineered fungal cell secretes the skin therapeutic for at least about 12 hours, at least about 24 hours, at least about 48 hours, at least about 60 hours, at least about 72 hours, at least about 84 hours, at least about 96 hours, at least about 108 hours, at least about 120 hours, at least about 132 hours, at least about 144 hours, at least about 156 hours, at least about 165 hours, at least about 8 days, at least about 9 days, at least about 10 days, at least about 11 days, at least about 12 days, at least about 13 days or at least about 14 days after administration to the subject.

In certain embodiments, the fungal cell is Saccharomyces cerevisiae or Pichia pastoris.

The present disclosure further provides a pharmaceutical composition that includes (i) a first live fungal cell genetically engineered to express and secrete a first skin therapeutic, (ii) a second live fungal cell genetically engineered to express and secrete a second skin therapeutic and (iii) a pharmaceutically acceptable carrier. In certain embodiments, the first skin therapeutic and the second skin therapeutic are different. In certain embodiments, the first skin therapeutic and the second skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof. In certain embodiments, the first skin therapeutic comprises a growth factor and the second skin therapeutic comprises a chemokine. In certain embodiments, the first skin therapeutic comprises a growth factor and the second skin therapeutic comprises a cytokine.

The present disclosure further provides a pharmaceutical composition that includes (i) a first live fungal cell genetically engineered to express and secrete a first skin therapeutic, (ii) a second live fungal cell genetically engineered to express and secrete a second skin therapeutic, (iii) a third live fungal cell genetically engineered to express and secrete a third skin therapeutic and (iv) a pharmaceutically acceptable carrier. In certain embodiments, the first skin therapeutic, the second skin therapeutic and the third skin therapeutic are different. In certain embodiments, the first skin therapeutic, the second skin therapeutic and the third skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

The present disclosure further provides a pharmaceutical composition that includes (i) a first live fungal cell genetically engineered to express and secrete a first skin therapeutic, (ii) a second live fungal cell genetically engineered to express and secrete a second skin therapeutic, (iii) a third live fungal cell genetically engineered to express and secrete a third skin therapeutic, (iv) a fourth live fungal cell genetically engineered to express and secrete a fourth skin therapeutic and (v) a pharmaceutically acceptable carrier. In certain embodiments, the first skin therapeutic, the second skin therapeutic, the third skin therapeutic and the fourth skin therapeutic are different. In certain embodiments, the first skin therapeutic, the second skin therapeutic, the third skin therapeutic and the fourth skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

The present disclosure further provides a pharmaceutical composition that includes (i) a first live fungal cell genetically engineered to express and secrete a first skin therapeutic, (ii) a second live fungal cell genetically engineered to express and secrete a second skin therapeutic, (iii) a third live fungal cell genetically engineered to express and secrete a third skin therapeutic, (iv) a fourth live fungal cell genetically engineered to express and secrete a fourth skin therapeutic, (v) a fifth live fungal cell genetically engineered to express and secrete a fifth skin therapeutic and (vi) a pharmaceutically acceptable carrier. In certain embodiments, the first skin therapeutic, the second skin therapeutic, the third skin therapeutic, the fourth skin therapeutic and the fifth skin therapeutic are different. In certain embodiments, the first skin therapeutic, the second skin therapeutic, the third skin therapeutic, the fourth skin therapeutic and the fifth skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

The present disclosure further provides a pharmaceutical composition that includes (i) a first live fungal cell genetically engineered to express and secrete a first skin therapeutic, (ii) a second live fungal cell genetically engineered to express and secrete a second skin therapeutic, (iii) a third live fungal cell genetically engineered to express and secrete a third skin therapeutic, (iv) a fourth live fungal cell genetically engineered to express and secrete a fourth skin therapeutic, (v) a fifth live fungal cell genetically engineered to express and secrete a fifth skin therapeutic, (vi) a sixth live fungal cell genetically engineered to express and secrete a sixth skin therapeutic and (vii) a pharmaceutically acceptable carrier. In certain embodiments, the first skin therapeutic, the second skin therapeutic, the third skin therapeutic, the fourth skin therapeutic, the fifth skin therapeutic and the sixth skin therapeutic are different. In certain embodiments, the first skin therapeutic, the second skin therapeutic, the third skin therapeutic, the fourth skin therapeutic, the fifth skin therapeutic and the sixth skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

The present disclosure further provides methods for treating a subject in need thereof. In certain embodiments, the method includes administering to the subject a pharmaceutical composition disclosed herein. In certain embodiments, the pharmaceutical composition is formulated for topical administration.

In certain embodiments, the pharmaceutical composition is administered to the subject to treat a skin condition or to perform a cosmetic procedure.

In certain embodiments, the cosmetic procedure is skin rejuvenation.

In certain embodiments, the skin condition is selected from the group consisting of a wound, an infection, acne, a fibrotic disorder, a blistering disorder, an inflammatory condition, a vascular lesion, a skin cancer, xeroderma pigmentosum, a pigment disorder and a combination thereof. In certain embodiments, the skin condition is a wound. In certain embodiments, the wound is a diabetic ulcer. In certain embodiments, the skin condition is an infection. In certain embodiments, the skin condition is acne. In certain embodiments, the skin condition is a fibrotic disorder. In certain embodiments, the fibrotic disorder is scleroderma. In certain embodiments, the skin condition is a blistering disorder. In certain embodiments, the blistering disorder is epidermolysis bullosa. In certain embodiments, the skin condition is an inflammatory condition. In certain embodiments, the inflammatory condition is psoriasis. In certain embodiments, the skin condition is a vascular lesion. In certain embodiments, the skin condition is a skin cancer. In certain embodiments, the skin condition is xeroderma pigmentosum. In certain embodiments, the skin condition is a pigment disorder.

In certain embodiments, the genetically engineered fungal cell administered by a method of the present disclosure secretes the skin therapeutic for at least about 12 hours, at least about 24 hours, at least about 48 hours, at least about 60 hours, at least about 72 hours, at least about 84 hours, at least about 96 hours, at least about 108 hours, at least about 120 hours, at least about 132 hours, at least about 144 hours, at least about 156 hours, at least about 165 hours, at least about 8 days, at least about 9 days, at least about 10 days, at least about 11 days, at least about 12 days, at least about 13 days or at least about 14 days after administration to the subject. In certain embodiments, the genetically engineered fungal cell secretes the skin therapeutic for at least about 72 hours after administration to the subject. In certain embodiments, the genetically engineered fungal cell secretes the skin therapeutic for at least about 7 days after administration to the subject. In certain embodiments, the genetically engineered fungal cell secretes the skin therapeutic for at least about 2 weeks after administration to the subject. In certain embodiments, administration of the pharmaceutical composition to the subject comprises applying the pharmaceutical composition to the affected area.

In certain embodiments, the pharmaceutical composition is applied no more than 5 times a week, no more than 4 times a week, no more than 3 times a week, no more than 2 times a week, no more than 1 time a week. In certain embodiments, the pharmaceutical composition is applied no more than 3 times a week. In certain embodiments, the pharmaceutical composition is applied no more than 2 times a week. In certain embodiments, the pharmaceutical composition is applied no more than once per day.

The present disclosure further provides a use of the pharmaceutical compositions disclosed herein for treating a skin condition or for performing a cosmetic procedure. In certain embodiments, the cosmetic procedure is skin rejuvenation. In certain embodiments, the skin condition is selected from the group consisting of a wound, an infection, acne, a fibrotic disorder, a blistering disorder, an inflammatory condition, a vascular lesion, a skin cancer, xeroderma pigmentosum, a pigment disorder and a combination thereof. In certain embodiments, the skin condition is a wound.

The present disclosure further provides a kit that includes a pharmaceutical composition disclosed herein.

The present disclosure further provides a kit for performing a method disclosed herein.

### BRIEF DESCRIPTION OF THE DRAWINGS

FIG. 1 illustrates an exemplary topical composition of the present disclosure and application thereof.

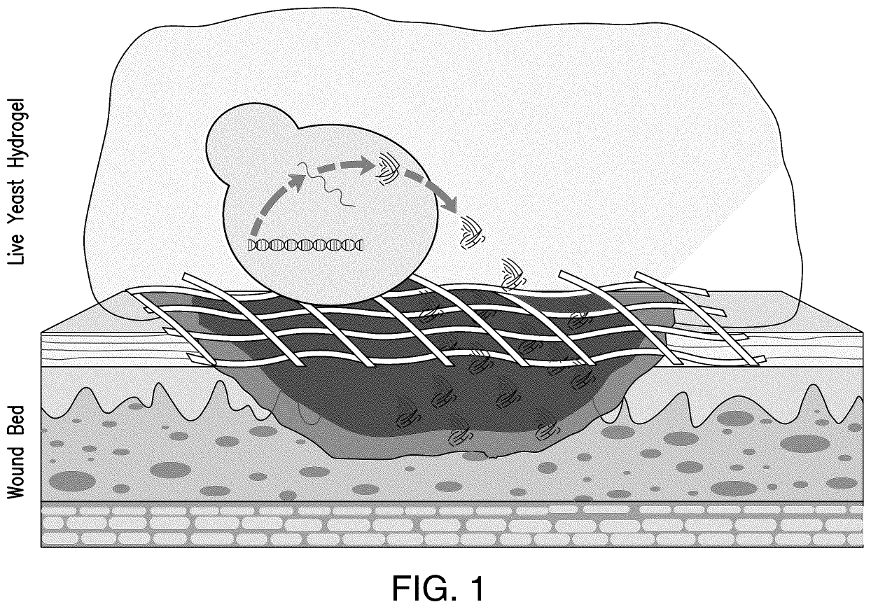

FIG. 2 illustrates an exemplary topical hydrogel composition of the present disclosure and application thereof.

FIG. 3 provides the results of quantitative Western Blots to measure the concentration of hEGF and hLEP secreted from genetically-engineered yeast. Smt3 was used as the positive control. The calibration curve was then used to figure out the concentration of the test protein in the test wells (here, FLAG-hEGF and FLAG-hLEP).

FIG. 4 provides the results of in vitro wound healing scratch assays on human skin fibroblast cells. Sup 07 is the supernatant from yAJ07, Sup 06 is the supernatant from yAJ06, Sup 34 is the supernatant from yAJ34.

FIG. 5A provides the results of in vitro wound healing scratch assays on human skin fibroblast cells, where genetically-engineered yeast secreting EGF (yAJ06) were co-cultured with the fibroblast cells.

FIG. 5B shows the results of an EGF-secreting live yeast (yAJ06) hydrogel dressing on cell migration and cell proliferation using a scratch wound-healing assay by co-culturing human skin fibroblast cells with the hydrogel dressing using a Boyden chamber setup.

FIG. 6 shows that the secreted proteins can diffuse freely in the hydrogel.

FIGS. 7A-7I provide the analysis of hydrogel compositions comprising genetically-engineered yeast. FIGS. 7A-7D show that the secreted skin therapeutics can diffuse through the 0.2 μm PTFE membrane. FIG. 7A is the legend, FIG. 7B is a schematic of the experiment, FIG. 7C is the system that is incubated for 24 hours and FIG. 7D is the blot developed using anti-FLAG antibodies. FIGS. 7E-7H show that the yeast cells and yeast spores cannot pass through the PTFE membrane. FIG. 7E is the schematic of the hydrogel composition, FIG. 7F is the system incubated for 24 hours, FIG. 7G is the removal of the yeast agar and the PTFE and FIG. 7H is the image of the plate after 48 hours of further incubation. FIG. 7I shows that yeast-secreted LEP and CXCL12 can diffuse through the hydrogel and the PTFE membrane.

FIGS. 8A-8D provide representative mouse images of the excisional wounds (FIG. 8A), the topical application of hydrogel (yeast hydrogel or the control hydrogel) on top of the PTFE membrane (FIG. 8B) and the TEGADERM™ dressing covering the hydrogel dressing (FIG. 8C). FIG. 8D provides a representation of a three-layer hydrogel dressing that includes (i) a polyurethane (PUR) membrane (TEGADERM™ film) on the top, acting as a physical barrier and adhering to the dressing above the wound, (ii) an agar hydrogel containing live yeast and nutrients needed for the yeast growth and (iii) a polytetrafluoroethylene (PTFE) membrane with pore size=0.2 μm as the barrier between the live yeast hydrogel and the wound bed.

FIGS. 9A-9B provide a time-lapse study of the in vivo wound healing assay on the Streptozotocin (STZ)-administered B6 diabetic mice. Wounds were treated daily with either EGF-secreting live yeast hydrogel dressing (containing yAJ28 for mEGF secretion) or the control hydrogel dressing (not containing yeast strains). FIG. 9A shows representative images for wounds treated with different conditions over time. FIG. 9B shows the % wound closure over time (* denotes a p-value less than 0.05. p-values where measured using a student t-test). n=4 for each test group.

FIG. 10 provides H&E staining from representative sections of a wound that was allowed to heal for 8 days and were either treated with the engineered live yeast secreting hEGF (top) or with control agar (bottom). Both sections are from the same mouse.

FIG. 11A provides the results of an ELISA assay for determining the titers of mCXCL12 secreted from yeast genetically engineered to express and secrete mCXCL12 (yAJ36). yAJ36 secretes mCXCL12 using the MF-α secretion signal peptide.

FIG. 11B provides the results of an ELISA assay for determining the titers of hCXCL12 secreted from yeast genetically engineered to express and secrete hCXCL12 (yAJ35). yAJ35 secretes hCXCL12 using the MF-α secretion signal peptide.

FIG. 12A provides the results of an ELISA assay for determining the titers of mEGF secreted from yeast genetically engineered to express and secrete mEGF (yAJ28). yAJ28 secretes mEGF using the MF-α secretion signal peptide.

FIG. 12B provides the results of an ELISA assay for determining the titers of hEGF secreted from yeast genetically engineered to express and secrete hEGF (yAJ39). yAJ39 secretes hEGF using the MF-α secretion signal peptide.

FIG. 13 provides the optical density of four (4) genetically engineered yeast strains during a 48-hour period. yAJ28, yAJ35, yAJ36 and yAJ39 secrete mEGF, hCXCL12, mCXCL12 and hEGF, respectively, using the MF-α secretion signal peptide.

FIG. 14 provides the range of wound sizes and the number of different sized wounds generated by using a 5 mm biopsy punch.

FIG. 15A provides the results of an in vivo wound healing assay on STZ-administered mice having 24 mm² wounds (n=3) treated with an mEGF secreting yeast hydrogel dressing, compared with 24 mm² wounds (n=3) treated with purified recombinant mEGF (100 ng/ml) dissolved in the hydrogel dressing, and 24 mm² wounds (n=3) treated with the control hydrogel dressing.

FIG. 15B provides the results of an in vivo wound healing assay on STZ-administered mice having a 24 mm² wounds (n=3) treated with an mCXCL12 secreting yeast hydrogel dressing, compared with 24 mm² wounds (n=3) treated with purified recombinant mCXCL12 (100 ng/ml) dissolved in the hydrogel dressing, and 24 mm² wounds (n=3) treated with the control hydrogel dressing.

FIG. 16A provides a comparison of the results of an in vivo wound healing assay on STZ-administered mice having a 24 mm² wound treated with a mCXCL12 secreting yeast hydrogel dressing or an mEGF secreting yeast hydrogel dressing. n=3.

FIG. 16B provides a comparison of the results of an in vivo wound healing assay on STZ-administered mice having a 24 mm² wound treated with recombinant mCXCL12 (100 ng/ml) dissolved in the hydrogel dressing, or recombinant mEGF (100 ng/ml) dissolved in the hydrogel dressing. n=3.

FIG. 17A provides a comparison of the results of an in vivo wound healing assay on STZ-administered mice having a 28 mm² wound treated with a mCXCL12 secreting yeast hydrogel dressing or an mEGF secreting yeast hydrogel dressing. n=2.

FIG. 17B provides a comparison of the results of an in vivo wound healing assay on STZ-administered B6 diabetic mice having a 28 mm² wound treated with recombinant mEGF (100 ng/ml) dissolved in the hydrogel dressing. n=1.

FIG. 17C provides a comparison of the results of an in vivo wound healing assay on STZ-administered mice having a 24 mm² wound treated with a control yeast hydrogel dressing or a control hydrogel dressing. n=2.

FIG. 18 provides a comparison of the results of an in vivo wound healing assay on STZ-administered mice having a 28 mm² wound treated with a hydrogel having a community of mCXCL12 secreting yeast and mEGF secreting yeast (“yeast community”), a combination of recombinant mCXCL12 (100 ng/ml) and mEGF (100 ng/ml) dissolved in the hydrogel dressing or a control hydrogel dressing. n=2.

FIG. 19A provides the results of an in vivo wound healing assay on STZ-administered mice having a 40 mm² wound treated with an mEGF secreting yeast hydrogel dressing. n=4.

FIG. 19B provides the results of an in vivo wound healing assay on STZ-administered mice having a 40 mm² wound treated with a mCXCL12 secreting yeast hydrogel. n=2.

FIG. 19C provides a comparison of the results of an in vivo wound healing assay on STZ-administered mice having a 40 mm² wound treated with a hydrogel dressing having a community of mCXCL12 secreting yeast and mEGF secreting yeast (“yeast community”), or a control hydrogel dressing. n=3.

FIG. 19D provides a comparison of the results of an in vivo wound healing assay on STZ-administered mice having a 40 mm² wound treated with a hydrogel dressing having a community of mCXCL12 secreting yeast and mEGF secreting yeast (“yeast community”), a mCXCL12 secreting yeast hydrogel dressing, an mEGF secreting yeast hydrogel dressing or a control hydrogel dressing. n=3.

FIG. 20A provides the results of an in vivo wound healing assay on STZ-administered mice having a 60 mm² wound treated with an mEGF secreting yeast hydrogel dressing. n=4.

FIG. 20B provides the results of an in vivo wound healing assay on STZ-administered mice having a 60 mm² wound treated with a mCXCL12 secreting yeast hydrogel dressing. n=4.

FIG. 20C provides the results of an in vivo wound healing assay on STZ-administered mice having a 60 mm² wound treated with an mCherry expressing yeast hydrogel dressing. n=4.

FIG. 21A provides the timeline of an in vivo wound healing assay on STZ-administered mice having a 60 mm² wound treated with a mCXCL12 secreting yeast hydrogel. n=4.

FIG. 21B provides the timeline of an in vivo wound healing assay on STZ-administered mice having a 60 mm² wound treated with an mEGF secreting yeast hydrogel. n=4.

FIG. 22 shows that the genetically engineered yeast does not migrate from the hydrogel to the blood stream of the treated mice. Genetically engineered yeast were modified to express RFP and then applied to mice in the same manner as the wound healing yeast. Blood from these mice was collected and fluorescence was measured to determine if any yeast entered circulation of the mice.

FIG. 23 provides the body weight and blood glucose level of SKH-1 mice treated with STZ five consecutive days. Mice were wounded 16 days post STZ treatment using a biopsy punch.

FIG. 24 provides the results of an in vivo wound healing assay on STZ-administered SKH-1 mice having a 50 mm² excisional wound treated EGF secreting yeast (yAJ28) hydrogel. n=4. The top panel shows the % wound closure over time (* denotes a p-value less than 0.05, ** denotes a p-value less than 0.01. p-values where measured using a student t-test). The bottom panel shows representative images for wounds treated with different conditions over time.

FIG. 25 provides the results of an in vivo wound healing assay on STZ-administered SKH-1 mice having a 50 mm² excisional wound treated CXCL12 secreting yeast (yAJ36) hydrogel. n=4. The top panel shows the % wound closure over time (* denotes a p-value less than 0.05, ** denotes a p-value less than 0.01. p-values where measured using a student t-test). n=4 for each test group. The bottom panel shows representative images for wounds treated with different conditions over time.

FIG. 26A shows the effect of initial wound size on in vivo wound healing rate of 24 mm², 28 mm² and 40 mm² wounds being treating with a hydrogel including mEGF secreting yeast (yAJ28).

FIG. 26B shows the effect of initial wound size on in vivo wound healing rate of 24 mm², 28 mm² and 40 mm² wounds being treating with a hydrogel including CXCL12 secreting yeast (yAJ36).

FIG. 26C shows the effect of initial wound size on in vivo wound healing rate of 24 mm², 28 mm² and 40 mm² wounds being treating with a control hydrogel.

FIG. 26D shows the effect of initial wound size on in vivo wound healing rate of 24 mm², 28 mm² and 40 mm² wounds being treating with a hydrogel having a community of mCXCL12 secreting yeast and mEGF secreting yeast (“yeast community”).

FIG. 27A provides the timeline of an in vivo wound healing assay on STZ-administered mice having a splint-assisted 50 mm² excisional wound treated with an mEGF secreting yeast (yAJ28) hydrogel. n=8.

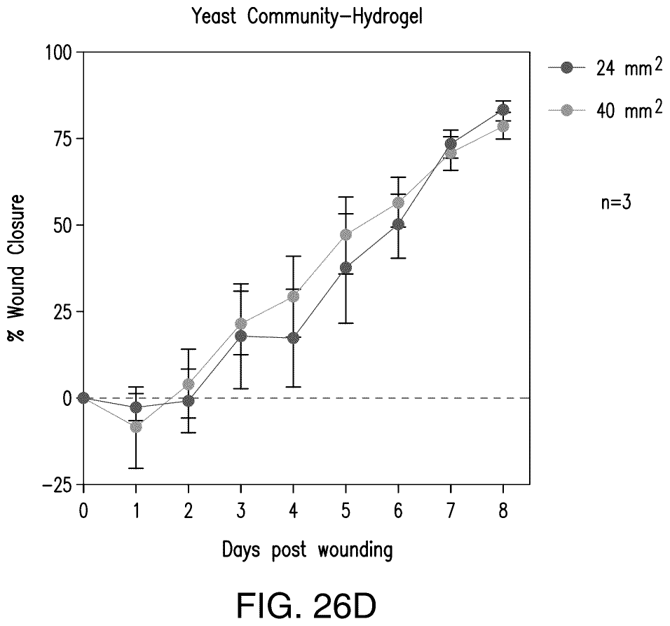

FIG. 27B provides the timeline of an in vivo wound healing assay on STZ-administered mice having a splint-assisted 50 mm² excisional wound treated with a mLEP secreting yeast (yAJ30) hydrogel. n=8.

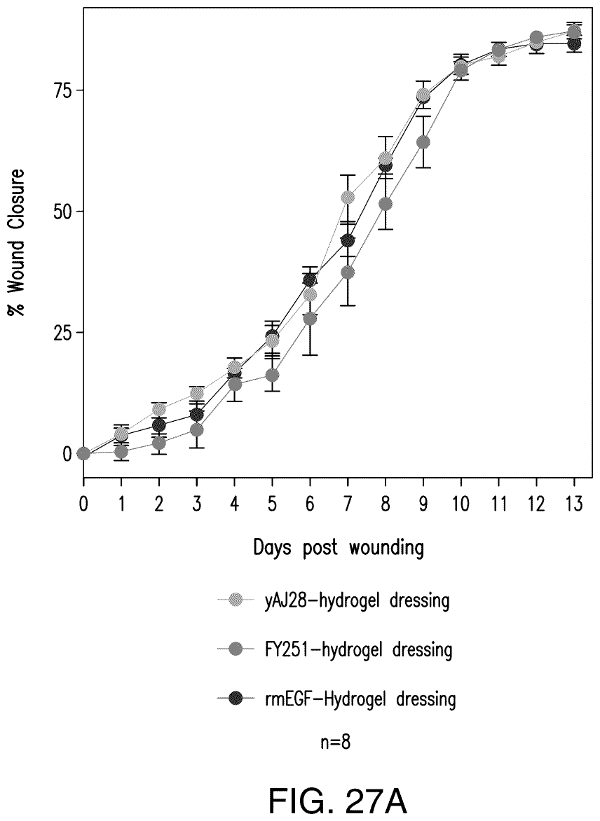

FIG. 28A provides the Western Blot of LEP-secreting yeast strains. yAJ33 and yAJ34 respectively secrete FLAG-mLEP and FLAG-hLEP using the MF-α secretion signal peptide. SMT3 denotes the 6×His-SMT3-FLAG protein that was used as the positive control. The placement of the black squares underneath the blot shows if the sample in each well was from a cell pellet (not secreted proteins) or culture supernatant (secreted proteins).

FIG. 28B provides the Western Blot of EGF-secreting yeast strains. yAJ26 secretes FLAG-hEGF using the MF-α secretion signal peptide and yAJ40 secretes FLAG-mEGF using the Sed1 secretion signal peptide. The placement of the black squares underneath the blot shows if the sample in each well was from a cell pellet (not secreted proteins) or culture supernatant (secreted proteins).

FIG. 28C provides the Western Blot of CXCL12-secreting yeast strains. yAJ35 and yAJ36 respectively secrete hCXCL12 and mCXCL12 using the MF-α secretion signal peptide. yAJ37 and yAJ38 respectively secrete FLAG-hCXCL12 and FLAG-mCXCL12 using the MF-α secretion signal peptide. The placement of the black squares underneath the blot shows if the sample in each well was from a cell pellet (not secreted proteins) or culture supernatant (secreted proteins).

FIG. 29 provides the results of an ELISA assay for determining the titers of hLEP secreted from yeast genetically engineered to express and secrete hLEP. yAJ27 secretes hLEP using the MF-α secretion signal peptide.

FIG. 30A provides the results of in vitro wound healing scratch assays on human skin fibroblast cells, where genetically-engineered yeast secreting hLEP (yAJ27) were co-cultured with the fibroblast cells.

FIG. 30B provides the results of in vitro wound healing scratch assays on human skin fibroblast cells, where genetically-engineered yeast secreting hCXCL12 (yAJ35) were co-cultured with the fibroblast cells.

FIG. 31A provides the results of in vitro proliferation assays on human skin fibroblast cells, where genetically-engineered yeast secreting hEGF (yAJ39) were co-cultured with the fibroblast cells.

FIG. 31B provides the results of in vitro proliferation assays on human skin fibroblast cells, where genetically-engineered yeast secreting hLEP (yAJ27) were co-cultured with the fibroblast cells.

FIG. 31C provides the results of in vitro proliferation assays on human skin fibroblast cells, where genetically-engineered yeast secreting hCXCL12 (yAJ35) were co-cultured with the fibroblast cells.

FIG. 32A provides the results of an in vivo wound healing assay on STZ-administered mice having a wound treated with a mLEP secreting yeast (yAJ30) hydrogel dressing. n=8. The mice were SKH-1 mice with splint-assisted excisional wounds.

FIG. 32B provides the results of an in vivo wound healing assay on STZ-administered mice having a wound treated with an mEGF secreting yeast (yAJ28) hydrogel dressing. n=8. The mice were SKH-1 mice with splint-assisted excisional wounds.

FIG. 33 provides K5 and CD31 immunohistochemistry of healthy and wounded mouse skin treated with hydrogel wound dressings containing mLEP secreting yeast (yAJ30) or hydrogel wound dressings containing mEGF secreting yeast (yAJ28). K5 shows the basal keratinocytes in red, CD31 shows the endothelial cells in green and DAPI shows the nuclei in blue.

FIG. 34 provides K14 and Ki67 immunohistochemistry of healthy and wounded mouse skin treated with hydrogel wound dressings containing mLEP secreting yeast (yAJ30) or hydrogel wound dressings containing mEGF secreting yeast (yAJ28). K14 shows the basal keratinocytes in red, Ki67 shows the proliferative cells in green and DAPI shows the nuclei in blue.

FIG. 35 provides K14 and K10 immunohistochemistry of healthy and wounded mouse skin treated with hydrogel wound dressings containing mLEP secreting yeast (yAJ30) or hydrogel wound dressings containing mEGF secreting yeast (yAJ28). K14 shows the basal keratinocytes in red, K10 shows the suprabasal keratinocytes in green and DAPI shows the nuclei in blue.

FIG. 36 provides loricrin and phalloidin immunohistochemistry of healthy and wounded mouse skin treated with hydrogel wound dressings containing mLEP secreting yeast (yAJ30) or hydrogel wound dressings containing mEGF secreting yeast (yAJ28). Loricrin shows the granular and cornified layers in green, phalloidin shows the cytoskeleton in red and DAPI shows the nuclei in blue.

### DETAILED DESCRIPTION

The present disclosure provides genetically-engineered fungal cells that autonomously generate and/or secrete skin therapeutics. The present further provides the application of such genetically-engineered fungal cells for treating a subject in need of thereof, e.g., a subject suffering from a skin condition, e.g., a wound.

The genetically-engineered fungal cells, the pharmaceutical compositions of such fungal cells and the methods described herein provide a more cost-effective method for administering a skin therapeutic to a subject in need thereof without requiring the purification of the skin therapeutic from the genetically-engineered cell prior to administration to the subject. For example, but not by way of limitation, the generation of a skin therapeutic by a genetically-engineered cell in situ and administration of such a cell can avoid, prevent and/or reduce the degradation of the skin therapeutic that can occur during the manufacturing, purification and/or storing process.

Application of the disclosed genetically-engineered fungal cells, e.g., yeast cells, which continuously secretes skin therapeutics, also has the potential to improve the healing of a skin condition, e.g., wound, by minimizing reliance on patient compliance on regular application of a product. In certain embodiments, the sustained delivery of one or more skin therapeutics by on-site expression, secretion and delivery via the disclosed genetically-engineered fungal cells increases the bioavailability of these agents and leads to enhanced wound healing. This sustained delivery is not achievable by daily topical administration of purified agents, whereas, the genetically-engineered yeast delivers such agents continuously to the affected area. Although prolonged continuous delivery is approachable by other techniques such as applications of smart nano- and macro-sized carriers for gradual release of skin therapeutics, such techniques would suffer from higher cost and storage problems and poor shelf-life.

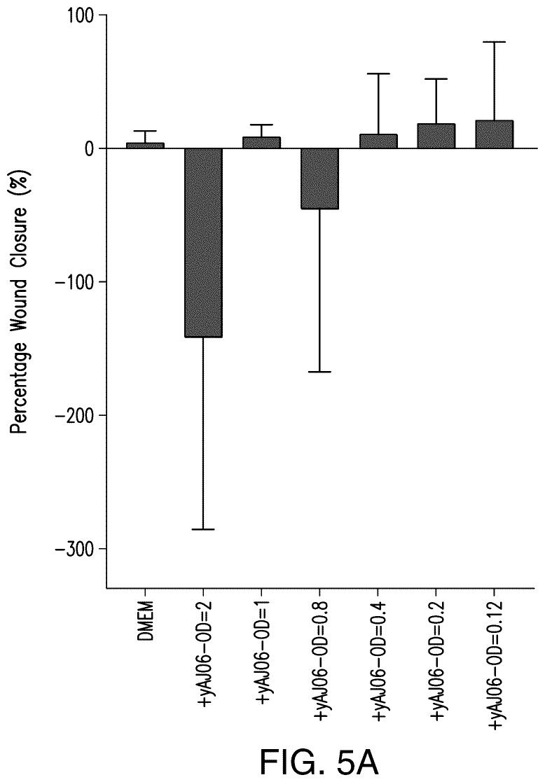

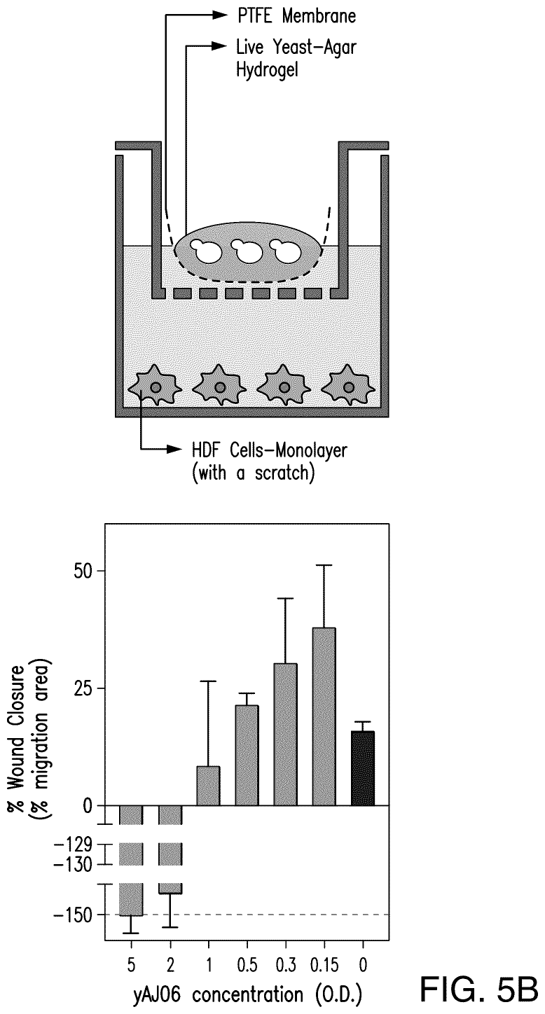

Fungal cells were used in the present disclosure as they are superior to bacterial cells for expressing therapeutic proteins. In particular, fungal cells such as yeast are superior to bacteria for expression and secretion of eukaryotic proteins because fungal cells have protein folding chaperones, disulfide-bond formation and post-translational machineries similar to those in mammalian cells. Having similar cellular machinery to mammalian cells ensures proper folding and post-translation modification of eukaryotic proteins and the generation of eukaryotic proteins that are biologically active. Bacteria do not include such machinery. As illustrated in Examples 2 and 4 and FIGS. 5B, 9A-9B and 31A-31C, the skin therapeutics expressed and secreted by the genetically-engineered fungal cells of the present disclosure are biologically active and enhance wound closure and healing. In addition, compared to bacteria, yeast can be fermented at large scale, and delivered and stored in dried form, thereby allowing increased shelf-life. Further, fungal cells such as Saccharomyces cerevisiae can be used safely by humans as they are recognized as safe (GRAS) organism unlike many bacteria.

For clarity, but not by way of limitation, the detailed description of the presently disclosed subject matter is divided into the following subsections:
I. Definitions;
II. Skin Therapeutics;
III. Genetically-Engineered Cells;
IV. Pharmaceutical Compositions;
V. Skin Conditions;
VI. Methods of Use;
VII. Kits and Products; and
VIII. Exemplary Embodiments.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “expression” or “expresses,” as used herein, refer to transcription and translation occurring within a cell, e.g., yeast cell. The level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

As used herein, “polypeptide” refers generally to peptides and proteins having about three or more amino acids. In certain embodiments, the polypeptide can be endogenous to the cell, or preferably, can be exogenous, meaning that it is heterologous, i.e., foreign, to the cell being utilized.

The term “protein” as used herein refers to a sequence of amino acids for which the chain length is sufficient to produce the higher levels of tertiary and/or quaternary structure. This is to distinguish from “peptides” that typically do not have such structure. Typically, the protein herein will have a molecular weight of at least about 4-100 kD, e.g., closer to about 15 kD. In certain embodiments, a protein can include at least about 20, about 30, about 40, about 50, about 60, about 70, about 80, about 90, about 100, about 200, about 300, about 400 or about 500 amino acids. Examples of proteins encompassed within the definition herein include all proteins, and, in general proteins that contain one or more disulfide bonds, including multi-chain polypeptides comprising one or more inter- and/or intrachain disulfide bonds. In certain embodiments, proteins can include other post-translation modifications including, but not limited to, glycosylation and lipidation. See, e.g., Prabakaran et al., WIREs Syst Biol Med (2012), which is incorporated herein by reference in its entirety.

The term “functional fragment thereof,” as used herein, refers to a fragment of a skin therapeutic, e.g., a protein or peptide, that retains at least a portion of the activity of the intact and/or full-length skin therapeutic, e.g., a protein or peptide. In certain embodiments, the functional fragment retains at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99% or at least about 100% of the activity of the intact and/or full-length skin therapeutic.

As used herein the terms “amino acid,” “amino acid monomer” or “amino acid residue” refer to organic compounds composed of amine and carboxylic acid functional groups, along with a side-chain specific to each amino acid. In particular, alpha- or α-amino acid refers to organic compounds in which the amine (—NH₂) is separated from the carboxylic acid (—COOH) by a methylene group (—CH₂), and a side-chain specific to each amino acid connected to this methylene group (—CH₂) which is alpha to the carboxylic acid (—COOH). Different amino acids have different side chains and have distinctive characteristics, such as charge, polarity, aromaticity, reduction potential, hydrophobicity and pKa. Amino acids can be covalently linked to form a polymer through peptide bonds by reactions between the carboxylic acid group of the first amino acid and the amine group of the second amino acid. Amino acid in the sense of the disclosure refers to any of the twenty plus naturally occurring amino acids, non-natural amino acids, and includes both D and L optical isomers.

The term “nucleic acid,” “nucleic acid molecule” or “polynucleotide” as used herein refers to any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby the bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of a nucleic acid of the disclosure in vitro and/or in vivo, e.g., in a yeast cell. For example, but not by way of limitation, a nucleic acid of the present disclosure can encode a skin therapeutic. Such DNA (e.g., cDNA) or RNA (e.g., mRNA) vectors can be unmodified or modified. For example, mRNA can be chemically modified to enhance the stability of the RNA vector and/or expression of the encoded molecule.

As used herein, the term “vector” refers to a nucleic acid molecule capable of transporting another nucleic acid to which it has been linked.

As used herein, the term “recombinant cell” refers to cells which have some genetic modification from the original parent cells from which they are derived. Such cells can also be referred to as “genetically-engineered cells.” Such genetic modification can be the result of an introduction of a heterologous gene (or nucleic acid) for expression of the gene product, e.g., a recombinant protein, e.g., a skin therapeutic.

As used herein, the term “recombinant protein” refers generally to peptides and proteins. Such recombinant proteins are “heterologous,” i.e., foreign to the cell being utilized, such as a heterologous skin therapeutic produced by a yeast cell.

As used herein, “sequence identity” or “identity” in the context of two polynucleotide or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

As used herein, the term “fusion protein” refers to a protein that includes all or a portion of a protein that is linked, e.g., at the N-terminus or C-terminus, to a second protein or a portion of the second protein. In certain embodiments, a portion of a protein can include a functional fragment of the protein.

As would be understood by those skilled in the art, the term “codon optimization,” as used herein, refers to the introduction of synonymous mutations into codons of a protein-coding gene in order to improve protein expression in expression systems of a particular organism, such as a cell of a species of the phylum Ascomycota, in accordance with the codon usage bias of that organism. The term “codon usage bias” refers to differences in the frequency of occurrence of synonymous codons in coding DNA. The genetic codes of different organisms are often biased towards using one of the several codons that encode a same amino acid over others-thus using the one codon with, a greater frequency than expected by chance. Optimized codons in microorganisms, such as Saccharomyces cerevisiae, reflect the composition of their respective genomic tRNA pool. The use of optimized codons can help to achieve faster translation rates and high accuracy.

In the field of bioinformatics and computational biology, many statistical methods have been discussed and used to analyze codon usage bias. Methods such as the ‘frequency of optimal codons’ (Fop), the Relative Codon Adaptation (RCA) or the ‘Codon Adaptation Index’ (CAI) are used to predict gene expression levels, while methods such as the ‘effective number of codons’ (Nc) and Shannon entropy from information theory are used to measure codon usage evenness. Multivariate statistical methods, such as correspondence analysis and principal component analysis, are widely used to analyze variations in codon usage among genes. There are many computer programs to implement the statistical analyses enumerated above, including CodonW, GCUA, INCA, and others identifiable by those skilled in the art. Several software packages are available online for codon optimization of gene sequences, including those offered by companies such as GenScript, EnCor Biotechnology, Integrated DNA Technologies, ThermoFisher Scientific, among others known those skilled in the art.

As used herein, “percentage of sequence identity” or “percentage of identity” means the value determined by comparing two optimally aligned sequences over a comparison window, wherein the portion of the polynucleotide sequence in the comparison window can include additions or deletions (gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As understood by those skilled in the art, determination of percent identity between any two sequences can be accomplished using certain well-known mathematical algorithms. Non-limiting examples of such mathematical algorithms are the algorithm of Myers and Miller, the local homology algorithm of Smith et al.; the homology alignment algorithm of Needleman and Wunsch; the search-for-similarity-method of Pearson and Lipman; the algorithm of Karlin and Altschul, modified as in Karlin and Altschul. Computer implementations of suitable mathematical algorithms can be utilized for comparison of sequences to determine sequence identity. Such implementations include, but are not limited to: CLUSTAL, ALIGN, GAP, BESTFIT, BLAST, FASTA, among others identifiable by skilled persons.

As used herein, “reference sequence” is a defined sequence used as a basis for sequence comparison. A reference sequence can be a subset or the entirety of a specified sequence; for example, as a segment of a full-length protein or protein fragment. A reference sequence can be, for example, a sequence identifiable in a database such as GenBank and UniProt and others identifiable to those skilled in the art.

The term “operative connection,” “operably linked” or “operatively linked,” as used herein, with regard to regulatory sequences of a gene indicate an arrangement of elements in a combination enabling production of an appropriate effect. With respect to genes and regulatory sequences, an operative connection indicates a configuration of the genes with respect to the regulatory sequence allowing the regulatory sequences to directly or indirectly increase or decrease transcription or translation of the genes. In particular, in certain embodiments, regulatory sequences directly increasing transcription of the operatively linked gene, comprise promoters typically located on a same strand and upstream on a DNA sequence (towards the 5′ region of the sense strand), adjacent to the transcription start site of the genes whose transcription they initiate. In certain embodiments, regulatory sequences directly increasing transcription of the operatively linked gene or gene cluster comprise enhancers that can be located more distally from the transcription start site compared to promoters, and either upstream or downstream from the regulated genes, as understood by those skilled in the art. Enhancers are typically short (50-1500 bp) regions of DNA that can be bound by transcriptional activators to increase transcription of a particular gene. Typically, enhancers can be located up to 1 Mbp away from the gene, upstream or downstream from the start site.

The term “secretable,” as used herein, means able to be secreted, wherein secretion in the present disclosure generally refers to transport or translocation from the interior of a cell, e.g., within the cytoplasm or cytosol of a cell, to its exterior, e.g., outside the plasma membrane of the cell. Secretion can include several procedures, including various cellular processing procedures such as enzymatic processing of the peptide. In certain embodiments, secretion can utilize the classical secretory pathways of yeast. In certain embodiments, secretion of a protein, e.g., a protein disclosed herein, can be continuous or induced.

The terms “detect” or “detection,” as used herein, indicates the determination of the existence and/or presence of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

The term “derived” or “derive” is used herein to mean to obtain from a specified source.

The term “molecule,” as used herein, refers a group of atoms bonded together, representing the smallest fundamental unit of a chemical compound that can take part in a chemical reaction.

As used interchangeably herein, the terms “wound healing agent” and “skin therapeutic” include any small molecule, protein and peptide that can be administered to a subject and provide a therapeutic effect, such as reduce, alleviate or eliminate symptoms or pathologies of a skin condition, including a wound. In certain embodiments, a skin therapeutic disclosed herein can be used for a cosmetic purpose.

The term “derivative,” as used herein with reference to a protein, e.g., a skin therapeutic, refers to a modified form of the protein, e.g., a skin therapeutic. Non-limiting examples of such derivatives include mutated forms of the protein. For example, but not by way of limitation, a skin therapeutic of the present disclosure can include one or more amino acid substitutions, e.g., 1 or more, 2 or more, 3 or more, 4 or more, 5 or more, 6 or more, 7 or more, 8 or more, 9 or more or 10 or more amino acid substitutions compared to the wild type form of the skin therapeutic.

“Pharmaceutically acceptable carrier,” as used herein, refers to a pharmaceutically acceptable material, composition or vehicle that is involved in carrying or transporting a compound or composition of interest from one tissue, organ, or portion of the body to another tissue, organ, or portion of the body. For example, the carrier can be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it can come in contact, meaning that it must not carry a risk of toxicity, irritation, allergic response, immunogenicity, or any other complication that excessively outweighs its therapeutic benefits.

As used herein the term “subject” refers to any animal (e.g., a mammal), including, but not limited to, humans, non-human primates, rodents, and the like, which is to be recipient of a particular treatment.

As used herein, the term “skin condition” refers to an injury or irregularity or abnormality of the epidermis, dermis or hypodermis. In certain embodiments, a skin condition includes an injury that affects the structural integrity of the epidermis, dermis or hypodermis. In certain embodiments, a skin condition includes an irregularity or abnormality of the structure of the epidermis, dermis or hypodermis. In certain embodiments, a skin condition includes the blistering of the epidermis, dermis or hypodermis, openings or cracks in any one of the dermal layers, swelling, hyperpigmentation, discoloration, scaling, dryness, thickening or scarring of a dermal layer and hair follicle blockage. In certain embodiments, the skin condition is a wound. In certain embodiments, the skin condition is cancer. In certain embodiments, the skin condition is a cosmetic condition.

A “therapeutically effective amount” or a “therapeutically effective level” refers to an amount of genetically-engineered cells and/or a skin therapeutic produced by the genetically-engineered cells that is able to prevent, decrease, alleviate or eliminate one or more symptoms of a condition, e.g., a skin condition.

As used herein, the term “modified” when referencing an organism, e.g., a cell, refers to an organism that does not exist in nature. The term is used interchangeably with “recombinant” or “engineered.”

### II. Skin Therapeutics

The present disclosure provides cells that express and/or secrete one or more skin therapeutics. For example, but not by way of limitation, a cell, e.g., a genetically engineered cell, of the present disclosure can produce and/or secrete one skin therapeutic. In certain embodiments, a cell, e.g., a genetically engineered cell, of the present disclosure can produce and/or secrete more than one skin therapeutic, e.g., two or more skin therapeutics, three or more skin therapeutics, four or more skin therapeutics or five or more skin therapeutics.

In certain embodiments, the skin therapeutic can be a protein or a derivative thereof or a functional fragment thereof. The proteins disclosed herein refer to a sequence of amino acids for which the chain length is sufficient to produce the higher levels of tertiary and/or quaternary structure. This is to distinguish from “peptides” that typically do not have such structure. In certain embodiments, the protein, e.g., protein therapeutic can have a molecular weight of at least about 5-100 kD, e.g., closer to about 15 kD. In certain embodiments, the protein therapeutic can include at least about 10, about 20, about 30, about 40, about 50, about 60, about 70, about 80, about 90, about 100, about 200, about 300, about 400, about 500 amino acids, about 1,000 amino acids, about 1,500 amino acids, about 2,000 amino acids, about 2,500 amino acids, about 3,000 amino acids, about 35,000 amino acids or about 40,000 amino acids. Non-limiting examples of protein therapeutics include all proteins, and, in general proteins that contain one or more disulfide bonds, including multi-chain polypeptides comprising one or more inter- and/or intrachain disulfide bonds. In certain embodiments, the protein therapeutic can include other post-translation modifications including, but not limited to, glycosylation and lipidation. See, e.g., Prabakaran et al., WIREs Syst Biol Med (2012), which is incorporated herein by reference in its entirety. In certain embodiments, the skin therapeutic can be a peptide or a derivative thereof or a functional fragment thereof.

In certain embodiments, the skin therapeutic can be a growth factor, a chemokine and/or cytokine or a functional fragment thereof. In certain embodiments, the skin therapeutic is a protease inhibitor or a functional fragment thereof. In certain embodiments, the skin therapeutic can be a derivative of, e.g., a mutated form of, a growth factor, a cytokine, e.g., a chemokine, and/or a protease inhibitor or a functional fragment thereof. In certain embodiments, the skin therapeutic can be an inhibitor of a growth factor and/or an inhibitor of a cytokine.

In certain embodiments, the skin therapeutic is a growth factor or a derivative thereof or a functional fragment thereof. For example, but not by way of limitation, the growth factor can be epithelial growth factor (EGF), platelet-derived growth factor (PDGF), vascular endothelial growth factor (VEGF) and/or transforming growth factor-beta (TGF-β). In certain embodiments, a genetically-engineered cell of the present disclosure expresses and/or secretes one or more growth factors, e.g., EGF, PDGF, VEGF, TGF, TGF-β, FGF (e.g., FGF2), KGF, NGF, GMCSF, GCSF, IGF, TPO, BMP, SGF, HGF, GDF, MSF, Erythropoietin and/or neurotrophins. In certain embodiments, a genetically-engineered cell of the present disclosure expresses and/or secretes EGF. In certain embodiments, a genetically-engineered cell of the present disclosure expresses and/or secretes PDGF. In certain embodiments, a genetically-engineered cell of the present disclosure expresses and/or secretes VEGF. In certain embodiments, a genetically-engineered cell of the present disclosure expresses and/or secretes TGF-β. In certain embodiments, a genetically-engineered cell of the present disclosure expresses and/or secretes two or more, three, four or more or all of EGF, PDGF, VEGF, TGF, TGF-β, FGF, KGF, NGF, GMCSF, GCSF, IGF, TPO, BMP, SGF, HGF, GDF, MSF, Erythropoietin and/or neurotrophins. In certain embodiments, a genetically-engineered cell of the present disclosure expresses and/or secretes EGF and PDGF.

In certain embodiments, the skin therapeutic is a cytokine or a derivative thereof or a functional fragment thereof. For example, but not by way of limitation, the cytokine can be Leptin or an interleukin, e.g., IL-4, IL-1β, IL-6, IL-10 and IL-12, and TNF-α. Additional non-limiting examples of cytokines are disclosed in Table 1 of Zhang and An, Int. Anesthesiol. Clin. 45(2):27-37 (2007), the contents of which are incorporated by reference herein. In certain embodiments, the cytokine can be a chemokine. In certain embodiments, the cytokine is an interleukin.

In certain embodiments, a genetically-engineered cell of the present disclosure expresses and/or secretes one or more cytokines, e.g., Leptin. In certain embodiments, a genetically-engineered cell of the present disclosure expresses and/or secretes Leptin.

In certain embodiments, a genetically-engineered cell of the present disclosure expresses and/or secretes an inhibitor of a growth factor. In certain embodiments, a genetically-engineered cell of the present disclosure expresses and/or secretes an inhibitor of VEGF. In certain embodiments, a genetically-engineered cell of the present disclosure expresses and/or secretes one or more interleukins, e.g., IL-4. In certain embodiments, a genetically-engineered cell of the present disclosure expresses and/or secretes IL-4. As described herein, IL-4 can function as an inhibitor of VEGF. Additional non-limiting examples of VEGF inhibitors are disclosed in Guryanov et al., Pharmaceutics. 13(9):1337 (2021) and Zhang et al., Signal Transduction and Targeted Therapy 2: Article number:17010 (2017), the contents of each are incorporated in their entireties by reference herein. In certain embodiments, the VEGF inhibitor is a peptide, e.g., HRHTKQRHTALH (SEQ ID NO: 52). As described herein, an inhibitor of VEGF can be used to treat a skin condition such as psoriasis.

In certain embodiments, the skin therapeutic is an inhibitor, e.g., a peptide inhibitor, of TGF-β. In certain embodiments, the skin therapeutic is an inhibitor of TGF-β isoform TGF-β1. In certain embodiments, the inhibitor of TGF-β1 is a peptide inhibitor of TGF-β1. Non-limiting examples of peptide inhibitors of TGF-β1 are disclosed in WO 2011/101478 (e.g., Table 1), the contents of which is incorporated by reference herein in its entirety. In certain embodiments, the peptide inhibitor comprises the amino acid sequence TSLDASIIWAMMQN (SEQ ID NO: 9). As described herein, an inhibitor of TGF-β1 can be used to treat skin fibrosis and/or scleroderma.

In certain embodiments, the skin therapeutic is a chemokine or a derivative thereof or a functional fragment thereof. For example, but not by way of limitation, the chemokine can be CXCL12, CXCL2, CXCL8, CXCL10, CXCL11, CXCL4, CXCL9, CCL2, CCL5, CXCL1, CCL3, CCL4, CCL5, CCL7, CCL8, CCL11, CCL21, CCL26, CCL13, CCL24, XCL1, XCL2 and/or CX3CL1. In certain embodiments, the chemokine can be CXCL12. In certain embodiments, a genetically-engineered cell of the present disclosure expresses and/or secretes two or more chemokines, e.g., CXCL12, CXCL2, CXCL8, CXCL10, CXCL11, CXCL4, CXCL9, CCL2, CCL5, CXCL1, CCL3, CCL4, CCL5, CCL7, CCL8, CCL11, CCL21, CCL26, CCL13, CCL24, XCL1, XCL2 and/or CX3CL1.

In certain embodiments, the skin therapeutic is a protease inhibitor or a derivative thereof or a functional fragment thereof. In certain embodiments, the protease inhibitor is an inhibitor of a protease that is normally present in wounds. Non-limiting examples of proteases that are present in wounds and involved in wound healing are disclosed in McCarthy and Percival, Adv. Wound Care 2(8):438-447 (2013) and Westby et al., Cochrane Database Syst. Rev. 2018(9):CD012841 (2018), the contents of which are incorporated herein in their entireties. In certain embodiments, the protease inhibitor is an inhibitor of a serine protease. In certain embodiments, the protease inhibitor is an inhibitor of a matrix metalloproteinase (MMP) and/or an inhibitor of a disintegrin and metalloproteinase (ADAMS and ADAMTSs). In certain embodiments, the protease inhibitor is TIMP1 and/or TIMP2.

In certain embodiments, the skin therapeutic is an antimicrobial and/or anti-inflammatory agent. In certain embodiments, the antimicrobial and/or anti-inflammatory agent is an antimicrobial and/or anti-inflammatory peptide or a functional fragment thereof. In certain embodiments, the antimicrobial and/or anti-inflammatory peptide is cathelicidin antimicrobial peptide (LL-37) or analogs thereof. Additional non-limiting examples of antimicrobial peptides include RcA1b-PepI, RcA1b-PepII, RcA1b-PepII, lucifensin, lucifensin II, lucilin, pexiganan acetate (MSI-78), D2A21/D4E1, granulysin and synthetic granulysin-derived peptides. In certain embodiments, the antimicrobial peptide is a defensin, e.g., an α-, β- or θ-defensin. Non-limiting examples of defensins include β-defensin-1 (hBD-1), β-defensin-2 (hBD-2), β-defensin-3 (hBD-3), neutrophil peptide 1 (HNP1), neutrophil peptide 2 (HNP2), neutrophil peptide 3 (HNP3), neutrophil peptide 4 (HNP4), human defensin 5 (HD5) and human defensin 6 (HD6).

In certain embodiments, the skin therapeutic is a toxin peptide. In certain embodiments, the toxin peptide is derived from a fungal cell. In certain embodiments, the toxin peptide is a K1, K2 or K28 toxin peptide derived from Saccharomyces cerevisiae.

In certain embodiments, the skin therapeutic is an inhibitor of RelA. In certain embodiments, the skin therapeutic is an RNAi targeting RelA.

In certain embodiments, the skin therapeutic is a xeroderma pigmentosum complementation protein (XP). Non-limiting examples of XPs include XPA, XPB, XPC (human RAD4), XPD, XPE, XPF and XPG.

In certain embodiments, the skin therapeutic is an extracellular matrix protein. Non-limiting examples of extracellular matrix proteins include collagen, proteoglycans, fibronectin and elastin (see, e.g., Yue, J. Glaucoma S20-S23 (2014), the contents of which are incorporated herein by reference). In certain embodiments, the skin therapeutic is a fragment of an extracellular matrix protein. In certain embodiments, the skin therapeutic is a collagen. Non-limiting examples of collagen include Collagen I, Collagen II, Collagen III, Collagen IV, Collagen V, Collagen VI, Collagen VII, Collagen VIII, Collagen IX, Collagen X, Collagen XI, Collagen XII, Collagen XIII, Collagen XIV, Collagen XV, Collagen XVI, Collagen XVII, Collagen XVIII, Collagen XIX, Collagen XX, Collagen XXI, Collagen XXII, Collagen XXIII, Collagen XIV, Collagen XXV, Collagen XXVI, Collagen XXVII and Collagen XXVIII (see, e.g., Yue, J. Glaucoma S20-S23 (2014), the contents of which are incorporated herein by reference). In certain embodiments, the skin therapeutic is Type VII Collagen or a fragment thereof. As described herein, an extracellular matrix protein can be used to treat epidermolysis bullosa. In certain embodiments, the skin therapeutic is elastin.

In certain embodiments, the skin therapeutic is azelaic acid.

In certain embodiments, the skin therapeutic is hyaluronic acid.

In certain embodiments, the skin therapeutic is a peptide or protein comprising an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or 100% homologous to a sequence comprising the sequence of any one of the above-noted peptides or proteins. In certain embodiments, the skin therapeutic is a peptide or protein comprising an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or 100% homologous to a sequence comprising the sequence of any one of the skin therapeutics disclosed herein. In certain embodiments, the skin therapeutic comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% or 100% homologous to a sequence provided in Table 1 or Table 2. In certain embodiments, the skin therapeutic comprises an amino acid sequence that is at least about 97% homologous to a sequence provided in Table 1 or Table 2. In certain embodiments, the skin therapeutic comprises an amino acid sequence that is at least about 98% homologous to a sequence provided in Table 1 or Table 2. In certain embodiments, the skin therapeutic comprises an amino acid sequence that is at least about 99% homologous to a sequence provided in Table 1 or Table 2. In certain embodiments, the skin therapeutic comprises an amino acid sequence that is from about 98% to about 100% homologous to a sequence provided in Table 1 or Table 2. In certain embodiments, the skin therapeutic comprises an amino acid sequence provided in Table 1 or Table 2.

In certain embodiments, the skin therapeutic is a peptide or protein comprising an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or 100% homologous, e.g., about 97% to about 100% homologous, to a sequence comprising the sequence of any one of SEQ ID NOs: 1-9, 11-21 and 53-56. In certain embodiments, the skin therapeutic comprises an amino acid sequence that is at least about 97% homologous to a sequence of SEQ ID NOs: 1-9, 11-21 and 53-56. In certain embodiments, the skin therapeutic comprises an amino acid sequence that is at least about 98% homologous to a sequence of SEQ ID NOs: 1-9, 11-21 and 53-56. In certain embodiments, the skin therapeutic comprises an amino acid sequence that is at least about 99% homologous to a sequence of SEQ ID NOs: 1-9, 11-21 and 53-56. In certain embodiments, the skin therapeutic comprises an amino acid sequence that is from about 98% to about 100% homologous to a sequence of SEQ ID NOs: 1-9, 11-21 and 53-56. In certain embodiments, the skin therapeutic comprises an amino acid sequence of SEQ ID NOs: 1-9, 11-21 and 53-56.

In certain embodiments, the skin therapeutic is a peptide or protein comprising an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or 100% homologous, e.g., about 97% to about 100% homologous, to a sequence comprising the sequence of SEQ ID NO: 1. In certain embodiments, the skin therapeutic is a peptide or protein comprising an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or 100% homologous, e.g., about 97% to about 100% homologous, to a sequence comprising the sequence of SEQ ID NO: 2. In certain embodiments, the skin therapeutic is a peptide or protein comprising an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or 100% homologous, e.g., about 97% to about 100% homologous, to a sequence comprising the sequence of SEQ ID NO: 3. In certain embodiments, the skin therapeutic is a peptide or protein comprising an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or 100% homologous, e.g., about 97% to about 100% homologous, to a sequence comprising the sequence of SEQ ID NO: 4. In certain embodiments, the skin therapeutic is a peptide or protein comprising an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or 100% homologous, e.g., about 97% to about 100% homologous, to a sequence comprising the sequence of SEQ ID NO: 5. In certain embodiments, the skin therapeutic is a peptide or protein comprising an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or 100% homologous, e.g., about 97% to about 100% homologous, to a sequence comprising the sequence of SEQ ID NO: 6. In certain embodiments, the skin therapeutic is a peptide or protein comprising an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or 100% homologous, e.g., about 97% to about 100% homologous, to a sequence comprising the sequence of SEQ ID NO: 7. In certain embodiments, the skin therapeutic is a peptide or protein comprising an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or 100% homologous, e.g., about 97% to about 100% homologous, to a sequence comprising the sequence of SEQ ID NO: 8.

TABLE 1

| Protein | Amino acid sequence |
|---|---|
| human PDGFb (hPDGFb) | SLGSLTIAEPAMIAECKTRTEVFEISRRLIDRTNANFL |
| | VWPPCVEVQRCSGCCNNRNVQCRPTQVQLRPVQVR |
| | KIEIVRKKPIFKKATVTLEDHLACKCETVAAARPVT |
| | (SEQ ID NO: 1) |
| mouse PDGFb (mPDGFb) | SLGSLAAAEPAVIAECKTRTEVFQISRNLIDRTNANFL |
| | VWPPCVEVQRCSGCCNNRNVQCRASQVQMRPVQV |
| | RKIEIVRKKPIFKKATVTLEDHLACKCETIVTPRPVT |
| | (SEQ ID NO: 2) |
| human EGF (hEGF) | NSDSECPLSHDGYCLHDGVCMYIEALDKYACNCVV |
| | GYIGERCQYRDLKWWELR (SEQ ID NO: 3) |
| mouse EGF (mEGF) | NSYPGCPSSYDGYCLNGGVCMHIESLDSYTCNCVIG |
| | YSGDRCQTRDLRWWELR (SEQ ID NO: 4) |
| human Leptin (hLeptin) | VPIQKVQDDTKTLIKTIVTRINDISHTQSVSSKQKVTG |
| | LDFIPGLHPILTLSKMDQTLAVYQQILTSMPSRNVIQI |
| | SNDLENLRDLLHVLAFSKSCHLPWASGLETLDSLGG |
| | VLEASGYSTEVVALSRLQGSLQDMLWQLDLSPGC |
| | (SEQ ID NO: 5) |
| mouse Leptin (mLeptin) | VPIQKVQDDTKTLIKTIVTRINDISHTQSVSAKQRVTG |
| | LDFIPGLHPILSLSKMDQTLAVYQQVLTSLPSQNVLQ |
| | IANDLENLRDLLHLLAFSKSCSLPQTSGLQKPESLDG |
| | VLEASLYSTEVVALSRLQGSLQDILQQLDVSPEC |
| | (SEQ ID NO: 6) |
| mouse CXCL12 (mCXCL12) | KPVSLSYRCPCRFFESHIARANVKHLKILNTPNCALQI |
| | VARLKNNNRQVCIDPKLKWIQEYLEKALNK (SEQ ID |
| | NO: 7) |
| human CXCL12 (hCXCL12) | KPVSLSYRCPCRFFESHVARANVKHLKILNTPNCAL |
| | QIVARLKNNNRQVCIDPKLKWIQEYLEKALNK (SEQ |
| | ID NO: 8) |

In certain embodiments, the skin therapeutic expressed by a genetically-engineered cell of the present disclosure is secretable. For example, but not by way of limitation, the skin therapeutic can be expressed intracellularly in a cell and subsequently transported to the plasma membrane of the cell and secreted to the exterior of the cell, e.g., outside the plasma membrane of the cell. In certain embodiments, the skin therapeutic expressed by a genetically-engineered cell of the present disclosure is continuously secreted by the cell. In certain embodiments, the skin therapeutic expressed by a genetically-engineered cell of the present disclosure is secreted by the cell upon induction.

In certain embodiments, secretion of the skin therapeutic can be performed using the conserved secretory pathway in fungal cells, e.g., yeast. For example, but not by way of limitation, a skin therapeutic is secretable because it is coupled to a secretion signal sequence. Non-limiting examples of secretion signal sequences can be obtained from proteins including mating factor alpha-1, alpha factor K, alpha factor T, SED1, PHO5, SUC2, glycoamylase, inulinase, invertase, lysozyme, serum albumin, alpha-amylase, killer protein and Table 6. In certain embodiments, the secretion signal peptide is mating factor alpha-1. In certain embodiments, the secretion signal peptide is SED1. In certain embodiments, the secretion signal peptide is PHO5. In certain embodiments, the secretion signal peptide is SUC2. In certain embodiments, the secretion signal sequence is a secretion signal sequence obtained from a yeast protein, such as a Saccharomyces cerevisiae protein. In certain embodiments, the secretion signal peptide is obtained from Saccharomyces cerevisiae mating factor alpha-1. In certain embodiments, the secretion signal peptide is SED1. Additionally, mutations, substitutions and truncations of any signal peptide are also within the scope of the present disclosure. The selection and design, including additional mutations and truncations of a signal peptide is within the ability and discretion of one of ordinary skill in the art. In certain embodiments, the one or more secretion signal sequences are located at the N-terminus of a skin therapeutic. In certain embodiments, a Kex2 processing site and/or a Ste13 processing site or a homolog thereof can be present between the amino acid sequence of the secretion signal sequence and the secretable peptide. Additional non-limiting examples of secretion signals are disclosed in U.S. Pat. No. 10,725,036, the contents of which is disclosed herein in its entirety.

### III. Genetically-Engineered Cells

The present disclosure provides cells for expressing, e.g., secreting, a skin therapeutic disclosed herein. In certain embodiments, the cells that have been genetically engineered to express and secrete a skin therapeutic can be administered to a subject for treating a skin condition, e.g., a wound. Non-limiting examples of skin therapeutics that can be produced by the cells of the present disclosure are disclosed in Section II.

The cells used for generating and/or secreting the skin therapeutics described herein can be, e.g., genetically engineered cells. The genetically-modified cells for use in generating a skin therapeutic can be a mammalian cell, a plant cell, a bacterial cell or a fungal cell. For example, but not by way of limitation, the cell can be a mammalian cell, e.g., a genetically engineered mammalian cell. In certain embodiments, the cell can be a plant cell, e.g., a genetically engineered plant cell. In certain embodiments, the cell can be a bacterial cell, e.g., a genetically engineered bacterial cell. In certain embodiments, the cell can be a fungal cell, e.g., a genetically engineered fungal cell. In certain embodiments, the cell is not a bacterial cell.

In certain embodiments, the cell for use in the present disclosure can be a cell that is designated by the United States Food and Drug Administration (FDA) as generally recognized as safe (GRAS).

In certain embodiments, the cell is a fungal cell. Any fungal strain can be used in the present disclosure. In certain embodiments, the fungal cell can be a species from a genus including, but not limited to, Cladosporium, Aureobasidium, Aspergillus, Saccharomyces, Malassezia, Epicoccum, Candida, Penicillium, Wallemia, Pichia, Phoma, Cryptococcus, Fusarium, Clavispora, Cyberlindnera and Kluyveromyces.

In certain embodiments, a genetically-engineered cell of the present disclosure can be a cell of Alternaria brassicicola, Arthrobotrys oligospora, Ashbya aceri, Ashbya gossypii, Aspergillus clavatus, Aspergillus flavus, Aspergillus fumigate, Aspergillus kawachii, Aspergillus nidulans, Aspergillus niger, Aspergillus oryzae, Aspergillus ruber, Aspergillus terreus, Baudoinia compniacensis, Beauveria bassiana, Botryosphaeria parva, Botrytis cinereal, Candida albicans, Candida dubliniensis, Candida glabrata, Candida guilliermondii, Candida lusitaniae, Candida parapsilosis, Candida tenuis, Candida tropicalis, Capronia coronate, Capronia epimyces, Chaetomium globosum, Chaetomium thermophilum, Chryphonectria parasitica, Claviceps purpurea, Coccidioides immitis, Colletotrichum gloeosporioides, Coniosporium apollinis, Dactylellina haptotyla, Debaryomyces hansenii, Endocarpon pusillum, Eremothecium cymbalariae, Fusarium oxysporum, Fusarium pseudograminearum, Gaeumannomyces graminis, Geotrichum candidum, Gibberella fujikuroi, Gibberella moniliformis, Gibberella zeae, Glarea lozoyensis, Grosmannia clavigera, Kazachstania africana, Kazachstania naganishii, Kluyveromyces lactis, Kluyveromyces marxianus, Kluyveromyces waltii, Komagataella pastoris, Kuraishia capsulate, Lachancea kluyveri, Lachancea thermotolerans, Lodderomyces elongisporus, Magnaporthe oryzae, Magnaporthe poae, Marssonina brunnea, Metarhizium acridum, Metarhizium anisopliae, Mycosphaerella graminicola, Mycosphaerella pini, Nectria haematococca, Neosartorya fischeri, Neurospora crassa, Neurospora tetrasperma, Ogataea parapolymorpha, Ophiostoma piceae, Paracoccidioides lutzii, Penicillium chrysogenum, Penicillium digitatum, Penicillium oxalicum, Penicillium roqueforti, Phaeosphaeria nodorum, Pichia sorbitophila, Podospora anserine, Pseudogymnoascus destructans, Pyrenophora teres f teres, Pyrenophora tritici-repentis, Saccharomyces bayanus, Saccharomyces castellii, Saccharomyces cerevisiae, Saccharomyces dairenensis, Saccharomyces mikatae, Saccharomyces paradoxis, Scheffersomyces stipites, Schizosaccharomyces japonicus, Schizosaccharomyces octosporus, Schizosaccharomyces pombe, Sclerotinia borealis, Sclerotinia sclerotiorum, Sordaria macrospora, Sporothrix schenckii, Tetrapisispora blattae, Tetrapisispora phaffii, Thielavia heterothallica, Togninia minima, Torulaspora delbrueckii, Trichoderma atroviridis, Trichoderma jecorina, Trichoderma virens, Tuber melanosporum, Vanderwaltozyma polyspora 1, Vanderwaltozyma polyspora 2, Verticillium alfalfae, Verticillium dahliae, Wickerhamomyces ciferrii, Yarrowia lipolytica, Zygosaccharomyces bailii, Zygosaccharomyces rouxii and combinations thereof.

In certain embodiments, the genetically engineered cell of the present disclosure is a species of phylum Ascomycota. In certain embodiments, the species of the phylum Ascomycota is selected from Saccharomyces cerevisiae, Saccharomyces castellii, Saccharomyces var boulardii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauveria bassiana, Neurospora crassa, Sporothrix scheckii, Magnaporthe oryzae, Dactylellina haptotyla, Fusarium graminearum, Capronia coronate and combinations thereof.

In certain embodiments, the genetically-engineered cell of the present disclosure is a species of the Saccharomyces genus. In certain embodiments, the species from the Saccharomyces genus is selected from the group consisting of Saccharomyces bayanus, Saccharomyces boulardii, Saccharomyces castellii, Saccharomyces cerevisiae, Saccharomyces dairenensis and Saccharomyces mikatae. In certain embodiments, the genetically-engineered cell of the present disclosure is Saccharomyces cerevisiae. In certain embodiments, the genetically-engineered cell of the present disclosure is Saccharomyces boulardii.

In certain embodiments, the genetically-engineered cell of the present disclosure is a species from the Pichia genus. In certain embodiments, species from the Pichia genus is selected from the group consisting of Pichia acacia, Pichia alni, Pichia americana, Pichia amethionina, Pichia amylophila, Pichia angophorae, Pichia angusta, Pichia anomala, Pichia antillensis, Pichia barkeri, Pichia besseyi, Pichia bimundalis, Pichia bispora, Pichia bovis, Pichia cactophila, Pichia canadensis, Pichia capsulate, Pichia caribaea, Pichia castillae, Pichia chambardii, Pichia ciferrii, Pichia delftensis, Pichia deserticola, Pichia dryadoides, Pichia euphorbiae, Pichia euphorbiiphila, Pichia fabianii, Pichia farinose, Pichia fermentans, Pichia finlandica, Pichia fluxuum, Pichia galaeiformis, Pichia glucozyma, Pichia guilliermondii, Pichia hampshirensis, Pichia haplophila, Pichia heedii, Pichia heimii, Pichia henricii, Pichia holstii, Pichia inositovora, Pichia jadinii, Pichia japonica, Pichia kluyveri, Pichia kodamae, Pichia lynferdii, Pichia maganishii, Pichia media, Pichia membranifaciens, Pichia methanolica, Pichia methylivoria, Pichia Mexicana, Pichia meyerae, Pichia minuta, Pichia mississippiensis, Pichia nakasei, Pichia nakazawae, Pichia norvegensis, Pichia ofunaensis, Pichia ohmeri, Pichia onychis, Pichia opuntiae, Pichia pastoris, Pichia petersonii, Pichia philodendra, Pichia philogaea, Pichia pijperi, Pichia pini, Pichia populi, Pichia pseudocactophila, Pichia quercuum, Pichia rabaulensis, Pichia rhodanensis, Pichia salicaria, Pichia scolyti, Pichia segobiensis, Pichia silvicola, Pichia spartinae, Pichia stipites, Pichia strasburgensis, Pichia subpelliculosa, Pichia sydowiorum, Pichia tannicola, Pichia thermotolerans, Pichia toletana, Pichia trehalophila, Pichia triangularis, Pichia veronae, Pichia wickerhamii and Pichia xylosa. In certain embodiments, the genetically-engineered cell of the present disclosure is Pichia pastoris.

In certain embodiments, the genetically-engineered cell of the present disclosure is a species of the Kluyveromyces genus. In certain embodiments, the genetically-engineered cell of the present disclosure is Kluyveromyces lactis.

In certain embodiments, the genetically-engineered cell of the present disclosure is a bacterial cell. Non-limiting examples of bacteria include Caulobacter crescentus, Rodhobacter sphaeroides, Pseudoalteromonas haloplanktis, Shewanella sp. strain Ac10, Pseudomonas fluorescens, Pseudomonas aeruginosa, Halomonas elongata, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, Lactobacillus gasseri and Escherichia coli. In certain embodiments, the bacteria cell is Escherichia coli. In certain embodiments, the genetically-engineered cell of the present disclosure is not a bacterial cell.

In certain embodiments, the genetically engineered cell of the present disclosure is a mammalian cell. Non-limiting examples of mammalian cells include monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; FS4 cells; MCF-7 cells; 3T3 cells; U2SO cells; Chinese hamster ovary (CHO) cells and myeloma cell lines such as Y0, NS0 and Sp2/0. In certain embodiments, the genetically-engineered cell of the present disclosure is not a mammalian cell.

In certain embodiments, a genetically-engineered cell, e.g., a genetically-engineered fungal cell, of the present disclosure expresses and/or secretes at least one growth factor or a derivative thereof, e.g., EGF and/or PDGF.

In certain embodiments, a genetically-engineered cell, e.g., a genetically-engineered fungal cell, of the present disclosure expresses and/or secretes at least one cytokine or a derivative thereof, e.g., Leptin.

In certain embodiments, a genetically-engineered cell, e.g., a genetically-engineered fungal cell, of the present disclosure expresses and/or secretes at least one cytokine or a derivative thereof, e.g., IL-12.

In certain embodiments, a genetically-engineered cell, e.g., a genetically-engineered fungal cell, of the present disclosure expresses and/or secretes at least one interferon or a derivative thereof.

In certain embodiments, a genetically-engineered cell, e.g., a genetically-engineered fungal cell, of the present disclosure expresses and/or secretes at least one chemokine or a derivative thereof, e.g., CXCL12.

In certain embodiments, a genetically-engineered cell, e.g., a genetically-engineered fungal cell, of the present disclosure expresses and/or secretes at least one protease inhibitor or a derivative thereof, e.g., TIMP1 and/or TIMP2.

In certain embodiments, a genetically-engineered cell, e.g., a genetically-engineered fungal cell, of the present disclosure expresses and/or secretes at least one antimicrobial agent.

In certain embodiments, a genetically-engineered cell of the present disclosure expresses and/or secretes (1) a growth factor or a derivative thereof, e.g., EGF and/or PDGF, and (2) a cytokine or a derivative thereof, e.g., Leptin.

In certain embodiments, a genetically-engineered cell of the present disclosure expresses and/or secretes (1) a growth factor or a derivative thereof, e.g., EGF and/or PDGF, and (2) a chemokine or a derivative thereof, e.g., CXCL12.

In certain embodiments, a genetically-engineered cell of the present disclosure expresses and/or secretes (1) a cytokine or a derivative thereof, e.g., Leptin, and (2) a chemokine or a derivative thereof, e.g., CXCL12.

In certain embodiments, a genetically-engineered cell of the present disclosure expresses and/or secretes (1) a growth factor or a derivative thereof, e.g., EGF and/or PDGF, and (2) a protease inhibitor or a derivative thereof, e.g., TIMP1 and/or TIMP2.

In certain embodiments, a genetically-engineered cell of the present disclosure expresses and/or secretes (1) a growth factor or a derivative thereof, e.g., EGF and/or PDGF, and (2) antimicrobial agent.

In certain embodiments, a genetically-engineered cell of the present disclosure expresses and/or secretes (1) a protease inhibitor or a derivative thereof, e.g., TIMP1 and/or TIMP2, and (2) a chemokine or a derivative thereof, e.g., CXCL12.

In certain embodiments, a genetically-engineered cell of the present disclosure expresses and/or secretes (1) a protease inhibitor or a derivative thereof, e.g., TIMP1 and/or TIMP2, and (2) an antimicrobial agent.

In certain embodiments, a genetically-engineered cell of the present disclosure expresses and/or secretes (1) a cytokine or a derivative thereof, e.g., Leptin, and (2) a protease inhibitor or a derivative thereof, e.g., TIMP1 and/or TIMP2.

In certain embodiments, a genetically-engineered cell of the present disclosure expresses and/or secretes (1) a cytokine or a derivative thereof, e.g., Leptin, and (2) an antimicrobial agent.

In certain embodiments, a genetically-engineered cell of the present disclosure expresses and/or secretes (1) a growth factor or a derivative thereof, e.g., EGF and/or PDGF, (2) a cytokine or a derivative thereof, e.g., Leptin, and (3) a chemokine or a derivative thereof, e.g., CXCL12.

In certain embodiments, a genetically-engineered cell of the present disclosure expresses and/or secretes (1) a protease inhibitor or a derivative thereof, e.g., TIMP1 and/or TIMP2, (2) a cytokine or a derivative thereof, e.g., Leptin, and (3) a chemokine or a derivative thereof, e.g., CXCL12.

In certain embodiments, a genetically-engineered cell of the present disclosure expresses and/or secretes (1) a growth factor or a derivative thereof, e.g., EGF and/or PDGF, (2) a protease inhibitor or a derivative thereof, e.g., TIMP1 and/or TIMP2, and (3) a chemokine or a derivative thereof, e.g., CXCL12.

In certain embodiments, a genetically-engineered cell of the present disclosure expresses and/or secretes (1) a growth factor or a derivative thereof, e.g., EGF and/or PDGF, (2) a cytokine or a derivative thereof, e.g., Leptin, and (3) a protease inhibitor or a derivative thereof, e.g., TIMP1 and/or TIMP2.

In certain embodiments, a genetically-engineered cell of the present disclosure expresses and/or secretes (1) a growth factor or a derivative thereof, e.g., EGF and/or PDGF, (2) a cytokine or a derivative thereof, e.g., Leptin, (3) a protease inhibitor or a derivative thereof, e.g., TIMP1 and/or TIMP2, and (4) a chemokine or a derivative thereof, e.g., CXCL12.

In certain embodiments, a genetically-engineered cell of the present disclosure expresses and/or secretes (1) a growth factor or a derivative thereof, e.g., EGF and/or PDGF, (2) a cytokine or a derivative thereof, e.g., Leptin, (3) a protease inhibitor or a derivative thereof, e.g., TIMP1 and/or TIMP2, and (4) an antimicrobial agent.

In certain embodiments, a genetically-engineered cell of the present disclosure expresses and/or secretes (1) a growth factor or a derivative thereof, e.g., EGF and/or PDGF, (2) a cytokine or a derivative thereof, e.g., Leptin, (3) a protease inhibitor or a derivative thereof, e.g., TIMP1 and/or TIMP2, (4) a chemokine or a derivative thereof, e.g., CXCL12, and (5) an antimicrobial agent.

In certain embodiments, a genetically-engineered cell of the present disclosure expresses and/or secretes a growth factor inhibitor. In certain embodiments, a genetically-engineered cell of the present disclosure expresses and/or secretes a VEGF inhibitor. In certain embodiments, a genetically-engineered cell of the present disclosure expresses and/or secretes a VEGF inhibitor, e.g., IL-4.

In certain embodiments, a genetically-engineered cell of the present disclosure expresses and/or secretes an TGF-β inhibitor, e.g., a TGF-β1 inhibitor. In certain embodiments, a genetically-engineered cell of the present disclosure expresses and/or secretes a peptide TGF-β1 inhibitor, e.g., TSLDASIIWAMMQN (SEQ ID NO: 9) or TSLDASIIWAMMQNA (SEQ ID NO: 12).

In certain embodiments, a genetically-engineered cell of the present disclosure expresses and/or secretes an extracellular matrix protein, e.g., a collagen. In certain embodiments, a genetically-engineered cell of the present disclosure expresses and/or secretes Type VII Collagen or a fragment thereof. In certain embodiments, a genetically-engineered cell of the present disclosure expresses and/or secretes elastin.

In certain embodiments, a genetically-engineered cell of the present disclosure expresses and/or secretes an antimicrobial agent. In certain embodiments, a genetically-engineered cell of the present disclosure expresses and/or secretes an antimicrobial peptide. In certain embodiments, a genetically-engineered cell of the present disclosure expresses and/or secretes an antimicrobial peptide selected from the group consisting of RcA1b-PepI, RcA1b-PepII, RcA1b-PepII, lucifensin, lucifensin II, lucilin, pexiganan acetate (MSI-78), D2A21/D4E1, granulysin, synthetic granulysin-derived peptides, cathelicidin antimicrobial peptide (LL-37) or analogs thereof and a combination thereof. In certain embodiments, a genetically-engineered cell of the present disclosure expresses and/or secretes cathelicidin antimicrobial peptide (LL-37).

In certain embodiments, a genetically-engineered cell of the present disclosure expresses and/or secretes a killer toxin. In certain embodiments, a genetically-engineered cell of the present disclosure expresses and/or secretes K1, K2 and/or K28 from Saccharomyces cerevisiae. In certain embodiments, a genetically-engineered cell of the present disclosure expresses and/or secretes K2.

In certain embodiments, a genetically-engineered cell of the present disclosure expresses and/or secretes a defensin, e.g., an α-, β- or θ-defensin. In certain embodiments, the antimicrobial peptide is β-defensin-1 (hBD-1), β-defensin-2 (hBD-2), β-defensin-3 (hBD-3), neutrophil peptide 1 (HNP1), neutrophil peptide 2 (HNP2), neutrophil peptide 3 (HNP3), neutrophil peptide 4 (HNP4), human defensin 5 (HD5) and/or human defensin 6 (HD6). In certain embodiments, a genetically-engineered cell of the present disclosure expresses and/or secretes β-defensin-3 (hBD-3).

In certain embodiments, a genetically-engineered cell of the present disclosure expresses and/or secretes an inhibitor of RelA. In certain embodiments, a genetically-engineered cell of the present disclosure expresses and/or secretes an RNAi targeting RelA.

In certain embodiments, a genetically-engineered cell of the present disclosure expresses and/or secretes an anti-staphylococcus bactericidal protein.

In certain embodiments, a genetically-engineered cell of the present disclosure expresses and/or secretes a xeroderma pigmentosum complementation protein (XP), e.g., XPA, XPB, XPC (human RAD4), XPD, XPE, XPF or XPG. In certain embodiments, a genetically-engineered cell of the present disclosure expresses and/or secretes XPA, XPB, XPC (human RAD4), XPD, XPE, XPF and/or XPG.

In certain embodiments, a genetically-engineered cell of the present disclosure expresses and/or secretes azelaic acid.

In certain embodiments, a genetically-engineered cell of the present disclosure expresses and/or secretes hyaluronic acid.

In certain embodiments, the genetically-engineered cells express and/or secrete a skin therapeutic at high levels. In certain embodiments, the genetically-engineered cells express and/or secrete a skin therapeutic at levels sufficient for treating the subject. In certain embodiments, the genetically-engineered cells express and/or secrete a therapeutically effective amount of a skin therapeutic. For example, but not by way of limitation, the amount of the skin therapeutic produced by a genetically-engineered cell, e.g., a population of genetically-engineered cells, in about 1 to about 24 hours is between about 1 pg and about 10 g. In certain embodiments, the amount of the skin therapeutic secreted by a genetically-engineered cell, e.g., a population of genetically-engineered cells, in about 1 to about 48 hours or in about 1 to about 24 hours is between about 1 pg/ml to about 200,000 pg/ml, e.g., between about 100 pg/ml to about 200,000 pg/ml, between about 1,000 pg/ml to about 200,000 pg/ml, between about 5,000 pg/ml to about 200,000 pg/ml, between about 10,000 pg/ml to about 200,000 pg/ml, between about 20,000 pg/ml to about 200,000 pg/ml, between about 30,000 pg/ml to about 200,000 pg/ml, between about 40,000 pg/ml to about 200,000 pg/ml, between about 50,000 pg/ml to about 200,000 pg/ml, between about 60,000 pg/ml to about 200,000 pg/ml, between about 70,000 pg/ml to about 200,000 pg/ml, between about 80,000 pg/ml to about 200,000 pg/ml, between about 90,000 pg/ml to about 200,000 pg/ml, between about 100,000 pg/ml to about 200,000 pg/ml, between about 110,000 pg/ml to about 200,000 pg/ml, between about 120,000 pg/ml to about 200,000 pg/ml, between about 130,000 pg/ml to about 200,000 pg/ml, between about 140,000 pg/ml to about 200,000 pg/ml, between about 150,000 pg/ml to about 200,000 pg/ml, between about 100 pg/ml to about 190,000 pg/ml, between about 100 pg/ml to about 180,000 pg/ml, between about 100 pg/ml to about 170,000 pg/ml, between about 100 pg/ml to about 160,000 pg/ml, between about 100 pg/ml to about 150,000 pg/ml, between about 100 pg/ml to about 140,000 pg/ml, between about 100 pg/ml to about 130,000 pg/ml, between about 100 pg/ml to about 120,000 pg/ml, between about 100 pg/ml to about 100,000 pg/ml, between about 100 pg/ml to about 90,000 pg/ml, between about 100 pg/ml to about 80,000 pg/ml, between about 100 pg/ml to about 70,000 pg/ml, between about 100 pg/ml to about 60,000 pg/ml, between about 100 pg/ml to about 50,000 pg/ml, between about 100 pg/ml to about 40,000 pg/ml, between about 100 pg/ml to about 30,000 pg/ml, between about 100 pg/ml to about 20,000 pg/ml, between about 100 pg/ml to about 10,000 pg/ml, between about 100 pg/ml to about 5,000 pg/ml, between about 1,000 pg/ml to about 100,000 pg/ml, between about 1,000 pg/ml to about 50,000 pg/ml, between about 1,000 pg/ml to about 25,000 pg/ml, between about 1,000 pg/ml to about 10,000 pg/ml or between about 1,000 pg/ml to about 5,000 pg/ml. In certain embodiments, the amount of the skin therapeutic secreted by a genetically-engineered cell, e.g., a population of genetically-engineered cells, in about 1 to about 48 hours or in about 1 to about 24 hours is between about 10 pg/ml to about 200,000 pg/ml. In certain embodiments, the amount of the skin therapeutic secreted by a genetically-engineered cell, e.g., a population of genetically-engineered cells, in about 1 to about 48 hours or in about 1 to about 24 hours is between about 10 pg/ml to about 25,000 pg/ml. In certain embodiments, the amount of the skin therapeutic secreted by a genetically-engineered cell, e.g., a population of genetically-engineered cells, in about 1 to about 48 hours or in about 1 to about 24 hours is between about 10 pg/ml to about 2,500 pg/ml, e.g., between about 100 pg/ml to about 2,500 pg/ml, between about 1,000 pg/ml to about 2,000 pg/ml or between about 1,500 pg/ml to about 2,500 pg/ml. In certain embodiments, the amount of the skin therapeutic secreted by a genetically-engineered cell, e.g., a population of genetically-engineered cells, in about 1 to about 48 hours or in about 1 to about 24 hours, e.g., in about 24 hours or less, is between about 0.1 ng/ml to about 1,000 ng/ml. In certain embodiments, the amount of the skin therapeutic secreted by a genetically-engineered cell, e.g., a population of genetically-engineered cells, in about 1 to about 48 hours or in about 1 to about 24 hours, e.g., in about 24 hours or less, is between about 0.1 ng/ml to about 500 ng/ml. In certain embodiments, the amount of the skin therapeutic secreted by a genetically-engineered cell, e.g., a population of genetically-engineered cells, in about 1 to about 48 hours or in about 1 to about 24 hours, e.g., in about 24 hours or less, is between about 1 ng/ml to about 400 ng/ml. In certain embodiments, the amount of the skin therapeutic secreted by a genetically-engineered cell, e.g., a population of genetically-engineered cells, in about 1 to about 48 hours or in about 1 to about 24 hours, e.g., in about 24 hours or less, is between about 1 ng/ml to about 350 ng/ml. In certain embodiments, the amount of the skin therapeutic secreted by a genetically-engineered cell, e.g., a population of genetically-engineered cells, in about 1 to about 48 hours or in about 1 to about 24 hours, e.g., in about 24 hours or less, is between about 1 ng/ml to about 300 ng/ml. In certain embodiments, the amount of the skin therapeutic secreted by a genetically-engineered cell, e.g., a population of genetically-engineered cells, in about 1 to about 48 hours or in about 1 to about 24 hours, e.g., in about 24 hours or less, is between about 1 ng/ml to about 250 ng/ml. In certain embodiments, the amount of the skin therapeutic secreted by a genetically-engineered cell, e.g., a population of genetically-engineered cells, in about 1 to about 48 hours or in about 1 to about 24 hours, e.g., in about 24 hours or less, is between about 1 ng/ml to about 200 ng/ml. In certain embodiments, the amount of the skin therapeutic secreted by a genetically-engineered cell, e.g., a population of genetically-engineered cells, in about 1 to about 48 hours or in about 1 to about 24 hours, e.g., in about 24 hours or less, is between about 10 ng/ml to about 300 ng/ml. In certain embodiments, the amount of the skin therapeutic secreted by a genetically-engineered cell, e.g., a population of genetically-engineered cells, in about 1 to about 48 hours or in about 1 to about 24 hours, e.g., in about 24 hours or less, is between about 50 ng/ml to about 250 ng/ml.

In certain embodiments, cells of the present disclosure can include a nucleic acid that encodes one or more skin therapeutics disclosed herein. For example, but not by way of limitation, nucleic acids of the present disclosure encoding one or more of the skin therapeutics can be introduced into cells, e.g., yeast cells, using vectors, such as plasmid vectors and cell transformation techniques such as electroporation, heat shock and others known to those skilled in the art and described herein. In certain embodiments, the genetic molecular components are introduced into the cell to persist as a plasmid or integrate into the genome. For example, but not by way of limitation, the nucleic acid can be incorporated into the genome of the genetically-engineered cell. In certain embodiments, the cells can be engineered to chromosomally integrate a polynucleotide of one or more genetic molecular components described herein, using methods identifiable to skilled persons upon reading the present disclosure. In certain embodiments, a nucleic acid encoding a skin therapeutic of the present disclosure, e.g., peptide and/or protein, can be inserted into the genome of a genetically engineered cell using a CRISPR/Cas9 system.

In certain embodiments, a nucleic acid encoding one or more skin therapeutics is introduced into the yeast cell either as a construct or a plasmid. In certain embodiments, a nucleic acid can comprise one or more regulatory regions such as promoters, transcription factor binding sites, operators, activator binding sites, repressor binding sites, enhancers, protein-protein binding domains, RNA binding domains, DNA binding domains, and other control elements known to a person skilled in the art. For example, but not by way of limitation, a nucleic acid encoding a skin therapeutic of the present disclosure is introduced into the yeast cell either as a construct or a plasmid in which it is operably linked to a promoter active in the yeast cell or such that it is inserted into the yeast cell genome at a location where it is operably linked to a suitable promoter. Non-limiting examples of suitable yeast promoters include, but are not limited to, constitutive promoters pTef1, pPgk1, pCyc1, pAdh1, pKex1, pTdh3, pTpi1, pPyk1 and pHxt7 and inducible promoters pGal1, pCup1, pMet15, pFig1, pFus1, GAP, P GCW14 and variants thereof. In certain embodiments, a variant of Tef1 is scTef1. In certain embodiments, a nucleic acid can include a constitutively active promoter, e.g., pTdh3. In certain embodiments, a nucleic acid can include an inducible promoter, e.g., pGal1, pFus1 or pFig1. In certain embodiments, a nucleic acid can include an inducible promoter, e.g., pGal1. In certain embodiments, a nucleic acid can include a constitutively active promoter, e.g., pCyc1. In certain embodiments, a nucleic acid can include a constitutively active promoter, e.g., pTef1. For example, but not by way of limitation, the nucleotide sequence encoding the skin therapeutic can be coupled to the pTef1 promoter or a variant thereof. In certain embodiments, a nucleic acid can include a constitutively active promoter, e.g., pAdh1. For example, but not by way of limitation, the nucleotide sequence encoding the skin therapeutic can be coupled to the pAdh1 promoter or a variant thereof. For example, but not by way of limitation, the nucleotide sequence encoding the skin therapeutic can be coupled to the pTdh3 promoter or a variant thereof. In certain embodiments, the skin therapeutic is continuously expressed by being under the control of a constitutively active promoter.

In certain embodiments, a nucleic acid encoding one or more of the skin therapeutics can further include a transcription factor for regulation expression of the skin therapeutic encoded by the nucleic acid. Alternatively and/or additionally, a second nucleic or an additional nucleic acid can be introduced into the cells to express a transcription factor for regulation expression of the skin therapeutic encoded by the nucleic acid. Non-limiting examples of such transcription factors include Abf1p, Aca1p, Ace2p, Adr1p, Aft1p, Aft2p, Arg80p, Arg81p, Arr1p, Ash1p, Azf1p, Bas1p, Cad1p, Cat8p, Cbf1p, Cha4p, Cha4p, Cin5p, Com2p, Crz1p, Cst6p, Cup2p, Da180p, Da181p, Da182p, Ecm22p, Fkh1p, Fkh2p, F1o8p, Fzf1p, Ga14p, Gat1p, Gcn4p, Gcr1p, Gis1p, G1n3p, Gon3p, Gsm1p, Gzf3p, Haa1p, Hac1p, Hap1p, Hap2p, Hap3p, Hap4p, Hap5p, Hcm1p, Hot1p, Hsf1p, Ime1p, Ino2p, Ino4p, Ino4p, Ixr1p, Kar4p, Leu3p, Lys14p, Mac 1p, Ma163p, Mbp1p, Mcm1p, Met31p, Met32p, Met4p, Mig1p, Mig2p, Mig3p, Mot2p, Mot3p, Msn2p, Msn4p, Mss11p, Ndt80p, Nrg1p, Nrg2p, Oaf1p, Pdr1p, Pdr3p, Pdr8p, Pho2p, Pho4p, Pip2p, Ppr1p, Put3p, Rap1p, Rcs1p, Rds1p, Reb1p, Rfx1p, Rgt1p, Rim101p, R1m1p, Rme1p, Rof1p, Rox1p, Rph1p, Rpn4p, Rtg1p, Rtg3p, Sf11p, Sip4p, Skn7p, Sko1p, Smp1p, Stb4p, Stb5p, Stb5p, Ste12p, Stp1p, Stp2p, Sumip, Swi4p, Swi5p, Tda9p, Tea1p, Tec1p, Tye7p, Uga3p, Ume6p, Upc2p, Usv1p, War1p, Xbp1p, YER130c, YFL052w, YHR177w, YJL103C, YML081w, YPL230w, Yap1p, Yap3p, Yap5p, Yrr1p, Zap1p and Znf1p. In certain embodiments, a nucleic acid introduced into a genetically-engineered cell of the present disclosure includes one or more DNA binding domains for a transcription factor. In certain embodiments, the DNA binding domain is a zinc finger DNA binding domain. In certain embodiments, the zinc finger DNA binding domain is ZF43-8. In certain embodiments, the transcription factor comprises one or more domains from different proteins. For example, but not by way of limitation, a transcription factor for use in the present disclosure can include an inducer binding domain, e.g., a β-estradiol binding domain, e.g., derived from the human estrogen receptor, and/or a transcription activation domain, e.g., derived from VP64.

In certain embodiments, a nucleic acid encoding one or more of the skin therapeutics can further include a secretion signal peptide encoded by the nucleic acid. Non-limiting examples of secretion signal peptides are disclosed herein in Section II, Table 5 and in Example 1. In certain embodiments, the secretion signal peptide is the mating factor alpha-1, SED1 or SUC2. In certain embodiments, a nucleic acid encoding one or more of the skin therapeutics can further include a mating factor alpha-1 secretion signal peptide encoded by the nucleic acid. In certain embodiments, a nucleic acid encoding one or more of the skin therapeutics can further include a SED1 secretion signal peptide encoded by the nucleic acid. In certain embodiments, a nucleic acid encoding one or more of the skin therapeutics can further include a SUC2 secretion signal peptide encoded by the nucleic acid.

In certain embodiments, a nucleic acid encoding one or more of the skin therapeutics can be inserted into the genome of the cell, e.g., yeast cell. For example, but not by way of limitation, one or more nucleic acids encoding a skin therapeutic of the present disclosure, e.g., peptide and/or protein, can be inserted into the Ste2, Ste3 and/or HO locus of the cell. In certain embodiments, the one or more nucleic acids can be inserted into one or more loci that minimally affects the cell, e.g., in an intergenic locus or a gene that is not essential and/or does not affect growth, proliferation and cell signaling.

In certain embodiments, one or more endogenous genes of the genetically-engineered cells can be knocked out and/or mutated, e.g., knocked out by a genetic engineering system. Alternatively or additionally, extra copies of endogenous genes of the genetically-engineered cells can be knocked in, e.g., knocked in by a genetic engineering system. Various genetic engineering systems known in the art can be used. Non-limiting examples of such systems include the Clustered regularly-interspaced short palindromic repeats (CRISPR)/Cas system, the zinc-finger nuclease (ZFN) system, the transcription activator-like effector nuclease (TALEN) system, use of yeast endogenous homologous recombination and the use of interfering RNAs.

In certain non-limiting embodiments, a CRISPR/Cas9 system is employed to knock out and/or knock in one or more endogenous genes in the genetically engineered cell. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9) and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule and referred to alternatively as chimeric) or modular (comprising more than one, and typically two, separate RNA molecules, such as a crRNA and a tracrRNA, which are usually associated with one another, for instance by duplexing).

In certain embodiments, a genetically-engineered cell of the present disclosure can be modified to have increased expression of proteins involved in disulfide bond formation, e.g., to increase secretion of wound-healing agents that have disulfide bonds. In certain embodiments, a genetically-engineered cell of the present disclosure can be modified to have increased expression of protein disulfide isomerase 1 (PDI1), e.g., by incorporating one or more additional copies of the PDI1 gene into the genetically-engineered cells. For example, but not by way of limitation, at least one additional copy of the PDI1 gene can be knocked into a genetically engineered cell described herein by a genome editing system, e.g., CRISPR/Cas. Alternatively, increased expression of such genes can be achieved by introducing a nucleic acid comprising the gene into the genetically-engineered cell. In certain embodiments, a fungal cell genetically engineered to express PDGF-b (which has three intrachain disulfide bonds), EGF (which has three disulfide bonds), CXCL12 (which has two disulfide bonds) and/or LEP (which has one intrachain disulfide bond) has been further modified to have increased expression of PDI1, e.g., by incorporating one or more nucleic acids, e.g., exogenous nucleic acids, encoding PDI1 into the genetically-engineered cells.

In certain embodiments, the expression of an endogenous gene can be downregulated, eliminated and/or decreased. For example, but not by way of limitation, the expression of genes that are involved in N-glycosylation can be downregulated, eliminated and/or decreased. In certain embodiments, expression of Och1p, Vacuolar Proteinase B (PRB1), BarI protease and/or Aspartic Protease 3 (YAP3) can be downregulated, eliminated and/or decreased. In certain embodiments, expression of Och1p can be downregulated, eliminated and/or decreased. In certain embodiments, expression of PRB1 can be downregulated, eliminated and/or decreased. In certain embodiments, expression of BarI protease can be downregulated, eliminated and/or decreased. In certain embodiments, the expression of these genes can be downregulated, eliminated and/or decreased by knocking out these genes by a genome editing system, e.g., CRISPR/Cas. In certain embodiments, the expression of specific genes can be downregulated by the overexpression of a downstream or modifying protein. In certain embodiments, expression of XIST can be upregulated, e.g., by the introduction of a nucleic acid that encodes for XIST, into the genetically-engineered fungal cells. For example, but not by way of limitation, overexpression of XIST leads to x-linked inactivation of numerous genes. In certain embodiments, expression of GCG can be upregulated, e.g., by the introduction of a nucleic acid that encodes for GCG, into the genetically-engineered fungal cells. In certain embodiments, the overexpression of GCG leads to decreased insulin production.

In certain embodiments, a homolog of a nucleotide sequence disclosed herein can be a polynucleotide having changes in one or more nucleotide bases that can result in substitution of one or more amino acids, but do not affect the functional properties of the polypeptide or protein encoded by the nucleotide sequence. Homologs can also include polynucleotides having modifications such as deletion, addition or insertion of nucleotides that do not substantially affect the functional properties of the resulting polynucleotide or transcript. Alterations in a polynucleotide that result in the production of a chemically equivalent amino acid at a given site, but do not affect the functional properties of the encoded polypeptide, are well known in the art.

In certain embodiments, a homolog of a peptide, polypeptide or protein disclosed herein can be a peptide, polypeptide or protein having changes in one or more amino acids but do not affect the functional properties of the skin therapeutic. Alterations in a peptide, polypeptide or protein that do not affect the functional properties of the peptide, polypeptide or protein, are well known in the art, e.g., conservative substitutions. It is therefore understood that the disclosure encompasses more than the specific exemplary polynucleotide or amino acid sequences and includes functional equivalents thereof.

The cells to be used in the present disclosure can be genetically engineered using recombinant techniques known to those of ordinary skill in the art. Production and manipulation of the polynucleotides described herein are within the skill in the art and can be carried out according to recombinant techniques described, for example, in Sambrook et al. 1989. Molecular Cloning: A Laboratory Manual, 2d ed., Cold Spring Harbor Laboratory Press, Cold Spring Harbor, N.Y. and Innis et al. (eds). 1995. PCR Strategies, Academic Press, Inc., San Diego.

### IV. Pharmaceutical Compositions

The present disclosure further provides pharmaceutical compositions comprising a genetically-engineered cell described herein for use according to the disclosed methods. In certain embodiments, the pharmaceutical compositions include one or more live and/or intact genetically-engineered cells, e.g., fungal cells, expressing one or more skin therapeutics.

In certain embodiments, a pharmaceutical composition for use accordingly to the present disclosure can be formulated for rectal administration, oral administration, vaginal administration or topical administration. In certain embodiments, the pharmaceutical composition is formulated for topical administration. In certain embodiments, a pharmaceutical composition of the present disclosure is not administered orally, i.e., not formulated for oral administration. In certain embodiments, a pharmaceutical composition of the present disclosure is not administered intraocularly, i.e., not formulated for intraocular administration.

In certain embodiments, the pharmaceutical composition includes a genetically-engineered cell, disclosed herein, and a pharmaceutically acceptable carrier. In certain embodiments, the pharmaceutically acceptable carrier includes any carrier which does not interfere with the effectiveness of the biological activity of the active ingredients, e.g., the genetically-engineered cell and/or the skin therapeutic, and that is not toxic to the patient to whom it is administered. Non-limiting examples of suitable pharmaceutical carriers include phosphate-buffered saline solutions, water, emulsions, such as oil/water emulsions, various types of wetting agents and sterile solutions. Additional non-limiting examples of pharmaceutically acceptable carriers can include polymers, gels, bioabsorbable matrix materials, implantation elements containing the yeast and/or any other suitable vehicle, delivery or dispensing means or material. Such carriers can be formulated by conventional methods and can be administered to the subject.

In certain embodiments, a pharmaceutical composition of the present disclosure can include a genetically-engineered cell, e.g., a genetically-engineered fungal cell, that expresses and/or secretes at least one growth factor or a derivative thereof, e.g., EGF and/or PDGF.

In certain embodiments, a pharmaceutical composition of the present disclosure can include a genetically-engineered cell, e.g., a genetically-engineered fungal cell, that expresses and/or secretes at least one cytokine or a derivative thereof, e.g., Leptin.

In certain embodiments, a pharmaceutical composition of the present disclosure can include a genetically-engineered cell, e.g., a genetically-engineered fungal cell, that expresses and/or secretes at least one chemokine or a derivative thereof, e.g., CXCL12.

In certain embodiments, a pharmaceutical composition of the present disclosure can include a genetically-engineered cell, e.g., a genetically-engineered fungal cell, that expresses and/or secretes at least one protease inhibitor or a derivative thereof, e.g., TIMP1 and/or TIMP2.

In certain embodiments, a pharmaceutical composition of the present disclosure can include a genetically-engineered cell that expresses and/or secretes (1) a growth factor or a derivative thereof, e.g., EGF and/or PDGF, and (2) a cytokine or a derivative thereof, e.g., Leptin.

In certain embodiments, a pharmaceutical composition of the present disclosure can include a genetically-engineered cell that expresses and/or secretes (1) a growth factor or a derivative thereof, e.g., EGF and/or PDGF, and (2) a chemokine or a derivative thereof, e.g., CXCL12.

In certain embodiments, a pharmaceutical composition of the present disclosure can include a genetically-engineered cell that expresses and/or secretes (1) a cytokine or a derivative thereof, e.g., Leptin, and (2) a chemokine or a derivative thereof, e.g., CXCL12.

In certain embodiments, a pharmaceutical composition of the present disclosure can include a genetically-engineered cell that expresses and/or secretes (1) a growth factor or a derivative thereof, e.g., EGF and/or PDGF, and (2) a protease inhibitor or a derivative thereof, e.g., TIMP1 and/or TIMP2.

In certain embodiments, a pharmaceutical composition of the present disclosure can include a genetically-engineered cell that expresses and/or secretes (1) a protease inhibitor or a derivative thereof, e.g., TIMP1 and/or TIMP2, and (2) a chemokine or a derivative thereof, e.g., CXCL12.

In certain embodiments, a pharmaceutical composition of the present disclosure can include a genetically-engineered cell that expresses and/or secretes (1) a cytokine or a derivative thereof, e.g., Leptin, and (2) a protease inhibitor or a derivative thereof, e.g., TIMP1 and/or TIMP2.

In certain embodiments, a pharmaceutical composition of the present disclosure can include a genetically-engineered cell that expresses and/or secretes (1) a growth factor or a derivative thereof, e.g., EGF and/or PDGF, (2) a cytokine or a derivative thereof, e.g., Leptin, and (3) a chemokine or a derivative thereof, e.g., CXCL12.

In certain embodiments, a pharmaceutical composition of the present disclosure can include a genetically-engineered cell that expresses and/or secretes (1) a protease inhibitor or a derivative thereof, e.g., TIMP1 and/or TIMP2, (2) a cytokine or a derivative thereof, e.g., Leptin, and (3) a chemokine or a derivative thereof, e.g., CXCL12.

In certain embodiments, a pharmaceutical composition of the present disclosure can include a genetically-engineered cell that expresses and/or secretes (1) a growth factor or a derivative thereof, e.g., EGF and/or PDGF, (2) a protease inhibitor or a derivative thereof, e.g., TIMP1 and/or TIMP2, and (3) a chemokine or a derivative thereof, e.g., CXCL12.

In certain embodiments, a pharmaceutical composition of the present disclosure can include a genetically-engineered cell that expresses and/or secretes (1) a growth factor or a derivative thereof, e.g., EGF and/or PDGF, (2) a cytokine or a derivative thereof, e.g., Leptin, and (3) a protease inhibitor or a derivative thereof, e.g., TIMP1 and/or TIMP2.

In certain embodiments, a pharmaceutical composition of the present disclosure can include a genetically-engineered cell that expresses and/or secretes (1) a growth factor or a derivative thereof, e.g., EGF and/or PDGF, (2) a cytokine or a derivative thereof, e.g., Leptin, (3) a protease inhibitor or a derivative thereof, e.g., TIMP1 and/or TIMP2, and (4) a chemokine or a derivative thereof, e.g., CXCL12.

In certain embodiments, a pharmaceutical composition of the present disclosure can include a genetically-engineered cell that expresses and/or secretes a growth factor inhibitor. In certain embodiments, a pharmaceutical composition of the present disclosure can include a genetically-engineered cell that expresses and/or secretes a VEGF inhibitor, e.g., IL-4.

In certain embodiments, a pharmaceutical composition of the present disclosure can include a genetically-engineered cell that expresses and/or secretes a TGF-β1 inhibitor. In certain embodiments, a pharmaceutical composition of the present disclosure can include a genetically-engineered cell that expresses and/or secretes a peptide TGF-β1 inhibitor, e.g., TSLDASIIWAMMQN (SEQ ID NO: 9) or TSLDASIIWAMMQNA (SEQ ID NO: 12).

In certain embodiments, a pharmaceutical composition of the present disclosure can include a genetically-engineered cell that expresses and/or secretes an extracellular matrix protein, e.g., a collagen. In certain embodiments, a pharmaceutical composition of the present disclosure can include a genetically-engineered cell that expresses and/or secretes Type VII Collagen or a fragment thereof. In certain embodiments, a pharmaceutical composition of the present disclosure includes a genetically-engineered cell that expresses and/or secretes elastin.

In certain embodiments, a pharmaceutical composition of the present disclosure includes a genetically-engineered cell of the present disclosure that expresses and/or secretes an antimicrobial agent. In certain embodiments, a pharmaceutical composition of the present disclosure includes a genetically-engineered that expresses and/or secretes an antimicrobial peptide. In certain embodiments, a pharmaceutical composition of the present disclosure includes a genetically-engineered cell that expresses and/or secretes an antimicrobial peptide selected from the group consisting of RcA1b-PepI, RcA1b-PepII, RcA1b-PepII, lucifensin, lucifensin II, lucilin, pexiganan acetate (MSI-78), D2A21/D4E1, granulysin, synthetic granulysin-derived peptides, cathelicidin antimicrobial peptide (LL-37) or analogs thereof and a combination thereof.

In certain embodiments, a pharmaceutical composition of the present disclosure includes a genetically-engineered cell that expresses and/or secretes a killer toxin. In certain embodiments, a pharmaceutical composition of the present disclosure includes a genetically-engineered cell that expresses and/or secretes K1, K2 and/or K28 from Saccharomyces cerevisiae.

In certain embodiments, a pharmaceutical composition of the present disclosure includes a genetically-engineered cell that expresses and/or secretes a defensin, e.g., an α-, β- or θ-defensin. In certain embodiments, a pharmaceutical composition of the present disclosure includes a genetically-engineered cell that expresses and/or secretes β-defensin-1 (hBD-1), β-defensin-2 (hBD-2), β-defensin-3 (hBD-3), neutrophil peptide 1 (HNP1), neutrophil peptide 2 (HNP2), neutrophil peptide 3 (HNP3), neutrophil peptide 4 (HNP4), human defensin 5 (HD5) and/or human defensin 6 (HD6). In certain embodiments, a pharmaceutical composition of the present disclosure includes a genetically-engineered cell that expresses and/or secretes β-defensin-3 (hBD-3).

In certain embodiments, a pharmaceutical composition of the present disclosure includes a genetically-engineered cell that expresses and/or secretes an inhibitor of RelA, e.g., an RNAi targeting RelA.

In certain embodiments, a pharmaceutical composition of the present disclosure includes a genetically-engineered cell that expresses and/or secretes an anti-staphylococcus bactericidal protein.

In certain embodiments, a pharmaceutical composition of the present disclosure includes a genetically-engineered cell that expresses and/or secretes a xeroderma pigmentosum complementation protein (XP), e.g., XPA, XPB, XPC (human RAD4), XPD, XPE, XPF or XPG.

In certain embodiments, a pharmaceutical composition of the present disclosure includes a genetically-engineered cell that expresses and/or secretes azelaic acid.

In certain embodiments, a pharmaceutical composition of the present disclosure includes a genetically-engineered cell that expresses and/or secretes hyaluronic acid.

In certain embodiments, a pharmaceutical composition of the present disclosure comprises a multi-cell system that can be used for the generation of skin therapeutics that require the assembly of multiple components in a coordinated manner, where each cell is configured to produce a component of a skin therapeutic. In certain embodiments, a multi-cell system can be used for the generation of multiple different skin therapeutics. In certain embodiments, a multi-cell system can be used for the generation and/or secretion of 2, 3, 4, 5, 6, 7, 8, 9 or 10 different skin therapeutics. Non-limiting examples of such multi-cell systems are disclosed PCT/US2020/030795, the contents of which is incorporated herein in its entirety.

In certain embodiments, a pharmaceutical composition of the present disclosure can include two or more genetically-engineered cells, e.g., three or more, four or more or five or more, that each express and/or secrete a different skin therapeutic. In certain embodiments, a pharmaceutical composition of the present disclosure including two or more genetically-engineered cells that each express and/or secrete a different skin therapeutic is more effective in treating a condition compared to a pharmaceutical composition that includes a genetically-engineered cell expressing and/or secreting a single skin therapeutic.

In certain embodiments, a pharmaceutical composition of the present disclosure includes (1) a first genetically-engineered cell that expresses and/or secretes a first skin therapeutic and (2) a second genetically-engineered cell that expresses and/or secretes a second skin therapeutic. For example, but not by way of limitation, a pharmaceutical composition of the present disclosure includes (1) a first genetically-engineered cell that expresses and/or secretes a first skin therapeutic disclosed herein in Section II and (2) a second genetically-engineered cell that expresses and/or secretes a second skin therapeutic disclosed herein in Section II, where the first and second skin therapeutic are different.

In certain embodiments, a pharmaceutical composition of the present disclosure includes (1) a first genetically-engineered cell that expresses and/or secretes a first skin therapeutic, (2) a second genetically-engineered cell that expresses and/or secretes a second skin therapeutic and (3) a third genetically-engineered cell that expresses and/or secretes a third skin therapeutic. For example, but not by way of limitation, a pharmaceutical composition of the present disclosure includes (1) a first genetically-engineered cell that expresses and/or secretes a first skin therapeutic disclosed herein in Section II, (2) a second genetically-engineered cell that expresses and/or secretes a second skin therapeutic disclosed herein in Section II and (3) a third genetically-engineered cell that expresses and/or secretes a third skin therapeutic disclosed herein in Section II, where the first, second and third skin therapeutic are different.

In certain embodiments, a pharmaceutical composition of the present disclosure includes (1) a first genetically-engineered cell that expresses and/or secretes a first skin therapeutic, (2) a second genetically-engineered cell that expresses and/or secretes a second skin therapeutic, (3) a third genetically-engineered cell that expresses and/or secretes a third skin therapeutic and (4) a fourth genetically-engineered cell that expresses and/or secretes a fourth skin therapeutic. For example, but not by way of limitation, a pharmaceutical composition of the present disclosure includes (1) a first genetically-engineered cell that expresses and/or secretes a first skin therapeutic disclosed herein in Section II, (2) a second genetically-engineered cell that expresses and/or secretes a second skin therapeutic disclosed herein in Section II, (3) a third genetically-engineered cell that expresses and/or secretes a third skin therapeutic disclosed herein in Section II and (4) a fourth genetically-engineered cell that expresses and/or secretes a fourth skin therapeutic disclosed herein in Section II, where the first, second, third and fourth skin therapeutic are different.

In certain embodiments, a pharmaceutical composition of the present disclosure includes (1) a first genetically-engineered cell that expresses and/or secretes a first skin therapeutic, (2) a second genetically-engineered cell that expresses and/or secretes a second skin therapeutic, (3) a third genetically-engineered cell that expresses and/or secretes a third skin therapeutic, (4) a fourth genetically-engineered cell that expresses and/or secretes a fourth skin therapeutic and (5) a fifth genetically-engineered cell that expresses and/or secretes a fifth skin therapeutic. For example, but not by way of limitation, a pharmaceutical composition of the present disclosure includes (1) a first genetically-engineered cell that expresses and/or secretes a first skin therapeutic disclosed herein in Section II, (2) a second genetically-engineered cell that expresses and/or secretes a second skin therapeutic disclosed herein in Section II, (3) a third genetically-engineered cell that expresses and/or secretes a third skin therapeutic disclosed herein in Section II, (4) a fourth genetically-engineered cell that expresses and/or secretes a fourth skin therapeutic disclosed herein in Section II and (5) a fifth genetically-engineered cell that expresses and/or secretes a fifth skin therapeutic disclosed herein in Section II, where the first, second, third, fourth and fifth skin therapeutic are different.

In certain embodiments, a pharmaceutical composition of the present disclosure includes (1) a first genetically-engineered cell that expresses and/or secretes a first skin therapeutic, (2) a second genetically-engineered cell that expresses and/or secretes a second skin therapeutic, (3) a third genetically-engineered cell that expresses and/or secretes a third skin therapeutic, (4) a fourth genetically-engineered cell that expresses and/or secretes a fourth skin therapeutic, (5) a fifth genetically-engineered cell that expresses and/or secretes a fifth skin therapeutic and (6) a sixth genetically-engineered cell that expresses and/or secretes a sixth skin therapeutic. For example, but not by way of limitation, a pharmaceutical composition of the present disclosure includes (1) a first genetically-engineered cell that expresses and/or secretes a first skin therapeutic disclosed herein in Section II, (2) a second genetically-engineered cell that expresses and/or secretes a second skin therapeutic disclosed herein in Section II, (3) a third genetically-engineered cell that expresses and/or secretes a third skin therapeutic disclosed herein in Section II, (4) a fourth genetically-engineered cell that expresses and/or secretes a fourth skin therapeutic disclosed herein in Section II, (5) a fifth genetically-engineered cell that expresses and/or secretes a fifth skin therapeutic disclosed herein in Section II and (6) a sixth genetically-engineered cell that expresses and/or secretes a sixth skin therapeutic disclosed herein in Section II, where the first, second, third, fourth and fifth skin therapeutic are different.

In certain embodiments, a pharmaceutical composition of the present disclosure includes a first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth and/or twenty-fifth genetically-engineered cell that expresses and/or secretes a fourth skin therapeutic disclosed herein in Section II, where each genetically-engineered cell expresses a different skin therapeutic.

In certain embodiments, a pharmaceutical composition of the present disclosure includes (1) a first genetically-engineered cell that expresses and/or secretes a growth factor or a derivative thereof, e.g., EGF and/or PDGF, and (2) a second genetically-engineered cell that expresses and/or secretes a chemokine or a derivative thereof, e.g., CXCL12. In certain embodiments, a pharmaceutical composition of the present disclosure includes (1) a first genetically-engineered cell that expresses and/or secretes a growth factor or a derivative thereof, e.g., EGF and/or PDGF, and (2) a second genetically-engineered cell that expresses and/or secretes a cytokine or a derivative thereof, e.g., Leptin. In certain embodiments, a pharmaceutical composition of the present disclosure includes (1) a first genetically-engineered cell that expresses and/or secretes a chemokine or a derivative thereof, e.g., CXCL12, and (2) a second genetically-engineered cell that expresses and/or secretes a cytokine or a derivative thereof, e.g., Leptin. In certain embodiments, a pharmaceutical composition of the present disclosure includes (1) a first genetically-engineered cell that expresses and/or secretes a growth factor or a derivative thereof, e.g., EGF and/or PDGF, and (2) a second genetically-engineered cell that expresses and/or secretes a protease inhibitor or a derivative thereof, e.g., TIMP1 and/or TIMP2. In certain embodiments, a pharmaceutical composition of the present disclosure includes (1) a first genetically-engineered cell that expresses and/or secretes a chemokine or a derivative thereof, e.g., CXCL12, and (2) a second genetically-engineered cell that expresses and/or secretes a protease inhibitor or a derivative thereof, e.g., TIMP1 and/or TIMP2. In certain embodiments, a pharmaceutical composition of the present disclosure includes (1) a first genetically-engineered cell that expresses and/or secretes a cytokine or a derivative thereof, e.g., Leptin, and (2) a second genetically-engineered cell that expresses and/or secretes a protease inhibitor or a derivative thereof, e.g., TIMP1 and/or TIMP2.

In certain embodiments, a pharmaceutical composition of the present disclosure includes (1) a first genetically-engineered cell that expresses and/or secretes EGF, and (2) a second genetically-engineered cell that expresses and/or secretes CXCL12.

In certain embodiments, a pharmaceutical composition of the present disclosure includes (1) a first genetically-engineered cell that expresses and/or secretes a growth factor or a derivative thereof, e.g., EGF and/or PDGF, (2) a second genetically-engineered cell that expresses and/or secretes a protease inhibitor or a derivative thereof, e.g., TIMP1 and/or TIMP2, and (3) a third genetically-engineered cell that expresses and/or secretes a cytokine or a derivative thereof, e.g., Leptin. In certain embodiments, a pharmaceutical composition of the present disclosure includes (1) a first genetically-engineered cell that expresses and/or secretes a growth factor or a derivative thereof, e.g., EGF and/or PDGF, (2) a second genetically-engineered cell that expresses and/or secretes a protease inhibitor or a derivative thereof, e.g., TIMP1 and/or TIMP2, and (3) a third genetically-engineered cell that expresses and/or secretes a chemokine or a derivative thereof, e.g., CXCL12.

In certain embodiments, a pharmaceutical composition of the present disclosure includes (1) a first genetically-engineered cell that expresses and/or secretes a growth factor or a derivative thereof, e.g., EGF and/or PDGF, (2) a second genetically-engineered cell that expresses and/or secretes a protease inhibitor or a derivative thereof, e.g., TIMP1 and/or TIMP2, (3) a third genetically-engineered cell that expresses and/or secretes a chemokine or a derivative thereof, e.g., CXCL12, and (4) a fourth genetically-engineered cell that expresses and/or secretes a cytokine or a derivative thereof, e.g., Leptin.

In certain embodiments, a pharmaceutical composition of the present disclosure can include nutrients for promoting the growth of the one or more genetically-engineered cells present in the composition. For example, but not by way of limitation, a pharmaceutical composition can include vitamins, e.g., water-soluble vitamins, carbohydrates, peptides, amino acids and/or salts.

In certain embodiments, the pharmaceutical compositions suitable for use in the present disclosure can include compositions where the genetically-engineered cells are contained in a therapeutically effective amount. The therapeutically effective amount of an active ingredient can vary depending on the active ingredient, e.g., the genetically-engineered cell and/or the skin therapeutic, formulation used, the condition and its severity, and the age, weight, etc., of the subject to be treated. In certain embodiments, the pharmaceutical composition can include at least about 10², about 10³, about 10⁴, about 10⁵, about 10⁶, about 10⁷, about 10⁸, about 10⁹ or about 10¹⁰ genetically-engineered cells. In certain embodiments, the pharmaceutical compositions can include genetically-engineered cells in concentrations of about 10² cells/ml, about 10³ cells/ml, about 10⁴ cells/ml, about 10⁵ cells/ml, about 10⁶ cells/ml, about 10⁷ cells/ml, about 10⁸ cells/ml, about 10⁹ cells/ml or about 10¹⁰ cells/ml. In certain embodiments, the pharmaceutical compositions can include genetically-engineered cells in concentrations from about 1×10³ cells/ml to about 1×10¹⁰ cells/ml. In certain embodiments, the pharmaceutical compositions can include genetically-engineered cells in concentrations from about 1×10⁴ cells/ml to about 1×10¹⁰ cells/ml of the live genetically-engineered fungal cells. In certain embodiments, the pharmaceutical compositions can include genetically-engineered cells in concentrations from about 1×10³ cells/ml to about 1×10⁹ cells/ml of the live genetically-engineered fungal cells. In certain embodiments, the pharmaceutical compositions can include genetically-engineered cells in concentrations from about 1×10⁴ cells/ml to about 1×10⁸ cells/ml, from about 1×10⁵ cells/ml to about 1×10⁸ cells/ml, from about 1×10⁶ cells/ml to about 1×10⁸ cells/ml, from about 1×10⁵ cells/ml to about 1×10⁷ cells/ml, from about 1×10⁶ cells/ml to about 1×10⁷ cells/ml or from about 1×10⁴ cells/ml to about 1×10⁷ cells/ml. In certain embodiments, the pharmaceutical compositions can include genetically-engineered cells in concentrations from about 1×10⁶ cells/ml to about 2×10⁷ cells/ml, as shown in FIG. 5B. In certain embodiments, the pharmaceutical compositions can include genetically-engineered cells in concentrations from about 2×10⁶ cells/ml to about 2×10⁷ cells/ml.

In certain embodiments, the pharmaceutical compositions suitable for use in the present disclosure can include compositions where the genetically-engineered cells express and/or secrete the skin therapeutic in a therapeutically effective amount. In certain embodiments, the pharmaceutical compositions can include a population of genetically-engineered cells that express and/or secrete from about 1 pg and about 10 g of the skin therapeutic in about 1 to about 24 hours. In certain embodiments, the pharmaceutical compositions can include a population of genetically-engineered cells that express and/or secrete from about 1 pg/ml to about 200,000 pg/ml, e.g., between about 100 pg/ml to about 200,000 pg/ml, between about 1,000 pg/ml to about 200,000 pg/ml, between about 5,000 pg/ml to about 200,000 pg/ml, between about 10,000 pg/ml to about 200,000 pg/ml, between about 20,000 pg/ml to about 200,000 pg/ml, between about 30,000 pg/ml to about 200,000 pg/ml, between about 40,000 pg/ml to about 200,000 pg/ml, between about 50,000 pg/ml to about 200,000 pg/ml, between about 60,000 pg/ml to about 200,000 pg/ml, between about 70,000 pg/ml to about 200,000 pg/ml, between about 80,000 pg/ml to about 200,000 pg/ml, between about 90,000 pg/ml to about 200,000 pg/ml, between about 100,000 pg/ml to about 200,000 pg/ml, between about 110,000 pg/ml to about 200,000 pg/ml, between about 120,000 pg/ml to about 200,000 pg/ml, between about 130,000 pg/ml to about 200,000 pg/ml, between about 140,000 pg/ml to about 200,000 pg/ml, between about 150,000 pg/ml to about 200,000 pg/ml, between about 100 pg/ml to about 190,000 pg/ml, between about 100 pg/ml to about 180,000 pg/ml, between about 100 pg/ml to about 170,000 pg/ml, between about 100 pg/ml to about 160,000 pg/ml, between about 100 pg/ml to about 150,000 pg/ml, between about 100 pg/ml to about 140,000 pg/ml, between about 100 pg/ml to about 130,000 pg/ml, between about 100 pg/ml to about 120,000 pg/ml, between about 100 pg/ml to about 100,000 pg/ml, between about 100 pg/ml to about 90,000 pg/ml, between about 100 pg/ml to about 80,000 pg/ml, between about 100 pg/ml to about 70,000 pg/ml, between about 100 pg/ml to about 60,000 pg/ml, between about 100 pg/ml to about 50,000 pg/ml, between about 100 pg/ml to about 40,000 pg/ml, between about 100 pg/ml to about 30,000 pg/ml, between about 100 pg/ml to about 20,000 pg/ml, between about 100 pg/ml to about 10,000 pg/ml, between about 100 pg/ml to about 5,000 pg/ml, between about 1,000 pg/ml to about 100,000 pg/ml, between about 1,000 pg/ml to about 50,000 pg/ml, between about 1,000 pg/ml to about 25,000 pg/ml, between about 1,000 pg/ml to about 10,000 pg/ml or between about 1,000 pg/ml to about 5,000 pg/ml of the skin therapeutic in about 1 to about 24 hours. In certain embodiments, the pharmaceutical compositions can include a population of genetically-engineered cells that express and/or secrete from about 1 pg/ml and about 2,500 pg/ml, e.g., between about 100 pg/ml to about 2,500 pg/ml, between about 1,000 pg/ml to about 2,000 pg/ml or between about 1,500 pg/ml to about 2,500 pg/ml, of the skin therapeutic in about 1 to about 24 hours. In certain embodiments, the pharmaceutical compositions can include a population of genetically-engineered cells that express and/or secrete from about 0.1 ng/ml to about 1,000 ng/ml of the skin therapeutic in about 1 to about 24 hours, e.g., in about 24 hours or less. In certain embodiments, the pharmaceutical compositions can include a population of genetically-engineered cells that express and/or secrete from about 0.1 ng/ml to about 500 ng/ml of the skin therapeutic in about 1 to about 24 hours, e.g., in about 24 hours or less. In certain embodiments, the pharmaceutical compositions can include a population of genetically-engineered cells that express and/or secrete from about 1 ng/ml to about 400 ng/ml of the skin therapeutic in about 1 to about 24 hours, e.g., in about 24 hours or less. In certain embodiments, the pharmaceutical compositions can include a population of genetically-engineered cells that express and/or secrete from about 1 ng/ml to about 350 ng/ml in about 1 to about 24 hours, e.g., in about 24 hours or less. In certain embodiments, the pharmaceutical compositions can include a population of genetically-engineered cells that express and/or secrete from about 1 ng/ml to about 300 ng/ml in about 1 to about 24 hours, e.g., in about 24 hours or less. In certain embodiments, the pharmaceutical compositions can include a population of genetically-engineered cells that express and/or secrete from about 1 ng/ml to about 250 ng/ml in about 1 to about 24 hours, e.g., in about 24 hours or less. In certain embodiments, the pharmaceutical compositions can include a population of genetically-engineered cells that express and/or secrete from about 1 ng/ml to about 200 ng/ml in about 1 to about 24 hours, e.g., in about 24 hours or less. In certain embodiments, the pharmaceutical compositions can include a population of genetically-engineered cells that express and/or secrete from about 10 ng/ml to about 300 ng/ml in about 1 to about 24 hours, e.g., in about 24 hours or less. In certain embodiments, the pharmaceutical compositions can include a population of genetically-engineered cells that express and/or secrete from about 50 ng/ml to about 250 ng/ml in about 1 to about 24 hours, e.g., in about 24 hours or less.

In certain non-limiting embodiments, the pharmaceutical compositions of the present disclosure can be formulated using pharmaceutically acceptable carriers well known in the art that are suitable for rectal and vaginal administration. Such carriers enable the pharmaceutical compositions to be formulated as tablets, pills, capsules, liquids (e.g., viscous liquids), pastes, gels, syrups, slurries, suspensions, suppositories and the like, for topical, rectal and/or oral administration to the patient to be treated.

In certain embodiments, the pharmaceutical compositions of the present disclosure can be formulated using pharmaceutically acceptable carriers well known in the art that are suitable for topical administration. Such carriers enable the pharmaceutical compositions to be formulated as liquids, gels, creams, syrups, pastes, slurries, dispersible powders, suspensions, lotions and the like, for topical administration to the patient to be treated.

In certain embodiments, one or more devices, e.g., an applicator, can be used to administer one or more of the disclosed pharmaceutical compositions.

In certain embodiments, a pharmaceutical composition can include one or more lyophilized or freeze-dried genetically-engineered cells of the present disclosure.

In certain embodiments, pharmaceutical compositions of the present disclosure can further include a second agent for treating a condition of the subject. For example, but not by way of limitation, a pharmaceutical composition of the present disclosure can further include an antimicrobial agent and/or anti-inflammatory agent that is distinct from the skin therapeutic that is expressed and/or secreted from the genetically-engineered cell in the pharmaceutical composition. In certain embodiments, a pharmaceutical composition of the present disclosure can further include an antimicrobial agent and/or anti-inflammatory agent that is the same as the skin therapeutic that is expressed and/or secreted from the genetically-engineered cell in the pharmaceutical composition.

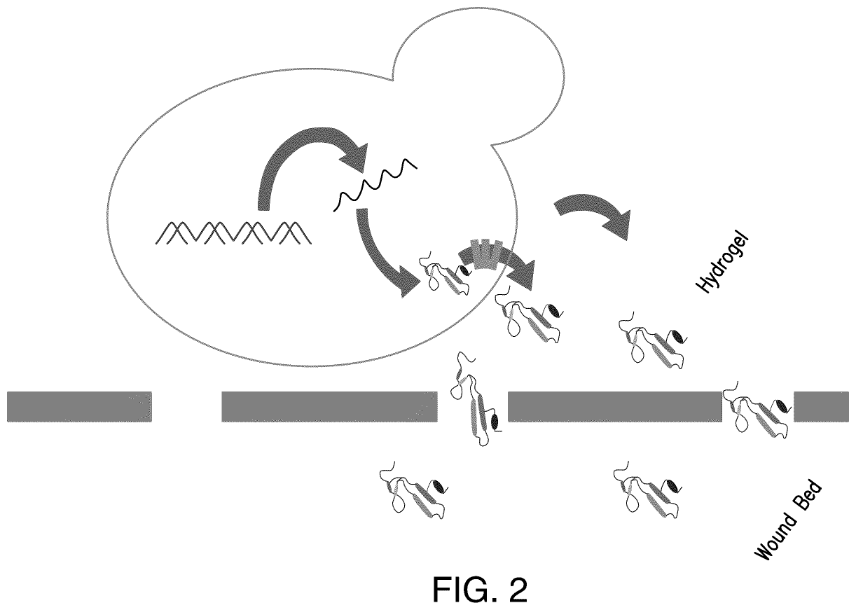

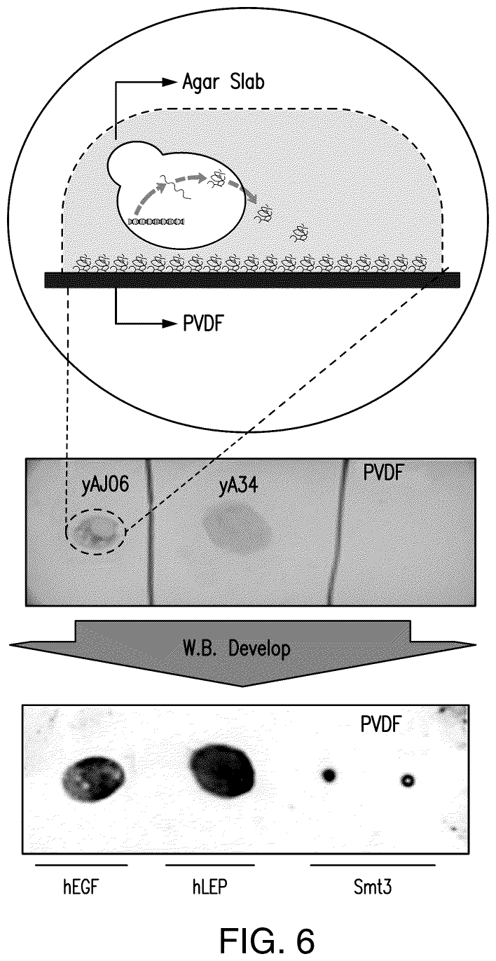

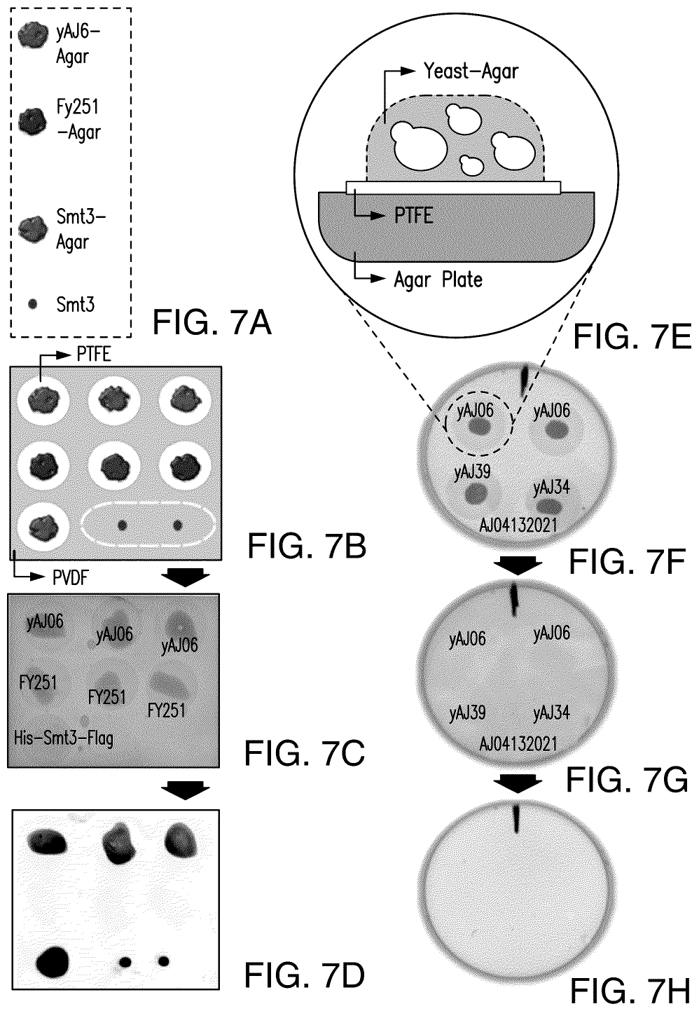

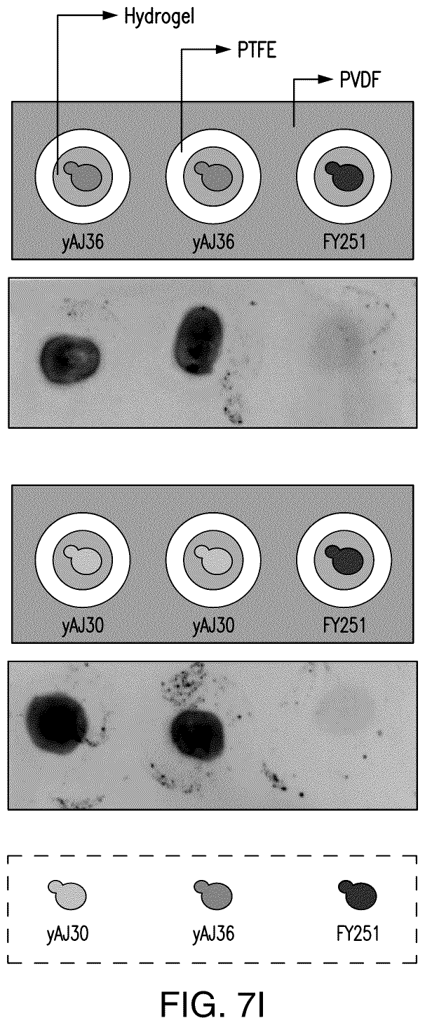

In certain embodiments, a pharmaceutical composition of the present disclosure can include a gel, e.g., a hydrogel, as shown in FIG. 2, FIG. 6, FIG. 7E and FIG. 8D. In certain embodiments, the genetically-engineered cells can be suspended within a hydrogel. In certain embodiments, the hydrogel can comprise a polysaccharide. Non-limiting examples of polysaccharides include agarose, starch, ulvan, carrageenan and porphyrin. In certain embodiments, the hydrogel can comprise synthetic polymers or synthetic polymer forming agents such as poly vinyl alcohol, poly hydroxyl alkyl methacrylate and polyacrylate. In certain embodiments, the hydrogel can comprise biopolymers such as collagen, chitosan or alginate. In certain embodiments, the hydrogel can comprise from about 0.1% w/v to about 10.0% w/v of a polysaccharide. In certain embodiments, the hydrogel can comprise from about 0.1% w/v to about 5.0% w/v of a polysaccharide, e.g., from about 0.1% w/v to about 1.0% w/v of a polysaccharide. In certain embodiments, the hydrogel can comprise from about 0.1% w/v to about 1.0% w/v of a polysaccharide. In certain embodiments, the hydrogel can comprise from about 0.5% w/v to about 1.0% w/v of a polysaccharide. In certain embodiments, the hydrogel can comprise from about 0.5% w/v to about 0.7% w/v of a polysaccharide. In certain embodiments, the hydrogel can comprise from about 0.1% w/v to about 10.0% w/v of agarose. In certain embodiments, the hydrogel can comprise from about 0.1% w/v to about 5.0% w/v of agarose, e.g., from about 0.1% w/v to about 1.0% w/v of agarose. In certain embodiments, the hydrogel can comprise from about 0.1% w/v to about 1.0% w/v of agarose. In certain embodiments, the hydrogel can comprise from about 0.5% w/v to about 1.0% w/v of agarose. In certain embodiments, the hydrogel can comprise from about 0.5% w/v to about 0.7% w/v of agarose.

A non-limiting exemplary embodiment of a pharmaceutical composition that includes a hydrogel is shown in FIG. 2, FIG. 7E and FIG. 8D. In certain embodiments, the hydrogel-based pharmaceutical composition can include (i) an outer and/or adhesive layer, (ii) a medium containing a live genetically-engineered fungal cell, e.g., a hydrogel containing a live genetically-engineered fungal cell, and (iii) a layer between the fungal cell-containing medium, e.g., hydrogel, and the wound bed or skin to be treated.

In certain embodiments, the outer and/or adhesive layer can function as a barrier between the open wound bed (or skin to be treated) and the outside environment. In certain embodiments, the outer and/or adhesive layer can function as a barrier to retain the genetically-engineered cells on the wound or skin to be treated. In certain embodiments, the outer and/or adhesive layer has a pore size that is smaller than the size of the individual genetically-engineered cells in the pharmaceutical composition. In certain embodiments, the outer and/or adhesive layer is polymer-containing material. In certain polymer-containing material is a polyurethane containing material. In certain polymer-containing material is a material or polymer comprising polyurethane or co-polymers thereof.

In certain embodiments, the fungal cell-containing medium, e.g., hydrogel, contains a population of live genetically-engineered fungal cells, nutrients, buffers and/or potential supplemental active ingredients such as antibiotics. In certain embodiments, the fungal cell-containing medium, e.g., hydrogel, allows for diffusion of the one or more therapeutics secreted from the genetically-engineered fungal cells to the wound bed but prevents the fungal cells from reaching the skin and wound bed. In certain embodiments, the fungal cell-containing medium, e.g., hydrogel, includes a polysaccharide. In certain embodiments, the polysaccharide is agarose.

In certain embodiments, the layer between the fungal cell-containing medium, e.g., hydrogel, and the wound bed or skin to be treated is permeable to the skin therapeutic expressed by the fungal cells in the medium. In certain embodiments, the layer between the fungal cell-containing medium, e.g., hydrogel, and the wound bed or skin to be treated is not permeable to the fungal cells in the medium. In certain embodiments, the layer between the fungal cell-containing medium, e.g., hydrogel, and the wound bed or skin to be treated is permeable to the skin therapeutic expressed by the fungal cells in the medium but is not permeable such fungal cells. In certain embodiments, the layer between the fungal cell-containing medium, e.g., hydrogel, and the wound bed or skin to be treated has a pore size of less than about 1.0 μm, e.g., less than about 0.9 μm, less than about 0.8 μm, less than about 0.7 μm, less than about 0.6 μm, less than about 0.5 μm, less than about 0.4 μm, less than about 0.3 μm, less than about 0.2 μm or less than about 0.1 μm. In certain embodiments, the layer between the fungal cell-containing medium, e.g., hydrogel, and the wound bed or skin to be treated has a pore size from about 0.01 to about 1.0 μm, e.g., about 0.2 μm. In certain embodiments, the layer between the fungal cell-containing medium, e.g., hydrogel, and the wound bed or skin to be treated has a pore size from about 0.01 to about 0.2 μm. In certain embodiments, the layer between the fungal cell-containing medium, e.g., hydrogel, and the wound bed or skin to be treated has a pore size from about 0.01 to about 0.5 μm. In certain embodiments, the layer between the fungal cell-containing medium, e.g., hydrogel, and the wound bed or skin to be treated is a polymer. In certain embodiments, the layer between the fungal cell-containing medium, e.g., hydrogel, and the wound bed or skin to be treated has a pore size from about 0.01 to about 1.0 μm, e.g., about 0.2 μm. In certain embodiments, the polymer is polytetrafluoroethylene (PTFE).

### V. Skin Conditions

The present disclosure further provides genetically-engineered cells and pharmaceutical compositions for treating one or more skin conditions.

In certain embodiments, the genetically-engineered cells disclosed herein can be administered to treat the skin. For example, but not by way of limitation, the genetically-engineered cells disclosed herein can be administered to treat various conditions, e.g., skin conditions and/or diseases. Any skin condition can be treated using the disclosed genetically-engineered cells.

In certain embodiments, the genetically-engineered cells disclosed herein (or pharmaceutical compositions thereof) can be administered to treat a wound. In certain embodiments, a wound is damage to the integrity of bodily tissue, e.g., skin, of a subject. In certain embodiments, the genetically-engineered cells disclosed herein can be administered to promote healing of chronic wounds. Non-limiting examples of chronic wounds include ulcers (e.g., diabetic ulcers (e.g., diabetic foot ulcers), venous ulcers, arterial ulcers and pressure ulcers), periodontal lesions, infectious wounds, ischemic wounds, surgical wounds, burn wounds, skin blistering (e.g., epidermolysis bullosa) and radiation poisoning wounds.

In certain embodiments, the genetically-engineered cells disclosed herein (or pharmaceutical compositions thereof) can be administered to promote healing of gastrointestinal conditions. In certain embodiments, the genetically-engineered cells disclosed herein can be administered to promote healing of stomach ulcers. Non-limiting examples of stomach ulcers include peptic ulcers, gastric ulcers, esophageal ulcers and duodenal ulcers. In certain embodiments, the condition is not a gastrointestinal condition.

In certain embodiments, the genetically-engineered cells disclosed herein (or pharmaceutical compositions thereof) can be administered to treat an infection, e.g., an infection of the skin. In certain embodiments, the genetically-engineered cells disclosed herein can be administered to treat a fungal infection (e.g., a yeast infection), a bacterial infection and/or a protozoan infection, e.g., a fungal infection (e.g., a yeast infection), a bacterial infection and/or a protozoan infection of the skin. Non-limiting examples of fungal (e.g., yeast) infections include onychomycosis, candidiasis, Athlete's foot, jock itch, dermatophytosis/ringworm and other infections associated with Trichophyton rubrum and other fungal species infection. Non-limiting examples of bacterial infections include cellulitis, erysipelas, impetigo, folliculitis, furuncles and carbuncles. Non-limiting examples of protozoan infections include malaria, giardia, ascaridosis and toxoplasmosis.

In certain embodiments, the genetically-engineered cells disclosed herein (or pharmaceutical compositions thereof) can be administered to treat acne. In certain embodiments, acne is disorder of the hair follicles and sebaceous glands of the skin. In certain embodiments, acne is considered an inflammatory skin condition.

In certain embodiments, the genetically-engineered cells (or pharmaceutical compositions thereof) disclosed herein can be administered to treat an inflammatory disorder. In certain embodiments, inflammatory disorder can be Steven Johnson syndrome or an inflammatory skin disorder. Non-limiting examples of inflammatory skin disorders include psoriasis, lupus, hidradenitis suppurativa, seborrheic dermatitis, pilonidal cysts and atopic dermatitis (e.g., eczema).

In certain embodiments, the genetically-engineered cells (or pharmaceutical compositions thereof) disclosed herein can be administered to treat fibrotic disorders. Non-limiting examples of fibrotic disorders include skin fibrosis, hypertrophic scars and scleroderma. In certain embodiments, the genetically-engineered cells (or pharmaceutical compositions thereof) disclosed herein can be administered to treat hypertrophic scars.

In certain embodiments, the genetically-engineered cells (or pharmaceutical compositions thereof) disclosed herein can be administered to treat a disorder associated with blistering. Non-limiting examples of blistering disorders include bullous pemphigoid, other pemphigoid variants, epidermolysis bullosa, bullous systemic lupus erythematosus, linear IgA bullous dermatosis, dermatitis herpetiformis, pemphigus vulgaris, Pemphigus foliaceus, other pemphigus variants (Pemphigus erythematosus, Pemphigus herpetiformis, Pemphigus vegetans and IgA pemphigus) and paraneoplastic autoimmune multiorgan syndrome (also known as paraneoplastic pemphigus).

In certain embodiments, the genetically-engineered cells (or pharmaceutical compositions thereof) disclosed herein can be administered to treat a vascular lesion. In certain embodiments, a vascular lesion is a vascular malformation. In certain embodiments, the genetically-engineered cells (or pharmaceutical compositions thereof) disclosed herein can be administered to treat a disorder associated with vascular lesions. Non-limiting examples of vascular lesions include congenital angiomas and cherry angiomas.

In certain embodiments, the genetically-engineered cells (or pharmaceutical compositions thereof) disclosed herein can be administered to treat a benign skin lesion. A benign skin lesion is a non-cancerous skin growth. Non-limiting examples of benign skin lesions include moles, freckles, skin tags, benign lentigines, solar lentigo, keloids and seborrhoeic keratosis.

In certain embodiments, the genetically-engineered cells (or pharmaceutical compositions thereof) disclosed herein can be administered to treat a skin cancer. Non-limiting examples of skin cancers include basal cell carcinoma, squamous cell carcinoma, sebaceous carcinoma, kaposi sarcoma, cutaneous angiosarcoma, melanoma, merkel cell carcinoma, dermatofibrosarcoma protuberans and cutaneous lymphoma.

In certain embodiments, the present disclosure provides methods for treating xeroderma pigmentosum.

In certain embodiments, the genetically-engineered cells (or pharmaceutical compositions thereof) disclosed herein can be administered to treat a pigmentation disorder. Non-limiting examples of pigmentation disorders include hyperpigmentation (e.g., post-inflammatory hyperpigmentation, melasma, solar lentigines, ephelides (freckles) and café au lait macules) and hypopigmentation (e.g., vitiligo).

In certain embodiments, the genetically-engineered cells (or pharmaceutical compositions thereof) disclosed herein can be administered to perform a cosmetic treatment. In certain embodiments, the genetically-engineered cells (or pharmaceutical compositions thereof) disclosed herein can be administered to rejuvenate the skin.

### VI. Methods of Use

The present disclosure further provides methods for using the genetically-engineered cells and pharmaceutical compositions of the present disclosure. The present disclosure provides methods for treating a subject in need thereof by administering one or more genetically-engineered cells of the present disclosure or a pharmaceutical composition thereof to the subject.

In certain embodiments, a method of the present disclosure includes administering one or more live and/or intact genetically-engineered cells, e.g., fungal cells, expressing one or more skin therapeutics, or a composition or pharmaceutical composition thereof. In certain embodiments, a live genetically-engineered cell refers to a cell that has an intact cell membrane and has the ability to proliferate and/or express a skin therapeutic. Non-limiting examples of compositions or pharmaceutical compositions of the presently disclosed genetically-engineered fungal cells are described in Section IV. Non-limiting examples of skin conditions that can be treated with the presently disclosed genetically-engineered fungal cells are described in Section V.

In certain embodiments, a method of the present disclosure includes administering to the subject in need thereof a cell genetically engineered (or a pharmaceutical composition thereof) to generate and secrete a skin therapeutic for treating the subject. In certain embodiments, the genetically-engineered is a fungal cell that produces a skin therapeutic in situ and secretes the skin therapeutic. The genetically-administered cell can be administered to the subject by any method relevant to the disorder and/or condition being treated. For example, but not by way of limitation, the genetically-engineered cell can be administered by rectal administration, vaginal administration, oral administration or topical administration. In certain embodiments, the genetically-engineered cell is not administered orally.

In certain embodiments, the genetically-engineered cell administered to a subject generates and secretes a skin therapeutic for treating the subject. Non-limiting examples of skin therapeutics that can be generated and secreted are disclosed herein in Section II. In certain embodiments, the skin therapeutic can be one or more of a growth factor, a chemokine, a cytokine, an antimicrobial peptide and/or a protease inhibitor. For example, but not by way of limitation, the skin therapeutic can be EGF, PDGF, VEGF, TGF-β, CXCL12, Leptin, TIMP1, TIMP2, IL-4, Collagen Type VII, a TGF-β1 inhibitor, RcA1b-PepI, RcA1b-PepII, RcA1b-PepII, lucifensin, lucifensin II, lucilin, pexiganan acetate (MSI-78), D2A21/D4E1, granulysin, synthetic granulysin-derived peptides, cathelicidin antimicrobial peptide (LL-37) or analogs thereof, K1 toxin peptide, K2 toxin peptide, K28 toxin peptide, an inhibitor of RelA, a defensin, an anti-staphylococcus bactericidal protein, a xeroderma pigmentosum complementation protein (XP), azelaic acid, hyaluronic acid, elastin or a combination thereof. In certain embodiments, the skin therapeutic can be EGF, PDGF, VEGF, TGF-β, CXCL12, Leptin, TIMP1, TIMP2, IL-4, Collagen Type VII and/or a TGF-β1 inhibitor. In certain embodiments, the skin therapeutic can be RcA1b-PepI, RcA1b-PepII, RcA1b-PepII, lucifensin, lucifensin II, lucilin, pexiganan acetate (MSI-78), D2A21/D4E1, granulysin, synthetic granulysin-derived peptides, cathelicidin antimicrobial peptide (LL-37) or analogs thereof, an inhibitor of RelA, a defensin, an anti-staphylococcus bactericidal protein, a xeroderma pigmentosum complementation protein (XP), azelaic acid, hyaluronic acid, elastin or a combination thereof.

In certain embodiments, the genetically-engineered cells disclosed herein can be administered to treat the skin. For example, but not by way of limitation, the genetically-engineered cells disclosed herein can be administered to treat various conditions, e.g., skin conditions and/or diseases. Any skin condition can be treated using the disclosed genetically-engineered cells. Non-limiting examples of skin conditions that can be treated using the disclosed genetically-engineered cells (or pharmaceutical compositions thereof) include wounds, infections, acne, fibrotic disorders, blistering disorders, inflammatory conditions (e.g., inflammatory skin conditions), vascular lesions, skin cancers, xeroderma pigmentosum, pigment disorders and cosmetic procedures.

In certain embodiments, the genetically-engineered cells disclosed herein can be administered to treat skin conditions including hemorrhoids, anal fissures, perianal abscesses, anal fistulas, perianal infections, sores, ulcers, wounds, acne (e.g., bacterially infected acne), inflammation caused by acne, cold sores, blisters, fungal nail infections, athlete's foot, eczema, actinic keratosis, keratosis pilaris, scleroderma, rashes, rosacea, scabies, carbuncle, psoriasis, epidermolysis bullosa, cellulitis, basal cell carcinoma, squamous cell carcinoma, melanoma, contact dermatitis, warts, skin fibrosis and combinations thereof. In certain embodiments, the genetically-engineered cells of the present disclosure can be used for skin and cosmetic treatments, e.g., facial and hair treatments.

In certain embodiments, the genetically-engineered cells disclosed herein (or pharmaceutical compositions thereof) can be administered to treat a wound. In certain embodiments, the genetically-engineered cells disclosed herein (or pharmaceutical compositions thereof) can be administered to promote wound healing. In certain embodiments, a wound is damage to the integrity of bodily tissue, e.g., skin, of a subject. In certain embodiments, the genetically-engineered cells disclosed herein can be administered to promote healing of open wounds. In certain embodiments, the genetically-engineered cells disclosed herein can be administered to promote healing of chronic wounds. In certain embodiments, chronic wounds are wounds that do not progress through the healing process in an orderly and timely manner. Non-limiting examples of chronic wounds include ulcers (e.g., diabetic ulcers (e.g., diabetic foot ulcers), venous ulcers, arterial ulcers and pressure ulcers), periodontal lesions, infectious wounds, ischemic wounds, surgical wounds, burn wounds, skin blistering (e.g., epidermolysis bullosa) and radiation poisoning wounds. In certain embodiments, the genetically-engineered cells disclosed herein (or pharmaceutical compositions thereof) can be administered to treat an ulcer. In certain embodiments, the genetically-engineered cells disclosed herein (or pharmaceutical compositions thereof) can be administered to treat a diabetic ulcer. In certain embodiments, the genetically-engineered cells disclosed herein that express and secrete a growth factor (e.g., EGF and/or PDGF), a cytokine (e.g., Leptin) and/or a protease inhibitor can be administered to promote healing of a wound. In certain embodiments, the present disclosure provides methods for treating a wound, e.g., a diabetic foot ulcer, that include administering a genetically-engineered cell that secretes a growth factor, e.g., EGF. In certain embodiments, the genetically-engineered cells disclosed herein that express and secrete EGF, PDGF or both can be administered to promote healing of a wound. In certain embodiments, the genetically-engineered cells disclosed herein that express and secrete a growth factor (e.g., EGF and/or PDGF) and a protease can be administered to promote healing of a wound. In certain embodiments, the genetically-engineered cells disclosed herein that express and secrete a Leptin and a protease can be administered to promote healing of a wound. In certain embodiments, the genetically-engineered cells disclosed herein that express and secrete a growth factor (e.g., EGF and/or PDGF) and a cytokine (e.g., Leptin) can be administered to promote healing of a wound.

In certain embodiments, the genetically-engineered cells disclosed herein (or pharmaceutical compositions thereof) can be administered to promote healing of gastrointestinal conditions. In certain embodiments, a gastrointestinal disorder is a disorder of the gastrointestinal tract of a subject. In certain embodiments, the genetically-engineered cells disclosed herein can be administered to promote healing of stomach ulcers. Non-limiting examples of stomach ulcers include peptic ulcers, gastric ulcers, esophageal ulcers and duodenal ulcers. In certain embodiments, the genetically-engineered cells disclosed herein are not used for treating a gastrointestinal condition. In certain embodiments, the genetically-engineered cells disclosed herein are not administered orally to treat a gastrointestinal condition.

In certain embodiments, the genetically-engineered cells disclosed herein (or pharmaceutical compositions thereof) can be administered to treat an infection, e.g., an infection of the skin. In certain embodiments, the genetically-engineered cells disclosed herein can be administered to treat a fungal infection (e.g., a yeast infection), a bacterial infection and/or a protozoan infection, e.g., a fungal infection (e.g., a yeast infection), a bacterial infection and/or a protozoan infection of the skin. In certain embodiments, the genetically-engineered cells disclosed herein that express and secrete one or more antimicrobial agents, e.g., antimicrobial peptides, can be administered to treat an infection. Non-limiting examples of antimicrobial peptides include RcA1b-PepI, RcA1b-PepII, RcA1b-PepII, lucifensin, lucifensin II, lucilin, pexiganan acetate (MSI-78) and D2A21/D4E1.

In certain embodiments, the genetically-engineered cells disclosed herein (or pharmaceutical compositions thereof) can be administered to treat a fungal (e.g., yeast) infection, e.g., a fungal infection (e.g., a yeast infection) of the skin. In certain embodiments, the genetically-engineered cells disclosed herein that express and secrete one or more antimicrobial agents can be administered to treat a fungal (e.g., yeast) infection, e.g., of the skin. Non-limiting examples of fungal (e.g., yeast) infections include onychomycosis, candidiasis, Athlete's foot, jock itch, dermatophytosis/ringworm and other infections associated with Trichophyton rubrum and other fungal species infection. In certain embodiments, antimicrobial agents for treating a fungal (e.g., yeast) infection can include antimicrobial peptides, e.g., RcA1b-PepI, RcA1b-PepII and RcA1b-PepII. In certain embodiments, the antimicrobial agent can be a killer toxin, e.g., K1, K2 and/or K28 from Saccharomyces cerevisiae.

In certain embodiments, the genetically-engineered cells disclosed herein (or pharmaceutical compositions thereof) can be administered to treat a bacterial infection, e.g., a bacterial infection of the skin. In certain embodiments, the genetically-engineered cells disclosed herein that express and secrete one or more antimicrobial agents can be administered to treat a bacterial infection, e.g., of the skin. Non-limiting examples of bacterial infections include cellulitis, erysipelas, impetigo, folliculitis, furuncles and carbuncles. In certain embodiments, antimicrobial agents, e.g., antimicrobial peptides, for treating a bacterial infection can include lucifensin, lucifensin II, lucilin, pexiganan acetate (MSI-78) and D2A21/D4E1.

In certain embodiments, the genetically-engineered cells disclosed herein (or pharmaceutical compositions thereof) can be administered to treat a protozoan infection, e.g., a protozoan infection of the skin. Non-limiting examples of protozoan infections include malaria, giardia, ascaridosis and toxoplasmosis.

In certain embodiments, the genetically-engineered cells disclosed herein (or pharmaceutical compositions thereof) can be administered to treat acne. In certain embodiments, acne is disorder of the hair follicles and sebaceous glands of the skin. In certain embodiments, acne is considered an inflammatory skin condition. In certain embodiments, the genetically-engineered cells disclosed herein that express and secrete one or more antimicrobial agents can be administered to treat Propionibacterium acnes that is associated with acne. In certain embodiments, the genetically-engineered cells disclosed herein that express and secrete one or more antimicrobial agents can be administered to treat acne. Non-limiting examples of antimicrobials for treating acne include granulysin, synthetic granulysin-derived peptides and melittin. In certain embodiments, the present disclosure provides methods for treating acne that include administering a genetically-engineered cell that secretes melittin. In certain embodiments, melittin comprises or consists of the amino acid sequence GIGAVLKVLTTGLPALISWIKRKRQQ (SEQ ID NO: 53). In certain embodiments, the present disclosure provides methods for treating acne that include administering a genetically-engineered cell that secretes an antimicrobial and/or anti-inflammatory peptide, e.g., the cathelicidin antimicrobial peptide (LL-37) or analogs thereof, and/or a growth factor, e.g., TGF-β. In certain embodiments, the cathelicidin antimicrobial peptide (LL-37) comprises or consists of the amino acid sequence LLGDFFRKSKEKIGKEFKRIVQRIKDFLRNLVPRTES (SEQ ID NO: 54). Additional non-limiting examples of LL-37 analogs and antimicrobials that can be expressed and secreted from the genetically-engineered cells of the present disclosure are provided in Kuroda et al., Front. Oncol. 5:144 (2015) (e.g., Table 1), the contents of which is incorporated by reference herein in its entirety.

In certain embodiments, the genetically-engineered cells (or pharmaceutical compositions thereof) disclosed herein can be administered to treat an inflammatory disorder. In certain embodiments, inflammatory disorder can be Steven Johnson syndrome or an inflammatory skin disorder. In certain embodiments, the genetically-engineered cells (or pharmaceutical compositions thereof) disclosed herein can be administered to treat an inflammatory skin disorder. Non-limiting examples of inflammatory skin disorders include psoriasis, lupus, hidradenitis suppurativa, seborrheic dermatitis, pilonidal cysts and atopic dermatitis (e.g., eczema). In certain embodiments, the genetically-engineered cells (or pharmaceutical compositions thereof) disclosed herein can be administered to treat a skin disorder associated with dry, itchy and/or flaky skin. In certain embodiments, the genetically-engineered cells (or pharmaceutical compositions thereof) disclosed herein can be administered to treat psoriasis. In certain embodiments, the present disclosure provides methods for treating an inflammatory skin condition that include administering a genetically-engineered cell that secretes a defensin, e.g., β-defensin-3 (hBD-3), EGF, an inhibitor of VEGF or an inhibitor of RelA. Non-limiting examples of inhibitors of RelA include RNAi targeting RelA, and non-limiting examples of inhibitors of VEGF include IL-4. In certain embodiments, the present disclosure provides methods for treating atopic dermatitis that include administering a genetically-engineered cell that secretes an inhibitor of RelA. In certain embodiments, the present disclosure provides methods for treating atopic dermatitis that include administering a genetically-engineered cell that secretes a defensin. In certain embodiments, the present disclosure provides methods for treating psoriasis that include administering a genetically-engineered cell that secretes an inhibitor of VEGF, e.g., IL-4. In certain embodiments, the present disclosure provides methods for treating Stevens Johnson syndrome that include administering a genetically-engineered cell that secretes EGF.

In certain embodiments, the genetically-engineered cells (or pharmaceutical compositions thereof) disclosed herein can be administered to treat fibrotic disorders. In certain embodiments, a fibrotic disorder is characterized by excessive deposition of collagen and other connective tissue components. Non-limiting examples of fibrotic disorders include skin fibrosis, hypertrophic scars and scleroderma. In certain embodiments, the genetically-engineered cells (or pharmaceutical compositions thereof) disclosed herein can be administered to treat hypertrophic scars. In certain embodiments, the present disclosure provides methods for treating fibrotic disorders (e.g., scars (e.g., hypertrophic scars), skin fibrosis and scleroderma) that include administering a genetically-engineered cell that secretes an inhibitor of TGF-β, e.g., TGF-β1. For example, but not by way of limitation, an inhibitor of TGF-β1 can be a peptide inhibitor of TGF-β1. A non-limiting example of a peptide inhibitor of TGF-31 includes a peptide comprising or consisting of the amino acid sequence TSLDASIIWAMMQN (SEQ ID NO: 9).

In certain embodiments, the genetically-engineered cells (or pharmaceutical compositions thereof) disclosed herein can be administered to treat a disorder associated with blistering. In certain embodiments, the genetically-engineered cells (or pharmaceutical compositions thereof) disclosed herein can be administered to treat blistering disorders, e.g., an autoimmune blistering disorder. In certain embodiments, an autoimmune blistering disorder occurs when a subject's immune system attacks their own skin and mucous membranes to form blisters. Non-limiting examples of blistering disorders include bullous pemphigoid, other pemphigoid variants, epidermolysis bullosa, bullous systemic lupus erythematosus, linear IgA bullous dermatosis, dermatitis herpetiformis, Pemphigus vulgaris, Pemphigus foliaceus, other pemphigus variants (pemphigus erythematosus, pemphigus herpetiformis, Pemphigus vegetans and IgA pemphigus) and paraneoplastic autoimmune multiorgan syndrome (also known as paraneoplastic pemphigus). In certain embodiments, the present disclosure provides methods for treating a blistering disorder that include administering a genetically-engineered cell that secretes an extracellular matrix protein, e.g., a collagen. In certain embodiments, the present disclosure provides methods for treating an autoimmune blistering disorder include administering a genetically-engineered cell that secretes an extracellular matrix protein, e.g., a collagen. In certain embodiments, the present disclosure provides methods for treating epidermolysis bullosa that include administering a genetically-engineered cell that secretes an extracellular matrix protein, e.g., a collagen. Non-limiting examples of a collagen that can be used for treating a blistering disorder are described herein. In certain embodiments, the collagen can include Type VII Collagen or a fragment thereof.

In certain embodiments, the present disclosure provides methods for treating a vascular lesion. In certain embodiments, a vascular lesion is a vascular malformation. In certain embodiments, the genetically-engineered cells (or pharmaceutical compositions thereof) disclosed herein can be administered to treat a disorder associated with vascular lesions. Non-limiting examples of vascular lesions include congenital angiomas and cherry angiomas. In certain embodiments, the present disclosure provides methods for treating a vascular lesion that include administering a genetically-engineered cell that secretes an inhibitor of VEGF.

In certain embodiments, the present disclosure provides methods for treating a benign skin lesion. In certain embodiments, a benign skin lesion is a non-cancerous skin growth. In certain embodiments, the genetically-engineered cells (or pharmaceutical compositions thereof) disclosed herein can be administered to treat a benign skin lesion. Non-limiting examples of benign skin lesions include moles, freckles, skin tags, benign lentigines, solar lentigo, keloids and seborrhoeic keratosis.

In certain embodiments, the present disclosure provides methods for treating a skin cancer. In certain embodiments, the genetically-engineered cells (or pharmaceutical compositions thereof) disclosed herein can be administered to treat skin cancer. In certain embodiments, the skin cancer is a cancer of the squamous cells of the skin. In certain embodiments, the skin cancer is a cancer of the basal cells of the skin. Non-limiting examples of skin cancers include basal cell carcinoma, squamous cell carcinoma, sebaceous carcinoma, kaposi sarcoma, cutaneous angiosarcoma, melanoma, merkel cell carcinoma, dermatofibrosarcoma protuberans and cutaneous lymphoma. In certain embodiments, the present disclosure provides methods for treating a skin cancer that include administering a genetically-engineered cell that secretes an agent for treating the skin cancer. Non-limiting examples of such agents include melittin, Toll-like receptor inhibitors, an inhibitor of VEGF, targeted therapies or small molecules, an interferon, an interleukin, e.g., IL-12, and anti-staphylococcus bactericidal proteins. In certain embodiments, the present disclosure provides methods for treating a basal cell carcinoma that include administering a genetically-engineered cell that secretes melittin. In certain embodiments, the present disclosure provides methods for treating a squamous cell carcinoma that include administering a genetically-engineered cell that secretes a Toll-like receptor inhibitor, e.g., imiquimod or resiquimod. In certain embodiments, the present disclosure provides methods for treating Kaposi Sarcoma or an angiosarcoma that include administering a genetically-engineered cell that secretes an inhibitor of VEGF. In certain embodiments, the present disclosure provides methods for treating a melanoma or a merkel cell carcinoma that include administering a genetically-engineered cell that secretes a targeted therapy or small molecule. In certain embodiments, the present disclosure provides methods for treating a cutaneous lymphoma that include administering a genetically-engineered cell that secretes an interferon, an interleukin, e.g., IL-12, and/or an anti-staphylococcus bactericidal protein.

In certain embodiments, the present disclosure provides methods for treating xeroderma pigmentosum. Non-limiting examples of agents for treating xeroderma pigmentosum include Xeroderma Pigmentosum group A (XPA), XPB, XPC (human RAD4), XPD, XPE, XPF and XPG. In certain embodiments, the present disclosure provides methods for treating xeroderma pigmentosum that include administering a genetically-engineered cell that secretes XPA, XPB, XPC (human RAD4), XPD, XPE, XPF and/or XPG.

In certain embodiments, the present disclosure provides methods for treating pigmentation disorders. In certain embodiments, the genetically-engineered cells (or pharmaceutical compositions thereof) disclosed herein can be administered to treat a pigmentation disorder. Non-limiting examples of pigmentation disorders include hyperpigmentation (e.g., post-inflammatory hyperpigmentation, melasma, solar lentigines, ephelides (freckles) and café au lait macules) and hypopigmentation (e.g., vitiligo). In certain embodiments, the genetically-engineered cells (or pharmaceutical compositions thereof) disclosed herein can be administered to treat a disorder associated with hyperpigmentation or hypopigmentation. Non-limiting examples of therapeutics for treating a pigmentation disorder include azelaic acid and lysosomal extracts from yeast, e.g., S. cerevisiae. In certain embodiments, the present disclosure provides methods for treating a pigmentation disorder that include administering a genetically-engineered cell that secretes azelaic acid.

In certain embodiments, the genetically-engineered cells (or pharmaceutical compositions thereof) disclosed herein can be administered to perform a cosmetic treatment. For example, but not by way of limitation, the genetically-engineered cells (or pharmaceutical compositions thereof) disclosed herein can be administered to alter and/or enhance the appearance of the skin. In certain embodiments, the genetically-engineered cells (or pharmaceutical compositions thereof) disclosed herein can be administered to rejuvenate the skin. In certain embodiments, the genetically-engineered cells (or pharmaceutical compositions thereof) disclosed herein can be administered to rejuvenate the skin. In certain embodiments, the present disclosure provides methods for rejuvenating the skin that include administering a genetically-engineered cell that secretes an extracellular matrix protein, e.g., collagen and/or elastin. In certain embodiments, the present disclosure provides methods for rejuvenating the skin that include administering a genetically-engineered cell that secretes hyaluronic acid.

In certain embodiments, a genetically-engineered cell disclosed herein (or pharmaceutical compositions thereof) can be topically administered. For example, but not by way of limitation, a genetically-engineered cell disclosed herein can be applied for treatment of skin diseases and/or conditions such as wounds and ulcers and to promote skin healing. In certain embodiments, a method of the present disclosure includes administration of a cell genetically engineered to express and/or secrete a skin therapeutic such as but limited to a growth factor, cytokine, chemokine and/or protease inhibitor to treat a subject with a skin condition, e.g., a wound, a sore or an ulcer. In certain embodiments, the genetically-engineered cell is applied directly to the area that needs to be treated, e.g., directly to the wound or directly to the affected skin.

In certain embodiments, a genetically-engineered cell disclosed herein (or pharmaceutical compositions thereof) can be administered once a day, twice a day, once every two days, once every three days, once every four days, once every five days, once every six days, once a week, twice a week, three times a week, four times a week, five times a week, six times a week, once every two weeks, once a month, twice a month, once every other month or once every third month. In certain embodiments, the genetically-engineered cell can be administered twice a week. For example, but not by way of limitation, the genetically-engineered cells of the present disclosure (or pharmaceutical compositions thereof) can be reapplied twice a week, three times a week, four times a week, five times a week, six times a week, once every two weeks, once a month, twice a month, once every other month or once every third month. In certain embodiments, the genetically-engineered cell can be administered once a day. In certain embodiments, the genetically-engineered cell can be administered once every two days. In certain embodiments, the genetically-engineered cell can be administered once every three days. In certain embodiments, a genetically-engineered cell disclosed herein be administered once a week. In certain embodiments, a genetically-engineered cell disclosed herein can be administered two times a week for about four weeks and then administered once a week for the remaining duration of the treatment.

In certain embodiments, administration of a genetically-engineered cell that expresses and/or secretes a skin therapeutic (or pharmaceutical compositions thereof) allows the continuous treatment of the subject with the skin therapeutic without the need for multiple administrations. For example, but not by way of limitation, the administration of a genetically-engineered cell of the present disclosure allows secretion, e.g., continuous secretion, of a skin therapeutic for at least about 12 hours, at least about 24 hours, at least about 48 hours, at least about 72 hours, at least about 96 hours, at least about 108 hours, at least about 120 hours, at least about 132 hours, at least about 144 hours, at least about 156 hours or at least about 165 hours after administration to the subject after administration of the genetically-engineered cell to the subject. In certain embodiments, the administration of a genetically-engineered cell of the present disclosure allows secretion, e.g., continuous secretion, of a skin therapeutic for at least about 8 days, at least about 9 days, at least about 10 days, at least about 11 days, at least about 12 days, at least about 13 days or at least about 14 days after administration to the subject after administration of the genetically-engineered cell to the subject. In certain embodiments, a genetically engineered cell of the present disclosure secretes the skin therapeutic for at least 48 hours after administration to the subject. In certain embodiments, a genetically engineered cell of the present disclosure secretes the skin therapeutic for at least 72 hours after administration to the subject. In certain embodiments, a genetically engineered cell of the present disclosure secretes the skin therapeutic for at least 96 hours after administration to the subject. In certain embodiments, a genetically engineered cell of the present disclosure secretes the skin therapeutic for at least 108 hours after administration to the subject. In certain embodiments, a genetically engineered cell of the present disclosure secretes the skin therapeutic for at least 120 hours after administration to the subject. In certain embodiments, a genetically engineered cell of the present disclosure secretes the skin therapeutic for at least 132 hours after administration to the subject. In certain embodiments, a genetically engineered cell of the present disclosure secretes the skin therapeutic for at least 144 hours after administration to the subject. In certain embodiments, a genetically engineered cell of the present disclosure secretes the skin therapeutic for at least 156 hours after administration to the subject. In certain embodiments, a genetically engineered cell of the present disclosure secretes the skin therapeutic for at least 168 hours after administration to the subject. In certain embodiments, the administration of a genetically-engineered cell of the present disclosure allows secretion, e.g., continuous secretion, of a skin therapeutic for at least about 8 days after administration to the subject after administration of the genetically-engineered cell to the subject. In certain embodiments, the administration of a genetically-engineered cell of the present disclosure allows secretion, e.g., continuous secretion, of a skin therapeutic for at least about 9 days after administration to the subject after administration of the genetically-engineered cell to the subject. In certain embodiments, the administration of a genetically-engineered cell of the present disclosure allows secretion, e.g., continuous secretion, of a skin therapeutic for at least about 10 days after administration to the subject after administration of the genetically-engineered cell to the subject. In certain embodiments, the administration of a genetically-engineered cell of the present disclosure allows secretion, e.g., continuous secretion, of a skin therapeutic for at least about 11 days after administration to the subject after administration of the genetically-engineered cell to the subject. In certain embodiments, the administration of a genetically-engineered cell of the present disclosure allows secretion, e.g., continuous secretion, of a skin therapeutic for at least about 12 days after administration to the subject after administration of the genetically-engineered cell to the subject. In certain embodiments, the administration of a genetically-engineered cell of the present disclosure allows secretion, e.g., continuous secretion, of a skin therapeutic for at least about 13 days after administration to the subject after administration of the genetically-engineered cell to the subject. In certain embodiments, the administration of a genetically-engineered cell of the present disclosure allows secretion, e.g., continuous secretion, of a skin therapeutic for at least about 14 days after administration to the subject after administration of the genetically-engineered cell to the subject. In certain embodiments, the administration of a genetically-engineered cell of the present disclosure allows secretion, e.g., continuous secretion, of a skin therapeutic for at least about 1 week after administration to the subject after administration of the genetically-engineered cell to the subject. In certain embodiments, the administration of a genetically-engineered cell of the present disclosure allows secretion, e.g., continuous secretion, of a skin therapeutic for at least about 2 weeks after administration to the subject after administration of the genetically-engineered cell to the subject.

### VII. Kits and Products

The present disclosure provides kits and products for use in the present disclosure. In certain embodiments, the present disclosure provides kits and products for treating a skin condition as described herein. For example, but not by way of limitation, the present disclosure provides kits and products for treating wounds, infections, acne, fibrotic disorders, blistering disorders, inflammatory conditions (e.g., inflammatory skin conditions), vascular lesions, skin cancers, xeroderma pigmentosum and pigment disorders and for performing cosmetic procedures.

In certain embodiments, a kit and/or product of the present disclosure can include one or more genetically-engineered cells, as described above. In certain embodiments, the genetically-engineered cell can be freeze-dried or lyophilized.

In certain embodiments, the kit and/or product can further include a food source, e.g., medium, sugar or agar, for activating the genetically-engineered cell. In certain embodiments, a kit and/or product of the present disclosure can include components to improve cell viability and proliferation, including one or more carbon sources, one or more nitrogen sources, one or more trace nutrient sources and/or one or more additional nutrient sources.

In certain embodiments, the kit and/or product can include a pharmaceutical composition including one or more genetically-engineered cells described herein. For example, but not by way of limitation, the pharmaceutical composition can be a hydrogel that includes one or more genetically-engineered cells described herein. In certain embodiments, the pharmaceutical composition can be formulated for topical administration.

In certain embodiments, a kit and/or product can include a nucleic acid for preparing a genetically-engineered cell of the present disclosure. For example, but not by way of limitation, the nucleic acid can include a nucleotide sequence that encodes one or more skin therapeutics disclosed herein.

In certain embodiments, the kit and/or product can further include instructions for using the genetically-engineered cells or pharmaceutical compositions including such cells.

### VIII. Exemplary Non-Limiting Embodiments

A. The present disclosure provides a pharmaceutical composition comprising
(i) a live fungal cell genetically engineered to express and secrete a skin therapeutic and
(ii) a pharmaceutically acceptable carrier.

A1. The pharmaceutical composition of A, wherein the skin therapeutic is secreted from the fungal cell by a secretory pathway of the fungal cell.

A2. The pharmaceutical composition of A or A1, wherein the skin therapeutic is a protein or a functional fragment thereof.

A3. The pharmaceutical composition of any one of A1-A2, wherein the skin therapeutic is selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof and a combination thereof.

A4. The pharmaceutical composition of A3, wherein the skin therapeutic is a growth factor or a derivative thereof or an inhibitor of a growth factor or a derivative thereof.

A5. The pharmaceutical composition of A4, wherein the growth factor is selected from the group consisting of epidermal growth factor (EGF), platelet-derived growth factor (PDGF), fibroblast growth factor (FGF), vascular endothelial growth factor (VEGF), transforming growth factor-beta (TGF-β) and a combination thereof.

A6. The pharmaceutical composition of A5, wherein the growth factor is EGF.

A7. The pharmaceutical composition of A5, wherein the growth factor is PDGF.

A8. The pharmaceutical composition of A5, wherein the growth factor is TGF-3.

A9. The pharmaceutical composition of A5, wherein the growth factor is VEGF.

A10. The pharmaceutical composition of A3, wherein the skin therapeutic is a chemokine or a derivative thereof.

A11. The pharmaceutical composition of A10, wherein the chemokine is CXCL12.

A12. The pharmaceutical composition of A3, wherein the skin therapeutic is a cytokine or a derivative thereof.

A13. The pharmaceutical composition of A12, wherein the cytokine is selected from the group consisting of Leptin, IL-4 and a combination thereof.

A14. The pharmaceutical composition of A3, wherein the skin therapeutic is a protease inhibitor or a derivative thereof.

A15. The pharmaceutical composition of A14, wherein the skin therapeutic is selected from the group consisting of TIMP1, TIMP2 and a combination thereof.

A16. The pharmaceutical composition of A3, wherein the skin therapeutic is an extracellular matrix protein or a derivative thereof.

A17. The pharmaceutical composition of A16, wherein the skin therapeutic is selected from the group consisting of Type VII Collagen, elastin and a combination thereof.

A18. The pharmaceutical composition of A or A1, wherein the skin therapeutic is an antimicrobial and/or an anti-inflammatory peptide.

A19. The pharmaceutical composition of A18, wherein the antimicrobial and/or anti-inflammatory peptide is selected from the group consisting of cathelicidin antimicrobial peptide (LL-37) or analogs thereof, RcA1b-PepI, RcA1b-PepII, RcA1b-PepII, lucifensin, lucifensin II, lucilin, pexiganan acetate (MSI-78), D2A21/D4E1, granulysin, a synthetic granulysin-derived peptide and a combination thereof.

A20. The pharmaceutical composition of any one of A-A19, wherein the fungal cell is a species from a genus selected from the group consisting of Cladosporium, Aureobasidium, Aspergillus, Saccharomyces, Malassezia, Epicoccum, Candida, Penicillium, Wallemia, Pichia, Phoma, Cryptococcus, Fusarium, Clavispora, Cyberlindnera, Kluyveromyces and a combination thereof.

A21. The pharmaceutical composition of any one of A-A20, wherein the fungal cell is Saccharomyces cerevisiae or Pichia pastoris.

A22. The pharmaceutical composition of any one of A-A21 further comprising a second live fungal cell genetically engineered to express and secrete a second skin therapeutic.

A23. The pharmaceutical composition of A22 further comprising a third live fungal cell genetically engineered to express and secrete a third skin therapeutic.

A24. The pharmaceutical composition of A23 further comprising a fourth live fungal cell genetically engineered to express and secrete a fourth skin therapeutic.

A25. The pharmaceutical composition of A24 further comprising a fifth live fungal cell genetically engineered to express and secrete a fifth skin therapeutic.

A26. The pharmaceutical composition of any one of A-A25, wherein the pharmaceutical composition is formulated for rectal administration, vaginal administration or topical administration.

A27. The pharmaceutical composition of A26, wherein the pharmaceutical composition is formulated for topical administration.

A28. The pharmaceutical composition of any one of A-A27, wherein the pharmaceutically acceptable carrier comprises a hydrogel.

A29. The pharmaceutical composition of A28, wherein the hydrogel comprises from about 0.1% w/v to about 5.0% w/v of a polysaccharide.

A30. The pharmaceutical composition of A29, wherein the hydrogel comprises from about 0.1% w/v to about 1.0% w/v of a polysaccharide.

A31. The pharmaceutical composition of A29 or A30, wherein the polysaccharide is agarose.

A32. The pharmaceutical composition of any one of A-A31, wherein the pharmaceutical compositions comprises a therapeutically effect amount of the live genetically-engineered fungal cell, wherein the therapeutically effective amount is from about 1×10⁴ cells/ml to about 1×10⁸ cells/ml of the live genetically-engineered fungal cells.

A33. The pharmaceutical composition of A32, wherein the therapeutically effective amount is from about 1×10⁶ cells/ml to about 2×10⁷ cells/ml of the live genetically-engineered fungal cells.

A34. The pharmaceutical composition of any one of A-A33, wherein the therapeutically effective amount of live genetically-engineered fungal cells comprises an amount of live genetically-engineered fungal cells that express and secrete from about 1 pg/ml and about 200,000 pg/ml of the skin therapeutic in about 24 hours or less.

A35. The pharmaceutical composition of A34, wherein the therapeutically effective amount of live genetically-engineered fungal cells comprises an amount of live genetically-engineered fungal cells that express and secrete from about 100 pg/ml to about 25,000 pg/ml of the skin therapeutic in about 24 hours or less.

A36. The pharmaceutical composition of any one of A-A35, wherein the live genetically-engineered fungal cell secretes and expresses the skin therapeutic for about 24 hours to about 2 weeks after administration.

A37. The pharmaceutical composition of A36, wherein the live genetically-engineered fungal cell secretes and expresses the skin therapeutic for at least about 48 hours after administration.

A38. The pharmaceutical composition of A37, wherein the live genetically-engineered fungal cell secretes and expresses the skin therapeutic for at least about 72 hours after administration.

A39. The pharmaceutical composition of A38, wherein the live genetically-engineered fungal cell secretes and expresses the skin therapeutic for at least about 96 hours after administration.

A40. The pharmaceutical composition of A39, wherein the live genetically-engineered fungal cell secretes and expresses the skin therapeutic for at least two weeks after administration.

A41. The pharmaceutical composition of any one of A-A40, wherein the live genetically-engineered fungal cell continuously secretes and expresses the skin therapeutic.

A42. The pharmaceutical composition of any one of A-A41, further comprising one or more nutrients for the one or more genetically engineered fungal cells.

B. The present disclosure provides a topical pharmaceutical composition comprising a hydrogel comprising a live fungal cell genetically engineered to express and secrete a skin therapeutic.

B1. The topical pharmaceutical composition of B, wherein the hydrogel comprises from about 0.1% w/v to about 5.0% w/v of a polysaccharide.

B2. The topical pharmaceutical composition of B1, wherein the hydrogel comprises from about 0.1% w/v to about 1.0% w/v of a polysaccharide.

B3. The topical pharmaceutical composition of B1 or B2, wherein the polysaccharide is agarose.

B4. The topical pharmaceutical composition of any one of B-B3 further comprising a bottom layer facing the skin and a top layer facing the air, wherein the hydrogel comprising the fungal cell genetically engineered to express and secrete the skin therapeutic is disposed between the bottom layer and the top layer.

B5. The topical pharmaceutical composition of B4, wherein the genetically-engineered fungal cell cannot pass through the bottom layer, and wherein the skin therapeutic can pass through the bottom layer.

B6. The topical pharmaceutical composition of B4 or B5, wherein the bottom layer has a pore size of about 2 μm.

B7. The topical pharmaceutical composition of any one of B-B6, wherein the topical pharmaceutical composition comprises a therapeutically effective amount of the live genetically-engineered fungal cell.

B8. The topical pharmaceutical composition of B7, wherein the therapeutically effective amount of live genetically-engineered fungal cells comprises from about 1×10³ cells/ml to about 1×10¹⁰ cells/ml of the live genetically-engineered fungal cells.

B9. The topical pharmaceutical composition of B7 or B8, wherein the therapeutically effective amount of live genetically-engineered fungal cells comprises an amount of live genetically-engineered fungal cells that express and secrete from about 1 pg/ml and about 200,000 pg/ml of the skin therapeutic in about 24 hours or less.

B9-1. The topical pharmaceutical composition of B7 or B8, wherein the therapeutically effective amount of live genetically-engineered fungal cells comprises an amount of live genetically-engineered fungal cells that express and secrete from about 100 pg/ml and about 25,000 pg/ml of the skin therapeutic in about 24 hours or less.

B9-2. The topical pharmaceutical composition of B7 or B8, wherein the therapeutically effective amount of live genetically-engineered fungal cells comprises an amount of live genetically-engineered fungal cells that express and secrete from about 100 pg/ml and about 10,000 pg/ml of the skin therapeutic in about 24 hours or less.

B10. The topical pharmaceutical composition of any one of B-B9-2, wherein the genetically engineered fungal cell secretes the skin therapeutic for at least about 12 hours, at least about 24 hours, at least about 48 hours, at least about 60 hours, at least about 72 hours, at least about 84 hours, at least about 96 hours, at least about 108 hours, at least about 120 hours, at least about 132 hours, at least about 144 hours, at least about 156 hours, at least about 165 hours, at least about 8 days, at least about 9 days, at least about 10 days, at least about 11 days, at least about 12 days, at least about 13 days or at least about 14 days after administration to the subject.

B11. The topical pharmaceutical composition of any one of B-B10, wherein the fungal cell is Saccharomyces cerevisiae or Pichia pastoris.

B12. The topical pharmaceutical composition of any one of B-B11, wherein the skin therapeutic is a protein or a functional fragment thereof.

B13. The topical pharmaceutical composition of B12, wherein the skin therapeutic is selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof and a combination thereof.

B14. The topical pharmaceutical composition of B13, wherein the skin therapeutic is a growth factor or a derivative thereof or an inhibitor of a growth factor or a derivative thereof.

B15. The topical pharmaceutical composition of B14, wherein the growth factor is selected from the group consisting of epidermal growth factor (EGF), platelet-derived growth factor (PDGF), fibroblast growth factor (FGF), vascular endothelial growth factor (VEGF), transforming growth factor-beta (TGF-β) and a combination thereof.

B16. The topical pharmaceutical composition of any one of B-B15, wherein the skin therapeutic is an antimicrobial and/or an anti-inflammatory peptide.

B17. The topical pharmaceutical composition of B16, wherein the antimicrobial and/or anti-inflammatory peptide is selected from the group consisting of cathelicidin antimicrobial peptide (LL-37) or analogs thereof, RcA1b-PepI, RcA1b-PepII, RcA1b-PepII, lucifensin, lucifensin II, lucilin, pexiganan acetate (MSI-78), D2A21/D4E1, granulysin, a synthetic granulysin-derived peptide and a combination thereof.

C. The present disclosure provides a pharmaceutical composition comprising: (i) a first live fungal cell genetically engineered to express and secrete a first skin therapeutic; (ii) a second live fungal cell genetically engineered to express and secrete a second skin therapeutic; and (iii) a pharmaceutically acceptable carrier, wherein the first skin therapeutic and the second skin therapeutic are different.

C1. The pharmaceutical composition of C, wherein the first skin therapeutic and the second skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

C2. The pharmaceutical composition of C1, wherein the first skin therapeutic comprises a growth factor and the second skin therapeutic comprises a chemokine.

C3. The pharmaceutical composition of C2, wherein the first skin therapeutic comprises a growth factor and the second skin therapeutic comprises a cytokine.

D. The present disclosure provides a pharmaceutical composition comprising: (i) a first live fungal cell genetically engineered to express and secrete a first skin therapeutic; (ii) a second live fungal cell genetically engineered to express and secrete a second skin therapeutic; (iii) a third live fungal cell genetically engineered to express and secrete a third skin therapeutic; and (iv) a pharmaceutically acceptable carrier, wherein the first skin therapeutic, the second skin therapeutic and the third skin therapeutic are different.

D1. The pharmaceutical composition of D, wherein the first skin therapeutic, the second skin therapeutic and the third skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

E. The present disclosure provides a pharmaceutical composition comprising: (i) a first live fungal cell genetically engineered to express and secrete a first skin therapeutic; (ii) a second live fungal cell genetically engineered to express and secrete a second skin therapeutic; (iii) a third live fungal cell genetically engineered to express and secrete a third skin therapeutic; (iv) a fourth live fungal cell genetically engineered to express and secrete a fourth skin therapeutic; and (v) a pharmaceutically acceptable carrier, wherein the first skin therapeutic, the second skin therapeutic, the third skin therapeutic and the fourth skin therapeutic are different.

E1. The pharmaceutical composition of E, wherein the first skin therapeutic, the second skin therapeutic, the third skin therapeutic and the fourth skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

F. The present disclosure provides a pharmaceutical composition comprising (i) a first live fungal cell genetically engineered to express and secrete a first skin therapeutic; (ii) a second live fungal cell genetically engineered to express and secrete a second skin therapeutic; (iii) a third live fungal cell genetically engineered to express and secrete a third skin therapeutic; (iv) a fourth live fungal cell genetically engineered to express and secrete a fourth skin therapeutic; (v) a fifth live fungal cell genetically engineered to express and secrete a fifth skin therapeutic; and (vi) a pharmaceutically acceptable carrier, wherein the first skin therapeutic, the second skin therapeutic, the third skin therapeutic, the fourth skin therapeutic and the fifth skin therapeutic are different

F1. The pharmaceutical composition of F, wherein the first skin therapeutic, the second skin therapeutic, the third skin therapeutic, the fourth skin therapeutic and the fifth skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

F2. The pharmaceutical composition of F, further comprising a sixth live fungal cell genetically engineered to express and secrete a sixth skin therapeutic, where the sixth skin therapeutic is selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

G. The present disclosure provides a method for treating a subject in need thereof comprising administering to the subject the pharmaceutical composition of any one of A-F2.

G1. The method of G, wherein the pharmaceutical composition is formulated for topical administration.

G2. The method of G or G1, wherein the pharmaceutical composition is administered to the subject to treat a skin condition or to perform a cosmetic procedure.

G3. The method of G2, wherein the skin condition is selected from the group consisting of a wound, an infection, acne, a fibrotic disorder, a blistering disorder, an inflammatory condition, a vascular lesion, a skin cancer, xeroderma pigmentosum, a pigment disorder and a combination thereof.

G4. The method of G3, wherein the skin condition is a wound.

G5. The method of G4, wherein the wound is a diabetic ulcer.

G6. The method of G3, wherein the skin condition is an infection.

G7. The method of G3, wherein the skin condition is acne.

G8. The method of G3, wherein the skin condition is a fibrotic disorder.

G9. The method of G8, wherein the fibrotic disorder is scleroderma.

G10. The method of G3, wherein the skin condition is a blistering disorder.

G11. The method of G10, wherein the blistering disorder is epidermolysis bullosa.

G12. The method of G3, wherein the skin condition is an inflammatory condition.

G13. The method of G12, wherein the inflammatory condition is psoriasis.

G14. The method of G3, wherein the skin condition is a vascular lesion.

G15. The method of G3, wherein the skin condition is a skin cancer.

G16. The method of G3, wherein the skin condition is xeroderma pigmentosum.

G17. The method of G3, wherein the skin condition is a pigment disorder.

G18. The method of any one of G-G17, wherein the genetically engineered fungal cell secretes the skin therapeutic for at least about 12 hours, at least about 24 hours, at least about 48 hours, at least about 60 hours, at least about 72 hours, at least about 84 hours, at least about 96 hours, at least about 108 hours, at least about 120 hours after administration to the subject, at least about 132 hours, at least about 144 hours, at least about 156 hours, at least about 165 hours, at least about 8 days, at least about 9 days, at least about 10 days, at least about 11 days, at least about 12 days, at least about 13 days or at least about 14 days after administration to the subject.

G19. The method of G18, wherein the genetically engineered fungal cell secretes the skin therapeutic for at least about 72 hours after administration to the subject.

G20. The method of any one of G-G17, wherein administration of the pharmaceutical composition to the subject comprises applying the pharmaceutical composition to the affected area.

G21. The method of G20, wherein the pharmaceutical composition is applied no more than 5 times a week, no more than 4 times a week, no more than 3 times a week, no more than 2 times a week, no more than 1 time a week.

G22. The method of G21, wherein the pharmaceutical composition is applied no more than 3 times a week.

G23. The method of G22, wherein the pharmaceutical composition is applied no more than 2 times a week.

H. The present disclosure provides a use of the pharmaceutical composition of any one of A-F2 for treating a skin condition or for performing a cosmetic procedure.

H1. The use of H, wherein the skin condition is selected from the group consisting of a wound, an infection, acne, a fibrotic disorder, a blistering disorder, an inflammatory condition, a vascular lesion, a skin cancer, xeroderma pigmentosum, a pigment disorder and a combination thereof.

H2. The use of H1, wherein the skin condition is a wound.

I. The present disclosure provides a kit comprising the pharmaceutical composition of A-F2.

J. The present disclosure provides a kit for performing the method of any one of G-G23.

### EXAMPLES

The following examples are merely illustrative of the presently disclosed subject matter and should not be considered as limitations in any way.

### Example 1: Genetic Modification of Yeast to Express and Secrete Skin Therapeutics

This Example shows the expression and secretion of the skin therapeutics, EGF and Leptin in fungal cells. Fungal cells such as yeast were chosen as they are superior to bacterial cells. In particular, yeast have been fermented at large scale, delivered and stored in dried form, and used safely by humans in their households for centuries. The key wound healing protein factors are eukaryotic proteins and yeast are well known to be superior to bacteria for expression and secretion of eukaryotic proteins because they have protein folding chaperons, disulfide-bond formation and post-translational machineries both naturally similar and engineered to mimic those in mammalian cells. Yeast are also ideal for mammalian protein production and have been harnessed for decades by industry for the large-scale commercial production of purified mammalian proteins. Similar to bacteria, yeasts are one of the simplest and most powerful organisms to manipulate in the laboratory and numerous synthetic biology tools have been developed for yeast.

Saccharomyces cerevisiae (S. cerevisiae) was chose for delivery of wound-healing protein factors because S. cerevisiae is a simple, unicellular eukaryotic organism; S. cerevisiae are powerful genetic tools, including a growing synthetic biology toolbox, for manipulating S. cerevisiae in the laboratory; S. cerevisiae can be fermented, dried, and distributed on a very large scale cheaply and quickly; S. cerevisiae is eukaryotic and thus is better suited and has been further engineered for expression of recombinant post-translationally modified human proteins, e.g., glycosylation; and for these reasons S. cerevisiae has been used for decades in industry for large-scale production of purified human proteins. In addition, S. cerevisiae is ideal as a topical therapeutic because it is generally recognized as safe (GRAS) organism unlike many bacteria.

A high copy yeast plasmid was designed with a strong constitutive alcohol dehydrogenase one promoter (pADH1), the signal peptide for the secretory pathway, an N-terminal FLAG tag (DYKDDDDK (SEQ ID NO: 10)) and the skin therapeutic. The following skin therapeutics were cloned into the high copy yeast plasmid: (1) mouse EGF (mEGF) and human EGF (hEGF), (2) mouse Leptin (mLeptin) and human Leptin (hLeptin),(3) mouse PDGF (mPDGF) and human PDGF (hPDGF) and (4) mouse CXCL12 (mCXCL12) and human CXCL12 (hCXCL12), as shown in Tables 2 and 3. Table 2 provides the amino acid and nucleotide sequences of the skin therapeutics in the generated plasmids, Table 3 provides a summary of the plasmids generated and Table 4 provides a summary of the strains used. Table 5 provides the nucleotide sequences of the promoters and Table 6 provides the amino acid and nucleotide sequences of the signal peptides.

TABLE 2

| Protein Factor | Amino Acid Sequence* | Nucleotide Sequence* |
|---|---|---|
| pAJ03: hPDGFb | SLGSLTIAEPAMIAECKTRTEVFE | AGCCTTGGGAGCCTGACCAT |
| (under the | ISRRLIDRTNANFLVWPPCVEVQ | CGCAGAGCCAGCAATGATT |
| mf_alpha_prepro | RCSGCCNNRNVQCRPTQVQLRP | GCTGAATGCAAAACGAGGA |
| secretion signal) | VQVRKIEIVRKKPIFKKATVTLE | CAGAAGTTTTTGAAATAAGC |
| | DHLACKCETVAAARPVT (SEQ | AGAAGACTGATAGATAGAA |
| | ID NO: 1) | CCAACGCTAATTTTTTGGTG |
| | | TGGCCCCCTTGCGTGGAGGT |
| | | ACAAAGGTGCTCTGGATGCT |
| | | GCAATAACAGGAACGTACA |
| | | ATGCAGGCCCACCCAGGTA |
| | | CAACTGAGGCCGGTGCAGG |
| | | TCAGAAAGATCGAGATAGT |
| | | CAGAAAGAAACCTATATTC |
| | | AAAAAGGCCACCGTCACCC |
| | | TTGAAGACCATTIGGCGTGC |
| | | AAGTGTGAGACCGTTGCGG |
| | | CGGCCAGACCCGTGACT |
| | | (SEQ ID NO: 22) |
| pAJ05: FLAG- | DYKDDDDKSLGSLTIAEPAMIAE | GACTACAAAGACGATGACG |
| hPDGFb (under | CKTRTEVFEISRRLIDRTNANFL | ACAAGAGCCTTGGGAGCCT |
| the | VWPPCVEVQRCSGCCNNRNVQ | GACCATCGCAGAGCCAGCA |
| mf_alpha_prepro | CRPTQVQLRPVQVRKIEIVRKKP | ATGATTGCTGAATGCAAAAC |
| secretion signal; | IFKKATVTLEDHLACKCETVAA | GAGGACAGAAGTTTTTGAA |
| FLAG sequence is | ARPVT (SEQ ID NO: 11) | ATAAGCAGAAGACTGATAG |
| underlined) | | ATAGAACCAACGCTAATTTT |
| | | TTGGTGTGGCCCCCTTGCGT |
| | | GGAGGTACAAAGGTGCTCT |
| | | GGATGCTGCAATAACAGGA |
| | | ACGTACAATGCAGGCCCAC |
| | | CCAGGTACAACTGAGGCCG |
| | | GTGCAGGTCAGAAAGATCG |
| | | AGATAGTCAGAAAGAAACC |
| | | TATATTCAAAAAGGCCACCG |
| | | TCACCCTTGAAGACCATTTG |
| | | GCGTGCAAGTGTGAGACCG |
| | | TTGCGGCGGCCAGACCCGTG |
| | | ACT (SEQ ID NO: 23) |
| pAJ27: mPDGFb | SLGSLAAAEPAVIAECKTRTEVF | AGCCTAGGCAGCTTAGCCGC |
| (under the | QISRNLIDRTNANFLVWPPCVEV | TGCGGAACCCGCCGTCATAG |
| mf_alpha_prepro | QRCSGCCNNRNVQCRASQVQM | CCGAGTGCAAGACCCGTACT |
| secretion signal) | RPVQVRKIEIVRKKPIFKKATVT | GAGGTCTTCCAAATCTCACG |
| | LEDHLACKCETIVTPRPVT (SEQ | TAATTTAATCGACAGGACCA |
| | ID NO: 2) | ACGCTAACTTCTTGGTATGG |
| | | CCGCCCTGTGTTGAAGTCCA |
| | | AAGGTGCAGCGGTTGTTGTA |
| | | ATAACAGAAACGTACAATG |
| | | CCGTGCGTCCCAGGTGCAAA |
| | | TGAGGCCTGTTCAGGTGAGA |
| | | AAAATTGAAATCGTCCGTAA |
| | | AAAGCCCATATTTAAGAAA |
| | | GCTACCGTCACTCTGGAAGA |
| | | CCACCTTGCGTGTAAGTGTG |
| | | AGACCATTGTGACACCGAG |
| | | ACCAGTTACC (SEQ ID NO: |
| | | 24) |
| pAJ35: FLAG- | DYKDDDDKSLGSLTIAEPAMIAE | GACTACAAAGACGATGACG |
| hPDGFb (under | CKTRTEVFEISRRLIDRTNANFL | ACAAGAGCCTTGGGAGCCT |
| the Sed1 secretion | VWPPCVEVQRCSGCCNNRNVQ | GACCATCGCAGAGCCAGCA |
| signal peptide; | CRPTQVQLRPVQVRKIEIVRKKP | ATGATTGCTGAATGCAAAAC |
| FLAG sequence is | IFKKATVTLEDHLACKCETVAA | GAGGACAGAAGTTTTTGAA |
| underlined) | ARPVT (SEQ ID NO: 13) | ATAAGCAGAAGACTGATAG |
| | | ATAGAACCAACGCTAATTTT |
| | | TTGGTGTGGCCCCCTTGCGT |
| | | GGAGGTACAAAGGTGCTCT |
| | | GGATGCTGCAATAACAGGA |
| | | ACGTACAATGCAGGCCCAC |
| | | CCAGGTACAACTGAGGCCG |
| | | GTGCAGGTCAGAAAGATCG |
| | | AGATAGTCAGAAAGAAACC |
| | | TATATTCAAAAAGGCCACCG |
| | | TCACCCTTGAAGACCATTTG |
| | | GCGTGCAAGTGTGAGACCG |
| | | TTGCGGCGGCCAGACCCGTG |
| | | ACT (SEQ ID NO: 25) |
| pAJ30: FLAG- | DYKDDDDKSLGSLAAAEPAVIA | GACTACAAAGACGATGACG |
| mPDGFb (under | ECKTRTEVFQISRNLIDRTNANF | ACAAGAGCCTAGGCAGCTT |
| the | LVWPPCVEVQRCSGCCNNRNV | AGCCGCTGCGGAACCCGCC |
| mf_alpha_prepro | QCRASQVQMRPVQVRKIEIVRK | GTCATAGCCGAGTGCAAGA |
| secretion signal; | KPIFKKATVTLEDHLACKCETIV | CCCGTACTGAGGTCTTCCAA |
| FLAG sequence is | TPRPVT (SEQ ID NO: 14) | ATCTCACGTAATTTAATCGA |
| underlined) | | CAGGACCAACGCTAACTTCT |
| | | TGGTATGGCCGCCCTGTGTT |
| | | GAAGTCCAAAGGTGCAGCG |
| | | GTTGTTGTAATAACAGAAAC |
| | | GTACAATGCCGTGCGTCCCA |
| | | GGTGCAAATGAGGCCTGTTC |
| | | AGGTGAGAAAAATTGAAAT |
| | | CGTCCGTAAAAAGCCCATAT |
| | | TTAAGAAAGCTACCGTCACT |
| | | CTGGAAGACCACCTTGCGTG |
| | | TAAGTGTGAGACCATTGTGA |
| | | CACCGAGACCAGTTACC |
| | | (SEQ ID NO: 26) |
| pAJ02: hEGF | NSDSECPLSHDGYCLHDGVCMY | AACAGTGACTCTGAGTGTCC |
| (under the | IEALDKYACNCVVGYIGERCQY | GTTATCTCACGACGGGTATT |
| mf_alpha_prepro | RDLKWWELR (SEQ ID NO: 3) | GCTTACACGACGGAGTGTGT |
| secretion signal) | | ATGTATATAGAGGCTCTGGA |
| | | CAAGTACGCCTGTAATTGTG |
| | | TAGTAGGGTATATCGGAGA |
| | | GAGATGTCAATATAGAGATT |
| | | TGAAGTGGTGGGAACTGAG |
| | | A (SEQ ID NO: 27) |
| pAJ06: FLAG- | DYKDDDDKNSDSECPLSHDGYC | GACTACAAAGACGATGACG |
| hEGF (under the | LHDGVCMYIEALDKYACNCVV | ACAAGAACAGTGACTCTGA |
| mf_alpha_prepro | GYIGERCQYRDLKWWELR (SEQ | GTGTCCGTTATCTCACGACG |
| secretion signal; | ID NO: 15) | GGTATTGCTTACACGACGGA |
| FLAG sequence is | | GTGTGTATGTATATAGAGGC |
| underlined) | | TCTGGACAAGTACGCCTGTA |
| | | ATTGTGTAGTAGGGTATATC |
| | | GGAGAGAGATGTCAATATA |
| | | GAGATTTGAAGTGGTGGGA |
| | | ACTGAGA (SEQ ID NO: 28) |
| pAJ26: mEGF | NSYPGCPSSYDGYCLNGGVCMH | AACTCTTATCCAGGATGTCC |
| (under the | IESLDSYTCNCVIGYSGDRCQTR | CTCTTCCTACGATGGGTACT |
| mf_alpha_prepro | DLRWWELR (SEQ ID NO: 4) | GCTTGAACGGGGGAGTGTG |
| secretion signal) | | TATGCATATTGAATCCCTTG |
| | | ATAGTTATACGTGTAATTGC |
| | | GTAATCGGGTACAGCGGTG |
| | | ATAGGTGTCAGACGCGTGAT |
| | | CTGAGGTGGTGGGAGTTGC |
| | | GC (SEQ ID NO: 29) |
| pAJ29: FLAG- | DYKDDDDKNSYPGCPSSYDGYC | GACTACAAAGACGATGACG |
| mEGF (under the | LNGGVCMHIESLDSYTCNCVIG | ACAAGAACTCTTATCCAGGA |
| mf_alpha_prepro | YSGDRCQTRDLRWWELR (SEQ | TGTCCCTCTTCCTACGATGG |
| secretion signal; | ID NO: 16) | GTACTGCTTGAACGGGGGA |
| FLAG sequence is | | GTGTGTATGCATATTGAATC |
| underlined) | | CCTTGATAGTTATACGTGTA |
| | | ATTGCGTAATCGGGTACAGC |
| | | GGTGATAGGTGTCAGACGC |
| | | GTGATCTGAGGTGGTGGGA |
| | | GTTGCGC (SEQ ID NO: 30) |
| pAJ33: FLAG- | DYKDDDDKNSYPGCPSSYDGYC | GACTACAAAGACGATGACG |
| mEGF (under the | LNGGVCMHIESLDSYTCNCVIG | ACAAGAACTCTTATCCAGGA |
| Sed1 secretion | YSGDRCQTRDLRWWELR (SEQ | TGTCCCTCTTCCTACGATGG |
| signal peptide; | ID NO: 16) | GTACTGCTTGAACGGGGGA |
| FLAG sequence is | | GTGTGTATGCATATTGAATC |
| underlined) | | CCTTGATAGTTATACGTGTA |
| | | ATTGCGTAATCGGGTACAGC |
| | | GGTGATAGGTGTCAGACGC |
| | | GTGATCTGAGGTGGTGGGA |
| | | GTTGCGC (SEQ ID NO: 31) |
| pAJ34: FLAG- | DYKDDDDKVPIQKVQDDTKTLI | GACTACAAAGACGATGACG |
| mLeptin (under | KTIVTRINDISHTQSVSAKQRVT | ACAAGGTGCCTATACAAAA |
| the Sed1 secretion | GLDFIPGLHPILSLSKMDQTLAV | GGTACAAGATGATACTAAG |
| signal peptide; | YQQVLTSLPSQNVLQIANDLENL | ACATTGATTAAAACGATTGT |
| FLAG sequence is | RDLLHLLAFSKSCSLPQTSGLQK | CACTCGTATCAACGACATTT |
| underlined) | PESLDGVLEASLYSTEVVALSRL | CCCATACACAATCAGTGTCA |
| | QGSLQDILQQLDVSPEC (SEQ ID | GCAAAGCAAAGGGTAACGG |
| | NO: 17) | GTCTTGATTTCATTCCCGGT |
| | | TTGCACCCGATATTATCATT |
| | | ATCCAAGATGGATCAGACCT |
| | | TAGCCGTATATCAACAAGTT |
| | | CTGACATCCCTTCCGTCCCA |
| | | GAATGTACTGCAAATCGCA |
| | | AATGACCTTGAAAACCTGCG |
| | | TGACCTTCTACATCTACTAG |
| | | CGTTCTCCAAATCTTGCTCT |
| | | TTACCACAGACATCAGGGCT |
| | | TCAAAAACCGGAGTCCTTAG |
| | | ACGGAGTCCTTGAAGCATCT |
| | | TTGTACTCTACGGAAGTTGT |
| | | TGCCCTAAGTAGGCTTCAAG |
| | | GCTCTTTACAAGACATCCTG |
| | | CAACAGCTTGACGTAAGCCC |
| | | AGAGTGC (SEQ ID NO: 32) |
| pAJ24: hLeptin | VPIQKVQDDTKTLIKTIVTRINDI | GTTCCTATTCAGAAAGTCCA |
| (under the | SHTQSVSSKQKVTGLDFIPGLHPI | AGACGACACAAAGACCCTT |
| mf_alpha_prepro | LTLSKMDQTLAVYQQILTSMPS | ATTAAAACTATTGTAACTAG |
| secretion signal) | RNVIQISNDLENLRDLLHVLAFS | AATAAATGATATAAGTCAC |
| | KSCHLPWASGLETLDSLGGVLE | ACACAATCAGTATCAAGTA |
| | ASGYSTEVVALSRLQGSLQDML | AGCAAAAAGTGACAGGGTT |
| | WQLDLSPGC (SEQ ID NO: 5) | GGACTTCATTCCCGGCCTTC |
| | | ACCCGATACTTACGCTATCT |
| | | AAGATGGACCAAACGCTTG |
| | | CTGTCTACCAGCAGATCTTG |
| | | ACTAGTATGCCGTCCCGTAA |
| | | TGTCATACAAATTTCCAACG |
| | | ACCTGGAGAATTTAAGAGA |
| | | TTTGCTGCACGTATTAGCGT |
| | | TCAGTAAGAGTTGCCATCTT |
| | | CCCTGGGCTAGTGGCTTAGA |
| | | AACTCTTGATTCCCTTGGCG |
| | | GTGTGTTAGAAGCGTCCGGC |
| | | TACAGCACAGAAGTAGTAG |
| | | CCCTGAGTAGGCTTCAGGGC |
| | | AGTTTACAAGACATGCTATG |
| | | GCAACTGGACCTATCTCCGG |
| | | GCTGC (SEQ ID NO: 33) |
| pAJ25: hLeptin | VPIQKVQDDTKTLIKTIVTRINDI | GTTCCTATTCAGAAAGTCCA |
| (under the Sed1 | SHTQSVSSKQKVTGLDFIPGLHPI | AGACGACACAAAGACCCTT |
| secretion signal | LTLSKMDQTLAVYQQILTSMPS | ATTAAAACTATTGTAACTAG |
| peptide) | RNVIQISNDLENLRDLLHVLAFS | AATAAATGATATAAGTCCAC |
| | KSCHLPWASGLETLDSLGGVLE | AATCAGTATCAAGTAAGCA |
| | ASGYSTEVVALSRLQGSLQDML | AAAAGTGACAGGGTTGGAC |
| | WQLDLSPGC (SEQ ID NO: 5) | TTCATTCCCGGCCTTCACCC |
| | | GATACTTACGCTATCTAAGA |
| | | TGGACCAAACGCTTGCTGTC |
| | | TACCAGCAGATCTTGACTAG |
| | | TATGCCGTCCCGTAATGTCA |
| | | TACAAATTTCCAACGACCTG |
| | | GAGAATTTAAGAGATTTGCT |
| | | GCACGTATTAGCGTTCAGTA |
| | | AGAGTTGCCATCTTCCCTGG |
| | | GCTAGTGGCTTAGAAACTCT |
| | | TGATTCCCTTGGCGGTGTGT |
| | | TAGAAGCGTCCGGCTACAG |
| | | CACAGAAGTAGTAGCCCTG |
| | | AGTAGGCTTCAGGGCAGTTT |
| | | ACAAGACATGCTATGGCAA |
| | | CTGGACCTATCTCCGGGCTG |
| | | C (SEQ ID NO: 34) |
| pAJ28: mLeptin | VPIQKVQDDTKTLIKTIVTRINDI | GTGCCTATACAAAAGGTAC |
| (under the | SHTQSVSAKQRVTGLDFIPGLHP | AAGATGATACTAAGACATT |
| mf_alpha_prepro | ILSLSKMDQTLAVYQQVLTSLPS | GATTAAAACGATTGTCACTC |
| secretion signal) | QNVLQIANDLENLRDLLHLLAFS | GTATCAACGACATTTCCCAT |
| | KSCSLPQTSGLQKPESLDGVLEA | ACACAATCAGTGTCAGCAA |
| | SLYSTEVVALSRLQGSLQDILQQ | AGCAAAGGGTAACGGGTCT |
| | LDVSPEC (SEQ ID NO: 6) | TGATTTCATTCCCGGTTTGC |
| | | ACCCGATATTATCATTATCC |
| | | AAGATGGATCAGACCTTAG |
| | | CCGTATATCAACAAGTTCTG |
| | | ACATCCCTTCCGTCCCAGAA |
| | | TGTACTGCAAATCGCAAATG |
| | | ACCTTGAAAACCTGCGTGAC |
| | | CTTCTACATCTACTAGCGTT |
| | | CTCCAAATCTTGCTCTTTAC |
| | | CACAGACATCAGGGCTTCA |
| | | AAAACCGGAGTCCTTAGAC |
| | | GGAGTCCTTGAAGCATCTTT |
| | | GTACTCTACGGAAGTTGTTG |
| | | CCCTAAGTAGGCTTCAAGGC |
| | | TCTTTACAAGACATCCTGCA |
| | | ACAGCTTGACGTAAGCCCA |
| | | GAGTGC (SEQ ID NO: 35) |
| pAJ31: FLAG- | DYKDDDDKVPIQKVQDDTKTLI | GACTACAAAGACGATGACG |
| mLeptin (under | KTIVTRINDISHTQSVSAKQRVT | ACAAGGTGCCTATACAAAA |
| the | GLDFIPGLHPILSLSKMDQTLAV | GGTACAAGATGATACTAAG |
| mf_alpha_prepro | YQQVLTSLPSQNVLQIANDLENL | ACATTGATTAAAACGATTGT |
| secretion signal; | RDLLHLLAFSKSCSLPQTSGLQK | CACTCGTATCAACGACATTT |
| FLAG sequence is | PESLDGVLEASLYSTEVVALSRL | CCCATACACAATCAGTGTCA |
| underlined) | QGSLQDILQQLDVSPEC (SEQ ID | GCAAAGCAAAGGGTAACGG |
| | NO: 18) | GTCTTGATTTCATTCCCGGT |
| | | TTGCACCCGATATTATCATT |
| | | ATCCAAGATGGATCAGACCT |
| | | TAGCCGTATATCAACAAGTT |
| | | CTGACATCCCTTCCGTCCCA |
| | | GAATGTACTGCAAATCGCA |
| | | AATGACCTTGAAAACCTGCG |
| | | TGACCTTCTACATCTACTAG |
| | | CGTTCTCCAAATCTTGCTCT |
| | | TTACCACAGACATCAGGGCT |
| | | TCAAAAACCGGAGTCCTTAG |
| | | ACGGAGTCCTTGAAGCATCT |
| | | TTGTACTCTACGGAAGTTGT |
| | | TGCCCTAAGTAGGCTTCAAG |
| | | GCTCTTTACAAGACATCCTG |
| | | CAACAGCTTGACGTAAGCCC |
| | | AGAGTGC (SEQ ID NO: 36) |
| pAJ32: FLAG- | DYKDDDDKVPIQKVQDDTKTLI | GACTACAAAGACGATGACG |
| hLeptin (under the | KTIVTRINDISHTQSVSSKQKVT | ACAAGGTTCCTATTCAGAAA |
| mf_alpha_prepro | GLDFIPGLHPILTLSKMDQTLAV | GTCCAAGACGACACAAAGA |
| secretion signal; | YQQILTSMPSRNVIQISNDLENL | CCCTTATTAAAACTATTGTA |
| FLAG sequence is | RDLLHVLAFSKSCHLPWASGLE | ACTAGAATAAATGATATAA |
| underlined) | TLDSLGGVLEASGYSTEVVALS | GTCACACACAATCAGTATCA |
| | RLQGSLQDMLWQLDLSPGC | AGTAAGCAAAAAGTGACAG |
| | (SEQ ID NO: 19) | GGTTGGACTTCATTCCCGGC |
| | | CTTCACCCGATACTTACGCT |
| | | ATCTAAGATGGACCAAACG |
| | | CTTGCTGTCTACCAGCAGAT |
| | | CTTGACTAGTATGCCGTCCC |
| | | GTAATGTCATACAAATTTCC |
| | | AACGACCTGGAGAATTTAA |
| | | GAGATTTGCTGCACGTATTA |
| | | GCGTTCAGTAAGAGTTGCCA |
| | | TCTTCCCTGGGCTAGTGGCT |
| | | TAGAAACTCTTGATTCCCTT |
| | | GGCGGTGTGTTAGAAGCGTC |
| | | CGGCTACAGCACAGAAGTA |
| | | GTAGCCCTGAGTAGGCTTCA |
| | | GGGCAGTTTACAAGACATG |
| | | CTATGGCAACTGGACCTATC |
| | | TCCGGGCTGC (SEQ ID NO: |
| | | 37) |
| pAJ39: FLAG- | DYKDDDDKKPVSLSYRCPCRFF | GACTACAAAGACGATGACG |
| mCXCL12 (under | ESHIARANVKHLKILNTPNCALQ | ACAAGAAACCTGTATCACTA |
| the | IVARLKNNNRQVCIDPKLKWIQ | TCATACAGATGTCCCTGTAG |
| mf_alpha_prepro | EYLEKALNK (SEQ ID NO: 20) | GTTCTTCGAGAGTCATATTG |
| secretion signal; | | CACGTGCCAATGTAAAACA |
| FLAG sequence is | | CCTTAAGATCCTAAATACAC |
| underlined) | | CCAACTGTGCCCTACAAATA |
| | | GTGGCACGTTTGAAGAATA |
| | | ATAATCGTCAAGTCTGCATC |
| | | GACCCCAAGTTGAAGTGGA |
| | | TACAGGAGTATCTTGAGAA |
| | | GGCACTGAACAAA (SEQ ID |
| | | NO: 38) |
| pAJ37: mCXCL12 | KPVSLSYRCPCRFFESHIARANV | AAACCTGTATCACTATCATA |
| (under the | KHLKILNTPNCALQIVARLKNN | CAGATGTCCCTGTAGGTTCT |
| mf_alpha_prepro | NRQVCIDPKLKWIQEYLEKALN | TCGAGAGTCATATTGCACGT |
| secretion signal) | K (SEQ ID NO: 7) | GCCAATGTAAAACACCTTAA |
| | | GATCCTAAATACACCCAACT |
| | | GTGCCCTACAAATAGTGGCA |
| | | CGTTTGAAGAATAATAATCG |
| | | TCAAGTCTGCATCGACCCCA |
| | | AGTTGAAGTGGATACAGGA |
| | | GTATCTTGAGAAGGCACTGA |
| | | ACAAA (SEQ ID NO: 39) |
| pAJ38: FLAG- | DYKDDDDKKPVSLSYRCPCRFF | GACTACAAAGACGATGACG |
| hCXCL12 (under | ESHVARANVKHLKILNTPNCAL | ACAAGAAACCTGTTAGCCTA |
| the | QIVARLKNNNRQVCIDPKLKWI | TCTTACCGTTGTCCATGTAG |
| mf_alpha_prepro | QEYLEKALNK (SEQ ID NO: 21) | GTTTTTTGAAAGTCACGTTG |
| secretion signal; | | CGAGGGCGAATGTGAAGCA |
| FLAG sequence is | | CTTAAAGATCCTAAATACAC |
| underlined) | | CGAATTGTGCACTACAAATT |
| | | GTCGCAAGACTAAAGAACA |
| | | ACAATAGGCAAGTATGTATT |
| | | GACCCCAAATTAAAATGGA |
| | | TACAAGAATACCTAGAAAA |
| | | GGCGTTGAACAAA (SEQ ID |
| | | NO: 40) |
| pAJ36: hCXCL12 | KPVSLSYRCPCRFFESHVARANV | AAACCTGTTAGCCTATCTTA |
| (under the | KHLKILNTPNCALQIVARLKNN | CCGTTGTCCATGTAGGTTTT |
| mf_alpha_prepro | NRQVCIDPKLKWIQEYLEKALN | TTGAAAGTCACGTTGCGAGG |
| secretion signal) | K (SEQ ID NO: 8) | GCGAATGTGAAGCACTTAA |
| | | AGATCCTAAATACACCGAAT |
| | | TGTGCACTACAAATTGTCGC |
| | | AAGACTAAAGAACAACAAT |
| | | AGGCAAGTATGTATTGACCC |
| | | CAAATTAAAATGGATACAA |
| | | GAATACCTAGAAAAGGCGT |
| | | TGAACAAA (SEQ ID NO: 41) |

*The amino acid sequences can begin with a methionine (M) and the nucleotide sequences can begin with the start codon ATG.

TABLE 3

| Plasmid Name | Description |
|---|---|
| pAJ01 | ADH1p-Mfalpha prepro-GFP |
| pAJ02 | ADH1p-MF alpha prepro-hEGF |
| pAJ03 | ADH1p-Mfallpha prepro-hPDGF b |
| pAJ04 | ADH1p-hEGF |
| pAJ05 | ADH1p-Mfalpha prepro-FLAG-hPDGF b |
| pAJ06 | ADH1p-Mfalpha prepro-FLAG-hEGF |
| pAJ07 | ADH1p-FLAG-hEGF |
| pAJ08 | ADH1p-FLAG-hPDGF b |
| pAJ09 | CAN.1 SgRNA |
| pAJ10 | His6X-SMT3-FLAG |
| pAJ11 | ADH1p-Mfalpha prepro-Sc alpha factor- hPDGF b |
| pAJ12 | ADH1p-Mfalpha prepro-Sc alpha factor-hEGF |
| pAJ13 | pHXT1-yEmRFP-tCyc1 |
| pAJ14 | UpHomology-pGPD-PDII-tSte2-DownHomology |
| pAJ15 | pHXT1-Ca alpha factor-CYCt |
| pAJ16 | Sed1p-Sed1 prepro-tCyc1 |
| pAJ17 | Sed1p-Sed1 prepro-eGFP-tCyc1 |
| pAJ18 | Sed1p-Sed1 prepro-Flag-GFP-Sc alpha factor-tCyc1 |
| pAJ19 | Sed1p-Sed1 prepro-alphaFa + D23ctor-GFP-Flag-tCyc1 |
| pAJ20 | Sed1p-Sed1 prepro-Flag-linker-GFP-linker-Sc alpha Factor-tCyc1 |
| pAJ21 | Sed1p-Sed1 prepro-Sc alpha factor-GGSGGGSGG-GFP-GGSGGGSGG-Flag-tCyc1 |
| pAJ22 | Sed1p-Sed1 prepro-Sc alpha factor-GGSGGGSGG-FLAG-tCyc1 |
| pAJ23 | Sed1p-Sed1 prepro-FLAG-GGSGGGSGG-Sc alpha factor-tCyc1 |
| pAJ24 | Mfalpha-prepro-hLEP-tCyc1 |
| pAJ25 | Sed1p-Sed1 prepro-hLEP-tCyc1 |
| pAJ26 | ADH1p-Mfalpha prepro- mEGF-tCyc1 |
| pAJ27 | ADH1p-Mfalpha prepro-mPDGFb-tCyc1 |
| pAJ28 | ADH1p-Mfalpha prepro-mLEP-tCyc1 |
| pAJ29 | ADH1p-Mfalpha prepro-FLAG-mEGF-tCyc1 |
| pAJ30 | ADH1p-Mfalpha prepro-FLAG-mPDGFb-tCyc1 |
| pAJ31 | ADH1p-Mfalpha prepro-FLAG-mLEP-tCyc1 |
| pAJ32 | ADH1p-Mfalpha prepro-FLAG-hLEP-tCyc1 |
| pAJ33 | Sed1p-Sed1 prepro-FLAG-mEGF-tCyc1 |
| pAJ34 | Sed1p-Sed1 prepro-FLAG-mLEP-tCyc1 |
| pAJ35 | Sed1p-Sed1 prepro-FLAG-hPDGF-tCyc1 |
| pAJ36 | ADH1p-Mfalpha prepro-hCXCL12-tCyc1 |
| pAJ37 | ADH1p-Mfalpha prepro-mCXCL12-tCyc1 |
| pAJ38 | ADH1p-Mfalpha prepro-FLAG-hCXCL12-tCyc1 |
| pAJ39 | ADH1p-Mfalpha prepro-FLAG-mCXCL12-tCyc1 |
| pAJ40 | ADH1p-MFalpha-prepro-rh-LEP-tCyc1-Sed1p-Sed1 prepro-Ca alpha factor-tADH1 |
| pAJ41 | ADH1p-Mfalpha prepro-mLEP-tCyc1-Sed1p-Sed1 prepro-Ca alpha factor-tADH1 |
| pAJ42 | ADH1p-Mfalpha prepro-mEGF-tCyc1-Sed1p-Sed1 prepro-Vp alpha factor-tADH1 |
| pAJ43 | ADH1p-Mfallpha prepro-hEGF-tCyc1-Sed1p-Sed1 prepro-Vp alpha factor-tADH1 |

TABLE 4

| | | | Sed1 | mf_alpha | | PDI1 | |
|---|---|---|---|---|---|---|---|
| | Parent | Plasmid | Secretion | Secretion | | (CRISPRed | |
| Yeast | yeast | from | Signal | Signal | FLAG | into | Protein |
| Strain | strain | Table 2 | Peptide | Peptide | Chimera | ARS208a) | Factor |
| yAJ5 | FY251 | pAJ5 | | Y | Y | | hPDGF-B |
| yAJ8 | FY251 | pAJ8 | | | Y | | |
| yAJ25 | yAJ23 | pAJ5 | | Y | Y | Y | |
| yAJ32 | FY251 | pAJ30 | | Y | Y | | mPDGF-B |
| yAJ29 | FY251 | pAJ27 | | Y | | | |
| yAJ27 | FY251 | pAJ24 | | Y | | | hLEP |
| yAJ34 | FY251 | pAJ32 | | Y | Y | | |
| yAJ30 | FY251 | pAJ28 | | Y | | | mLEP |
| yAJ33 | FY251 | pAJ31 | | Y | Y | | |
| yAJ35 | FY251 | pAJ36 | | Y | | | hCXCL12 |
| yAJ37 | FY251 | pAJ38 | | Y | Y | | |
| yAJ36 | FY251 | pAJ37 | | Y | | | mCXCL12 |
| yAJ38 | FY251 | pAJ39 | | Y | Y | | |
| yAJ2 | FY251 | pAJ4 | | | | | hEGF |
| yAJ6 | FY251 | pAJ6 | | Y | Y | | |
| yAJ7 | FY251 | pAJ7 | | | Y | | |
| yAJ26 | yAJ23 | pAJ6 | | Y | Y | Y | |
| yAJ39 | FY251 | pAJ2 | | Y | | | |
| yAJ28 | FY251 | pAJ26 | | Y | Y | | mEGF |
| yAJ31 | FY251 | pAJ29 | | Y | Y | | |
| yAJ40 | FY251 | pAJ33 | Y | | Y | | |

TABLE 5

| Promoter | Sequence |
|---|---|
| pGPD | AGTTTATCATTATCAATACTGCCATTTCAAAGAATACGTAAATAATTAAT |
| | AGTAGTGATTTTCCTAACTTTATTTAGTCAAAAAATTAGCCTTTTAATTCT |
| | GCTGTAACCCGTACATGCCCAAAATAGGGGGCGGGTTACACAGAATATA |
| | TAACATCGTAGGTGTCTGGGTGAACAGTTTATTCCTGGCATCCACTAAAT |
| | ATAATGGAGCCCGCTTTTTAAGCTGGCATCCAGAAAAAAAAAGAATCCC |
| | AGCACCAAAATATTGTTTTCTTCACCAACCATCAGTTCATAGGTCCATTC |
| | TCTTAGCGCAACTACAGAGAACAGGGGCACAAACAGGCAAAAAACGGG |
| | CACAACCTCAATGGAGTGATGCAACCTGCCTGGAGTAAATGATGACACA |
| | AGGCAATTGACCCACGCATGTATCTATCTCATTTTCTTACACCTTCTATTA |
| | CCTTCTGCTCTCTCTGATTTGGAAAAAGCTGAAAAAAAAGGTTGAAACC |
| | AGTTCCCTGAAATTATTCCCCTACTTGACTAATAAGTATATAAAGACGGT |
| | AGGTATTGATTGTAATTCTGTAAATCTATTTCTTAAACTTCTTAAATTCTA |
| | CTTTTATAGTTAGTCTTTTTTTTAGTTTTAAAACACCAAGAACTTAGTTTC |
| | GACGGAT (SEQ ID NO: 42) |
| pADH1 | GGGTGTACAATATGGACTTCCTCTTTTCTGGCAACCAAACCCATACATCG |
| | GGATTCCTATAATACCTTCGTTGGTCTCCCTAACATGTAGGTGGCGGAGG |
| | GGAGATATACAATAGAACAGATACCAGACAAGACATAATGGGCTAAAC |
| | AAGACTACACCAATTACACTGCCTCATTGATGGTGGTACATAACGAACT |
| | AATACTGTAGCCCTAGACTTGATAGCCATCATCATATCGAAGTTTCACTA |
| | CCCTTTTTCCATTTGCCATCTATTGAAGTAATAATAGGCGCATGCAACTT |
| | CTTTTCTTTTTTTTTCTTTTCTCTCTCCCCCGTTGTTGTCTCACCATATCCG |
| | CAATGACAAAAAAATGATGGAAGACACTAAAGGAAAAAATTAACGACA |
| | AAGACAGCACCAACAGATGTCGTTGTTCCAGAGCTGATGAGGGGTATCT |
| | CGAAGCACACGAAACTTTTTCCTTCCTTCATTCACGCACACTACTCTCTA |
| | ATGAGCAACGGTATACGGCCTTCCTTCCAGTTACTTGAATTTGAAATAAA |
| | AAAAAGTTTGCTGTCTTGCTATCAAGTATAAATAGACCTGCAATTATTAA |
| | TCTTTTGTTTCCTCGTCATTGTTCTCGTTCCCTTTCTTCCTTGTTTCTTTTTC |
| | TGCACAATATTTCAAGCTATACCAAGCATACAATCAACT (SEQ ID NO: 43) |
| pSed1 | ATTGGATATAGAAAATTAACGTAAGGCAGTATCTTTTCACAATGTACTTG |
| | CAACGCGGCGACTTAAAGTTGAAGTACAACCTGCAGCAGCGGCTTTTTG |
| | TACGGTACGCCAAACTGTCAATGGATAATATTGCGTAGACCGAAAAAGG |
| | TAATCCTCAACACTACCCGTGGTGGATGACCTAAAGCAGTAATATTGGTT |
| | GGAATTATCTCCCAGACGGCACCGTCTCCCCGAGAAAGCTTAGCCCCGA |
| | GGTCTACCTTCCATACACCACTGATTGCTCCACGTCATGCGGCCTTCTTTC |
| | GAGGACAAAAAGGCATATATCGCTAAAATTAGCCATCAGAACCGTTATT |
| | GTTATTATATTTTCATTACGAAAGAGGAGAGGGCCCAGCGCGCCAGAGC |
| | ACACACGGTCATTGATTACTTTATTTGGCTAAAGATCCATCCCTTCTCGA |
| | TGTCATCTCTTTCCATTCTTGTGTATTTTTGATTGAAAATGATTTTTTGTCC |
| | ACTAATTTCTAAAAATAAGACAAAAAGCCTTTAAGCAGTTTTTCATCCAT |
| | TTTACTACGGTAAAATGAATTAGTACGGTATGGCTCCCAGTCGCATTATT |
| | TTTAGATTGGCCGTAGGGGCTGGGGTAGAACTAGAGTAAGGAACATTGC |
| | TCTGCCCTCTTTTGAACTGTCATATAAATACCTGACCTATTTTATTCTCCA |
| | TTATCGTATTATCTCACCTCTCTTTTTCTATTCTCTTGTAATTATTGATTTA |
| | TAGTCGTAACTACAAAGACAAGCAAAATAAAATACGTTCGCTCTATTAA |
| | G (SEQ ID NO: 44) |
| pHXT1 | GGCCACAATGAAACTTCAATTCATATCGACCGACTATTTTTCTCCGAACC |
| | AAAAAAATAGCAGGGCGAGATTGGAGCTGCGGAAAAAAGAGGAAAAAA |
| | TTTTTTCGTAGTTTTCTTGTGCAAATTAGGGTGTAAGGTTTCTAGGGCTTA |
| | TTGGTTCAAGCAGAAGAGACAACAATTGTAGGTCCTAAATTCAAGGCGG |
| | ATGTAAGGAGTATTGGTTTCGAAAGTTTTTCCGAAGCGGCATGGCAGGG |
| | ACTACTTCGCATGCGCTCGGATTATCTTCATTTTTGCTTGCAAAAACGTA |
| | GAATCATGGTAAATTACATGAAGAATTCTCTTTTTTTTTTTTTTTTTTTTTT |
| | TTTTACCTCTAAAGAGTGTTGACCAACTGAAAAAACCCTTCTTCAAGAGA |
| | GTTAAACTAAGACTAACCATCATAACTTCCAAGGAATTAATCGATATCTT |
| | GCACTCCTGATTTTTCTTCAAAGAGACAGCGCAAAGGATTATGACACTGT |
| | TGCATTGAGTCAAAAGTTTTTCCGAAGTGACCCAGTGCTCTTTTTTTTTTT |
| | CCGTGAAGGACTGACAAATATGCGCACAAGATCCAATACGTAATGGAAA |
| | TTCGGAAAAACTAGGAAGAAATGCTGCAGGGCATTGCCGTGCCGATCTT |
| | TTGTCTTTCAGATATATGAGAAAAAGAATATTCATCAAGTGCTGATAGA |
| | AGAATACCACTCATATGACGTGGGCAGAAGACAGCAAACGTAAACATG |
| | AGCTGCTGCGACATTTGATGGCTTTTATCCGACAAGCCAGGAAACTCCAC |
| | CATTATCTAATGTAGCAAAATATTTCTTAACACCCGAAGTTGCGTGTCCC |
| | CCTCACGTTTTTAATCATTTGAATTAGTATATTGAAATTATATATAAAGG |
| | CAACAATGTCCCCATAATCAATTCCATCTGGGGTCTCATGTTCTTTCCCC |
| | ACCTTAAAATCTATAAAGATATCATAATCGTCAACTAGTTGATATACGTA |
| | AAATC (SEQ ID NO: 45) |
| TDH2 | CAGTTCGAGTTTATCATTATCAATACTGCCATTTCAAAGAATACGTAAAT |
| | AATTAATAGTAGTGATTTTCCTAACTTTATTTAGTCAAAAAATTAGCCTT |
| | TTAATTCTGCTGTAACCCGTACATGCCCAAAATAGGGGGCGGGTTACAC |
| | AGAATATATAACATCGTAGGTGTCTGGGTGAACAGTTTATTCCTGGCATC |
| | CACTAAATATAATGGAGCCCGCTTTTTAAGCTGGCATCCAGAAAAAAAA |
| | AGAATCCCAGCACCAAAATATTGTTTTCTTCACCAACCATCAGTTCATAG |
| | GTCCATTCTCTTAGCGCAACTACAGAGAACAGGGGCACAAACAGGCAAA |
| | AAACGGGCACAACCTCAATGGAGTGATGCAACCTGCCTGGAGTAAATGA |
| | TGACACAAGGCAATTGACCCACGCATGTATCTATCTCATTTTCTTACACC |
| | TTCTATTACCTTCTGCTCTCTCTGATTTGGAAAAAGCTGAAAAAAAAGGT |
| | TGAAACCAGTTCCCTGAAATTATTCCCCTACTTGACTAATAAGTATATAA |
| | AGACGGTAGGTATTGATTGTAATTCTGTAAATCTATTTCTTAAACTTCTT |
| | AAATTCTACTTTTATAGTTAGTCTTTTTTTTAGTTTTAAAACACCAAGAAC |
| | TTAGTTTCGAATAAACACACATAAACAAACAAA (SEQ ID NO: 62) |

TABLE 6

| Secretion | | |
|---|---|---|
| peptide | Nucleotide Sequence | Amino Acid Sequence |
| MF(alpha)1 | ATGAGATTTCCTTCAATTTTTACTG | MRFPSIFTAVLFAASSALAAPV |
| | CAGTTTTATTCGCAGCATCCTCCG | NTTTEDETAQIPAEAVIGYLDL |
| | CATTAGCTGCTCCAGTCAACACTA | EGDFDVAVLPFSNSTNNGLLFI |
| | CAACAGAAGATGAAACGGCACAA | NTTIASIAAKEEGV (SEQ ID |
| | ATTCCGGCTGAAGCTGTCATCGGT | NO: 49) |
| | TACTTAGATTTAGAAGGGGATTTC | |
| | GATGTTGCTGTTTTGCCATTTTCCA | |
| | ACAGCACAAATAACGGGTTATTGT | |
| | TTATAAATACTACTATTGCCAGCA | |
| | TTGCTGCTAAAGAAGAAGGGGTA | |
| | (SEQ ID NO: 46) | |
| MF(alpha)1 + | ATGAGATTTCCTTCAATTTTTACTG | MRFPSIFTAVLFAASSALAAPV |
| KREAEA | CAGTTTTATTCGCAGCATCCTCCG | NTTTEDETAQIPAEAVIGYLDL |
| | CATTAGCTGCTCCAGTCAACACTA | EGDFDVAVLPFSNSTNNGLLFI |
| | CAACAGAAGATGAAACGGCACAA | NTTIASIAAKEEGVSLDKREAE |
| | ATTCCGGCTGAAGCTGTCATCGGT | A (SEQ ID NO: 50) |
| | TACTTAGATTTAGAAGGGGATTTC | |
| | GATGTTGCTGTTTTGCCATTTTCCA | |
| | ACAGCACAAATAACGGGTTATTGT | |
| | TTATAAATACTACTATTGCCAGCA | |
| | TTGCTGCTAAAGAAGAAGGGGTAT | |
| | CTTTGGATAAAAGAGAGGCTGAA | |
| | GCT (SEQ ID NO: 47) | |
| Sed1 | ATGAAATTATCAACTGTCCTATTA | MKLSTVLLSAGLASTTLAQ |
| | TCTGCCGGTTTAGCCTCGACTACT | (SEQ ID NO: 51) |
| | TTGGCCCAA (SEQ ID NO: 48) | |
| Pre-Pro- | ATGAGATTTCCTTCAATTTTTACTG | MRFPSIFTAVLFAASSALAAPV |
| (Kex2) | CAGTTTTATTCGCAGCATCCTCCG | NTTTEDETAQIPAEAVIGYLDL |
| | CATTAGCTGCTCCAGTCAACACTA | EGDFDVAVLPFSNSTNNGLLFI |
| | CAACAGAAGATGAAACGGCACAA | NTTIASIAAKEEGVSLDKR |
| | ATTCCGGCTGAAGCTGTCATCGGT | (SEQ ID NO: 58) |
| | TACTTAGATTTAGAAGGGGATTTC | |
| | GATGTTGCTGTTTTGCCATTTTCCA | |
| | ACAGCACAAATAACGGGTTATTGT | |
| | TTATAAATACTACTATTGCCAGCA | |
| | TTGCTGCTAAAGAAGAAGGGGTAT | |
| | CTTTGGATAAAAGA (SEQ ID NO: | |
| | 63) | |

Saccharomyces cerevisiae strain FY251 was transformed with high copy number plasmids for secretion of (1) a FLAG-tag-human recombinant EGF chimera and (2) a FLAG-tag-human Leptin chimera under the constitutive transcriptional control of pADH1. Similarly, Saccharomyces cerevisiae strain FY251 was transformed with high copy number plasmids for secretion of mouse and human CXCL12. FY251 (MATa leu2Δ1 trpΔ63 ura3-52 his3-200) was used as parent yeast strain because it does not secrete or express recombinant proteins. A modified version of the lithium acetate method was used to transform yeast cells. In the secretion vectors, the cDNA for the alpha-factor signal peptide is positioned between the promoter and the cDNA for the skin therapeutic to be secreted. The transformed yeast were grown in synthetic complete media under standard conditions for 24 hours, and protein expression levels in the supernatant were subsequently analyzed. Quantitative western blots were performed to quantify the concentration of the secreted proteins over time using serial dilutions of 6×His-Smt3-FLAG protein, which was expressed in E. coli, purified using Ni column and quantified via Bradford Assay.

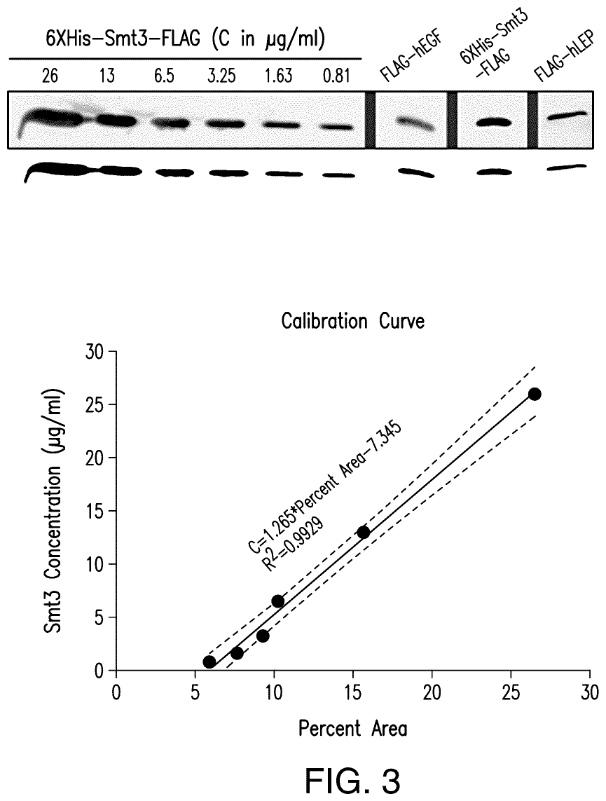

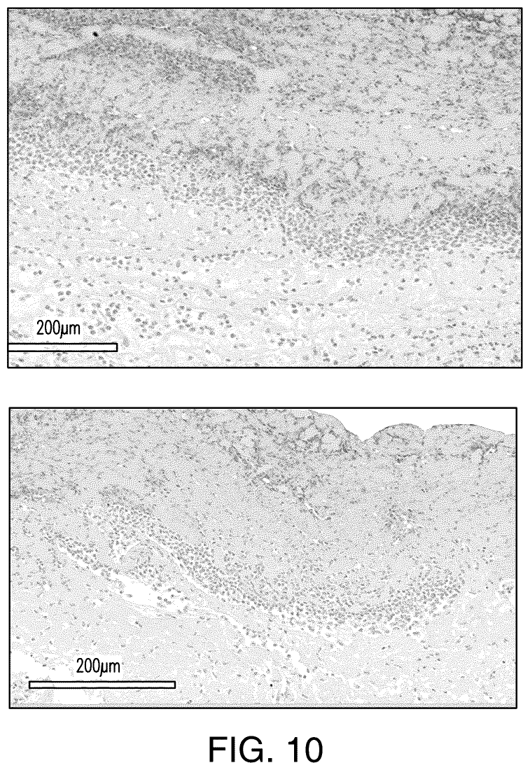

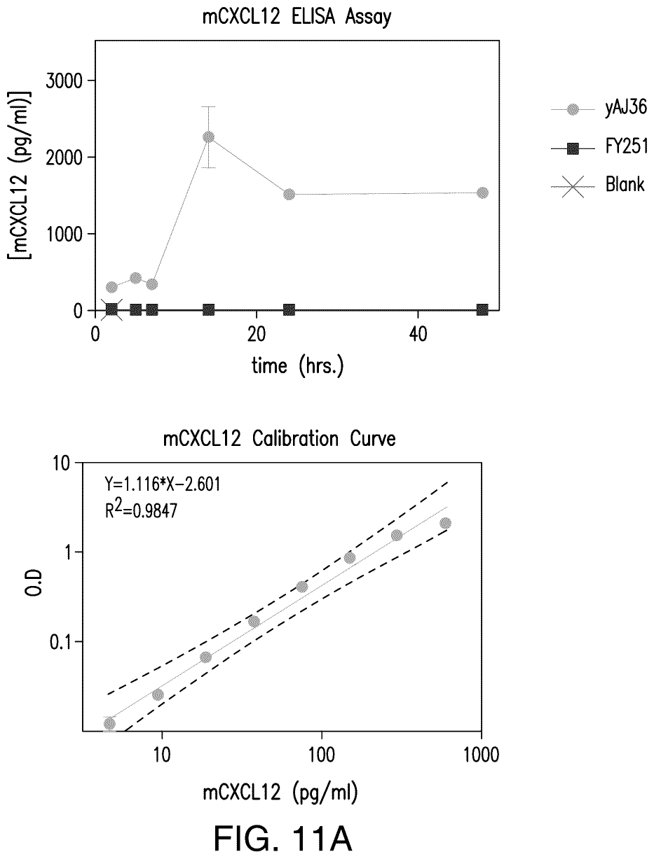

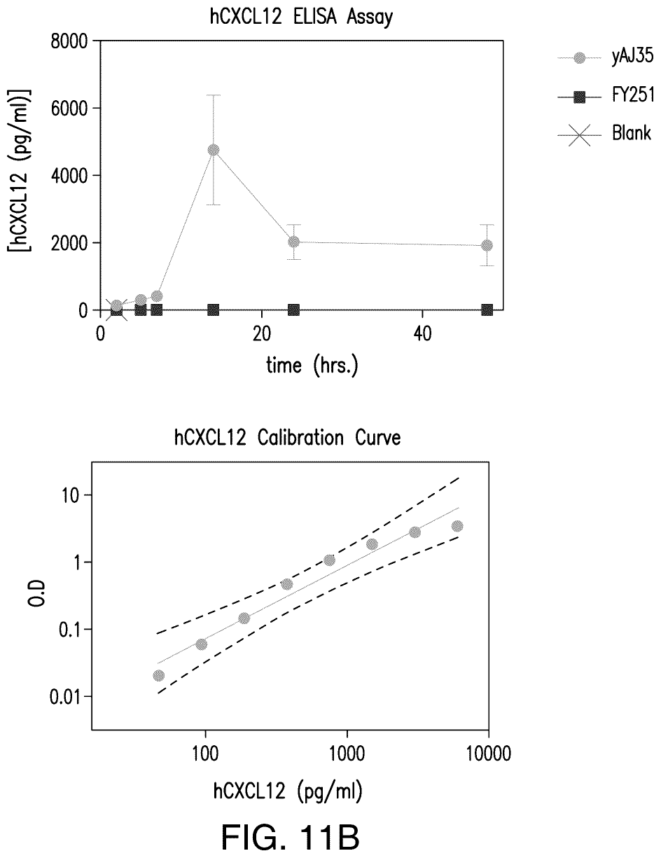

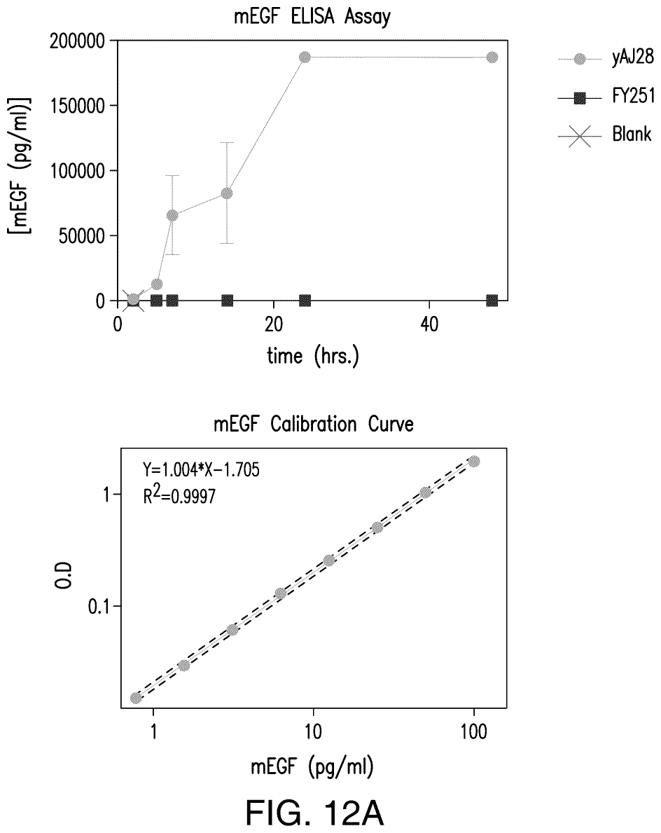

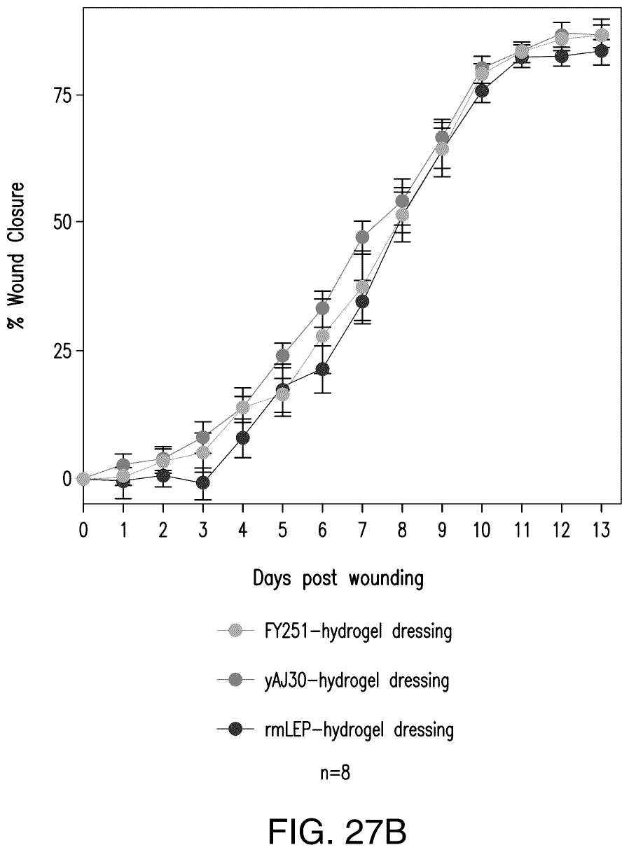

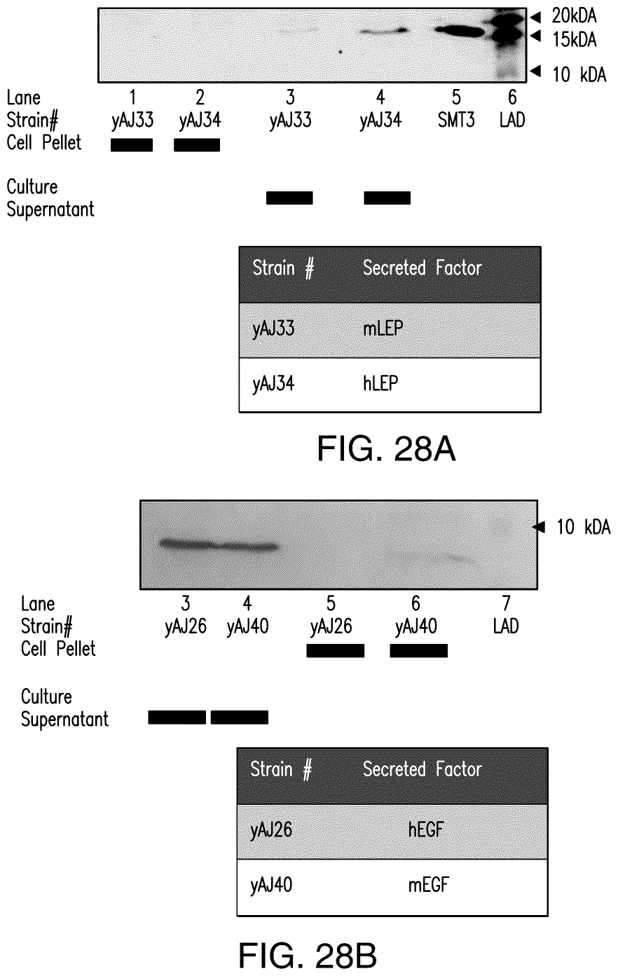

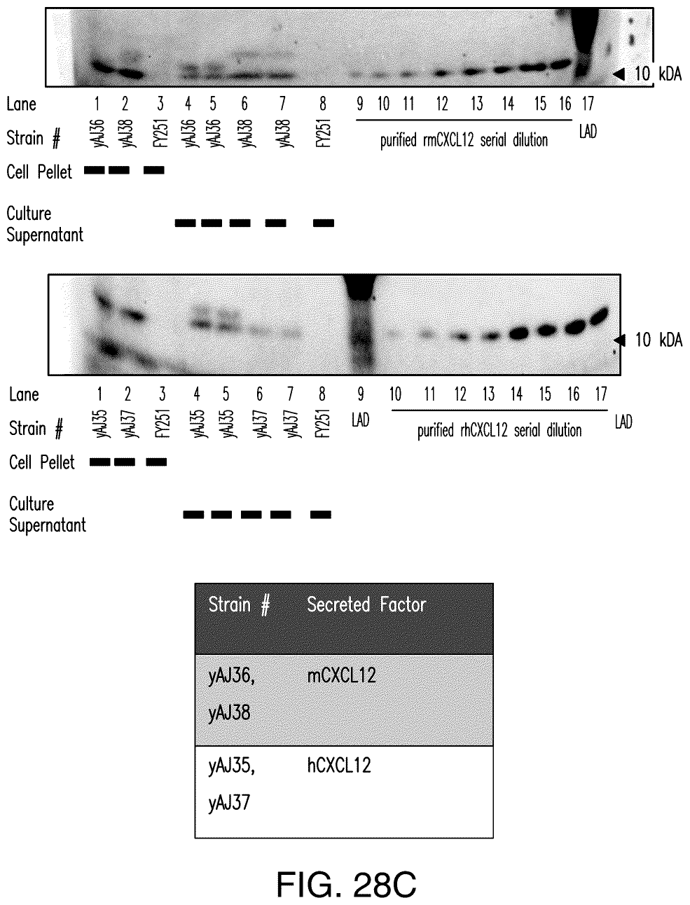

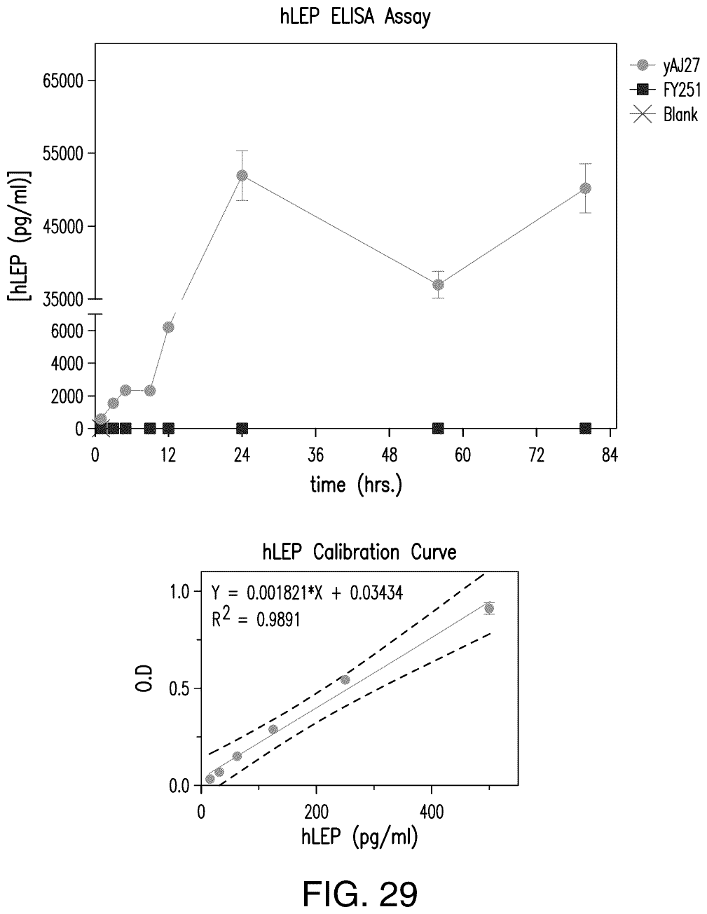

As shown in FIG. 3, FIG. 11A, FIG. 11B, FIG. 12A, FIG. 12B, FIG. 28A, FIG. 28B, FIG. 28C and FIG. 29 the engineered yeast strains secrete EGF, CXCL12 and Leptin in high enough titers needed for the wound healing formulations. The concentrations of hEGF was 80 ng/ml, the concentration of mEGF was 200 ng/ml, the concentration of hCXCL12 4,000 pg/ml, the concentration of mCXCL12 was 2,000 pg/ml, and the concentration of hLEP was 55 ng/ml.

ELISA assays were performed to further analyze the secretion of mCXCL12, hCXCL12, mLEP, hLEP, mEGF and hEGF from genetically engineered yeast. A mouse CXCL12 ELISA Kit (Ref: ab100741), a human CXCL12 ELISA Kit (Ref: ab100637), a mouse EGF ELISA Kit (Ref: ab234560) and a human EGF ELISA Kit (Ref: ab100504) were obtained from Abcam. Mouse Leptin ELISA Kit (KMC2281) and human Leptin ELISA Kit (KAC2281) were obtained from Fisher Scientific (Invitrogen). DYKDDDDK-Tag Protein ELISA Kit (E4700) was obtained from BioVision. All assays were done following the manufacturer's instructions.

The yeast strains were first incubated in yeast selective media for 24 hours to reach an OD of 0.8 and were subsequently diluted to a starting OD of 0.1 and incubated for 48 hours at 37° C. Samples were collected at 2, 5, 7, 14, 24 and 48 hours post incubated, the OD was measured, and the supernatant containing the secreted proteins was collected and stored at −20° C. post centrifugation. For measuring the secretion titer of mEGF, the supernatant was collected from yAJ28 (FY251 transformed with pAJ26), for measuring the secretion titer of hEGF, the supernatant was collected from yAJ39 (FY251 transformed with pAJ02), for measuring the secretion titer of mCXCL12, the supernatant was collected from yAJ36 (FY251 transformed with pAJ37), for measuring the secretion titer of hLEP, the supernatant was collected from yAJ27 (FY251 transformed with pAJ25) and for measuring the secretion titer of hCXCL12, the supernatant was collected from yAJ35 (FY251 transformed with pAJ36). All tests were run in 3 biological replicates and all samples were handled and stored in low-binding Eppendorf tubes. To run the ELISA assays, the collected supernatant samples were further diluted between 10 to 10,000 times in the corresponding dilution buffer provided by the assay kits. As shown in FIGS. 11A and 11B, the genetically engineered yeast secreted high titers of mCXCL12 and hCXCL12, respectively. Similarly, the genetically engineered yeast secreted high titers of mEGF and hEGF as shown in FIGS. 12A and 12B, respectively. The genetically engineered yeast also secreted high titers of hLEP as shown in FIG. 29.

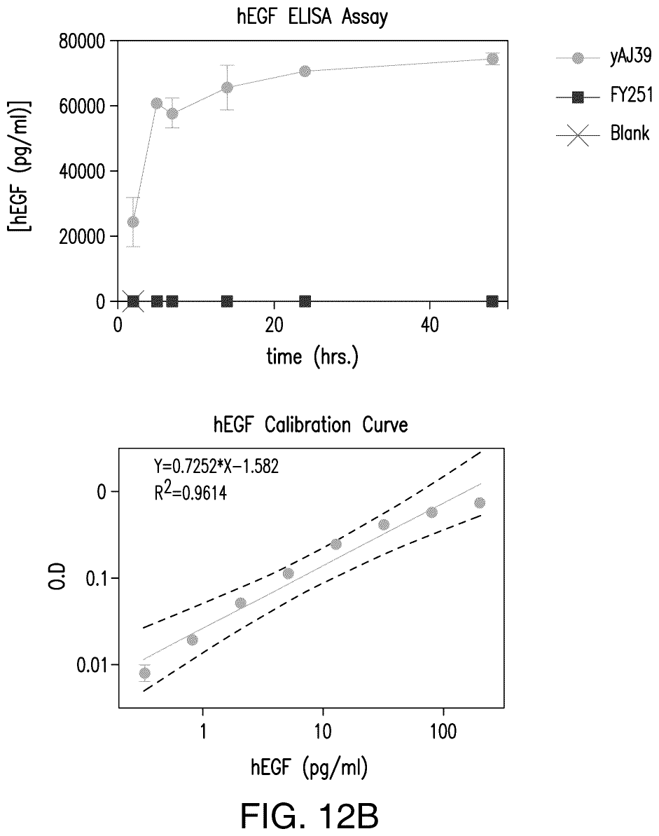

Further analysis was performed to analyze the growth of the genetically engineered yeast during a 48-hour period. As shown in FIG. 13, all four engineered yeast are in the lag phase for the first 5-6 hours, are in the log phase from the 5^{th}-6^{th} hours of incubation to 14 hours of incubation and reach the stationary stage after 14 hours of incubation. When compared with the secretion titers shown in FIGS. 11A, 11B, 12A and 12B, the secretion titers of the recombinant proteins correlated with and is proportionate to the yeast growth phase.

### Example 2: Biological Activity of Secreted Skin Therapeutics In Vitro

This Example shows that the skin therapeutics expressed and secreted by transformed yeast were biologically active in vitro.

To detect the biological activity of secreted skin therapeutics of interest, in vitro cell-based assays on purified and sterile-filtered secreted proteins using skin Fibroblast and keratinocyte cells were performed. In vitro wound-healing scratch assays and transwell migration assays were performed as previously described. BrdU cell proliferation assays will be performed to study the effect of target agents on the proliferation of the cells.

To test the biological activity of the secreted FLAG-hEGF and FLAG-LEP chimeric proteins, the engineered yeast strains were incubated in 5 ml selective media at 30° C. for 24 hours. The yeast culture was centrifuged and the supernatant containing the secreted agents was collected. Amicon centrifugal filters (3 kDa) were used to concentrate and buffer exchange the secreted proteins. The concentrated solutions were then diluted in DMEM to a final concentration of 5 ml.

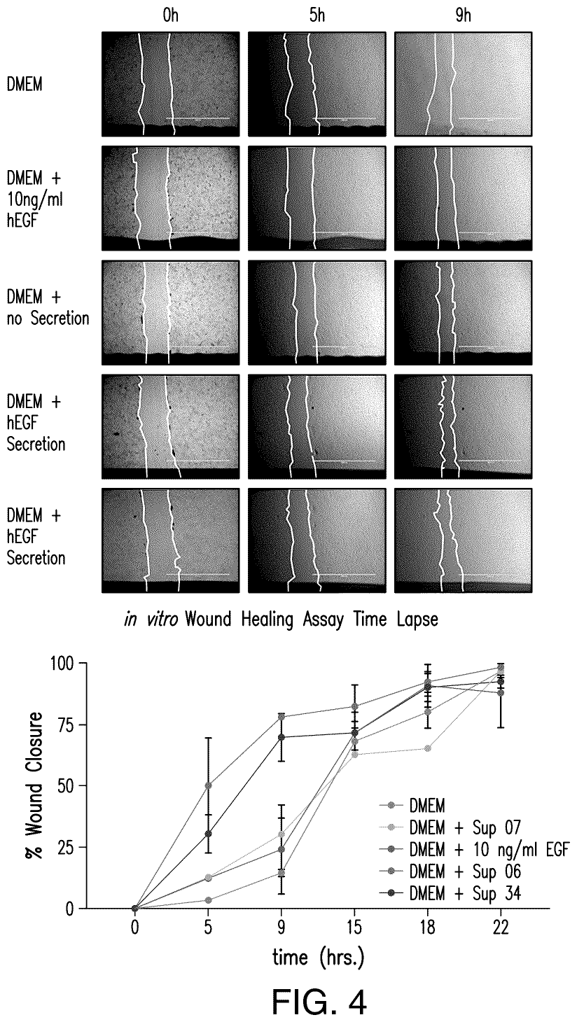

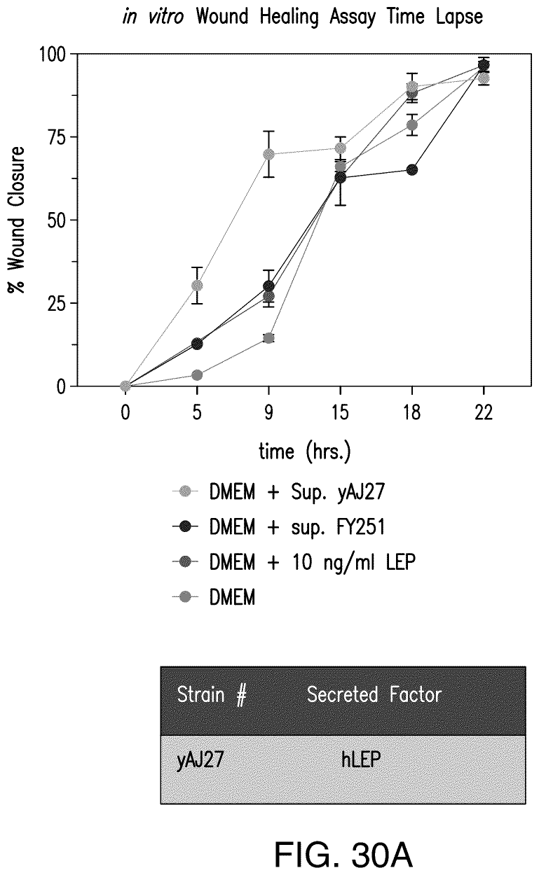

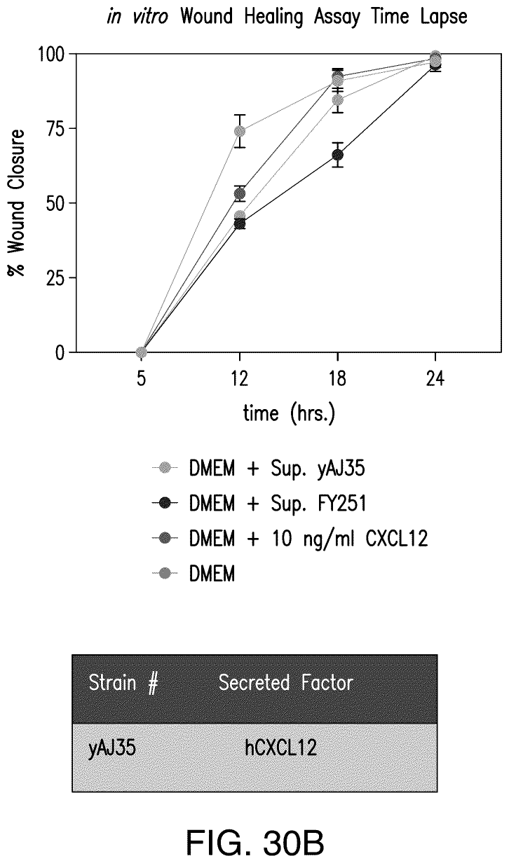

A scratch assay was performed on primary dermal fibroblast cells (PCS-201-010) in tissue-culture-treated six-well plates. The seeding density was 0.3×10⁶ cells per well, and the cells were grown in DMEM+10% FBS media for 24 hours to reach 70-80% confluency. The purified chimeric proteins, FLAG-hEGF and FLAG-LEP, obtained from the supernatant of genetically engineered yeast were subsequently added to the fibroblast cells. As shown in FIG. 4 and FIG. 30A, hEGF and hLEP enhanced the migration of the fibroblast cells compared to the cells incubated in media alone. In FIG. 4, Sup 07 is the supernatant from yAJ07 (hEGF), Sup 06 is the supernatant from yAJ06 (hEGF), Sup 34 is the supernatant from yAJ34 (hLEP). Similarly, hCXCL12 enhanced the migration of the fibroblast cells compared to the cells incubated in media alone, as shown in FIG. 30B.

Additional scratch assays were performed by coculturing the yeast expressing and secreting the skin therapeutics with fibroblast cells. For this assay, the fibroblast cells were incubated for 48 hours to reach 80% confluency in 24-well plates. Next, a scratch was formed in each well using a 200 μl pipette tip, the cells were washed with sterile PBS, and new growth media was added to each well. Next, cell culture inserts (pore size=0.45 μm) were put inside the wells, and the engineered yeast dissolved in the fibroblast growth media was added inside the inserts. The closure of the scratch was monitored after 24 hours of co-culture of the yeast and the fibroblast cells. As shown in FIG. 5B, genetically-engineered yeast secreting EGF (yAJ06) at an OD between 0.12 (3.6×10⁶) and 0.4 (1.2×10⁷) improved the closing of wounds compared to control (yeast culture with OD=1, contains 3×10⁷ yeast cells per ml).

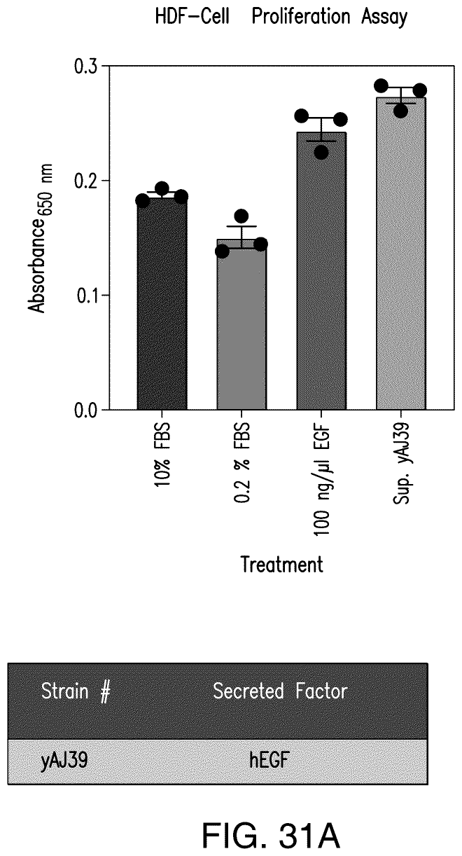

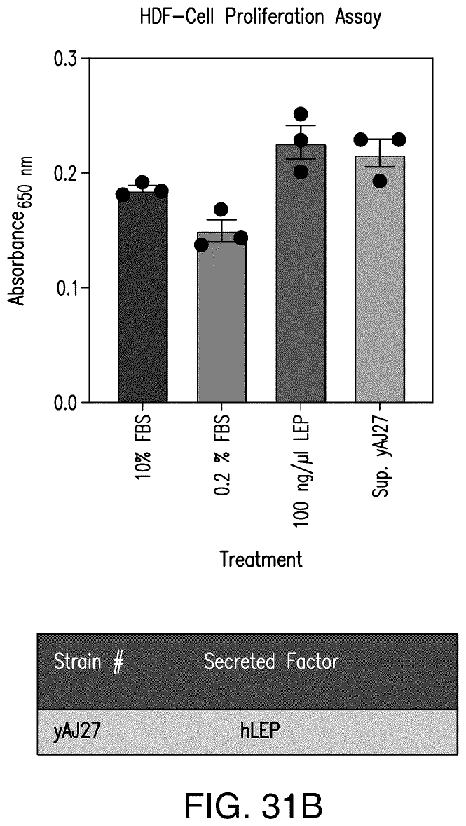

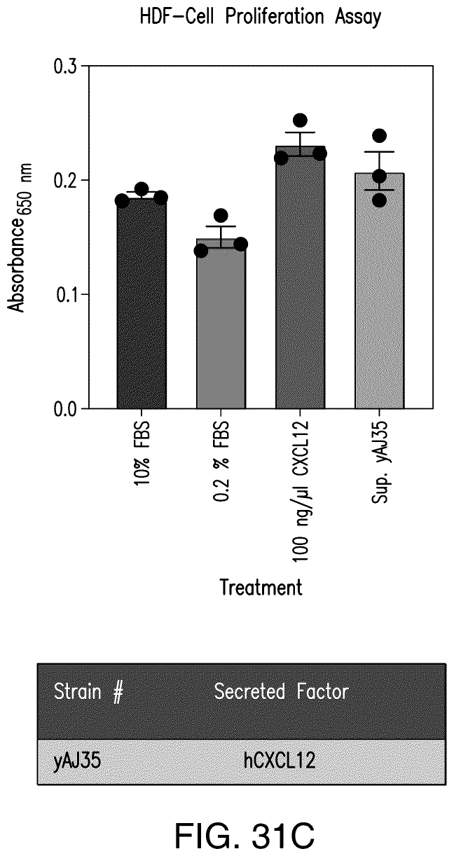

Cell proliferation assays were further performed to study the bioactivity of the factors of interest. The proliferation assay was performed on primary skin fibroblasts cells. As shown in FIGS. 31A-31C, hEGF, hLEP and hCXCL12 from the supernatant of genetically engineered yeast (yAJ39, yAJ27 and yAJ35, respectively) enhanced the proliferation of the fibroblasts compared to control confirming that the secreted proteins are biologically active and promote cell proliferation, which is important for wound healing and treatment of skin conditions.

### Example 3: Generation of a Yeast Hydrogel

This Example describes the generation of a live yeast agar hydrogel that includes a bottom layer between the yeast hydrogel and the wound bed. The bottom layer is permeable to the proteins secreted from yeast but not permeable to the yeast cells. This layer will reduce the chance for direct contact of the yeast with the wound bed even though the chosen yeast strains are not pathogenic and are GRAS. Hydrophilic polytetrafluoroethylene (PTFE) with a 0.2 μm pore size was selected for the bottom layer as PTFE is inert and biocompatible and has been widely used in medicine and the construction of medical devices.

Yeast secreting hEGF from Example 1 were incubated in selective media for 24 hours at 30° C., and then incorporated into a paste via centrifugation. Next, a 0.7% w/v agar hydrogel was prepared by dissolving this paste in freshly prepared selective media containing 0.7% w/v agar (T<40° C.) under sterile conditions. The 0.7% w/v agar solution formed a hydrogel when reaching ambient temperature (FIG. 6). To prepare the live yeast hydrogel dressing for in vivo experiments, the yeast containing agar solution was pipetted out and solidified on autoclaved hydrophilic PTFE membranes (pore size=0.2 μm).

To test the free diffusion of the secreted proteins in the agar hydrogel, live yeast containing a 0.7% w/v agar slab containing live yeast was incubated on top of a polyvinylidene difluoride (PVDF) membrane for 24 hours at 30° C. The PVDF membrane has an unspecific and high affinity for amino acid and protein binding and can bind and immobilize the secreted proteins that diffuse through the hydrogel. Next, western blot using anti-FLAG antibodies were used to examine protein diffusion. The yeast hydrogel was removed, and the PVDF membrane was developed using established western blot protocols and anti-FLAG antibodies. As shown in FIG. 6, the secreted proteins passed through the hydrogel in high titers within the 24-hour timeframe. These data show that FLAG-hEGF and FLAG-hLEP can diffuse freely in the agar hydrogel.

A similar experiment was performed under the same conditions disclosed above, but a PTFE membrane was placed between the yeast agar hydrogel and the PVDF membrane. The yeast hydrogel and the PTFE membrane were removed, and the PVDF membrane was developed. As shown in FIGS. 7A-D, hEGF readily passed through not only the agar hydrogel, but also the PTFE membrane that will be required for application to mice. Similarly, as shown in FIG. 7I, LEP and CXCL12 can freely diffuse through the hydrogel and the PTFE membrane.

Next, the impermeability of hydrophilic PTFE membrane (pore size=0.2 μm) towards yeast cells and yeast spores was analyzed. A PTFE membrane was sandwiched between the yeast hydrogel and agar plate (FIG. 7E). The yeast hydrogel and the agar plate contained the same yeast nutrition formulation and agar percentage. This system was incubated for 24 hours at 30° C., and yeast hydrogel and PTFE membrane were subsequently removed, and the agar was incubated for another 48 hours at 30° C. If the yeast can pass through the PTFE membrane, then yeast colonies will grow on the agar plate. As shown in FIGS. 7F-H, even after 72 hours of growth, there were no yeast colonies visible on the agar plates. These data show that the PTFE membrane is not permeable to the yeast spores and cells.

To confirm if the proteins secreted via the three-layered hydrogel dressing are biologically active, in vitro scratch assays were performed on human skin fibroblast cells (HDFs). Well-inserts usually used in Boyden chamber assays to study cell migration were used. The HDF cells were cultured in the lower chamber and a scratch was formed after they reached 70-80% confluency. At that point, a live yeast dressing was placed on the top chamber and the system was incubated for 24 hours before measuring the gap (scratch) size in the lower chamber. As shown in FIG. 5B, in high concentrations, the yeast cells were toxic to mammalian cells. This result was not surprising since yeast cells as a result of their metabolism, and fast doubling—would (i) deplete the nutrients out of the culture media, thus depriving the mammalian cells of growth, (ii) over time, make the pH of the media more acidic and non-ideal for the mammalian cells and (iii) produce ethanol which is toxic to the sensitive cultured mammalian cells. However, it was surprising that at lower yeast concentrations, the bioactivity of the yeast-secreted protein factors overcame the yeast growth toxicity to enhance mammalian cell migration and proliferation rates (FIG. 5B). As shown in FIG. 5B, concentrations equal to or less than 1.5×10⁷ cells/ml were beneficial to wound healing.

Based on the results of the above experiments, a live-yeast hydrogel dressing having three layers was prepared. The live yeast hydrogel dressing includes (i) an outer/adhesive layer which would be the barrier between the open wound bed and the outside environment, (ii) a live yeast-containing medium that would contain the live yeast, nutrients, buffers and potential supplemental active ingredients such as antibiotics (this medium allows for diffusion of the secreted wound healing factors), and (iii) a semipermeable layer between the live yeast medium and the wound bed which would allow the diffusion of the secreted factors to the wound-bed but would block yeast cells from reaching to the skin and wound-bed.

In this Example, as shown in FIG. 8B, a live-yeast hydrogel dressing was included with the following three layers: a) a TEGADERM™ film on the top (which is composed of a polyurethane membrane), which acted as a physical barrier and helped to hold the dressing in place on top of the wound, b) the live yeast hydrogel which secretes the skin therapeutics in situ and c) a PTFE membrane with a pore size of 0.2 μm that sits as the border of the wound bed and the yeast hydrogel. As shown in FIG. 7, a PTFE membrane with a pore size of 0.2 μm was permeable to secreted proteins but impermeable to the yeast cells.

### Example 4: Treatment of Wounds in Mice with the Yeast Hydrogel Dressing

This Example shows the use of the 3-layer yeast hydrogel dressing of Example 3 (schematic in FIG. 8D) for treating wounds in a diabetic mouse model. Streptozotocin (STZ)-treated diabetic mice is a well-established model for diabetes and wound healing studies. In particular, the STZ-administered mice are an excellent model as these mice have impaired wound healing.

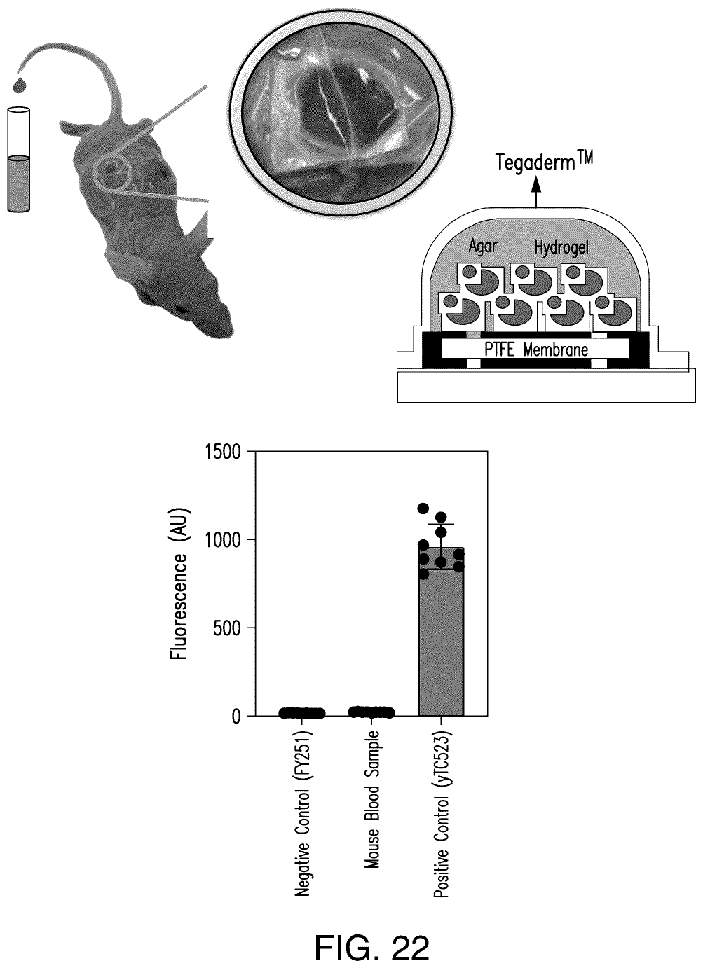

To investigate the changes in the healing dynamics of diabetic wounds under the effect of topical administration of the live-yeast formulation of Example 3, diabetes was induced in 8 C57BL/6 (B6) mice by intraperitoneal injection of STZ (40 mg/kg) for five consecutive days. C57BL/6J mice (Stock no: 000664|B6) were obtained from Jax. During this time, mice were given 10% sucrose in water to drink ad-lib in case of mice becoming hypoglycemic, and were maintained on regular chow diet. As shown in FIG. 23, an increase in body weight over time and blood glucose concentrations were observed in mice administered STZ, confirming that such mice successfully became diabetic.

The mice were shaved, anesthetized using isoflurane and subjected to a full-thickness excisional wound 8 mm in diameter on each side along the dorsal midline region 14 days after the STZ treatment (FIG. 8A). To evaluate wound healing efficacy, mice were dosed topically with either negative control agar hydrogel on PTFE or an agar hydrogel including EGF-secreting yeast on PTFE (FIGS. 8B-8C). The TEGADERM™ dressing was used on the top of the hydrogel to keep the formulation in place and prevent natural skin contraction.

In 5 of the 8 mice, the TEGADERM™ dressing did not stay in place for more than three days. As a result of this, the wound surface stayed open to the air, and the topical dressing of the yeast treatment or the control treatment fell off, leading to wound closure as a result of natural skin contraction and not because of healing and formation of new skin layers. The data from these 5 mice were not considered and the data from the wounds of the remaining 3 mice, which had the TEGADERM™ stay in place, were analyzed. The wound condition and the mice's well-being were monitored every 24 hours, and a new formulation/dressing was applied to each mouse daily after removing the existing dressing and cleaning the skin. Photographs were captured of each mouse each day i) before removal of the old dressing and ii) each wound site after removal the old dressings to quantify the wound area. Wound closure was expressed as the relative change in wound area compared to day 0. On day eight post-surgery, animals were euthanized, and the wounds were excised and collected for histological evaluation.

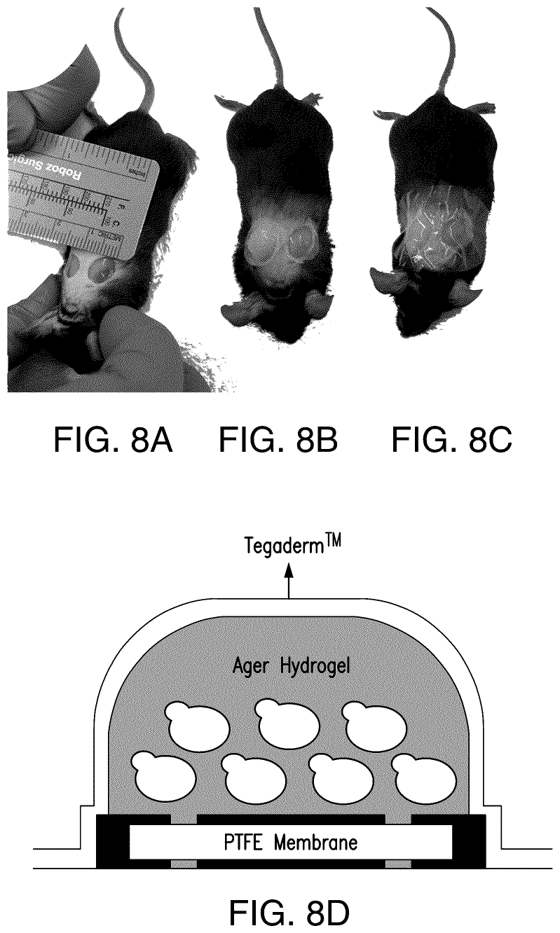

As shown in FIGS. 9A-9B, the topical application of transgenic EGF-secreting yeast did not cause any infection, inflammation or harsh immune response in the treated mice. In addition, the live yeast wound healing formulation enhanced the wound healing in the diabetic mice.

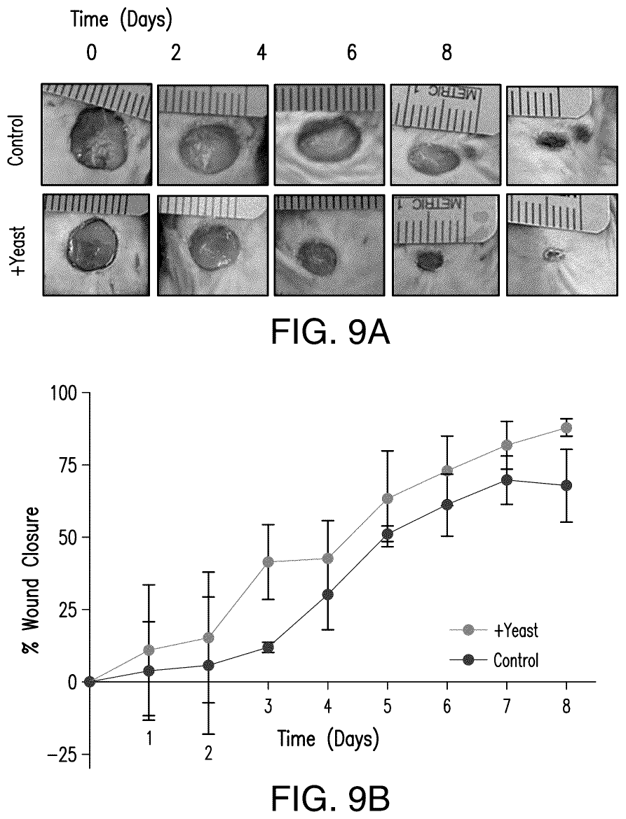

To assess the quality of the wound repair resulting from topical application of the transgenic yeast compared to the topical application of the control agar, hematoxylin-and-eosin (H&E) staining were performed on the excised mouse wounds on the 8th day of the experiment. Images from the H&E staining in FIG. 10 provided information regarding cell proliferation, collagen formation, deposition and re-epithelialization. The results of H&E-stained sections showed better cell proliferation, recruitment and more orderly tissue patterns in samples with the EGF-secreting yeast treatment (FIG. 10A) compared to controls (FIG. 10B).

These data show that the following: (i) engineered yeast can secrete wound-healing factors in high titers in a hydrogel formulation; (ii) the secreted factors can diffuse freely in the hydrogel; (iii) the factors can diffuse through a PTFE membrane; (iv) the PTFE membrane is impermeable to both the yeast cells and yeast spores; (v) the live-yeast hydrogel formulation can be used for topical application on dorsal mouse wounds; (vi) topical application of yeast does not cause infection, inflammation, or drastic immune response in the mice and at the wound site; (vii) wound healing progression in mice that are bilaterally wounded can be quantified and control and treated wounds can be compared on the same mouse; and (viii) topical application of the engineered yeast hydrogel dressing can accelerate wound healing in diabetic mice.

### Example 5: Whole-Transcriptome Analysis of Wound Lesions

This Example provides the analysis of mRNA from wound lesions at different time points throughout the wound healing process. This analysis provides information regarding the mechanism of action of the applied formulations by determining which pathways are activated and which processes are turned down and gives a deeper understanding of the healing process with a higher resolution.

Wound/tissue samples will be cut for mRNA sequencing (before paraformaldehyde fixation of the lesions). The samples will be homogenized, and mRNAs will be enriched from the total RNA following established poly(A) selection. Illumina TruSeq RNA prep kit will be used for the library preparation, and Illumina Hiseq 2000 will be used for the sequencing. The human genome will be used as the reference for mapping the reads. The gene expression data will be assessed in the form of signaling pathways by extrapolating genomic information and gene set enrichments using the database for annotation, visualization, and integrated discovery (DAVID). This will illustrate what pathway activities are up/down-regulated compared to the normal conditions in response to the topical application of each transgenic yeast formulation and each wound-healing factor that is secreted by the transgenic yeast.

### Example 6: Yeast Communities

A key advantage of the live yeast hydrogel formulations for wound healing is modularity of design stemming from advances in the field of synthetic biology. Co-delivery of two or three wound healing factors that act via complementary pathways can improve overall wound healing using the formulation. For example, phosphorylation of PDGFR upon PDGF binding and dimerization triggers the PLCγ, PI3K and several MAPK pathways, and EGF binding to the ErbB1 results in the activation of MAPK/ERK1/2, PI3K, Rac- and ERK-dependent pathways depending on the cell type. Activation of LEPR upon binding to LEP triggers the JAK-STAT, PI3K/AKT, and ERK1/2 pathways and binding of CXCL12 to the CXCR4 activates the Erk/ELK, AP-1, and JAK-STAT pathways.

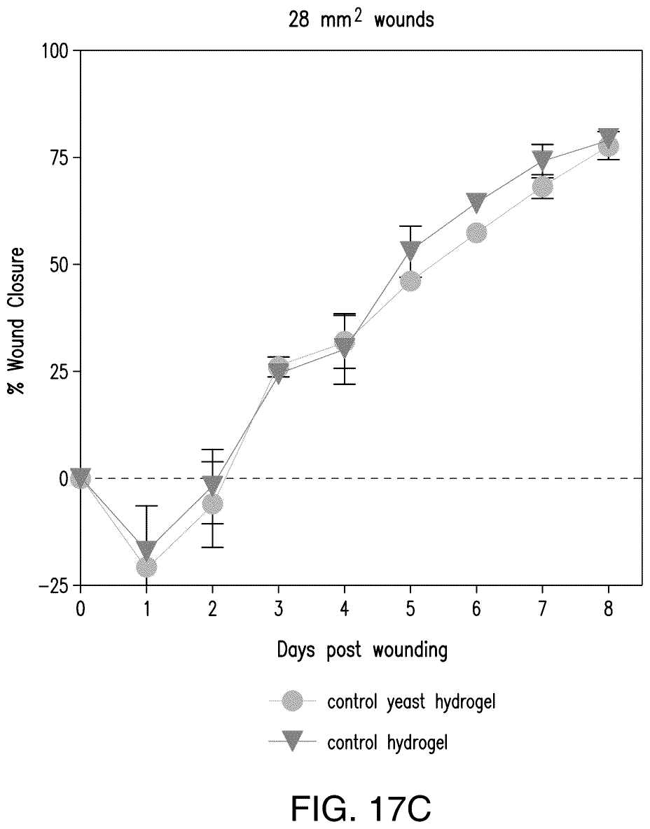

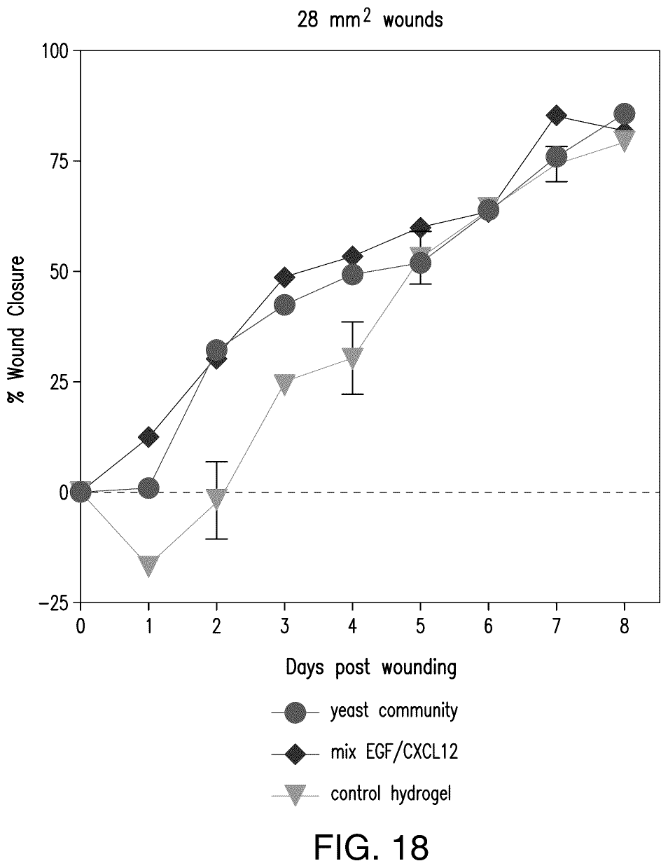

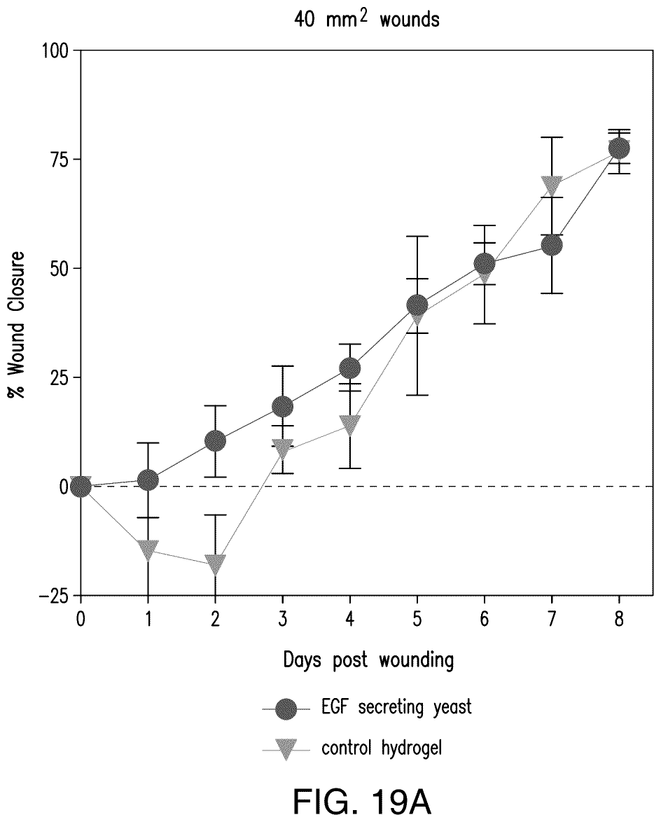

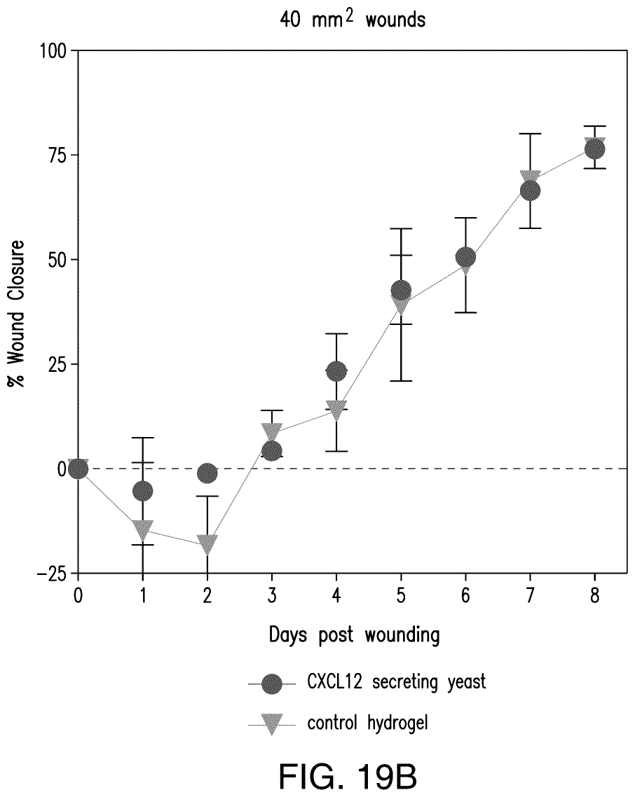

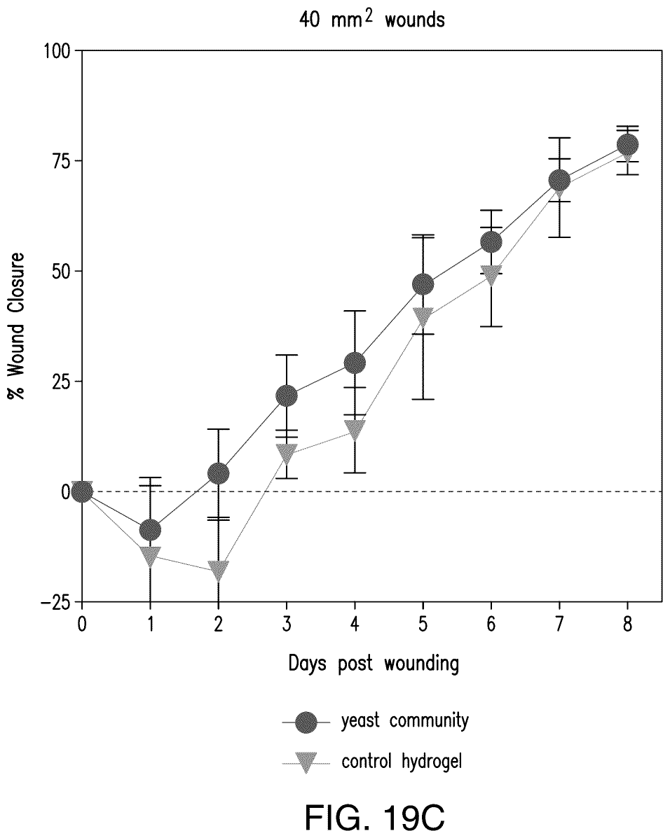

Yeast communities that include at least two yeast strains that each secrete a different skin therapeutic or a yeast strain that secretes two or more wound healing factors can be administered to stimulate more wound healing signaling pathways to treat a skin condition. For example, co-delivery of EGF and CXCL12 or PDGF and Leptin can activate more wound healing signaling pathways. As shown in FIG. 18, 28 mm² wounds treated with a yeast community containing a mixture of the mEGF-secreting yeast and the mCXCL12-secreting yeast healed much faster than the wounds treated with the control hydrogel. Similarly, for wounds having an initial size of 40 mm², treatment with the yeast community (mixture of the mEGF secreting yeast and the mCXCL12 secreting yeast) healed much faster than the wounds receiving the control hydrogel (FIGS. 19C and 19D). These results show that wounds treated with the yeast community had a better and steadier healing rate than the wounds treated with only one recombinant wound agent-secreting yeast strain (FIG. 19D).

### Example 7: Treatment of Wounds of Diabetic Mice

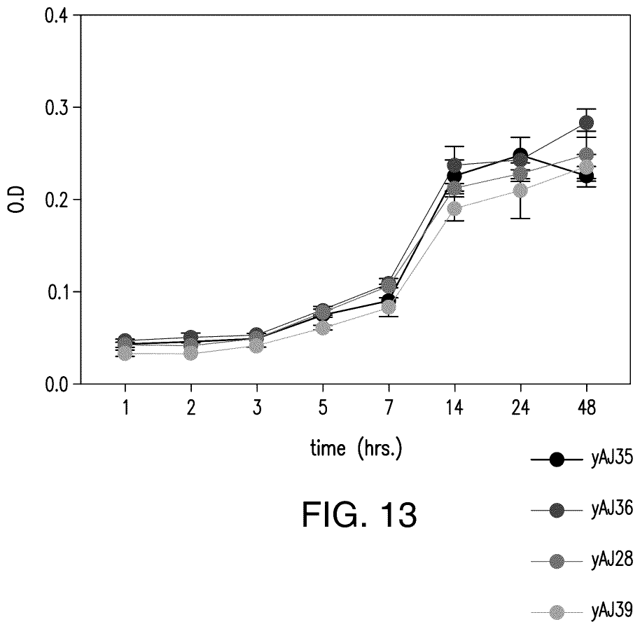

Additional experiments were performed to analyze the effectiveness of applying genetically engineered yeast that express and secrete CXCL12 or EGF to treat wounds of diabetic mice. 15 SKH1-Elite mice—a hairless mouse frequently used in dermatology studies—were treated with STZ to induce diabetes as described in Example 4. Two weeks later, four excisional wounds were cut on the back of each mice using a 5 mm biopsy punch, generating a total of 60 excisional wounds, ranging from 18 mm² to 50 mm² as shown in FIG. 14. For ten mice, on each mouse, one wound was treated with the mEGF secreting yeast hydrogel dressing, one with the mCXCL12 secreting hydrogel dressing, one with the hydrogel dressing containing a yeast community (mix of mEGF secreting yeast and mCXCL12 secreting yeast) and a control agar hydrogel dressing. For five mice, on each mouse one wound was treated with purified recombinant mEGF (100 ng/ml) dissolved in the hydrogel dressing, one wound was treated with purified recombinant mCXCL12 (100 ng/ml) dissolved in the hydrogel dressing, one wound was treated with a mixture of both purified recombinant mEGF (100 ng/ml) and purified recombinant mCXCL12 (100 ng/ml) dissolved in the hydrogel dressing, and one wound was treated with control yeast hydrogel dressing; as described in Example 4.

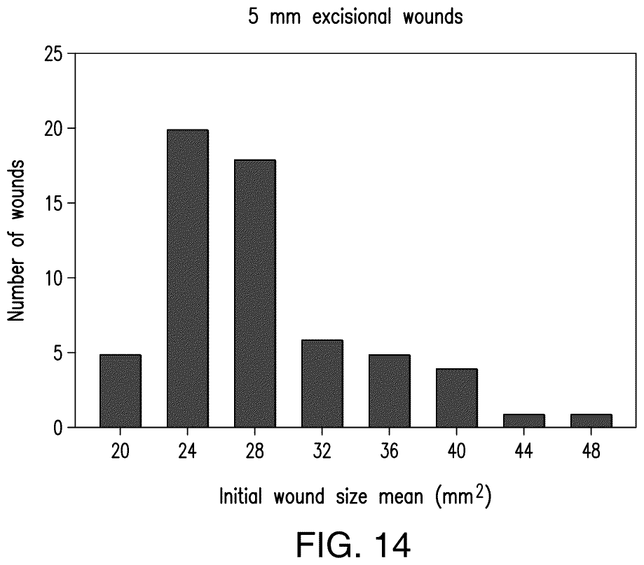

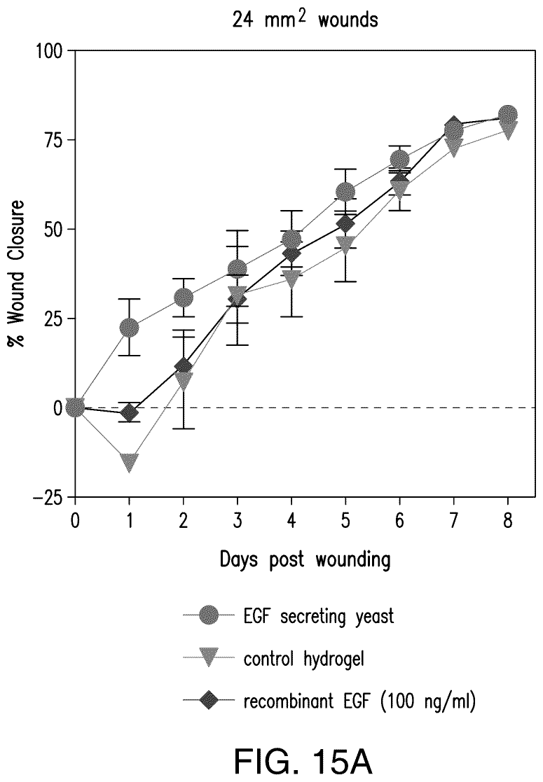

For wounds that had an initial size of 24 mm², treatment with an mEGF secreting yeast hydrogel or a mCXCL12 secreting yeast hydrogel, healed faster and better than the wounds receiving the control hydrogel (FIGS. 15A-15B). In addition, wounds treated with the purified recombinant mEGF (100 ng/ml) in hydrogel or the purified recombinant mCXCL12 (100 ng/ml) in hydrogel also healed faster and better than the wounds receiving the control hydrogel (FIGS. 15A-15B). Also, as shown in FIG. 15A, wounds treated with the mEGF secreting yeast hydrogel healed faster than wounds treated with the purified recombinant mEGF (100 ng/ml) in hydrogel. Similarly, as shown in FIG. 15B, wounds treated with the mCXCL12 secreting yeast hydrogel healed faster than wounds treated with the purified recombinant mCXCL12 (100 ng/ml) in hydrogel.

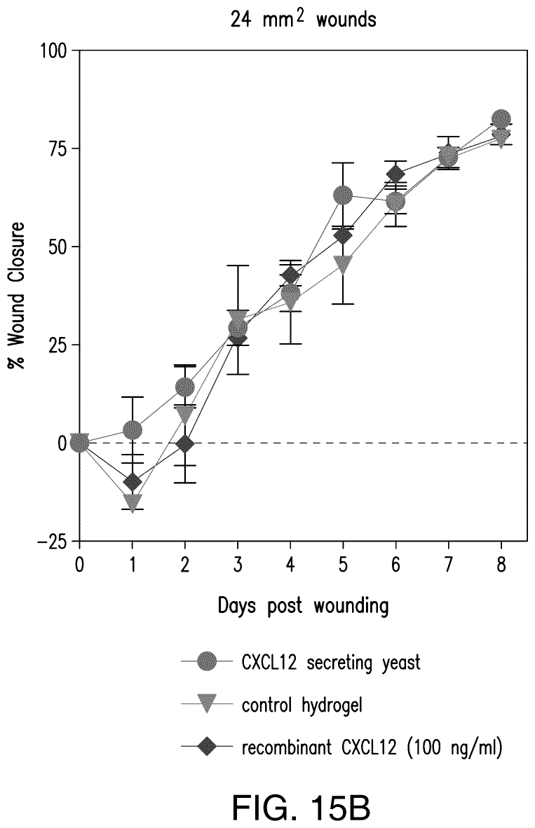

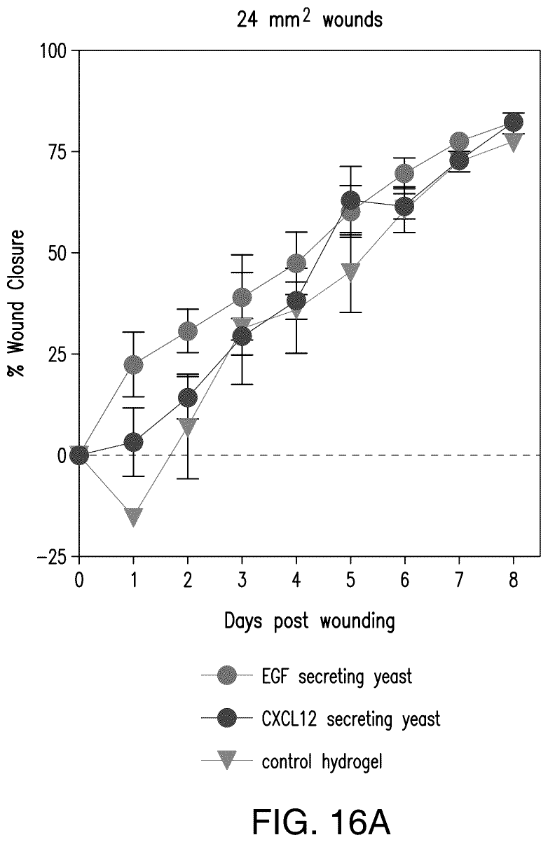

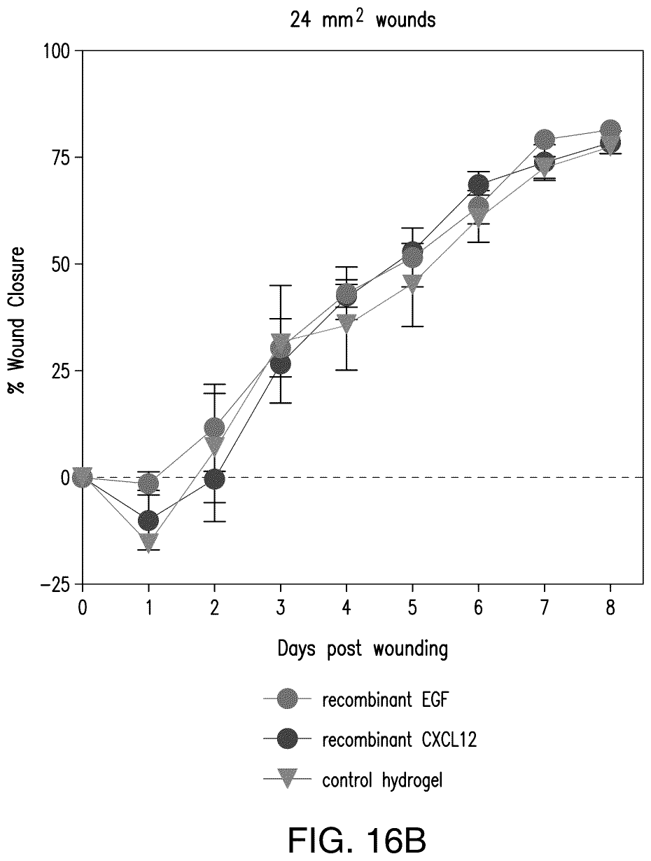

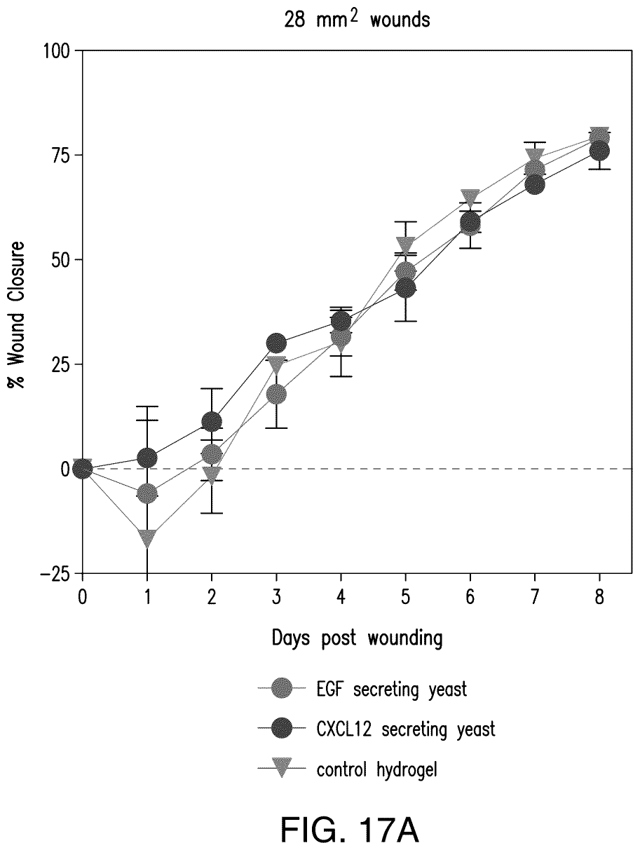

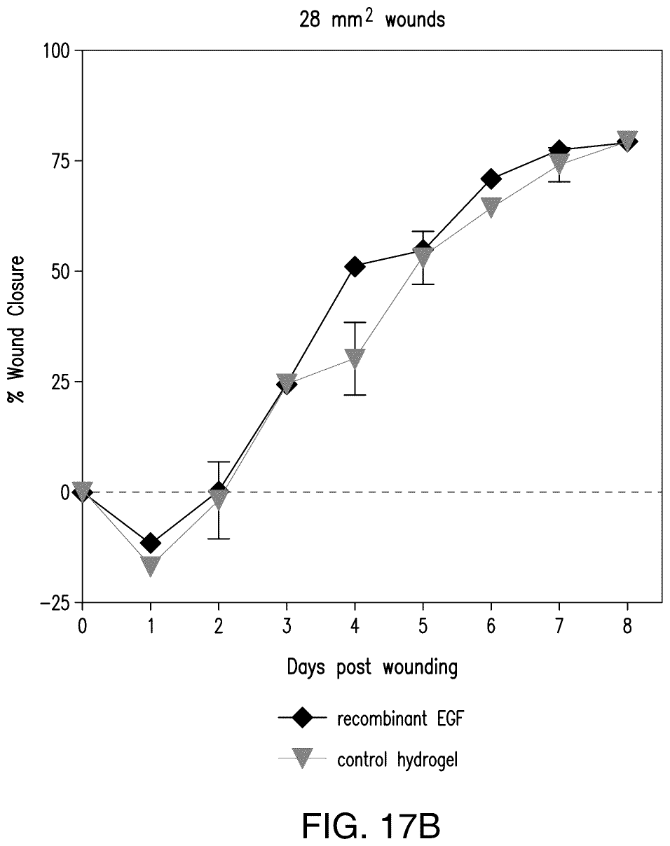

The efficacy of mCXCL12 secreting yeast was compared with mEGF secreting yeast. As shown in FIG. 16A, for wounds with the initial size of 24 mm², treatment with mEGF secreting yeast healed faster than wounds treated with mCXCL12 secreting yeast. Similarly, wounds treated with recombinant mEGF healed faster than the wounds treated with recombinant mCXCL12 (FIG. 16B). For wounds with an initial size of 28 mm², treatment with mEGF secreting yeast hydrogel or the mCXCL12 secreting yeast hydrogel, healed faster than the wounds treated with the control agar hydrogel (FIG. 17A). In addition, wounds receiving recombinant mEGF, healed faster than wounds receiving control hydrogel (FIG. 17B) and wounds treated with the control yeast hydrogel, healed slightly faster than the wounds receiving agar hydrogel (FIG. 17C).

Further experiments were performed to analyze the effectiveness of administration of more than one skin therapeutic. As shown in FIG. 18, wounds treated with a yeast community containing a mixture of the mEGF secreting yeast and the mCXCL12 secreting yeast healed much faster than the wounds receiving the control hydrogel. Wounds treated with the mixture of both recombinant factors (a mixture of the recombinant mEGF (100 ng/ml) and the recombinant mCXCL12 (100 ng/ml)) heal much faster than the wounds receiving the control hydrogel (FIG. 18).

For wounds having an initial size of 40 mm², treatment with the mEGF secreting yeast hydrogel or the mCXCL12 secreting yeast hydrogel, healed faster and better than the wounds receiving the control hydrogel (FIGS. 19A-19B). In addition, wounds treated with the yeast community (mixture of the mEGF secreting yeast and the mCXCL12 secreting yeast) healed much faster than the wounds receiving the control hydrogel (FIGS. 19C and 19D). Wounds treated with the yeast community had a better and steadier healing rate than the wounds treated with only one recombinant wound agent-secreting yeast strain (FIG. 19D).

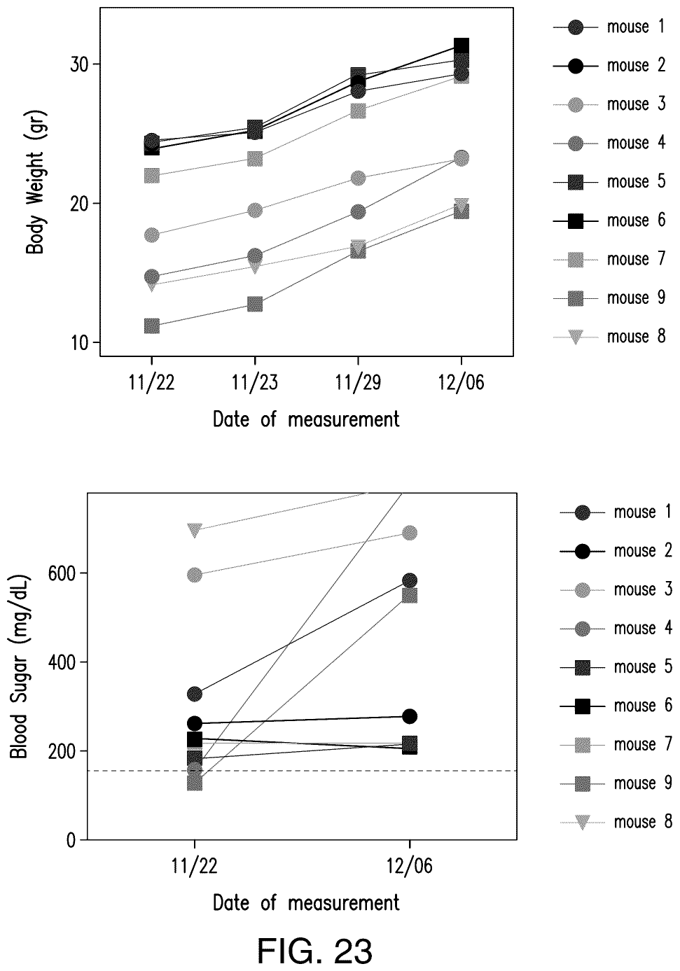

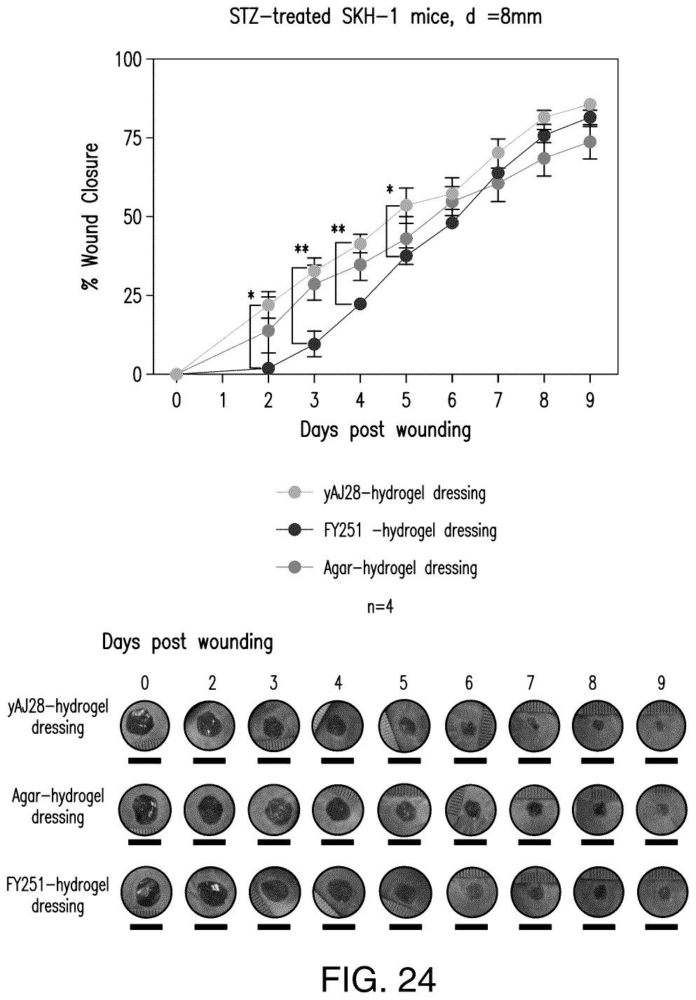

For wounds having an initial size of 50 mm², four different conditions (n=4 for each group) on full-thickness wounds were tested. The tested groups were (i) yAJ28-hydrogel dressing, containing the mEGF secreting yeast, (ii) yAJ36-hydrogel dressing, containing the mCXCL12 secreting yeast, (iii) FY251-hydrogel dressing, our control live yeast hydrogel dressing and (iv) agar-hydrogel dressing, which did not contain yeast. FIG. 24 shows that wounds treated with the EGF-secreting yeast hydrogel dressing healed much faster than wounds treated with the control yeast hydrogel dressing. Similarly, FIG. 25 shows that wounds treated with a CXCL12 secreting yeast hydrogel dressing also heal faster than the ones treated with the control yeast hydrogel dressing.

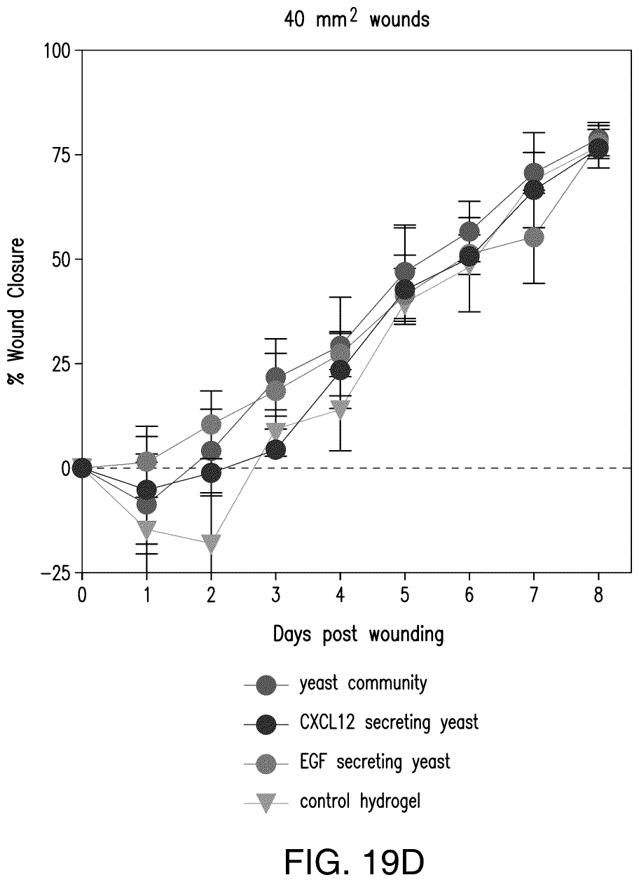

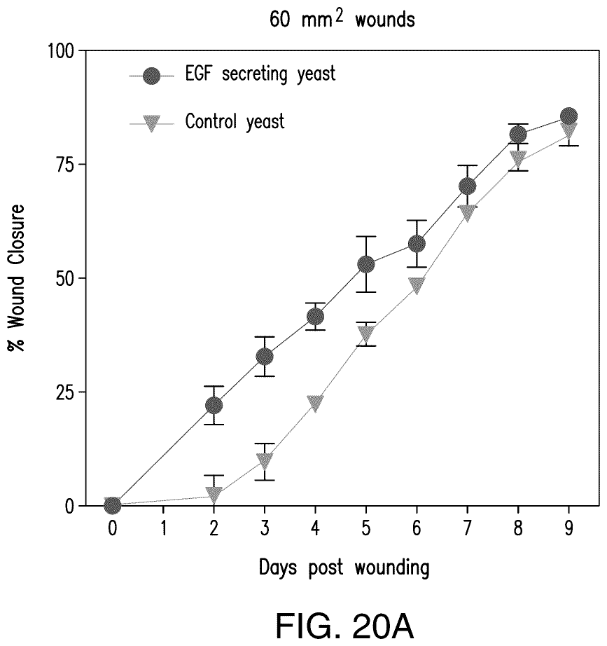

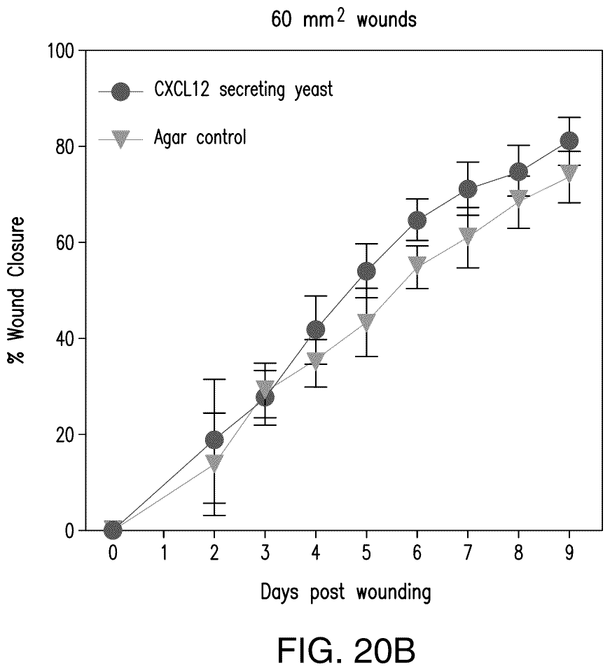

For wounds having an initial size of 60 mm², treatment with the mEGF secreting yeast hydrogel or the mCXCL12 secreting yeast hydrogel, healed faster and better than the wounds receiving the control hydrogel (FIGS. 20A-20B). The data provided in FIG. 20C compares the healing of wounds receiving the control yeast hydrogel versus the control agar hydrogel. The control yeast expresses the mCherry fluorescent protein but does not secrete any recombinant proteins.

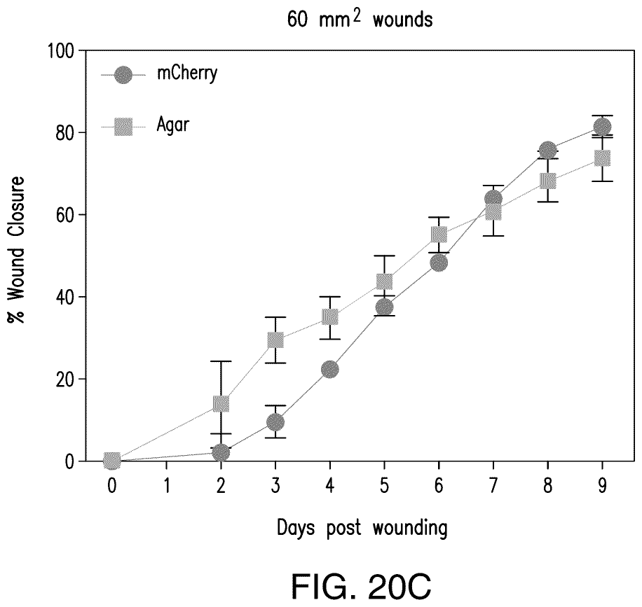

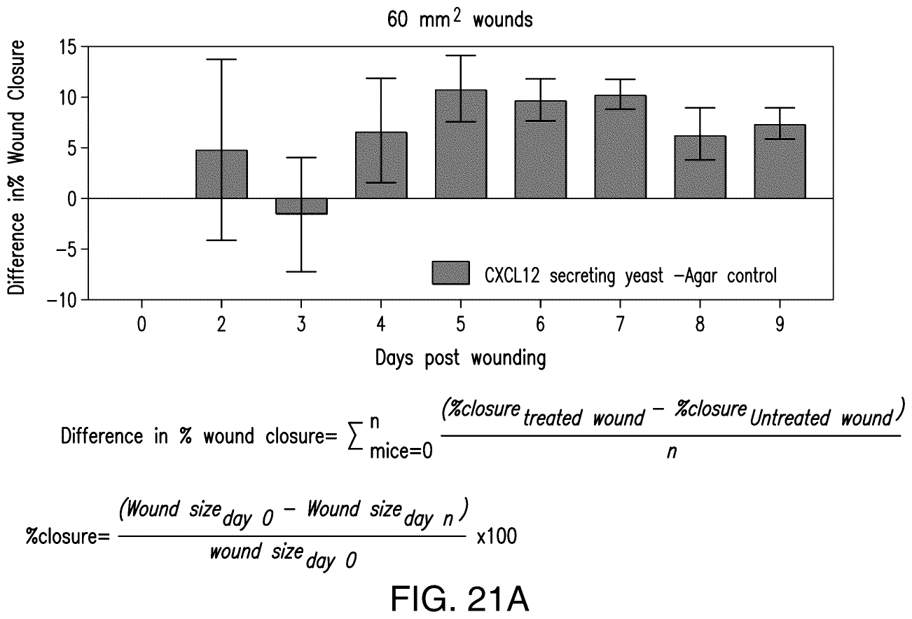

A timeline of the wound healing process in diabetic mice treated with mCXCL12 secreting yeast showed that, at each time point, the wound receiving the mCXCL12 secreting yeast hydrogel, healed faster than the wounds treated with the control (FIG. 21A). Similarly, at each time point during the timeline of a wound healing process in diabetic mice treated with mEGF secreting yeast, the wound healed faster than the wounds treated with the control.

To further study the timing of the different healing stages and the quality of the cellular processes at different stages of healing, e.g., to confirm proper healing resulting from the topical application of the transgenic yeast-wound lesions were collected for staining and immunohistochemistry (IHC) analysis from each mice study group was performed at different time points (2nd day, fourth day, etc.).

To collect these wound lesions at different time points, a mouse from each group was randomly chosen for excision of the wound lesions after scarification—the selected mice were out of the experiment for the future time points. The wound lesions were assessed with DAPI staining, as well as IHC analysis based on K14 staining, K10 staining, loricrin, phalloidin, Ki67 staining and CD31 staining. Ki67 staining was used to visualize cell proliferation at the wound site, and the CD31 immunostaining-targeting the macrophages in the lesions—was used to analyze the inflammatory response of the animal at the wound site. K14 was used to study basal keratinocytes, K10 was used to study suprabasal keratinocytes, loricrin was used to study granular and cornified layers, Ki47 was used to study proliferative cells and phalloidin was used to study the F-Actin cytoskeleton.

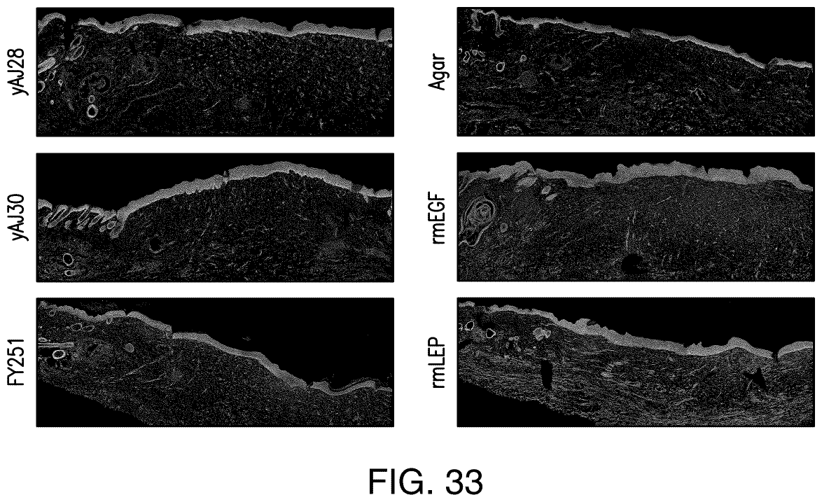

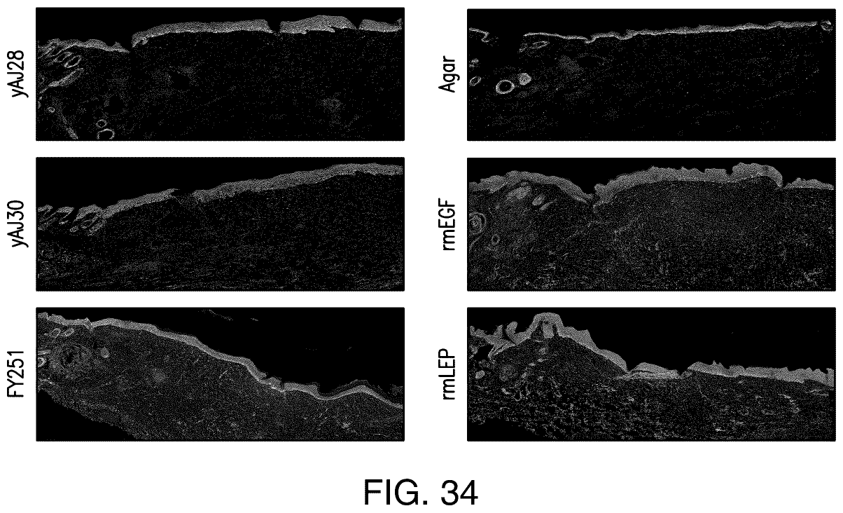

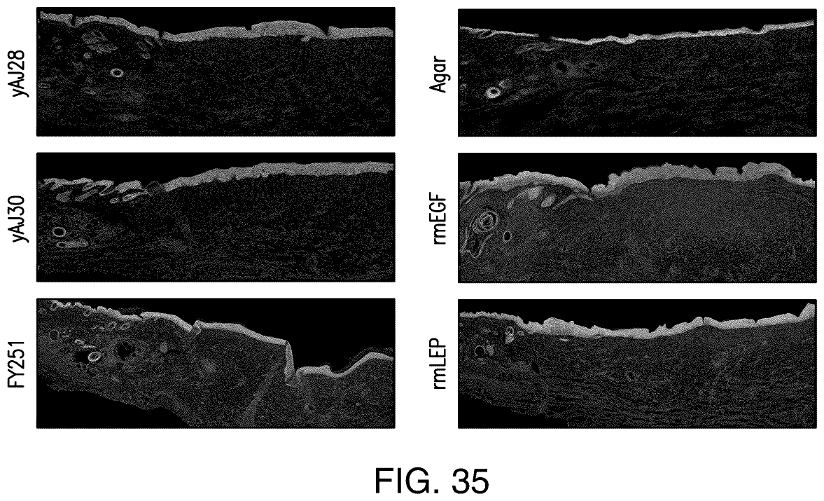

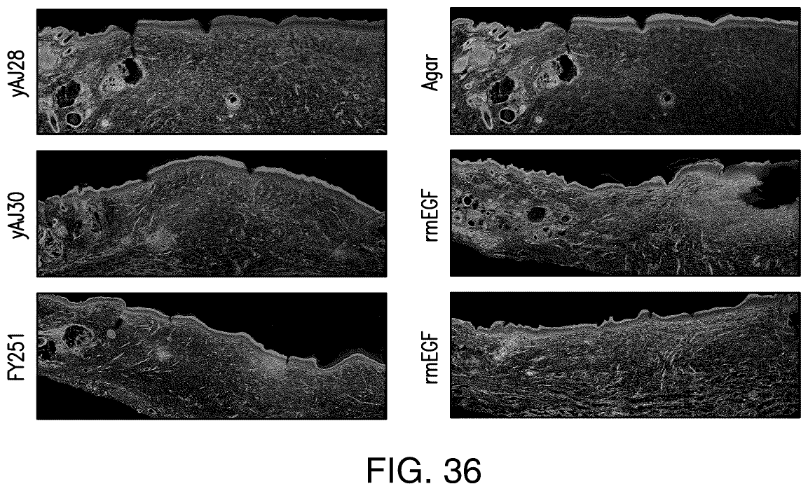

K5, K10 and K14 stainings showed that wounds treated with EGF-secreting yeast hydrogel dressings and LEP-secreting hydrogel dressings have a thicker epidermis than wounds treated with the control hydrogel dressings (FIGS. 33-35). Loricrin staining shows proper formation of the cornified layer in for all treatment conditions (FIG. 36). CD31 staining showed that wounds treated with the recombinant protein hydrogel dressing contained the highest vascularization density and wounds treated with the control yeast hydrogel dressing had the least amount of vascularization (FIG. 33). Similarly, wounds treated with EGF-secreting yeast had better vascularization than the ones treated with LEP-secreting yeast (FIG. 33).

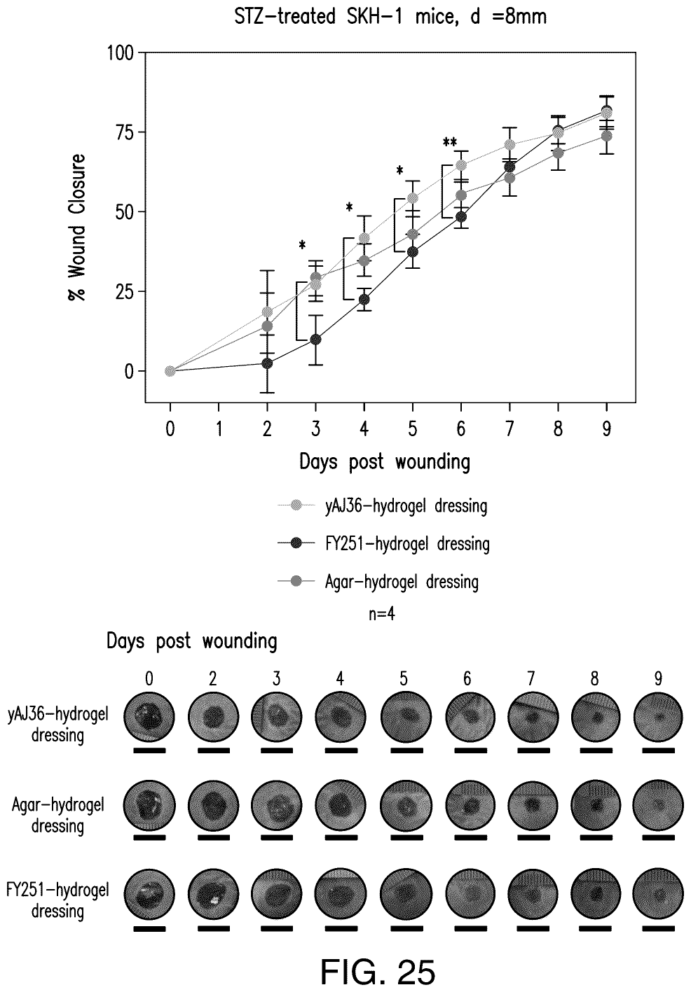

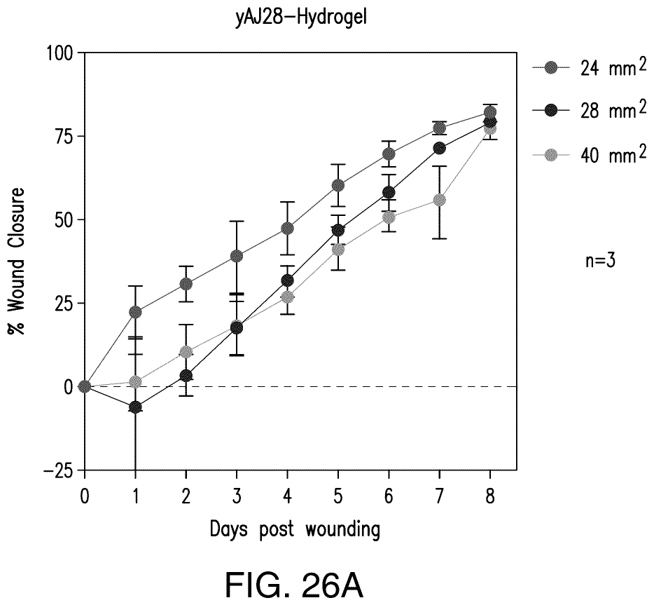

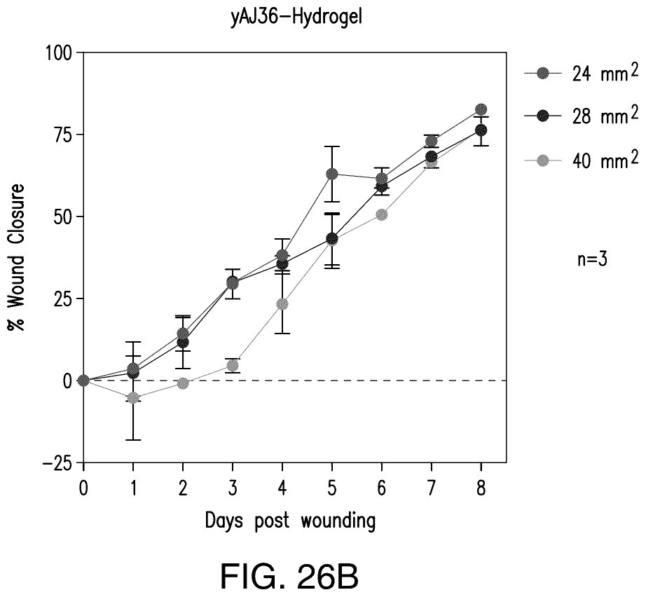

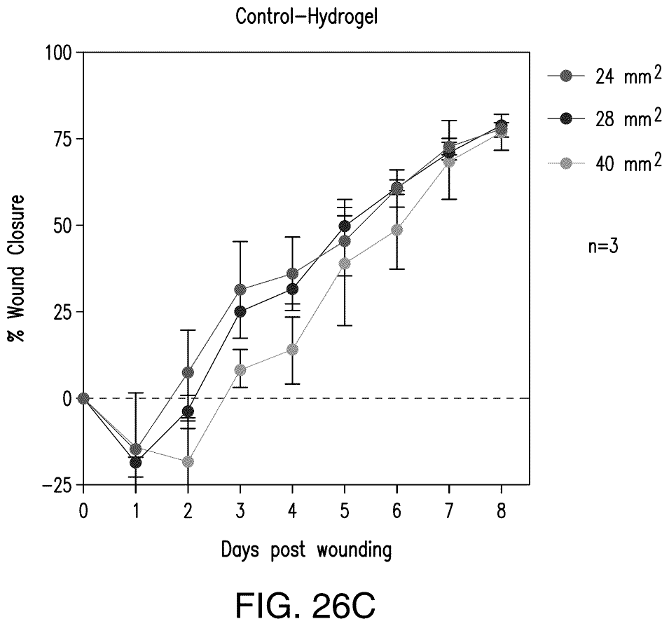

To visualize the effect of the initial wound size on the rate of healing for each treatment, the wound size progression for groups of initial wound sizes were graphed or each treatment. As expected, the wounds with smaller wound sizes (24 mm²) healed much faster than wounds with larger initial wound sizes (40 mm²) (FIGS. 26A-26D).

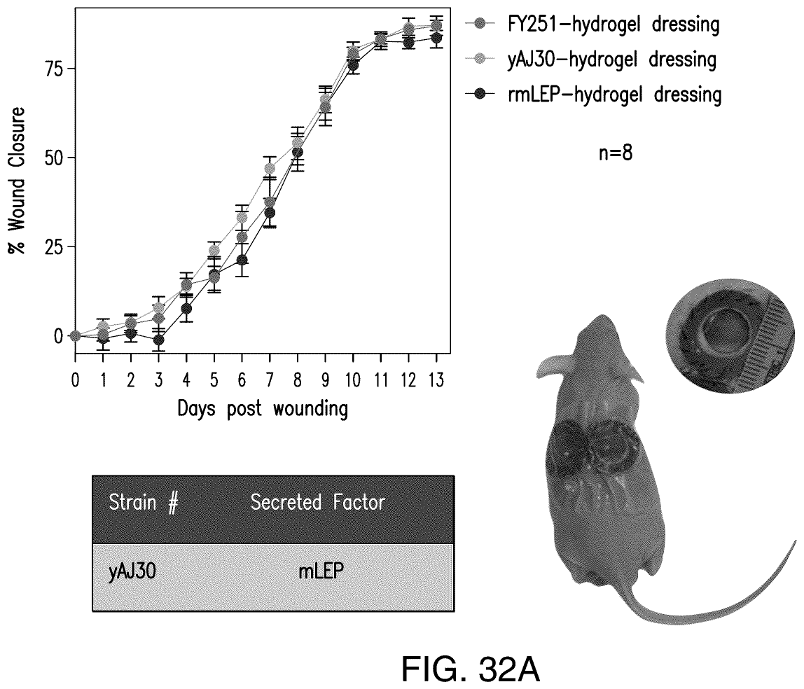

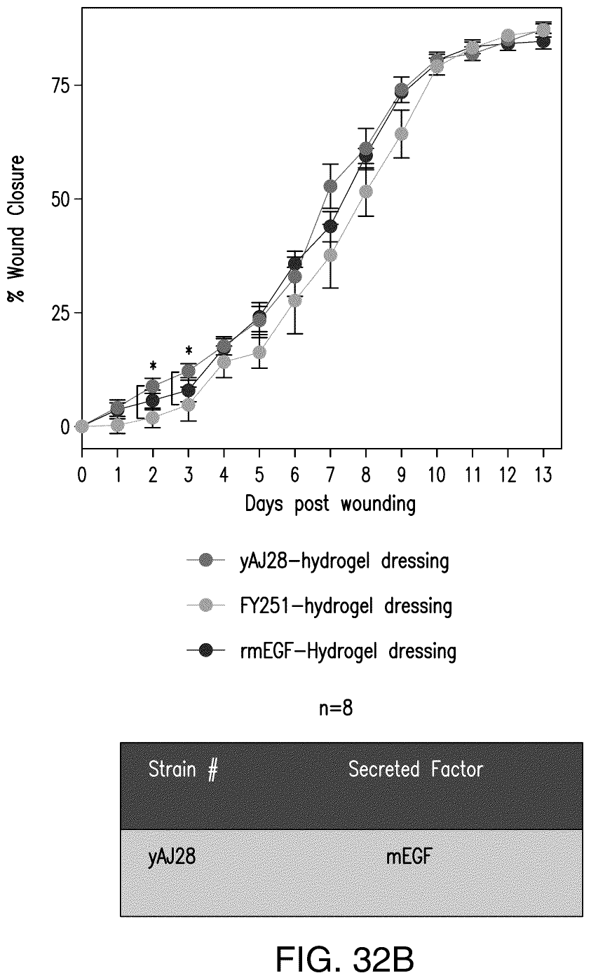

To ensure that the enhanced healing of the wounds treated with disclosed engineered yeast results from mechanisms such as increased cell proliferation and cell migration rather than skin contraction (which occurs during wound healing in mice and not humans), the hydrogel wound dressings were placed on splinted wound models. Six different dressings were tested: (i) yAJ28-hydrogel dressing (EGF secreting yeast), (ii) yAJ30-hydrogel dressing (LEP secreting yeast), (iii) FY251-hydrogel dressing (control yeast), (iv) agar-hydrogel dressing, (v) rmEGF hydrogel dressing and (vi) rmLEP hydrogel dressing on 50 mm² excisional wounds where each wound was supported with a medical grade silicone ring (splint) sutured around it (FIG. 32A). Similar to previous experiments, SKH-1 mice were STZ-treated and stably diabetic before wounding, and two full-thickness wounds were excised on the back of each mouse. Each test group was studied in eight replicates. The results (FIGS. 32A-32B) showed that wounds treated with the engineered yeast dressings (containing yAJ28 or yAJ30) shrank more rapidly than wounds treated with the control yeast dressing. Moreover, wounds treated with the engineered yeast dressings closed slightly faster than ones treated with dressings containing 100 ng/ml of recombinant proteins. Moreover, in this study (splinted wounds), the formation of an outer layer on the top of the excisional wound during the healing process was observed. This layer was formed between the first and fifth day post wounding. Without being limited to a particular theory, this layer can be either granulation tissue or a scab. Interestingly, this layer showed up earlier on the wounds treated with the engineered yeast hydrogel dressings (yAJ28 or yAJ30 containing dressings) than on wounds treated with the agar hydrogel dressing or the control yeast hydrogel dressing. Without being limited to a particular theory, the formation of this layer can be a sign of active healing.

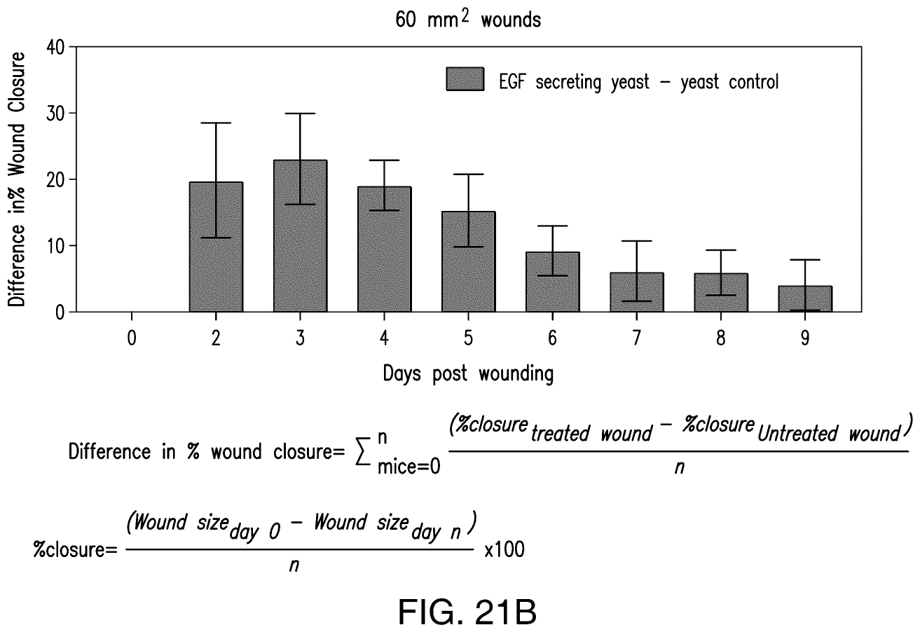

Further analysis was performed to determine if the yeast present in the hydrogel diffused from hydrogel through the wound bed and into the blood stream of the treated mice. If the yeast from the treatment diffused through the wound bed to the mouse body, it would be present in the blood and could be detected by measuring the mCherry florescence since the control yeast expresses mCherry. After 8 days of treatment, 100 μl of blood was collected from the submandibular vein of four mice that were treated with the mCherry yeast control hydrogel, the blood was mixed in 5 ml of yeast growth media and incubated at 30° C. for 24 and 48 hours and the fluorescence was measured at each timepoint. As shown in FIG. 22, no mCherry fluorescence was detected from the mouse blood sample, confirming that the yeast did not enter circulation via the wound bed.

The results from the experiments performed in this example show that wounds treated with the engineered live yeast dressings heal faster than those treated with the control dressings. In addition, staining of the treated tissue show that application of the engineered live yeast hydrogel dressing leads to microscopic level improvements in the wound bed, the newly formed skin layers and the healing process overall.

### Example 8: Treatment of Psoriasis

Psoriasis is a typical autoimmune disease caused by a deregulation of the Th1/Th2 balance, and immunotherapy for psoriasis has been shown to be clinically efficacious. Vascular endothelial growth factor (VEGF) is a potent mediator of angiogenesis. Psoriasis therapies typically include antibody-based or fusion protein-based biological therapies. These include biological agents interfering with T-cell function, TNF-α antagonists, anti-IL-17 agents and agents preventing the action of IL-12 and IL-23 by binding their mutual subunit p40.

This Example shows the use of IL-4 for the treatment of psoriasis. IL-4 is a protein of 129 amino acids and is glycosylated at two arginine residues (positions 38 and 105) and includes 3 disulfide bonds. IL-4 is locally administered to affected areas by topically applied yeast genetically engineered to express and secrete IL-4. To express and secrete IL-4, yeast are transformed with a high copy yeast plasmid designed with a strong constitutive alcohol dehydrogenase one promoter (pADH1) or other yeast promoters, a signal peptide for the secretory pathway such as the yeast alpha factor prepro signal peptide, and the gene encoding for the IL-4. To study the protein secretion and the secretion titers, western blotting and ELISA assays are utilized. To assess the biological activity, cell proliferation assays are run using TF-1 human or mouse erythroleukemic cells.

### Example 9: Treatment of Epidermolysis Bullosa

Epidermolysis bullosa is a family of disorders caused by genetic defects in the structural proteins of the skin, resulting in unusually fragile skin and mucous membranes that break and blister very easily. Based on statistics collected through the National Epidermolysis Bullosa Registry, EB is estimated to occur in 20 newborns per 1 million live births in the United States. EB prevalence is estimated at 1/18,000 live births. The exact number of persons with EB is unclear, but estimates suggest that 25,000-50,000 people in the United States have EB.

Major types of epidermolysis bullosa include epidermolysis bullosa simplex, hemidesmosomal epidermolysis bullosa, junctional epidermolysis bullosa, and dystrophic epidermolysis bullosa. One particularly severe form of EB known as recessive dystrophic EB (RDEB) occurs when the gene that codes for a protein called Type VII collagen is defective or missing. Without this protein, the skin's two main layers do not adhere properly at the dermal-epidermal junction (DEJ) and tend to separate, causing the blisters and risk of infection.

This Example shows the use of Type VII Collagen for the treatment of EB. Collagen Type VII is locally administered to affected areas by topically applied yeast genetically engineered to express and secrete Collagen Type VII.

### Example 10: Treatment of Skin Fibrosis and Scleroderma

Skin fibrosis is a devastating clinical condition commonly seen in skin-restricted and systemic disorders. The goal of skin fibrosis treatment is the restoration of abnormally activated dermal fibroblasts producing the excessive amount of extracellular matrix. Skin fibrosis can be treated with peptide inhibitor of TGF-β1. The peptide inhibitor can have the amino acid sequence TSLDASIIWAMMQN (SEQ ID NO: 9) or TSLDASIIWAMMQNA (SEQ ID NO: 12)

Yeast were genetically engineered to express TSLDASIIWAMMQ under control of the TDH3 promoter and secreted from the cell using a Pre-Pro-(Kex2) signal peptide. The nucleotide sequence that encodes TSLDASIIWAMMQNA (SEQ ID NO: 12) is ACTTCATTAGACGCCTCAATAATCTGGGCGATGATGCAGAATGCT (SEQ ID NO: 59).

This Example shows the use of peptide inhibitor of TGF-β1 for treating skin fibrosis and scleroderma. The peptide inhibitor of TGF-β1 having the amino acid sequence TSLDASIIWAMMQN (SEQ ID NO: 9) or TSLDASIIWAMMQNA (SEQ ID NO: 12) is locally administered to affected areas by topically applied yeast genetically engineered to express and secrete this peptide.

### Example 11: Treatment of Fungal and Yeast Infections

Yeast were genetically engineered to express RcALB-PepI under control of the ADH1 promoter and secreted from the cell using the mating factor alpha-1 to treat a fungal, e.g., yeast, infection. The amino acid sequence for RcALB-PepI is AKLIPTIAL (SEQ ID NO: 55), which is encoded by the nucleotide sequence GCTAAATTGATTCCAACTATTGCTTTGACT (SEQ ID NO: 60).

### Example 12: Treatment of Bacterial Infections

Yeast were genetically engineered to express pexiganan acetate (MSI-78) under control of the ADH1 promoter and secreted from the cell using the mating factor alpha-1 to treat a bacterial infection. The amino acid sequence for pexiganan acetate (MSI-78) that is expressed by the genetically engineered yeast is GIGKFLKKAKKFGKAFVKILKKG (SEQ ID NO: 56), which is encoded by the nucleotide sequence GGTATTGGAAAATTTTTGAAGAAAGCTAAAAAGTTT GGTAAGGCTTTTGTAAAAATACTGAAAAAGGGT (SEQ ID NO: 64).

### Example 13: Treatment of Acne

Yeast were genetically engineered to express melittin under control of the TDH3 promoter and secreted from the yeast cell using a Pre-Pro-(Kex2) signal peptide to treat acne. The amino acid sequence for melittin that is expressed by the genetically engineered yeast is GIGAVLKVLTTGLPALISWIKRKRQQ (SEQ ID NO: 53), which is encoded by the nucleotide sequence GGGATTGGGGCCGTGTTGAAAGTTCTTACGAC TGGTTTACCGGCCCTAATCTCATGGATCAAAAGGAAGAGGCAGCAG (SEQ ID NO: 61).

### REFERENCES

1. Enoch S, Leaper D J. Basic science surgery 26:2 31 Basic science of wound healing. 2008; 26(2):37-42.
2. Of B, Healing W. Cutaneous wound healing. N Engl J Med. 1999;
3. Martin P. Wound healing—Aiming for perfect skin regeneration. Science (80-). 1997; 276(5309):75-81.
4. Rodrigues M, Kosaric N, Bonham C A, Gurtner G C. Wound healing: A cellular perspective. Physiol Rev. 2019; 99(1):665-706.
5. Gonzalez A C D O, Andrade Z D A, Costa T F, Medrado A R A P. Wound healing—A literature review. An Bras Dermatol. 2016; 91(5):614-20.
6. Frykberg R G, Banks J. Challenges in the Treatment of Chronic Wounds. Adv Wound Care. 2015; 4(9):560-82.
7. Martin P, Nunan R. Cellular and molecular mechanisms of repair in acute and chronic wound healing. Br J Dermatol. 2015; 173(2):370-8.
8. Falanga V. Wound healing and its impairment in the diabetic foot. Lancet. 2005; 366(9498):1736-43.
9. Lau H C, Kim A. Pharmaceutical perspectives of impaired wound healing in diabetic foot ulcer. J Pharm Investig. 2016; 46(5):403-23.
10. Öhnstedt E, Lofton Tomenius H, Vagesja E, Phillipson M. The discovery and development of topical medicines for wound healing. Expert Opin Drug Discov [Internet]. 2019; 14(5):485-97. Available from: https://doi.org/10.1080/17460441.2019.1588879
11. Barrientos S, Stojadinovic O, Golinko M S, Brem H, Tomic-Canic M. Growth factors and cytokines in wound healing. Wound Repair Regen. 2008; 16(5):585-601.
12. Badiu D, Vasile M, Teren O. Regulation of wound healing by growth factors and cytokines. Wound Heal Process Phases Promot. 2011; 73-93.
13. Mast B A, Schultz G S. Interactions of cytokines, growth factors, and proteases in acute and chronic wounds. Wound Repair Regen. 1996; 4(4):411-20.
14. Meyer-Ingold W. Wound therapy: growth factors as agents to promote healing. Trends Biotechnol. 1993; 11(9):387-92.
15. Robson M C. Invited discussion: Recombinant human epidermal growth factor (EGF) to enhance healing for diabetic foot ulcers. Ann Plast Surg. 2006; 56(4):399-400.
16. Mustoe T A. Clinical evaluation of recombinant human platelet-derived growth factor for the treatment of lower extremity ulcers. Plast Reconstr Surg. 2006; 117(7 SUPPL.):71-81.
17. Gillitzer R, Goebeler M. Chemokines in cutaneous wound healing. J Leukoc Biol [Internet]. 2001; 69(4):513-21. Available from: http://www.ncbi.nlm.nih.gov/pubmed/11310836
18. Sánchez-Martín L, Estecha A, Samaniego R, Sánchez-Ramón S, Vega M A, Sánchez-Mateos P. The chemokine CXCL12 regulates monocyte-macrophage differentiation and RUNX3 expression. Blood. 2011; 117(1):88-97.
19. Feng G, Hao D, Chai J. Processing of CXCL12 impedes the recruitment of endothelial progenitor cells in diabetic wound healing. FEBS J. 2014; 281(22):5054-62.
20. Nakamura Y, Ishikawa H, Kawai K, Tabata Y, Suzuki S. Enhanced wound healing by topical administration of mesenchymal stem cells transfected with stromal cell-derived factor-1. Biomaterials [Internet]. 2013; 34(37):9393-400. Available from: http://dx.doi.org/10.1016/j.biomaterials.2013.08.053
21. Vågesjö E, Öhnstedt E, Mortier A, Lofton H, Huss F, Proost P, et al. Accelerated wound healing in mice by on-site production and delivery of CXCL12 by transformed lactic acid bacteria. Proc Natl Acad Sci USA. 2018; 115(8):1895-900.
22. Badillo A T, Chung S, Zhang L, Zoltick P, Liechty K W. Lentiviral Gene Transfer of SDF-1α to Wounds Improves Diabetic Wound Healing. J Surg Res. 2007; 143(1):35-42.
23. Murad A, Nath A K, Cha S-T, Demir E, Floras-Riveras J, Sierra-Honigmann M R. Leptin is an autocrine/paracrine regulator of wound healing. FASEB J. 2003; 17(13):1-15.
24. La Cava A. Leptin in inflammation and autoimmunity. Cytokine [Internet]. 2017; 98:51-8. Available from: http://dx.doi.org/10.1016/j.cyto.2016.10.011
25. Tadokoro S, Ide S, Tokuyama R, Umeki H, Tatehara S, Kataoka S, et al. Leptin promotes wound healing in the skin. PLoS One [Internet]. 2015; 10(3):1-16. Available from: http://dx.doi.org/10.1371/journal.pone.0121242
26. Frank S, Stallmeyer B, Kampfer H, Kolb N, Pfeilschifter J. Leptin enhances wound re-epithelialization and constitutes a direct function of leptin in skin repair. J Clin Invest. 2000; 106(4):501-9.
27. Hou J, Tyo K E J, Liu Z, Petranovic D, Nielsen J. Metabolic engineering of recombinant protein secretion by Saccharomyces cerevisiae. FEMS Yeast Res. 2012; 12(5):491-510.
28. Baghban R, Farajnia S, Rajabibazl M, Ghasemi Y, Mafi A A, Hoseinpoor R, et al. Yeast Expression Systems: Overview and Recent Advances. Mol Biotechnol [Internet]. 2019; 61(5):365-84. Available from: http://dx.doi.org/10.1007/s12033-019-00164-8
29. Han M, Yu X. Enhanced expression of heterologous proteins in yeast cells via the modification of N-glycosylation sites. Bioengineered. 2015; 6(2):115-8.
30. Smith R A, Duncan M J, Moir D T. Heterologous Protein Secretion from Yeast. Adv Sci. 1985; 229(4719):1219-24.
31. Nielsen J. Production of biopharmaceutical proteins by yeast: Advances through metabolic engineering. Bioengineered. 2013; 4(4):207-11.
32. Wildt S, Gerngross T U. The humanization of N-glycosylation pathways in yeast. Nat Rev Microbiol. 2005; 3(2):119-28.
33. Laurent J M, Young J H, Kachroo A H, Marcotte E M. Efforts to make and apply humanized yeast. Brief Funct Genomics. 2016; 15(2):155-63.
34. Kis Z, Shattock R, Shah N, Kontoravdi C. Emerging Technologies for Low-Cost, Rapid Vaccine Manufacture. Biotechnol J. 2019; 14(1):1-14.
35. Broyer L, Goetsch L, Broussas M. Glycosylation Engineering of Biopharmaceuticals. Vol. 988, Methods in Molecular Biology. 2013. 319-329 p.
36. Sarcoma S, One V, Between I S, Encoding S, Derived P, Factor G, et al.

Expression of biologically active PDGF analogs in yeast. 2005; (19).
37. Buckholz R G, Gleeson M A G. Yeast Systems for the commercial production of heterologous proteins. 1991; 9(November):1067-72.
38. Shpilman M, Niv-Spector L, Katz M, Varol C, Solomon G, Ayalon-Soffer M, et al. Development and characterization of high affinity leptins and leptin antagonists. J Biol Chem [Internet]. 2011; 286(6):4429-42. Available from: http://dx.doi.org/10.1074/jbc.M110.196402
39. Huang M, Bao J, Nielsen J. Biopharmaceutical protein production by Saccharomyces cerevisiae: current state and future prospects. Pharm Bioprocess. 2014; 2(2):167-82.
40. Brake A J, Merryweather J P, Coit D G, Heberlein U A, Masiarz F R, Mullenbach G T, et al. α-Factor-directed synthesis and secretion of mature foreign proteins in Saccharomyces cerevisiae. Proc Natl Acad Sci USA. 1984; 81(15 I):4642-6.
41. Bitter G A, Chen K K, Banks A R, Por-Hsiung Lai. Secretion of foreign proteins from Saccharomyces cerevisiae directed by α-factor gene fusions. Proc Natl Acad Sci USA [Internet]. 1984 Sep. 1 [cited 2020 Sep. 30]; 81(17 I):5330-4. Available from: https://www.pnas.org/content/81/17/5330
42. sebo, K. M., Lu, H. S., Fieschko, J. C., Goldstein, L., Davis, J., Duker, K., & Bitter G A. Protein secretion from Saccharomyces cerevisiae directed by the prepro-alpha-factor leader region. J Biol Chem. 1986; 261(13):5858-65.
43. Delic M, Valli M, Graf A B, Pfeffer M, Mattanovich D, Gasser B. The secretory pathway: Exploring yeast diversity. FEMS Microbiol Rev. 2013; 37(6):872-914.
44. Billerbeck S, Brisbois J, Agmon N, Jimenez M, Temple J, Shen M, et al. A scalable peptide-GPCR language for engineering multicellular communication. Nat Commun [Internet]. 2018 Dec. 1 [cited 2020 Sep. 29]; 9(1):1-12. Available from: www.nature.com/naturecommunications
45. Besada-Lombana P B, Da Silva N A. Engineering the early secretory pathway for increased protein secretion in Saccharomyces cerevisiae. Metab Eng [Internet]. 2019; 55(January):142-51. Available from: https://doi.org/10.1016/j.ymben.2019.06.010
46. Fitzgerald I, Glick B S. Secretion of a foreign protein from budding yeasts is enhanced by cotranslational translocation and by suppression of vacuolar targeting. Microb Cell Fact. 2014; 13(1):1-12.
47. Kim M D, Han K C, Kang H A, Rhee S K, Seo J H. Coexpression of BiP increased antithrombotic hirudin production in recombinant Saccharomyces cerevisiae. J Biotechnol. 2003; 101(1):81-7.
48. Robinson A S, Hines V, Wittrup K D. Protein Disulfide Isomerase Overexpression Increases Secretion of Foreign Proteins in Saccharomyces cerevisiae Anne. Biotechnology. 1994; 12(April):381-4.
49. Morozkina E V., Marchenko A N, Keruchenko J S, Keruchenko I D, Khotchenkov V P, Popov V O, et al. Proteinase B disruption is required for high level production of human mechano-growth factor in Saccharomyces cerevisiae. J Mol Microbiol Biotechnol. 2010; 18(3):188-94.
50. Cho E Y, Cheon S A, Kim H, Choo J, Lee D J, Ryu H M, et al. Multiple-yapsin-deficient mutant strains for high-level production of intact recombinant proteins in Saccharomyces cerevisiae. J Biotechnol [Internet]. 2010; 149(1-2):1-7. Available from: http://dx.doi.org/10.1016/j.jbiotec.2010.06.014
51. Kondo A, Hasunuma T, Inokuma K. Secretion Signal Peptide, And Protein Secretory Production And Cell Surface Display Using Said Secretion Signal Peptide. Google Patents; 2017.
52. Jonkman J E N, Cathcart J A, Xu F, Bartolini M E, Amon J E, Stevens K M, et al. An introduction to the wound healing assay using livecell microscopy An introduction to the wound healing assay using livecell microscopy. Cell Adhes Migr. 2014; 8(5):440-51.
53. Feiner R C, Penne I, Müller B, Müller K M. EGF-mCherry Fusion Protein Expressed in E. coli Shows Product Heterogeneity but a High Biological Activity. Biochemistry. 2019; 58(8):1043-7.
54. Webb D J, Zhang H, Horwitz A F. Cell migration: an overview. Vol. 294, Methods in molecular biology (Clifton, N. J.). 2005. 3-11 p.
55. Yin H, Chen C Y, Liu Y W, Tan Y J, Deng Z L, Yang F, et al. Synechococcus elongatus PCC7942 secretes extracellular vesicles to accelerate cutaneous wound healing by promoting angiogenesis. Theranostics. 2019; 9(9):2678-93.
56. Kalghatgi S, Friedman G, Fridman A, Clyne A M. Endothelial cell proliferation is enhanced by low dose non-thermal plasma through fibroblast growth factor-2 release. Ann Biomed Eng. 2010; 38(3):748-57.
57. Huo Y, Qiu W Y, Pan Q, Yao Y F, Xing K, Lou M F. Reactive oxygen species (ROS) are essential mediators in epidermal growth factor (EGF)-stimulated corneal epithelial cell proliferation, adhesion, migration, and wound healing. Exp Eye Res [Internet]. 2009; 89(6):876-86. Available from: http://dx.doi.org/10.1016/j.exer.2009.07.012
58. Yoon J E, Kim Y, Kwon S, Kim M, Kim Y H, Kim J H, et al. Senescent fibroblasts drive ageing pigmentation: A potential therapeutic target for senile lentigo. Theranostics. 2018; 8(17):4620-32.
59. Orrapin S, Rekasem K. Role of Topical Biological Therapies and Dressings in Healing Ischemic Wounds. Int J Low Extrem Wounds. 2018; 17(4):236-46.
60. Ribeiro D M L, Júnior A R C, de Macedo G H R V, Chagas V L, Silva L D S, Cutrim B da S, et al. Polysaccharide-based formulations for healing of skin-related wound infections: Lessons from animal models and clinical trials. Biomolecules. 2020; 10(1):1-16.
61. Boateng J S, Matthews K H, Stevens H N E, Eccleston G M. Wound healing dressings and drug delivery systems: A review. J Pharm Sci [Internet]. 2008; 97(8):2892-923. Available from: http://dx.doi.org/10.1002/jps.21210
62. Abdelrahman T, Newton H. Wound dressings: Principles and practice. Surgery [Internet]. 2011; 29(10):491-5. Available from: http://dx.doi.org/10.1016/j.mpsur.2011.06.007
63. Vowden K, Vowden P. Wound dressings: principles and practice. Surg (United Kingdom) [Internet]. 2017; 35(9):489-94. Available from: http://dx.doi.org/10.1016/j.mpsur.2017.06.005
64. Opt Veld R C, Walboomers X F, Jansen J A, Wagener F A D T G. Design Considerations for Hydrogel Wound Dressings: Strategic and Molecular Advances. Tissue Eng—Part B Rev. 2020; 26(3):230-48.
65. Waycaster C, Milne C T. Clinical and economic benefit of enzymatic debridement of pressure ulcers compared to autolytic debridement with a hydrogel dressing. J Med Econ. 2013; 16(7):976-86.
66. Saghazadeh S, Rinoldi C, Schot M, Kashaf S S, Sharifi F, Jalilian E, et al. Drug delivery systems and materials for wound healing applications. Adv Drug Deliv Rev. 2018; 127:138-66.
67. Miller K J, Cao W, Ibrahim M M, Levinson H. The effect of microporous polysaccharide hemispheres on wound healing and scarring in wild-type and db/db mice. Adv Ski Wound Care. 2017; 30(4):169-80.
68. Chen H, Cheng Y, Tian J, Yang P, Zhang X, Chen Y, et al. Dissolved oxygen from microalgae-gel patch promotes chronic wound healing in diabetes. Sci Adv. 2020; 6(20).
69. Tandon B, Magaz A, Balint R, Blaker J J, Cartmell S H. Electroactive biomaterials: Vehicles for controlled delivery of therapeutic agents for drug delivery and tissue regeneration. Adv Drug Deliv Rev [Internet]. 2018; 129:148-68. Available from: https://doi.org/10.1016/j.addr.2017.12.012
70. Sattar M M, Patel M, Alani A. Clinical applications of polytetrafluoroethylene (PTFE) tape in restorative dentistry. Br Dent J. 2017; 222(3):151-8.

The contents of all figures and all references, patents and published patent applications and Accession numbers cited throughout this application are expressly incorporated herein by reference.

1. A pharmaceutical composition comprising: (i) a live fungal cell genetically engineered to express and secrete a skin therapeutic and (ii) a pharmaceutically acceptable carrier.

2. The pharmaceutical composition of claim 1, wherein the skin therapeutic is secreted from the fungal cell by a secretory pathway of the fungal cell.

3. The pharmaceutical composition of claim 1 or 2, wherein the skin therapeutic is a protein or a functional fragment thereof.

4. The pharmaceutical composition of any one of claims 1-3, wherein the skin therapeutic is selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof and a combination thereof.

5. The pharmaceutical composition of claim 4, wherein the skin therapeutic is a growth factor or a derivative thereof or an inhibitor of a growth factor or a derivative thereof.

6. The pharmaceutical composition of claim 5, wherein the growth factor is selected from the group consisting of epidermal growth factor (EGF), platelet-derived growth factor (PDGF), fibroblast growth factor (FGF), vascular endothelial growth factor (VEGF), transforming growth factor-beta (TGF-β) and a combination thereof.

7. The pharmaceutical composition of claim 6, wherein the growth factor is EGF.

8. The pharmaceutical composition of claim 6, wherein the growth factor is PDGF.

9. The pharmaceutical composition of claim 6, wherein the growth factor is TGF-β.

10. The pharmaceutical composition of claim 6, wherein the growth factor is VEGF.

11. The pharmaceutical composition of claim 4, wherein the skin therapeutic is a chemokine or a derivative thereof.

12. The pharmaceutical composition of claim 11, wherein the chemokine is CXCL12.

13. The pharmaceutical composition of claim 4, wherein the skin therapeutic is a cytokine or a derivative thereof.

14. The pharmaceutical composition of claim 13, wherein the cytokine is selected from the group consisting of Leptin, IL-4 and a combination thereof.

15. The pharmaceutical composition of claim 4, wherein the skin therapeutic is a protease inhibitor or a derivative thereof.

16. The pharmaceutical composition of claim 15, wherein the skin therapeutic is selected from the group consisting of TIMP1, TIMP2 and a combination thereof.

17. The pharmaceutical composition of claim 4, wherein the skin therapeutic is an extracellular matrix protein or a derivative thereof.

18. The pharmaceutical composition of claim 17, wherein the skin therapeutic is selected from the group consisting of Type VII Collagen, elastin and a combination thereof.

19. The pharmaceutical composition of claim 1 or 2, wherein the skin therapeutic is an antimicrobial and/or an anti-inflammatory peptide.

20. The pharmaceutical composition of claim 19, wherein the antimicrobial and/or anti-inflammatory peptide is selected from the group consisting of cathelicidin antimicrobial peptide (LL-37) or analogs thereof, RcA1b-PepI, RcA1b-PepII, RcA1b-PepII, lucifensin, lucifensin II, lucilin, pexiganan acetate (MSI-78), D2A21/D4E1, granulysin, a synthetic granulysin-derived peptide and a combination thereof.

21. The pharmaceutical composition of any one of claims 1-20, wherein the fungal cell is a species from a genus selected from the group consisting of Cladosporium, Aureobasidium, Aspergillus, Saccharomyces, Malassezia, Epicoccum, Candida, Penicillium, Wallemia, Pichia, Phoma, Cryptococcus, Fusarium, Clavispora, Cyberlindnera, Kluyveromyces and a combination thereof.

22. The pharmaceutical composition of any one of claims 1-21, wherein the fungal cell is Saccharomyces cerevisiae or Pichia pastoris.

23. The pharmaceutical composition of any one of claims 1-22 further comprising a second live fungal cell genetically engineered to express and secrete a second skin therapeutic.

24. The pharmaceutical composition of claim 23 further comprising a third live fungal cell genetically engineered to express and secrete a third skin therapeutic.

25. The pharmaceutical composition of claim 24 further comprising a fourth live fungal cell genetically engineered to express and secrete a fourth skin therapeutic.

26. The pharmaceutical composition of claim 25 further comprising a fifth live fungal cell genetically engineered to express and secrete a fifth skin therapeutic and/or further comprising a sixth live fungal cell genetically engineered to express and secrete a sixth skin therapeutic.

27. The pharmaceutical composition of any one of claims 1-26, wherein the pharmaceutical composition is formulated for rectal administration, vaginal administration or topical administration.

28. The pharmaceutical composition of claim 27, wherein the pharmaceutical composition is formulated for topical administration.

29. The pharmaceutical composition of any one of claims 1-28, wherein the pharmaceutically acceptable carrier comprises a hydrogel.

30. The pharmaceutical composition of claim 29, wherein the hydrogel comprises from about 0.1% w/v to about 5.0% w/v of a polysaccharide.

31. The pharmaceutical composition of claim 30, wherein the hydrogel comprises from about 0.1% w/v to about 1.0% w/v of a polysaccharide.

32. The pharmaceutical composition of claim 30 or 31, wherein the polysaccharide is agarose.

33. The pharmaceutical composition of any one of claims 1-32, wherein the pharmaceutical compositions comprises a therapeutically effect amount of the live genetically-engineered fungal cell, wherein the therapeutically effective amount is from about 1×10³ cells/ml to about 1×10¹⁰ cells/ml of the live genetically-engineered fungal cells.

34. The pharmaceutical composition of claim 33, wherein the therapeutically effective amount is from about 1×10⁶ cells/ml to about 2×10⁷ cells/ml of the live genetically-engineered fungal cells.

35. The pharmaceutical composition of any one of claims 1-34, wherein the therapeutically effective amount of live genetically-engineered fungal cells comprises an amount of live genetically-engineered fungal cells that express and secrete from about 1 pg/ml and about 200,000 pg/ml of the skin therapeutic in about 24 hours or less.

36. The pharmaceutical composition of claim 35, wherein the therapeutically effective amount of live genetically-engineered fungal cells comprises an amount of live genetically-engineered fungal cells that express and secrete from about 100 pg/ml to about 25,000 pg/ml of the skin therapeutic in about 24 hours or less.

37. The pharmaceutical composition of any one of claims 1-36, wherein the live genetically-engineered fungal cell secretes and expresses the skin therapeutic for about 24 hours to about 2 weeks after administration.

38. The pharmaceutical composition of claim 37, wherein the live genetically-engineered fungal cell secretes and expresses the skin therapeutic for at least about 48 hours after administration.

39. The pharmaceutical composition of claim 38, wherein the live genetically-engineered fungal cell secretes and expresses the skin therapeutic for at least about 72 hours after administration.

40. The pharmaceutical composition of claim 39, wherein the live genetically-engineered fungal cell secretes and expresses the skin therapeutic for at least about 96 hours after administration.

41. The pharmaceutical composition of any one of claims 1-40, wherein the live genetically-engineered fungal cell continuously secretes and expresses the skin therapeutic.

42. The pharmaceutical composition of any one of claims 1-41, further comprising one or more nutrients for the one or more genetically engineered fungal cells.

43. A topical pharmaceutical composition comprising a hydrogel comprising a live fungal cell genetically engineered to express and secrete a skin therapeutic.

44. The topical pharmaceutical composition of claim 43, wherein the hydrogel comprises from about 0.1% w/v to about 5.0% w/v of a polysaccharide.

45. The topical pharmaceutical composition of claim 44, wherein the hydrogel comprises from about 0.1% w/v to about 1.0% w/v of a polysaccharide.

46. The topical pharmaceutical composition of claim 44 or 45, wherein the polysaccharide is agarose.

47. The topical pharmaceutical composition of any one of claims 43-46 further comprising a bottom layer facing the skin and a top layer facing the air, wherein the hydrogel comprising the fungal cell genetically engineered to express and secrete the skin therapeutic is disposed between the bottom layer and the top layer.

48. The topical pharmaceutical composition of claim 47, wherein the genetically-engineered fungal cell cannot pass through the bottom layer, and wherein the skin therapeutic can pass through the bottom layer.

49. The topical pharmaceutical composition of claim 47 or 48, wherein the bottom layer has a pore size of about 0.01 to about 1.0 μm.

50. The topical pharmaceutical composition of any one of claims 43-49, wherein the topical pharmaceutical composition comprises a therapeutically effective amount of the live genetically-engineered fungal cell.

51. The topical pharmaceutical composition of claim 50, wherein the therapeutically effective amount of live genetically-engineered fungal cells comprises from about 1×10³ cells/ml to about 1×10¹⁰ cells/ml of the live genetically-engineered fungal cells.

52. The topical pharmaceutical composition of claim 50 or 51, wherein the therapeutically effective amount of live genetically-engineered fungal cells comprises an amount of live genetically-engineered fungal cells that express and secrete from about 1 pg/ml and about 200,000 pg/ml of the skin therapeutic in about 24 hours or less.

53. The topical pharmaceutical composition of any one of claims 43-52, wherein the genetically engineered fungal cell secretes the skin therapeutic for at least about 12 hours, at least about 24 hours, at least about 48 hours, at least about 60 hours, at least about 72 hours, at least about 84 hours, at least about 96 hours, at least about 108 hours, at least about 120 hours, at least about 132 hours, at least about 144 hours, at least about 156 hours, at least about 165 hours, at least about 8 days, at least about 9 days, at least about 10 days, at least about 11 days, at least about 12 days, at least about 13 days or at least about 14 days after administration to the subject.

54. The topical pharmaceutical composition of any one of claims 43-53, wherein the fungal cell is Saccharomyces cerevisiae or Pichia pastoris.

55. The topical pharmaceutical composition of any one of claims 43-54, wherein the skin therapeutic is a protein or a functional fragment thereof.

56. The topical pharmaceutical composition of claim 55, wherein the skin therapeutic is selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof and a combination thereof.

57. The topical pharmaceutical composition of claim 56, wherein the skin therapeutic is a growth factor or a derivative thereof or an inhibitor of a growth factor or a derivative thereof.

58. The topical pharmaceutical composition of claim 57, wherein the growth factor is selected from the group consisting of epidermal growth factor (EGF), platelet-derived growth factor (PDGF), fibroblast growth factor (FGF), vascular endothelial growth factor (VEGF), transforming growth factor-beta (TGF-β) and a combination thereof.

59. The topical pharmaceutical composition of any one of claims 43-54, wherein the skin therapeutic is an antimicrobial and/or an anti-inflammatory peptide.

60. The topical pharmaceutical composition of claim 59, wherein the antimicrobial and/or anti-inflammatory peptide is selected from the group consisting of cathelicidin antimicrobial peptide (LL-37) or analogs thereof, RcA1b-PepI, RcA1b-PepII, RcA1b-PepII, lucifensin, lucifensin II, lucilin, pexiganan acetate (MSI-78), D2A21/D4E1, granulysin, a synthetic granulysin-derived peptide and a combination thereof.

61. A pharmaceutical composition comprising:
(i) a first live fungal cell genetically engineered to express and secrete a first skin therapeutic;
(ii) a second live fungal cell genetically engineered to express and secrete a second skin therapeutic; and
(iii) a pharmaceutically acceptable carrier,
wherein the first skin therapeutic and the second skin therapeutic are different.

62. The pharmaceutical composition of claim 61, wherein the first skin therapeutic and the second skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

63. The pharmaceutical composition of claim 62, wherein the first skin therapeutic comprises a growth factor and the second skin therapeutic comprises a chemokine.

64. The pharmaceutical composition of claim 62, wherein the first skin therapeutic comprises a growth factor and the second skin therapeutic comprises a cytokine.

65. A pharmaceutical composition comprising:
(i) a first live fungal cell genetically engineered to express and secrete a first skin therapeutic;
(ii) a second live fungal cell genetically engineered to express and secrete a second skin therapeutic;
(iii) a third live fungal cell genetically engineered to express and secrete a third skin therapeutic; and
(iv) a pharmaceutically acceptable carrier,
wherein the first skin therapeutic, the second skin therapeutic and the third skin therapeutic are different.

66. The pharmaceutical composition of claim 65, wherein the first skin therapeutic, the second skin therapeutic and the third skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

67. A pharmaceutical composition comprising:
(i) a first live fungal cell genetically engineered to express and secrete a first skin therapeutic;
(ii) a second live fungal cell genetically engineered to express and secrete a second skin therapeutic;
(iii) a third live fungal cell genetically engineered to express and secrete a third skin therapeutic;
(iv) a fourth live fungal cell genetically engineered to express and secrete a fourth skin therapeutic; and
(v) a pharmaceutically acceptable carrier,
wherein the first skin therapeutic, the second skin therapeutic, the third skin therapeutic and the fourth skin therapeutic are different.

68. The pharmaceutical composition of claim 67, wherein the first skin therapeutic, the second skin therapeutic, the third skin therapeutic and the fourth skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

69. A pharmaceutical composition comprising:
(i) a first live fungal cell genetically engineered to express and secrete a first skin therapeutic;
(ii) a second live fungal cell genetically engineered to express and secrete a second skin therapeutic;
(iii) a third live fungal cell genetically engineered to express and secrete a third skin therapeutic;
(iv) a fourth live fungal cell genetically engineered to express and secrete a fourth skin therapeutic;
(v) a fifth live fungal cell genetically engineered to express and secrete a fifth skin therapeutic; and
(vi) a pharmaceutically acceptable carrier,
wherein the first skin therapeutic, the second skin therapeutic, the third skin therapeutic, the fourth skin therapeutic and the fifth skin therapeutic are different.

70. The pharmaceutical composition of claim 69, wherein the first skin therapeutic, the second skin therapeutic, the third skin therapeutic, the fourth skin therapeutic and the fifth skin therapeutic are independently selected from the group consisting of a growth factor or a derivative thereof, a cytokine or a derivative thereof, a chemokine or a derivative thereof, a protease inhibitor or a derivative thereof, an extracellular matrix protein or a derivative thereof, an inhibitor of a growth factor or a derivative thereof, an antimicrobial and/or an anti-inflammatory peptide and a combination thereof.

71. A method for treating a subject in need thereof comprising administering to the subject the pharmaceutical composition of any one of claims 1-70.

72. The method of claim 71, wherein the pharmaceutical composition is formulated for topical administration.

73. The method of claim 71 or 72, wherein the pharmaceutical composition is administered to the subject to treat a skin condition or to perform a cosmetic procedure.

74. The method of claim 73, wherein the skin condition is selected from the group consisting of a wound, an infection, acne, a fibrotic disorder, a blistering disorder, an inflammatory condition, a vascular lesion, a skin cancer, xeroderma pigmentosum, a pigment disorder and a combination thereof.

75. The method of claim 74, wherein the skin condition is a wound.

76. The method of claim 75, wherein the wound is a diabetic ulcer.

77. The method of claim 74, wherein the skin condition is an infection.

78. The method of claim 74, wherein the skin condition is acne.

79. The method of claim 74, wherein the skin condition is a fibrotic disorder.

80. The method of claim 79, wherein the fibrotic disorder is scleroderma.

81. The method of claim 74, wherein the skin condition is a blistering disorder.

82. The method of claim 81, wherein the blistering disorder is epidermolysis bullosa.

83. The method of claim 74, wherein the skin condition is an inflammatory condition.

84. The method of claim 83, wherein the inflammatory condition is psoriasis.

85. The method of claim 74, wherein the skin condition is a vascular lesion.

86. The method of claim 74, wherein the skin condition is a skin cancer.

87. The method of claim 74, wherein the skin condition is xeroderma pigmentosum.

88. The method of claim 74, wherein the skin condition is a pigment disorder.

89. The method of any one of claims 71-88, wherein the genetically engineered fungal cell secretes the skin therapeutic for at least about 12 hours, at least about 24 hours, at least about 48 hours, at least about 60 hours, at least about 72 hours, at least about 84 hours, at least about 96 hours, at least about 108 hours, at least about 120 hours, at least about 132 hours, at least about 144 hours, at least about 156 hours, at least about 165 hours, at least about 8 days, at least about 9 days, at least about 10 days, at least about 11 days, at least about 12 days, at least about 13 days or at least about 14 days.

90. The method of claim 89, wherein the genetically engineered fungal cell secretes the skin therapeutic for at least about 72 hours after administration to the subject.

91. The method of any one of claims 71-90, wherein administration of the pharmaceutical composition to the subject comprises applying the pharmaceutical composition to the affected area.

92. The method of claim 91, wherein the pharmaceutical composition is applied no more than 5 times a week, no more than 4 times a week, no more than 3 times a week, no more than 2 times a week, no more than 1 time a week.

93. The method of claim 92, wherein the pharmaceutical composition is applied no more than 3 times a week.

94. The method of claim 93, wherein the pharmaceutical composition is applied no more than 2 times a week.

95. Use of the pharmaceutical composition of any one of claims 1-70 for treating a skin condition or for performing a cosmetic procedure.

96. The use of claim 95, wherein the skin condition is selected from the group consisting of a wound, an infection, acne, a fibrotic disorder, a blistering disorder, an inflammatory condition, a vascular lesion, a skin cancer, xeroderma pigmentosum, a pigment disorder and a combination thereof.

97. The use of claim 96, wherein the skin condition is a wound.

98. A kit comprising the pharmaceutical composition of any one of claims 1-70.

99. A kit for performing the method of any one of claims 71-94.


SEQUENCE LISTING (65 sequences)

SEQ ID NO: 1 - protein, 109 aa, synthetic construct
SLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVT

SEQ ID NO: 2 - protein, 109 aa, synthetic construct
SLGSLAAAEPAVIAECKTRTEVFQISRNLIDRTNANFLVWPPCVEVQRCSGCCNNRNVQCRASQVQMRPVQVRKIEIVRKKPIFKKATVTLEDHLACKCETIVTPRPVT

SEQ ID NO: 3 - protein, 53 aa, synthetic construct
NSDSECPLSHDGYCLHDGVCMYIEALDKYACNCVVGYIGERCQYRDLKWWELR

SEQ ID NO: 4 - protein, 53 aa, synthetic construct
NSYPGCPSSYDGYCLNGGVCMHIESLDSYTCNCVIGYSGDRCQTRDLRWWELR

SEQ ID NO: 5 - protein, 146 aa, synthetic construct
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 6 - protein, 146 aa, synthetic construct
VPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC

SEQ ID NO: 7 - protein, 68 aa, synthetic construct
KPVSLSYRCPCRFFESHIARANVKHLKILNTPNCALQIVARLKNNNRQVCIDPKLKWIQEYLEKALNK

SEQ ID NO: 8 - protein, 68 aa, synthetic construct
KPVSLSYRCPCRFFESHVARANVKHLKILNTPNCALQIVARLKNNNRQVCIDPKLKWIQEYLEKALNK

SEQ ID NO: 9 - protein, 14 aa, synthetic construct
TSLDASIIWAMMQN

SEQ ID NO: 10 - protein, 8 aa, synthetic construct
DYKDDDDK

SEQ ID NO: 11 - protein, 117 aa, synthetic construct
DYKDDDDKSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVT

SEQ ID NO: 12 - protein, 15 aa, synthetic construct
TSLDASIIWAMMQNA

SEQ ID NO: 13 - protein, 117 aa, synthetic construct; identical to SEQ ID NO: 11

SEQ ID NO: 14 - protein, 117 aa, synthetic construct
DYKDDDDKSLGSLAAAEPAVIAECKTRTEVFQISRNLIDRTNANFLVWPPCVEVQRCSGCCNNRNVQCRASQVQMRPVQVRKIEIVRKKPIFKKATVTLEDHLACKCETIVTPRPVT

SEQ ID NO: 15 - protein, 61 aa, synthetic construct
DYKDDDDKNSDSECPLSHDGYCLHDGVCMYIEALDKYACNCVVGYIGERCQYRDLKWWELR

SEQ ID NO: 16 - protein, 61 aa, synthetic construct
DYKDDDDKNSYPGCPSSYDGYCLNGGVCMHIESLDSYTCNCVIGYSGDRCQTRDLRWWELR

SEQ ID NO: 17 - protein, 154 aa, synthetic construct
DYKDDDDKVPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC

SEQ ID NO: 18 - protein, 154 aa, synthetic construct; identical to SEQ ID NO: 17

SEQ ID NO: 19 - protein, 154 aa, synthetic construct
DYKDDDDKVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 20 - protein, 76 aa, synthetic construct
DYKDDDDKKPVSLSYRCPCRFFESHIARANVKHLKILNTPNCALQIVARLKNNNRQVCIDPKLKWIQEYLEKALNK

SEQ ID NO: 21 - protein, 76 aa, synthetic construct
DYKDDDDKKPVSLSYRCPCRFFESHVARANVKHLKILNTPNCALQIVARLKNNNRQVCIDPKLKWIQEYLEKALNK

SEQ ID NO: 22 - DNA, 327 bp, synthetic construct
  source 1..327: mol_type = other DNA
agccttgggagcctgaccatcgcagagccagcaatgattgctgaatgcaaaacgaggacagaagtttttgaaataagcagaagactgatagatagaaccaacgctaattttttggtgtggcccccttgcgtggaggtacaaaggtgctctggatgctgcaataacaggaacgtacaatgcaggcccacccaggtacaactgaggccggtgcaggtcagaaagatcgagatagtcagaaagaaacctatattcaaaaaggccaccgtcacccttgaagaccatttggcgtgcaagtgtgagaccgttgcggcggccagacccgtgact

SEQ ID NO: 23 - DNA, 351 bp, synthetic construct
  source 1..351: mol_type = other DNA
gactacaaagacgatgacgacaagagccttgggagcctgaccatcgcagagccagcaatgattgctgaatgcaaaacgaggacagaagtttttgaaataagcagaagactgatagatagaaccaacgctaattttttggtgtggcccccttgcgtggaggtacaaaggtgctctggatgctgcaataacaggaacgtacaatgcaggcccacccaggtacaactgaggccggtgcaggtcagaaagatcgagatagtcagaaagaaacctatattcaaaaaggccaccgtcacccttgaagaccatttggcgtgcaagtgtgagaccgttgcggcggccagacccgtgact

SEQ ID NO: 24 - DNA, 327 bp, synthetic construct
  source 1..327: mol_type = other DNA
agcctaggcagcttagccgctgcggaacccgccgtcatagccgagtgcaagacccgtactgaggtcttccaaatctcacgtaatttaatcgacaggaccaacgctaacttcttggtatggccgccctgtgttgaagtccaaaggtgcagcggttgttgtaataacagaaacgtacaatgccgtgcgtcccaggtgcaaatgaggcctgttcaggtgagaaaaattgaaatcgtccgtaaaaagcccatatttaagaaagctaccgtcactctggaagaccaccttgcgtgtaagtgtgagaccattgtgacaccgagaccagttacc

SEQ ID NO: 25 - DNA, 351 bp, synthetic construct; identical to SEQ ID NO: 23
  source 1..351: mol_type = other DNA

SEQ ID NO: 26 - DNA, 351 bp, synthetic construct
  source 1..351: mol_type = other DNA
gactacaaagacgatgacgacaagagcctaggcagcttagccgctgcggaacccgccgtcatagccgagtgcaagacccgtactgaggtcttccaaatctcacgtaatttaatcgacaggaccaacgctaacttcttggtatggccgccctgtgttgaagtccaaaggtgcagcggttgttgtaataacagaaacgtacaatgccgtgcgtcccaggtgcaaatgaggcctgttcaggtgagaaaaattgaaatcgtccgtaaaaagcccatatttaagaaagctaccgtcactctggaagaccaccttgcgtgtaagtgtgagaccattgtgacaccgagaccagttacc

SEQ ID NO: 27 - DNA, 159 bp, synthetic construct
  source 1..159: mol_type = other DNA
aacagtgactctgagtgtccgttatctcacgacgggtattgcttacacgacggagtgtgtatgtatatagaggctctggacaagtacgcctgtaattgtgtagtagggtatatcggagagagatgtcaatatagagatttgaagtggtgggaactgaga

SEQ ID NO: 28 - DNA, 183 bp, synthetic construct
  source 1..183: mol_type = other DNA
gactacaaagacgatgacgacaagaacagtgactctgagtgtccgttatctcacgacgggtattgcttacacgacggagtgtgtatgtatatagaggctctggacaagtacgcctgtaattgtgtagtagggtatatcggagagagatgtcaatatagagatttgaagtggtgggaactgaga

SEQ ID NO: 29 - DNA, 159 bp, synthetic construct
  source 1..159: mol_type = other DNA
aactcttatccaggatgtccctcttcctacgatgggtactgcttgaacgggggagtgtgtatgcatattgaatcccttgatagttatacgtgtaattgcgtaatcgggtacagcggtgataggtgtcagacgcgtgatctgaggtggtgggagttgcgc

SEQ ID NO: 30 - DNA, 183 bp, synthetic construct
  source 1..183: mol_type = other DNA
gactacaaagacgatgacgacaagaactcttatccaggatgtccctcttcctacgatgggtactgcttgaacgggggagtgtgtatgcatattgaatcccttgatagttatacgtgtaattgcgtaatcgggtacagcggtgataggtgtcagacgcgtgatctgaggtggtgggagttgcgc

SEQ ID NO: 31 - DNA, 183 bp, synthetic construct; identical to SEQ ID NO: 30
  source 1..183: mol_type = other DNA

SEQ ID NO: 32 - DNA, 462 bp, synthetic construct
  source 1..462: mol_type = other DNA
gactacaaagacgatgacgacaaggtgcctatacaaaaggtacaagatgatactaagacattgattaaaacgattgtcactcgtatcaacgacatttcccatacacaatcagtgtcagcaaagcaaagggtaacgggtcttgatttcattcccggtttgcacccgatattatcattatccaagatggatcagaccttagccgtatatcaacaagttctgacatcccttccgtcccagaatgtactgcaaatcgcaaatgaccttgaaaacctgcgtgaccttctacatctactagcgttctccaaatcttgctctttaccacagacatcagggcttcaaaaaccggagtccttagacggagtccttgaagcatctttgtactctacggaagttgttgccctaagtaggcttcaaggctctttacaagacatcctgcaacagcttgacgtaagcccagagtgc

SEQ ID NO: 33 - DNA, 438 bp, synthetic construct
  source 1..438: mol_type = other DNA
gttcctattcagaaagtccaagacgacacaaagacccttattaaaactattgtaactagaataaatgatataagtcacacacaatcagtatcaagtaagcaaaaagtgacagggttggacttcattcccggccttcacccgatacttacgctatctaagatggaccaaacgcttgctgtctaccagcagatcttgactagtatgccgtcccgtaatgtcatacaaatttccaacgacctggagaatttaagagatttgctgcacgtattagcgttcagtaagagttgccatcttccctgggctagtggcttagaaactcttgattcccttggcggtgtgttagaagcgtccggctacagcacagaagtagtagccctgagtaggcttcagggcagtttacaagacatgctatggcaactggacctatctccgggctgc

SEQ ID NO: 34 - DNA, 435 bp, synthetic construct
  source 1..435: mol_type = other DNA
gttcctattcagaaagtccaagacgacacaaagacccttattaaaactattgtaactagaataaatgatataagtccacaatcagtatcaagtaagcaaaaagtgacagggttggacttcattcccggccttcacccgatacttacgctatctaagatggaccaaacgcttgctgtctaccagcagatcttgactagtatgccgtcccgtaatgtcatacaaatttccaacgacctggagaatttaagagatttgctgcacgtattagcgttcagtaagagttgccatcttccctgggctagtggcttagaaactcttgattcccttggcggtgtgttagaagcgtccggctacagcacagaagtagtagccctgagtaggcttcagggcagtttacaagacatgctatggcaactggacctatctccgggctgc

SEQ ID NO: 35 - DNA, 438 bp, synthetic construct
  source 1..438: mol_type = other DNA
gtgcctatacaaaaggtacaagatgatactaagacattgattaaaacgattgtcactcgtatcaacgacatttcccatacacaatcagtgtcagcaaagcaaagggtaacgggtcttgatttcattcccggtttgcacccgatattatcattatccaagatggatcagaccttagccgtatatcaacaagttctgacatcccttccgtcccagaatgtactgcaaatcgcaaatgaccttgaaaacctgcgtgaccttctacatctactagcgttctccaaatcttgctctttaccacagacatcagggcttcaaaaaccggagtccttagacggagtccttgaagcatctttgtactctacggaagttgttgccctaagtaggcttcaaggctctttacaagacatcctgcaacagcttgacgtaagcccagagtgc

SEQ ID NO: 36 - DNA, 462 bp, synthetic construct; identical to SEQ ID NO: 32
  source 1..462: mol_type = other DNA

SEQ ID NO: 37 - DNA, 462 bp, synthetic construct
  source 1..462: mol_type = other DNA
gactacaaagacgatgacgacaaggttcctattcagaaagtccaagacgacacaaagacccttattaaaactattgtaactagaataaatgatataagtcacacacaatcagtatcaagtaagcaaaaagtgacagggttggacttcattcccggccttcacccgatacttacgctatctaagatggaccaaacgcttgctgtctaccagcagatcttgactagtatgccgtcccgtaatgtcatacaaatttccaacgacctggagaatttaagagatttgctgcacgtattagcgttcagtaagagttgccatcttccctgggctagtggcttagaaactcttgattcccttggcggtgtgttagaagcgtccggctacagcacagaagtagtagccctgagtaggcttcagggcagtttacaagacatgctatggcaactggacctatctccgggctgc

SEQ ID NO: 38 - DNA, 228 bp, synthetic construct
  source 1..228: mol_type = other DNA
gactacaaagacgatgacgacaagaaacctgtatcactatcatacagatgtccctgtaggttcttcgagagtcatattgcacgtgccaatgtaaaacaccttaagatcctaaatacacccaactgtgccctacaaatagtggcacgtttgaagaataataatcgtcaagtctgcatcgaccccaagttgaagtggatacaggagtatcttgagaaggcactgaacaaa

SEQ ID NO: 39 - DNA, 204 bp, synthetic construct
  source 1..204: mol_type = other DNA
aaacctgtatcactatcatacagatgtccctgtaggttcttcgagagtcatattgcacgtgccaatgtaaaacaccttaagatcctaaatacacccaactgtgccctacaaatagtggcacgtttgaagaataataatcgtcaagtctgcatcgaccccaagttgaagtggatacaggagtatcttgagaaggcactgaacaaa

SEQ ID NO: 40 - DNA, 228 bp, synthetic construct
  source 1..228: mol_type = other DNA
gactacaaagacgatgacgacaagaaacctgttagcctatcttaccgttgtccatgtaggttttttgaaagtcacgttgcgagggcgaatgtgaagcacttaaagatcctaaatacaccgaattgtgcactacaaattgtcgcaagactaaagaacaacaataggcaagtatgtattgaccccaaattaaaatggatacaagaatacctagaaaaggcgttgaacaaa

SEQ ID NO: 41 - DNA, 204 bp, synthetic construct
  source 1..204: mol_type = other DNA
aaacctgttagcctatcttaccgttgtccatgtaggttttttgaaagtcacgttgcgagggcgaatgtgaagcacttaaagatcctaaatacaccgaattgtgcactacaaattgtcgcaagactaaagaacaacaataggcaagtatgtattgaccccaaattaaaatggatacaagaatacctagaaaaggcgttgaacaaa

SEQ ID NO: 42 - DNA, 655 bp, synthetic construct
  source 1..655: mol_type = other DNA
agtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggat

SEQ ID NO: 43 - DNA, 688 bp, synthetic construct
  source 1..688: mol_type = other DNA
gggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaact

SEQ ID NO: 44 - DNA, 800 bp, synthetic construct
  source 1..800: mol_type = other DNA
attggatatagaaaattaacgtaaggcagtatcttttcacaatgtacttgcaacgcggcgacttaaagttgaagtacaacctgcagcagcggctttttgtacggtacgccaaactgtcaatggataatattgcgtagaccgaaaaaggtaatcctcaacactacccgtggtggatgacctaaagcagtaatattggttggaattatctcccagacggcaccgtctccccgagaaagcttagccccgaggtctaccttccatacaccactgattgctccacgtcatgcggccttctttcgaggacaaaaaggcatatatcgctaaaattagccatcagaaccgttattgttattatattttcattacgaaagaggagagggcccagcgcgccagagcacacacggtcattgattactttatttggctaaagatccatcccttctcgatgtcatctctttccattcttgtgtatttttgattgaaaatgattttttgtccactaatttctaaaaataagacaaaaagcctttaagcagtttttcatccattttactacggtaaaatgaattagtacggtatggctcccagtcgcattatttttagattggccgtaggggctggggtagaactagagtaaggaacattgctctgccctcttttgaactgtcatataaatacctgacctattttattctccattatcgtattatctcacctctctttttctattctcttgtaattattgatttatagtcgtaactacaaagacaagcaaaataaaatacgttcgctctattaag

SEQ ID NO: 45 - DNA, 1000 bp, synthetic construct
  source 1..1000: mol_type = other DNA
ggccacaatgaaacttcaattcatatcgaccgactatttttctccgaaccaaaaaaatagcagggcgagattggagctgcggaaaaaagaggaaaaaattttttcgtagttttcttgtgcaaattagggtgtaaggtttctagggcttattggttcaagcagaagagacaacaattgtaggtcctaaattcaaggcggatgtaaggagtattggtttcgaaagtttttccgaagcggcatggcagggactacttcgcatgcgctcggattatcttcatttttgcttgcaaaaacgtagaatcatggtaaattacatgaagaattctcttttttttttttttttttttttttttacctctaaagagtgttgaccaactgaaaaaacccttcttcaagagagttaaactaagactaaccatcataacttccaaggaattaatcgatatcttgcactcctgatttttcttcaaagagacagcgcaaaggattatgacactgttgcattgagtcaaaagtttttccgaagtgacccagtgctctttttttttttccgtgaaggactgacaaatatgcgcacaagatccaatacgtaatggaaattcggaaaaactaggaagaaatgctgcagggcattgccgtgccgatcttttgtctttcagatatatgagaaaaagaatattcatcaagtgctgatagaagaataccactcatatgacgtgggcagaagacagcaaacgtaaacatgagctgctgcgacatttgatggcttttatccgacaagccaggaaactccaccattatctaatgtagcaaaatatttcttaacacccgaagttgcgtgtccccctcacgtttttaatcatttgaattagtatattgaaattatatataaaggcaacaatgtccccataatcaattccatctggggtctcatgttctttccccaccttaaaatctataaagatatcataatcgtcaactagttgatatacgtaaaatc

SEQ ID NO: 46 - DNA, 240 bp, synthetic construct
  source 1..240: mol_type = other DNA
atgagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggta

SEQ ID NO: 47 - DNA, 267 bp, synthetic construct
  source 1..267: mol_type = other DNA
atgagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagaggctgaagct

SEQ ID NO: 48 - DNA, 57 bp, synthetic construct
  source 1..57: mol_type = other DNA
atgaaattatcaactgtcctattatctgccggtttagcctcgactactttggcccaa

SEQ ID NO: 49 - protein, 80 aa, synthetic construct
MRFPSIFTAVLFAASSALAAPVNTTTEDETAQIPAEAVIGYLDLEGDFDVAVLPFSNSTNNGLLFINTTIASIAAKEEGV

SEQ ID NO: 50 - protein, 89 aa, synthetic construct
MRFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 51 - protein, 19 aa, synthetic construct
MKLSTVLLSAGLASTTLAQ

SEQ ID NO: 52 - protein, 12 aa, synthetic construct
HRHTKQRHTALH

SEQ ID NO: 53 - protein, 26 aa, synthetic construct
GIGAVLKVLTTGLPALISWIKRKRQQ

SEQ ID NO: 54 - protein, 37 aa, synthetic construct
LLGDFFRKSKEKIGKEFKRIVQRIKDFLRNLVPRTES

SEQ ID NO: 55 - protein, 9 aa, synthetic construct
AKLIPTIAL

SEQ ID NO: 56 - protein, 23 aa, synthetic construct
GIGKFLKKAKKFGKAFVKILKKG

SEQ ID NO: 57 - skipped

SEQ ID NO: 58 - protein, 85 aa, synthetic construct
MRFPSIFTAVLFAASSALAAPVNTTTEDETAQIPAEAVIGYLDLEGDFDVAVLPFSNSTNNGLLFINTTIASIAAKEEGVSLDKR

SEQ ID NO: 59 - DNA, 45 bp, synthetic construct
  source 1..45: mol_type = other DNA
acttcattagacgcctcaataatctgggcgatgatgcagaatgct

SEQ ID NO: 60 - DNA, 30 bp, synthetic construct
  source 1..30: mol_type = other DNA
gctaaattgattccaactattgctttgact

SEQ ID NO: 61 - DNA, 78 bp, synthetic construct
  source 1..78: mol_type = other DNA
gggattggggccgtgttgaaagttcttacgactggtttaccggccctaatctcatggatcaaaaggaagaggcagcag

SEQ ID NO: 62 - DNA, 680 bp, synthetic construct
  source 1..680: mol_type = other DNA
cagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 63 - DNA, 255 bp, synthetic construct
  source 1..255: mol_type = other DNA
atgagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaaga

SEQ ID NO: 64 - DNA, 69 bp, synthetic construct
  source 1..69: mol_type = other DNA
ggtattggaaaatttttgaagaaagctaaaaagtttggtaaggcttttgtaaaaatactgaaaaagggt

SEQ ID NO: 65 - protein, 9 aa, synthetic construct
GGSGGGSGG